(19) United States
(12) Patent Application Publication
(10) Pub. No.: US 2013/0030034 A9
(43) Pub. Date: Jan. 31, 2013

(54) MODULATION OF TIMP1 AND TIMP2 EXPRESSION

(71) Applicants: Yoshiro Niitsu, Sapporo (JP); Hirokazu Takahashi, Sapporo (JP); Yasunobu Tanaka, Sapporo (JP); Elena Feinstein, Rehovot (IL); Sharon Avkin-Nachum, Nes Ziona (IL); Hagar Kalinski, Rishon Le-Zion (IL); Igor Mett, Rehovot (IL)

(72) Inventors: Yoshiro Niitsu, Sapporo (JP); Hirokazu Takahashi, Sapporo (JP); Yasunobu Tanaka, Sapporo (JP); Elena Feinstein, Rehovot (IL); Sharon Avkin-Nachum, Nes Ziona (IL); Hagar Kalinski, Rishon Le-Zion (IL); Igor Mett, Rehovot (IL)

(73) Assignees: Quark Pharmaceuticals, Inc.; Nitto Denko Technical Corporation

(21) Appl. No.: 13/246,621

(22) Filed: Sep. 27, 2011

Related U.S. Application Data
(60) Provisional application No. 61/388,572, filed on Sep. 30, 2010.

(51) Int. Cl. A61K 31/7088; A61P 43/00; C07H 21/02

(52) U.S. Cl. 514/44 A; 536/24.5

(57) ABSTRACT

Provided herein are compositions, methods and kits for modulating expression of target genes, particularly of tissue inhibitor of metalloproteinase 1 and of tissue inhibitor of metalloproteinase 2 (TIMP1 and TIMP2, respectively). The compositions, methods and kits may include nucleic acid molecules (for example, short interfering nucleic acid (siNA), short interfering RNA (siRNA), double-stranded RNA (dsRNA), micro-RNA (miRNA) or short hairpin RNA (shRNA)) that modulate a gene encoding TIMP1 and TIMP2, for example, the gene encoding human TIMP1 and TIMP2. The composition and methods disclosed herein may also be used in treating conditions and disorders associated with TIMP1 and TIMP2 including fibrotic diseases and disorders including liver fibrosis, pulmonary fibrosis, peritoneal fibrosis and kidney fibrosis.

### RELATED PATENT APPLICATIONS

This application claims the benefit of U.S. Provisional Application Ser. No. 61/388,572 filed Sep. 30, 2010 entitled “Modulation of TIMP1 and TIMP2 Expression” and which is incorporated herein by reference in its entirety and for all purposes.

### SEQUENCE LISTING

The instant application contains a Sequence Listing which is entitled 224-PCT1_ST25.txt, said ASCII copy, created on Aug. 24, 2011 and is 910 kb in size, is hereby incorporated by reference in its entirety.

### FIELD OF THE INVENTION

Provided herein are compositions and methods for modulating expression of TIMP1 and TIMP2.

### BACKGROUND OF THE INVENTION

Sato, Y., et al. disclose the administration of vitamin A-coupled liposomes to deliver small interfering RNA (siRNA) against gp46, the rat homolog of human heat shock protein 47, to liver cirrhosis rat animal models. Sato, Y., et al., Nature Biotechnology, vol. 26(4), p. 431-442 (2008).

Chen, J-J., et al. disclose transfecting human keloid samples with HSP-47-shRNA (small hairpin RNA) to examine proliferation of keloid fibroblast cells. Chen, J-J., et al., British Journal of Dermatology, vol. 156, p. 1188-1195 (2007).

PCT Patent Publication No. WO 2006/068232 discloses an astrocyte specific drug carrier which includes a retinoid derivative and/or a vitamin A analog.

PCT Patent Publication Nos. WO 2008/104978 and WO 2007/091269 disclose siRNA structures and compounds.

PCT Patent Publication No. WO 2011/072082 discloses double stranded RNA compounds targeting HSP47 (SERPINH1).

### SUMMARY OF THE INVENTION

Compositions, methods and kits for modulating expression of target genes are provided herein. In various aspects and embodiments, compositions, methods and kits provided herein modulate expression of tissue inhibitor of metalloproteinases 1 and tissue inhibitor of metalloproteinases 2 also known as TIMP1 and TIMP2, respectively. The compositions, methods and kits may involve use of nucleic acid molecules (for example, short interfering nucleic acid (siNA), short interfering RNA (siRNA), double-stranded RNA (dsRNA), micro-RNA (miRNA) or short hairpin RNA (shRNA)) that bind a nucleotide sequence (such as an mRNA sequence) encoding TIMP1 and TIMP2, for example, the mRNA coding sequence for human TIMP1 exemplified by SEQ ID NO:1 and the mRNA coding sequence for human TIMP2 exemplified by SEQ ID NO:2. In certain preferred embodiments, the compositions, methods and kits disclosed herein inhibit expression of TIMP1 or TIMP2. For example, siNA molecules (e.g., RISC length dsNA molecules or Dicer length dsNA molecules) are provided that down regulate, reduce or inhibit TIMP1 or TIMP2 expression. Also provided are compositions, methods and kits for treating and/or preventing diseases, conditions or disorders associated with TIMP1 and TIMP2, including organ specific fibrosis associated with at least one of brain, skin fibrosis, lung fibrosis, liver fibrosis, kidney fibrosis, heart fibrosis, vascular fibrosis, bone marrow fibrosis, eye fibrosis, intestinal fibrosis, vocal cord fibrosis or other fibrosis. Specific indications include liver fibrosis, cirrhosis, pulmonary fibrosis including Interstitial lung fibrosis (ILF), kidney fibrosis resulting from any condition (e.g., CKD including ESRD), peritoneal fibrosis, chronic hepatic damage, fibrillogenesis, fibrotic diseases in other organs, abnormal scarring (keloids) associated with all possible types of skin injury accidental and jatrogenic (operations); scleroderma; cardiofibrosis, failure of glaucoma filtering operation; brain fibrosis associated with cerebral infarction; and intestinal adhesions and Crohn's disease. The compounds are useful in treating organ specific indications including those shown in Table I infra.

In one aspect, provided are nucleic acid molecules (e.g., siNA molecules) in which (a) the nucleic acid molecule includes a sense strand (passenger strand) and an antisense strand (guide strand); (b) each strand of the nucleic acid molecule is independently 15 to 49 nucleotides in length; (c) a 15 to 49 nucleotide sequence of the antisense strand is complementary to a sequence of an mRNA encoding a human TIMP (e.g., SEQ ID NO: 1 or SEQ ID NO:2); and (d) a 15 to 49 nucleotide sequence of the sense strand is complementary to the sequence of the antisense strand and includes a 15 to 49 nucleotide sequence of an mRNA encoding human TIMP1 or TIMP2 (e.g., SEQ ID NO: 1 or SEQ ID NO:2, respectively). In various embodiments the sense and antisense strands generate a 15 to 49 base pair duplex.

In certain embodiments, the sequence of the antisense strand that is complementary to a sequence of an mRNA encoding human TIMP1 includes a sequence complimentary to a sequence between nucleotides 193-813 or 1-192; or 813-893 of SEQ ID NO: 1; or between nucleotides 1-200; or 800-893 of SEQ ID NO: 1.

In certain embodiments the sequence of the antisense comprises an antisense sequence set forth in any one of Tables A1-A8 or C. In preferred embodiments the sequence of the antisense comprises an antisense sequence set forth in Tables A3, A4, A7, A8, or C. In some embodiments the antisense and sense strands are selected from the sequence pairs set forth in Table A3 or Table A4. In some embodiments the antisense and sense strands are selected from the sequence pairs set forth in Table A7 or Table A8. In some embodiments the antisense and sense strands are selected from the sequence pairs set forth in Table C.

In certain embodiments, the sequence of the antisense strand that is complementary to a sequence of an mRNA encoding human TIMP2 includes a sequence complimentary to a sequence between nucleotides 303-962 or 1-303; or 962-3369; of SEQ ID NO: 2; or between nucleotides 1-350; or 950-3369 of SEQ ID NO: 2.

In certain embodiments the sequence of the antisense comprises an antisense sequence set forth in any one of Tables B1-B8 or D. In preferred embodiments the sequence of the antisense comprises an antisense sequence set forth in Tables B3, B4, B7, B8, D. In some embodiments the antisense and sense strands are selected from the sequence pairs set forth in Table B3 or Table B4. In some embodiments the antisense and sense strands are selected from the sequence pairs set forth in Table B7 or Table B8. In some embodiments the antisense and sense strands are selected from the sequence pairs set forth in Table D.

In some embodiments, the antisense strand includes a sequence that is complementary to a sequence of an mRNA encoding human TIMP1 corresponding to nucleotides 355-373 of SEQ ID NO: 1 or a portion thereof; or nucleotides 620-638 of SEQ ID NO: 1 or a portion thereof; or nucleotides 640-658 of SEQ ID NO: 1 or a portion thereof.

In some embodiments, the antisense strand includes a sequence that is complementary to a sequence of an mRNA encoding human TIMP2 corresponding to nucleotides 421-439 of SEQ ID NO: 2 or a portion thereof; or nucleotides 502-520 of SEQ ID NO: 2 or a portion thereof; or nucleotides 523-541 of SEQ ID NO: 2 or a portion thereof; or nucleotides 625-643 of SEQ ID NO: 2 or a portion thereof; or nucleotides 629-647 of SEQ ID NO: 2 or a portion thereof.

In some embodiments, the antisense strand of a nucleic acid molecule (e.g., a siNA molecule) as disclosed herein includes a sequence corresponding to any one of the antisense sequences shown in Table A1 or A5. In certain preferred embodiments the antisense strand and the strand are selected from the sequence pairs shown in Table A1. In certain preferred embodiments the antisense strand and the sense strand are selected from the sequence pairs shown in Table A5. In some preferred embodiments the antisense and sense strands are selected from the sequence pairs shown in Table A3 or Table A7.

In certain embodiments, the antisense strand of a nucleic acid molecule (e.g., a siNA molecule) as disclosed herein includes a sequence corresponding to any one of the antisense sequences shown in Table C.

In various embodiments of nucleic acid molecules (e.g., siNA molecules) as disclosed herein, the antisense strand may be 15 to 49 nucleotides in length (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 nucleotides in length); or 17-35 nucleotides in length; or 17-30 nucleotides in length; or 15-25 nucleotides in length; or 18-25 nucleotides in length; or 18-23 nucleotides in length; or 19-21 nucleotides in length; or 25-30 nucleotides in length; or 26-28 nucleotides in length. Similarly the sense strand of nucleic acid molecules (e.g., siNA molecules) as disclosed herein may be 15 to 49 nucleotides in length (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 nucleotides in length); or 17-35 nucleotides in length; or 17-30 nucleotides in length; or 15-25 nucleotides in length; or 18-25 nucleotides in length; or 18-23 nucleotides in length; or 19-21 nucleotides in length; or 25-30 nucleotides in length; or 26-28 nucleotides in length. The duplex region of the nucleic acid molecules (e.g., siNA molecules) as disclosed herein may be 15-49 nucleotides in length (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 nucleotides in length); 18-40 nucleotides in length; or 15-35 nucleotides in length; or 15-30 nucleotides in length; or about 15-25 nucleotides in length; or 17-25 nucleotides in length; or 17-23 nucleotides in length; or 17-21 nucleotides in length; or 19-21 nucleotides in length, or 25-30 nucleotides in length; or 25-28 nucleotides in length. In some embodiments the duplex region of the nucleic acid molecules (e.g., siNA molecules) is 19 nucleotides in length.

In certain embodiments, the sense and antisense strands of a nucleic acid (e.g., an siNA nucleic acid molecule) as provided herein are separate polynucleotide strands. In some embodiments, the separate antisense and sense strands form a double stranded structure via hydrogen bonding, for example, Watson-Crick base pairing. In some embodiments the sense and antisense strands are two separate strands that are covalently linked to each other. In other embodiments, the sense and antisense strands are part of a single polynucleotide strand having both a sense and antisense region; in some preferred embodiments the polynucleotide strand has a hairpin structure.

In certain embodiments, the nucleic acid molecule (e.g., siNA molecule) is a double stranded nucleic acid (dsNA) molecule that is symmetrical with regard to overhangs, and has a blunt end on both ends. In other embodiments the nucleic acid molecule (e.g., siNA molecule) is a dsNA molecule that is symmetrical with regard to overhangs, and has an overhang on both ends of the dsNA molecule; preferably the molecule has overhangs of 1, 2, 3, 4, 5, 6, 7, or 8 nucleotides; preferably the molecule has 2 nucleotide overhangs. In some embodiments the overhangs are 5′ overhangs; in alternative embodiments the overhangs are 3′ overhangs. In certain embodiments, the overhang nucleotides are modified with modifications as disclosed herein. In some embodiments the overhang nucleotides are 2′-deoxyribonucleotides.

In some embodiments the molecules comprise non-nucleotide overhangs at one or more of the 5′ or 3′ terminus of the sense and/or antisense strands. Such non-nucleotide overhangs include abasic ribo- and deoxyribo-nucleotide moieties, alkyl moieties including C3-C3 moieties and amino carbon chains.

In certain preferred embodiments, the nucleic acid molecule (e.g., siNA molecule) is a dsNA molecule that is asymmetrical with regard to overhangs, and has a blunt end on one end of the molecule and an overhang on the other end of the molecule. In certain embodiments the overhang is 1, 2, 3, 4, 5, 6, 7, or 8 nucleotides; preferably the overhang is 2 nucleotides. In some preferred embodiments an asymmetrical dsNA molecule has a 3′-overhang (for example a two nucleotide 3′-overhang) on one side of a duplex occurring on the sense strand; and a blunt end on the other side of the molecule. In some preferred embodiments an asymmetrical dsNA molecule has a 5′-overhang (for example a two nucleotide 5′-overhang) on one side of a duplex occurring on the sense strand; and a blunt end on the other side of the molecule. In other preferred embodiments an asymmetrical dsNA molecule has a 3′-overhang (for example a two nucleotide 3′-overhang) on one side of a duplex occurring on the antisense strand; and a blunt end on the other side of the molecule. In some preferred embodiments an asymmetrical dsNA molecule has a 5′-overhang (for example a two nucleotide 5′-overhang) on one side of a duplex occurring on the antisense strand; and a blunt end on the other side of the molecule. In certain preferred embodiments, the overhangs are 2′-deoxyribonucleotides. Examples of siNA compounds having a terminal dTdT are found in Tables C and D, infra.

In some embodiments, the nucleic acid molecule (e.g., siNA molecule) has a hairpin structure (having the sense strand and antisense strand on one polynucleotide), with a loop structure on one end and a blunt end on the other end. In some embodiments, the nucleic acid molecule has a hairpin structure, with a loop structure on one end and an overhang end on the other end (for example a 1, 2, 3, 4, 5, 6, 7, or 8 nucleotide overhang); in certain embodiments, the overhang is a 3′-overhang; in certain embodiments the overhang is a 5′-overhang; in certain embodiments the overhang is on the sense strand; in certain embodiments the overhang is on the antisense strand.

The nucleic acid molecules (e.g., siNA molecule) disclosed herein may include one or more modifications or modified nucleotides such as described herein. For example, a nucleic acid molecule (e.g., siNA molecule) as provided herein may include a modified nucleotide having a modified sugar; a modified nucleotide having a modified nucleobase; or a modified nucleotide having a modified phosphate group. Similarly, a nucleic acid molecule (e.g., siNA molecule) as provided herein may include a modified phosphodiester backbone and/or may include a modified terminal phosphate group.

Nucleic acid molecules (e.g., siNA molecules) as provided may have one or more nucleotides that include a modified sugar moiety, for example as described herein. In some preferred embodiments the modified sugar moiety is selected from the group consisting of 2′-O-methyl, 2′-methoxyethoxy, 2′-deoxy, 2′-fluoro, 2′-allyl, 2′-O-[2-(methylamino)-2-oxoethyl], 4′-thio, 4′-(CH₂)₂—O-2′-bridge, 2′-locked nucleic acid, and 2′-O—(N-methylcarbamate).

Nucleic acid molecules (e.g., siNA molecules) as provided may have one or more modified nucleobase(s) for example as described herein, which preferably may be one selected from the group consisting of xanthine, hypoxanthine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 8-halo, amino, thiol, thioalkyl, hydroxyl and other 8-substituted adenines and guanines, 5-trifluoromethyl and other 5-substituted uracils and cytosines, 7-methylguanine, and acyclonucleotides.

Nucleic acid molecules (e.g., siNA molecules) as provided may have one or more modifications to the phosphodiester backbone, for example as described herein. In some preferred embodiments the phosphodiester bond is modified by substituting the phosphodiester bond with a phosphorothioate, 3′-(or -5′)deoxy-3′-(or -5′)thio-phosphorothioate, phosphorodithioate, phosphoroselenates, 3′-(or -5′)deoxy phosphinates, borano phosphates, 3′-(or -5′)deoxy-3′-(or 5′-)amino phosphoramidates, hydrogen phosphonates, borano phosphate esters, phosphoramidates, alkyl or aryl phosphonates and phosphotriester or phosphorus linkages.

In various embodiments, the provided nucleic acid molecules (e.g., siNA molecules) may include one or modifications in the sense strand but not the antisense strand; in other embodiments the provided nucleic acid molecules (e.g., siNA molecules) include one or more modifications in the antisense strand but not the sense strand; in yet other embodiments, the provided nucleic acid molecules (e.g., siNA molecules) include one or more modifications in the both the sense strand and the antisense strand.

In some embodiments in which the provided nucleic acid molecules (e.g., siNA molecules) have modifications, the sense strand includes a pattern of alternating modified and unmodified nucleotides, and/or the antisense strand includes a pattern of alternating modified and unmodified nucleotides; in some preferred versions of such embodiments the modification is a 2′-O-methyl (2′ methoxy or 2′OMe) sugar moiety. The pattern of alternating modified and unmodified nucleotides may start with a modified nucleotide at the 5′ end or 3′ end of one of the strands; for example the pattern of alternating modified and unmodified nucleotides may start with a modified nucleotide at the 5′ end or 3′ end of the sense strand and/or the pattern of alternating modified and unmodified nucleotides may start with a modified nucleotide at the 5′ end or 3′ end of the antisense strand. When both the antisense and sense strand include a pattern of alternating modified nucleotides, the pattern of modified nucleotides may be configured such that modified nucleotides in the sense strand are opposite modified nucleotides in the antisense strand; or there may be a phase shift in the pattern such that modified nucleotides of the sense strand are opposite unmodified nucleotides in the antisense strand and vice-versa.

The nucleic acid molecules (e.g., siNA molecules) as provided herein may include 1-3 (i.e., 1, 2 or 3) deoxyribonucleotides at the 3′ end of the sense and/or the antisense strand.

The nucleic acid molecules (e.g., siNA molecules) as provided herein may include a phosphate group at the 5′ end of the sense and/or the antisense strand.

In one aspect, provided are double stranded nucleic acid molecules having the structure (A1):

(A1) 5′(N)x - Z 3′ (antisense strand)
3′ Z′-(N′)y -z″ 5′ (sense strand)

wherein each of N and N′ is a nucleotide which may be unmodified or modified, or an unconventional moiety;
wherein each of (N)x and (N′)y is an oligonucleotide in which each consecutive N or N′ is joined to the next N or N′ by a covalent bond; wherein each of Z and Z′ is independently present or absent, but if present independently includes 1-5 consecutive nucleotides or non-nucleotide moieties or a combination thereof covalently attached at the 3′ terminus of the strand in which it is present;
wherein z″ may be present or absent, but if present is a capping moiety covalently attached at the 5′ terminus of (N′)y;
each of x and y is independently an integer from 18 to 40;
wherein the sequence of (N′)y has complementarity to the sequence of (N)x; and wherein (N)x includes an antisense sequence to SEQ ID NO:1 or to SEQ ID NO:2.

In some embodiments (N)x includes an antisense sequence to SEQ ID NO:1. In some embodiments (N)x includes an antisense oligonucleotide present in any one of Tables A1, A2, A3 or A4. In other embodiments (N)x is selected from an antisense oligonucleotide present in Tables A3 or A4.

In certain preferred embodiments, the antisense strand of a nucleic acid molecule (e.g., a siNA molecule) as disclosed herein includes a sequence corresponding to any one of the antisense sequences shown on Table A1. In certain preferred embodiments the antisense strand and the strand are selected from the sequence pairs shown in Table A2. In certain preferred embodiments the antisense strand and the strand are active in more than one species (human and at least one other species) and are selected from the sequence pairs shown in Table A2. In certain preferred embodiments the antisense strand and the strand are selected from the sequence pairs shown in Table A3, and preferably in Table A4. In some embodiments the antisense and sense strands are selected from the sequence pairs set forth in duplexes siTIMP1_p2; siTIMP1_p6; siTIMP1_p14; siTIMP1_p16; siTIMP1_p17; siTIMP1_p19; siTIMP1_p20; siTIMP1_p21; siTIMP1_p23; siTIMP1_p24; siTIMP1_p27; siTIMP1_p29; siTIMP1_p31; siTIMP1_p33; siTIMP1_p38; siTIMP1_p42; siTIMP1_p43; siTIMP1_p45; siTIMP1_p49; siTIMP1_p60; siTIMP1_p71; siTIMP1_p73; siTIMP1_p77; siTIMP1_p78; siTIMP1_p79; siTIMP1_p85; siTIMP1_p89; siTIMP1_p91; siTIMP1_p96; siTIMP1_p98; siTIMP1_p99 and siTIMP1_p108, shown in Table A3 infra.

In some embodiments the antisense and sense strands are selected from the sequence pairs set forth in siTIMP1_p2 (SEQ ID NOS:267 and 299); siTIMP1_p6 (SEQ ID NOS:268 and 300); siTIMP1_p14 (SEQ ID NOS:269 and 301); siTIMP1_p16 (SEQ ID NOS:270 and 302); siTIMP1_p17 (SEQ ID NOS:271 and 303); siTIMP1_p19 (SEQ ID NOS:272 and 304); siTIMP1_p20 (SEQ ID NOS:273 and 305); siTIMP1_p21 (SEQ ID NOS:274 and 306); siTIMP1_p23 (SEQ ID NOS:275 and 307; siTIMP1_p29 (278 and 310); siTIMP1_p33 (280 and 312); siTIMP1_p38 (SEQ ID NOS:281 and 313); siTIMP1_p42 (282 and 314); siTIMP1_p43 (SEQ ID NOS:283 and 315); siTIMP1_p45 (284 and 316); siTIMP1_p60 (SEQ ID NOS:286 and 318); siTIMP1_p71 (SEQ ID NOS:287 and 319); siTIMP1_p73 (SEQ ID NOS:288 and 320); siTIMP1_p78 (290 and 322); siTIMP1_p79 (SEQ ID NOS:291 and 323); siTIMP1_p85 (SEQ ID NOS:292 and 324); siTIMP1_p89 (SEQ ID NOS:293 and 325); siTIMP1_p91 (SEQ ID NOS:294 and 326); siTIMP1_p96 (SEQ ID NOS:295 and 327); siTIMP1_p98 (SEQ ID NOS:296 and 328); siTIMP1_p99 (SEQ ID NOS:297 and 329) and siTIMP1_p108 (SEQ ID NOS:298 and 330), shown in Table A4, infra.

In some embodiments the antisense and sense strands of a nucleic acid molecule (e.g., a siNA molecule) as disclosed herein includes the sequence pairs set forth in siTIMP1_p2 (SEQ ID NOS:267 and 299). In some embodiments the antisense and sense strands of a nucleic acid molecule (e.g., a siNA molecule) as disclosed herein includes the sequence pairs set forth in siTIMP1_p6 (SEQ ID NOS:268 and 300). In some embodiments the antisense and sense strands of a nucleic acid molecule (e.g., a siNA molecule) as disclosed herein includes the sequence pairs set forth in siTIMP1_p14 (SEQ ID NOS:269 and 301). In some embodiments the antisense and sense strands of a nucleic acid molecule (e.g., a siNA molecule) as disclosed herein includes the sequence pairs set forth in siTIMP1_p16 (SEQ ID NOS:270 and 302). In some embodiments the antisense and sense strands of a nucleic acid molecule (e.g., a siNA molecule) as disclosed herein includes the sequence pairs set forth in siTIMP1_p17 (SEQ ID NOS:271 and 303). In some embodiments the antisense and sense strands of a nucleic acid molecule (e.g., a siNA molecule) as disclosed herein includes the sequence pairs set forth in siTIMP1_p19 (SEQ ID NOS:272 and 304). In some embodiments the antisense and sense strands of a nucleic acid molecule (e.g., a siNA molecule) as disclosed herein includes the sequence pairs set forth in siTIMP1_p20 (SEQ ID NOS:273 and 305). In some embodiments the antisense and sense strands of a nucleic acid molecule (e.g., a siNA molecule) as disclosed herein includes the sequence pairs set forth in siTIMP1_p21 (SEQ ID NOS:274 and 306). In some embodiments the antisense and sense strands of a nucleic acid molecule (e.g., a siNA molecule) as disclosed herein includes the sequence pairs set forth in siTIMP1_p23 (SEQ ID NOS:275 and 307. In some embodiments the antisense and sense strands of a nucleic acid molecule (e.g., a siNA molecule) as disclosed herein includes the sequence pairs set forth in siTIMP1_p29 (278 and 310). In some embodiments the antisense and sense strands of a nucleic acid molecule (e.g., a siNA molecule) as disclosed herein includes the sequence pairs set forth in siTIMP1_p33 (280 and 312). In some embodiments the antisense and sense strands of a nucleic acid molecule (e.g., a siNA molecule) as disclosed herein includes the sequence pairs set forth in siTIMP1_p38 (SEQ ID NOS:281 and 313). In some embodiments the antisense and sense strands of a nucleic acid molecule (e.g., a siNA molecule) as disclosed herein includes the sequence pairs set forth in siTIMP1_p42 (282 and 314). In some embodiments the antisense and sense strands of a nucleic acid molecule (e.g., a siNA molecule) as disclosed herein includes the sequence pairs set forth in siTIMP1_p43 (SEQ ID NOS:283 and 315). In some embodiments the antisense and sense strands of a nucleic acid molecule (e.g., a siNA molecule) as disclosed herein includes the sequence pairs set forth in siTIMP1_p45 (284 and 316). In some embodiments the antisense and sense strands of a nucleic acid molecule (e.g., a siNA molecule) as disclosed herein includes the sequence pairs set forth in siTIMP1_p60 (SEQ ID NOS:286 and 318). In some embodiments the antisense and sense strands of a nucleic acid molecule (e.g., a siNA molecule) as disclosed herein includes the sequence pairs set forth in siTIMP1_p71 (SEQ ID NOS:287 and 319). In some embodiments the antisense and sense strands of a nucleic acid molecule (e.g., a siNA molecule) as disclosed herein includes the sequence pairs set forth in siTIMP1_p73 (SEQ ID NOS:288 and 320). In some embodiments the antisense and sense strands of a nucleic acid molecule (e.g., a siNA molecule) as disclosed herein includes the sequence pairs set forth in siTIMP1_p78 (290 and 322). In some embodiments the antisense and sense strands of a nucleic acid molecule (e.g., a siNA molecule) as disclosed herein includes the sequence pairs set forth in siTIMP1_p79 (SEQ ID NOS:291 and 323). In some embodiments the antisense and sense strands of a nucleic acid molecule (e.g., a siNA molecule) as disclosed herein includes the sequence pairs set forth in siTIMP1_p85 (SEQ ID NOS:292 and 324). In some embodiments the antisense and sense strands of a nucleic acid molecule (e.g., a siNA molecule) as disclosed herein includes the sequence pairs set forth in siTIMP1_p89 (SEQ ID NOS:293 and 325). In some embodiments the antisense and sense strands of a nucleic acid molecule (e.g., a siNA molecule) as disclosed herein includes the sequence pairs set forth in siTIMP1_p91 (SEQ ID NOS:294 and 326). In some embodiments the antisense and sense strands of a nucleic acid molecule (e.g., a siNA molecule) as disclosed herein includes the sequence pairs set forth in siTIMP1_p96 (SEQ ID NOS:295 and 327). In some embodiments the antisense and sense strands of a nucleic acid molecule (e.g., a siNA molecule) as disclosed herein includes the sequence pairs set forth in siTIMP1_p98 (SEQ ID NOS:296 and 328). In some embodiments the antisense and sense strands of a nucleic acid molecule (e.g., a siNA molecule) as disclosed herein includes the sequence pairs set forth in siTIMP1_p99 (SEQ ID NOS:297 and 329). In some embodiments the antisense and sense strands of a nucleic acid molecule (e.g., a siNA molecule) as disclosed herein includes the sequence pairs set forth in siTIMP1_p108 (SEQ ID NOS:298 and 330), shown in Table A4.

In some preferred embodiments the nucleic acid molecule (e.g., a siNA molecule) as disclosed herein includes the antisense strand and the sense strand of a sequence pair set forth in siTIMP1_p2 (SEQ ID NOS:267 and 299). In some preferred embodiments the nucleic acid molecule (e.g., a siNA molecule) as disclosed herein includes the antisense strand and the sense strand of a sequence pair set forth in siTIMP1_p6 (SEQ ID NOS:268 and 300). In some preferred embodiments the nucleic acid molecule (e.g., a siNA molecule) as disclosed herein includes the antisense strand and the sense strand of a sequence pair set forth in siTIMP1_p16 (SEQ ID NOS:270 and 302). In some preferred embodiments the nucleic acid molecule (e.g., a siNA molecule) as disclosed herein includes the antisense strand and the sense strand of a sequence pair set forth in siTIMP1_p17 (SEQ ID NOS:271 and 303). In some preferred embodiments the nucleic acid molecule (e.g., a siNA molecule) as disclosed herein includes the antisense strand and the sense strand of a sequence pair set forth in siTIMP1_p19 (SEQ ID NOS:272 and 304). I In some preferred embodiments the nucleic acid molecule (e.g., a siNA molecule) as disclosed herein includes the antisense strand and the sense strand of a sequence pair set forth in siTIMP1_p20 (SEQ ID NOS:273 and 305). In some preferred embodiments the nucleic acid molecule (e.g., a siNA molecule) as disclosed herein includes the antisense strand and the sense strand of a sequence pair set forth in siTIMP1_p21 (SEQ ID NOS:274 and 306). In some preferred embodiments the nucleic acid molecule (e.g., a siNA molecule) as disclosed herein includes the antisense strand and the sense strand of a sequence pair set forth in siTIMP1_p38 (SEQ ID NOS:281 and 313).

In some embodiments (N)x includes an antisense sequence to SEQ ID NO:2. In some embodiments (N)x includes an antisense oligonucleotide present in any one of Tables B1, B2, B3 or B4. In other embodiments (N)x is selected from an antisense oligonucleotide present in Tables B3 or B4.

In certain preferred embodiments, the antisense strand of a nucleic acid molecule (e.g., a siNA molecule) as disclosed herein includes a sequence corresponding to any one of the antisense sequences shown on Table B1. In certain preferred embodiments the antisense strand and the strand are selected from the sequence pairs shown in Table B2. In certain preferred embodiments the antisense strand and the strand are active in more than one species (human and at least one other species) and are selected from the sequence pairs shown in Table B2. In certain preferred embodiments the antisense strand and the strand are selected from the sequence pairs shown in Table B3, and preferably in Table B4.

In some embodiments the antisense and sense strands are selected from the sequence pairs set forth in siTIMP2_p4; siTIMP2_p16; siTIMP2_p17; siTIMP2_p18; siTIMP2_p20; siTIMP2_p24; siTIMP2_p25; siTIMP2_p27; siTIMP2_p29; siTIMP2_p30; siTIMP2_p33; siTIMP2_p35; siTIMP2_p37; siTIMP2_p38; siTIMP2_p39; siTIMP2_p40; siTIMP2_p41; siTIMP2_p44; siTIMP2_p46; siTIMP2_p51; siTIMP2_p55; siTIMP2_p61; siTIMP2_p62; siTIMP2_p64; siTIMP2_p65; siTIMP2_p67; siTIMP2_p68; siTIMP2_p69; siTIMP2_p71; siTIMP2_p75; siTIMP2_p76; siTIMP2_p78; siTIMP2_p79; siTIMP2_p82; siTIMP2_p83; siTIMP2_p84; siTIMP2_p85; siTIMP2_p86; siTIMP2_p87; siTIMP2_p88; siTIMP2_p89; siTIMP2_p90; siTIMP2_p91; siTIMP2_p92; siTIMP2_p93; siTIMP2_p94; siTIMP2_p95; siTIMP2_p96; siTIMP2_p97; siTIMP2_p98; siTIMP2_p99; siTIMP2_p100; and siTIMP2_p101 and siTIMP2_p102, shown in Table B3, infra.

In some embodiments the antisense and sense strands are selected from the sequence pairs set forth in siTIMP2_p27 (SEQ ID NOS:2478 and 2531); siTIMP2_p29 (SEQ ID NOS:2479 and 2532); siTIMP2_p30 (SEQ ID NOS:2480 and 2533); siTIMP2_p39 (SEQ ID NOS:2485 and 2538); siTIMP2_p40 (SEQ ID NOS:2486 and 2539); siTIMP2_p41 (SEQ ID NOS:2487 and 2540); siTIMP2_p46 (SEQ ID NOS:2489 and 2542); siTIMP2_p55 (SEQ ID NOS:2491 and 2544); siTIMP2_p62 (SEQ ID NOS:2493 and 2546); siTIMP2_p68 (SEQ ID NOS:2497 and 2550); siTIMP2_p69 (SEQ ID NOS:2498 and 2551); siTIMP2_p71 (SEQ ID NOS:2499 and 2552); siTIMP2_p76 (SEQ ID NOS:2501 and 2554); siTIMP2_p78 (SEQ ID NOS:2502 and 2555); siTIMP2_p89 (SEQ ID NOS:2511 and 2564); siTIMP2_p91 (SEQ ID NOS:2513 and 2566); siTIMP2_p93 (SEQ ID NOS:2515 and 2568); siTIMP2_p95 (SEQ ID NOS:2517 and 2570); siTIMP2_p97 (SEQ ID NOS:2519 and 2572); siTIMP2_p98 (SEQ ID NOS:2520 and 2573); and siTIMP2_p100 (SEQ ID NOS:2522 and 2575), shown in Table B4, infra

In some embodiments the antisense and sense strands of a nucleic acid molecule (e.g., a siNA molecule) as disclosed herein includes the sequence pairs set forth in siTIMP2_p27 (SEQ ID NOS:2478 and 2531). In some embodiments the antisense and sense strands of a nucleic acid molecule (e.g., a siNA molecule) as disclosed herein includes the sequence pairs set forth in siTIMP2_p29 (SEQ ID NOS:2479 and 2532). In some embodiments the antisense and sense strands of a nucleic acid molecule (e.g., a siNA molecule) as disclosed herein includes the sequence pairs set forth in siTIMP2_p30 (SEQ ID NOS:2480 and 2533). In some embodiments the antisense and sense strands of a nucleic acid molecule (e.g., a siNA molecule) as disclosed herein includes the sequence pairs set forth in siTIMP2_p39 (SEQ ID NOS:2485 and 2538). In some embodiments the antisense and sense strands of a nucleic acid molecule (e.g., a siNA molecule) as disclosed herein includes the sequence pairs set forth in siTIMP2_p40 (SEQ ID NOS:2486 and 2539). In some embodiments the antisense and sense strands of a nucleic acid molecule (e.g., a siNA molecule) as disclosed herein includes the sequence pairs set forth in siTIMP2_p41 (SEQ ID NOS:2487 and 2540). In some embodiments the antisense and sense strands of a nucleic acid molecule (e.g., a siNA molecule) as disclosed herein includes the sequence pairs set forth in siTIMP2_p46 (SEQ ID NOS:2489 and 2542). In some embodiments the antisense and sense strands of a nucleic acid molecule (e.g., a siNA molecule) as disclosed herein includes the sequence pairs set forth in siTIMP2_p55 (SEQ ID NOS:2491 and 2544). In some embodiments the antisense and sense strands of a nucleic acid molecule (e.g., a siNA molecule) as disclosed herein includes the sequence pairs set forth in siTIMP2_p62 (SEQ ID NOS:2493 and 2546). In some embodiments the antisense and sense strands of a nucleic acid molecule (e.g., a siNA molecule) as disclosed herein includes the sequence pairs set forth in siTIMP2_p68 (SEQ ID NOS:2497 and 2550). In some embodiments the antisense and sense strands of a nucleic acid molecule (e.g., a siNA molecule) as disclosed herein includes the sequence pairs set forth in siTIMP2_p69 (SEQ ID NOS:2498 and 2551). In some embodiments the antisense and sense strands of a nucleic acid molecule (e.g., a siNA molecule) as disclosed herein includes the sequence pairs set forth in siTIMP2_p71 (SEQ ID NOS:2499 and 2552). In some embodiments the antisense and sense strands of a nucleic acid molecule (e.g., a siNA molecule) as disclosed herein includes the sequence pairs set forth in siTIMP2_p76 (SEQ ID NOS:2501 and 2554). In some embodiments the antisense and sense strands of a nucleic acid molecule (e.g., a siNA molecule) as disclosed herein includes the sequence pairs set forth in siTIMP2_p78 (SEQ ID NOS:2502 and 2555). In some embodiments the antisense and sense strands of a nucleic acid molecule (e.g., a siNA molecule) as disclosed herein includes the sequence pairs set forth in siTIMP2_p89 (SEQ ID NOS:2511 and 2564). In some embodiments the antisense and sense strands of a nucleic acid molecule (e.g., a siNA molecule) as disclosed herein includes the sequence pairs set forth in siTIMP2_p91 (SEQ ID NOS:2513 and 2566). In some embodiments the antisense and sense strands of a nucleic acid molecule (e.g., a siNA molecule) as disclosed herein includes the sequence pairs set forth in siTIMP2_p93 (SEQ ID NOS:2515 and 2568). In some embodiments the antisense and sense strands of a nucleic acid molecule (e.g., a siNA molecule) as disclosed herein includes the sequence pairs set forth in siTIMP2_p95 (SEQ ID NOS:2517 and 2570). In some embodiments the antisense and sense strands of a nucleic acid molecule (e.g., a siNA molecule) as disclosed herein includes the sequence pairs set forth in siTIMP2_p97 (SEQ ID NOS:2519 and 2572). In some embodiments the antisense and sense strands of a nucleic acid molecule (e.g., a siNA molecule) as disclosed herein includes the sequence pairs set forth in siTIMP2_p98 (SEQ ID NOS:2520 and 2573). In some embodiments the antisense and sense strands of a nucleic acid molecule (e.g., a siNA molecule) as disclosed herein includes the sequence pairs set forth in siTIMP2_p100 (SEQ ID NOS:2522 and 2575). In some embodiments the antisense and sense strands of a nucleic acid molecule (e.g., a siNA molecule) as disclosed herein includes the sequence pairs set forth in siTIMP2_p102 (SEQ ID NOS:1007 and 1622).

In some embodiments the covalent bond joining each consecutive N or N′ is a phosphodiester bond.

In some embodiments x=y and each of x and y is 19, 20, 21, 22 or 23. In various embodiments x=y=19. In some embodiments the antisense and sense strands form a duplex by base pairing.

According to one embodiment provided are modified nucleic acid molecules having a structure (A2) set forth below:

| | | |
|---|---|---|
| (A2) | 5′ N1-(N)x-Z | 3′ (antisense strand) |
| | 3′ Z′-N2-(N′)y-z″ | 5′ (sense strand) |

wherein each of N2, N and N′ is independently an unmodified or modified nucleotide, or an unconventional moiety;
wherein each of (N)x and (N′)y is an oligonucleotide in which each consecutive N or N′ is joined to the adjacent N or N′ by a covalent bond;
wherein each of x and y is independently an integer of from 17 to 39;
wherein the sequence of (N′)y has complementarity to the sequence of (N)x and (N)x has complementarity to a consecutive sequence in a target mRNA selected from SEQ ID NO:1 and SEQ ID NO:2;
wherein N1 is covalently bound to (N)x and is mismatched to SEQ ID NO:1 or to SEQ ID NO:2,
wherein N1 is a moiety selected from the group consisting of uridine, modified uridine, ribothymidine, modified ribothymidine, deoxyribothymidine, modified deoxyribothymidine, riboadenine, modified riboadenine, deoxyriboadenine or modified deoxyriboadenine;
wherein N1 and N2 form a base pair;
wherein each of Z and Z′ is independently present or absent, but if present is independently 1-5 consecutive nucleotides or non-nucleotide moieties or a combination thereof covalently attached at the 3′ terminus of the strand in which it is present; and
wherein z″ may be present or absent, but if present is a capping moiety covalently attached at the 5′ terminus of (N′)y.

Molecules covered by the description of Structure A2 are also referred to herein as “18+1” or “18+1 mer”. In some embodiments the N2-(N′)y and N1-(N)x oligonucleotide strands useful in generating dsRNA compounds are presented in Tables A5, A6, A7, A8, B5, B6, B7 or B8. In some embodiments (N)x has complementarity to a consecutive sequence in SEQ ID NO:1 (human TIMP1 mRNA). In some embodiments (N)x includes an antisense oligonucleotide present in any one of Tables A5, A6, A7, and A8. In some embodiments x=y=18 and N1-(N)x includes an antisense oligonucleotide present in any one of Tables A3 or A4. In some embodiments x=y=19 or x=y=20. In certain preferred embodiments x=y=18.

In certain preferred embodiments, the antisense strand of a nucleic acid molecule (e.g., a siNA molecule) as disclosed herein includes a sequence corresponding to any one of the antisense sequences shown on Table A5. In certain preferred embodiments the antisense strand and the strand are selected from the sequence pairs shown in Table A6. In certain preferred embodiments the antisense strand and the strand are active in more than one species (human and at least one other species) and are selected from the sequence pairs shown in Table A6. In certain preferred embodiments the antisense strand and the strand are selected from the sequence pairs shown in Table A7, and preferably in Table A8.

In some embodiments the antisense and sense strands are selected from the sequence pairs set forth in siTIMP1_p1; siTIMP1_p3; siTIMP1_p4; siTIMP1_p5; siTIMP1_p7; siTIMP1_p8; siTIMP1_p9; siTIMP1_p10; siTIMP1_p11; siTIMP1_p12; siTIMP1_p13; siTIMP1_p15; siTIMP1_p18; siTIMP1_p22; siTIMP1_p25; siTIMP1_p26; siTIMP1_p28; siTIMP1_p30; siTIMP1_p32; siTIMP1_p34; siTIMP1_p35; siTIMP1_p36; siTIMP1_p37; siTIMP1_p39; siTIMP1_p40; siTIMP1_p41; siTIMP1_p44; siTIMP1_p46; siTIMP1_p47; siTIMP1_p48; siTIMP1_p50; siTIMP1_p51; siTIMP1_p52; siTIMP1_p53; siTIMP1_p54; siTIMP1_p55; siTIMP1_p56; siTIMP1_p57; siTIMP1_p58; siTIMP1_p59; siTIMP1_p61; siTIMP1_p62; siTIMP1_p63; siTIMP1_p64; siTIMP1_p65; siTIMP1_p66; siTIMP1_p67; siTIMP1_p68; siTIMP1_p69; siTIMP1_p70; siTIMP1_p72; siTIMP1_p74; siTIMP1_p75; siTIMP1_p76; siTIMP1_p80; siTIMP1_p81; siTIMP1_p82; siTIMP1_p83; siTIMP1_p84; siTIMP1_p86; siTIMP1_p87; siTIMP1_p88; siTIMP1_p90; siTIMP1_p92; siTIMP1_p93; siTIMP1_p94; siTIMP1_p95; siTIMP1_p97; siTIMP1_p100; siTIMP1_p101; siTIMP1_p102; siTIMP1_p103; siTIMP1_p104; siTIMP1_p105; siTIMP1_p106; siTIMP1_p109; siTIMP1_p110; siTIMP1_p111; siTIMP1_p112; siTIMP1_p113 and siTIMP1_p114, shown in Table A7, infra.

In some embodiments the antisense and sense strands are selected from the sequence pairs set forth in siTIMP1_p1 (SEQ ID NOS:845 and 926); siTIMP1_p4 (SEQ ID NOS:847 and 928; siTIMP1_p5 (SEQ ID NOS:848 and 929); siTIMP1_p7 (SEQ ID NOS:849 and 930); siTIMP1_p8 (SEQ ID NOS:850 and 931); siTIMP1_p9 (SEQ ID NOS:850 and 931); siTIMP1_p10 (SEQ ID NOS:852 and 933); siTIMP1_p11 (SEQ ID NOS:853 and 934); siTIMP1_p12 (SEQ ID NOS:854 and 935); siTIMP1_p13 (SEQ ID NOS:855 and 936); siTIMP1_p15 (SEQ ID NOS:856 and 937); siTIMP1_p18 (SEQ ID NOS:857 and 938); siTIMP1_p22 (SEQ ID NOS:858 and 939); siTIMP1_p26 (SEQ ID NOS:860 and 941); siTIMP1_p36 (SEQ ID NOS:866 and 947); siTIMP1_p37 (SEQ ID NOS:867 and 948); siTIMP1_p39 (SEQ ID NOS:868 and 949); siTIMP1_p40 (SEQ ID NOS:869 and 950); siTIMP1_p41 (SEQ ID NOS:870 and 951); siTIMP1_p44 (SEQ ID NOS:871 and 952); siTIMP1_p47 (SEQ ID NOS:873 and 954); siTIMP1_p48 (SEQ ID NOS:874 and 955); siTIMP1_p50 (SEQ ID NOS:875 and 956); siTIMP1_p51 (SEQ ID NOS:876 and 957); siTIMP1_p52 (SEQ ID NOS:877 and 958); siTIMP1_p55 (SEQ ID NOS:880 and 961); siTIMP1_p56 (SEQ ID NOS:881 and 962); siTIMP1_p58 (SEQ ID NOS:883 and 964); siTIMP1_p61 (SEQ ID NOS:885 and 966); siTIMP1_p64 (SEQ ID NOS:888 and 969); siTIMP1_p66 (SEQ ID NOS:890 and 971); siTIMP1_p68 (SEQ ID NOS:892 and 973); siTIMP1_p70 (SEQ ID NOS:894 and 975); siTIMP1_p75 (SEQ ID NOS:897 and 978); siTIMP1_p83 (SEQ ID NOS:902 and 983); siTIMP1_p86 (SEQ ID NOS:904 and 985); siTIMP1_p88 (SEQ ID NOS:906 and 987); siTIMP1_p92 (SEQ ID NOS:908 and 989); siTIMP1_p93 (SEQ ID NOS:909 and 990); siTIMP1_p95 (SEQ ID NOS:911 and 992); siTIMP1_p97 (SEQ ID NOS:912 and 993); siTIMP1_p102 (SEQ ID NOS:915 and 996); siTIMP1_p104 (SEQ ID NOS:917 and 998); siTIMP1_p105 (SEQ ID NOS:918 and 999); siTIMP1_p106 (SEQ ID NOS:919 and 1000); siTIMP1_p110 (SEQ ID NOS:921 and 1002) and siTIMP1_p112 (SEQ ID NOS:923 and 1004), shown in Table A8, infra.

In some embodiments the antisense and sense strands of a nucleic acid molecule (e.g., a siNA molecule) as disclosed herein includes the sequence pairs set forth in siTIMP1_p1 (SEQ ID NOS:845 and 926). In some embodiments the antisense and sense strands of a nucleic acid molecule (e.g., a siNA molecule) as disclosed herein includes the sequence pairs set forth in siTIMP1_p4 (SEQ ID NOS:847 and 928. In some embodiments the antisense and sense strands of a nucleic acid molecule (e.g., a siNA molecule) as disclosed herein includes the sequence pairs set forth in siTIMP1_p5 (SEQ ID NOS:848 and 929). In some embodiments the antisense and sense strands of a nucleic acid molecule (e.g., a siNA molecule) as disclosed herein includes the sequence pairs set forth in siTIMP1_p7 (SEQ ID NOS:849 and 930). In some embodiments the antisense and sense strands of a nucleic acid molecule (e.g., a siNA molecule) as disclosed herein includes the sequence pairs set forth in siTIMP1_p8 (SEQ ID NOS:850 and 931). In some embodiments the antisense and sense strands of a nucleic acid molecule (e.g., a siNA molecule) as disclosed herein includes the sequence pairs set forth in siTIMP1_p9 (SEQ ID NOS:850 and 931). In some embodiments the antisense and sense strands of a nucleic acid molecule (e.g., a siNA molecule) as disclosed herein includes the sequence pairs set forth in siTIMP1_p10 (SEQ ID NOS:852 and 933). In some embodiments the antisense and sense strands of a nucleic acid molecule (e.g., a siNA molecule) as disclosed herein includes the sequence pairs set forth in siTIMP1_p11 (SEQ ID NOS:853 and 934). In some embodiments the antisense and sense strands of a nucleic acid molecule (e.g., a siNA molecule) as disclosed herein includes the sequence pairs set forth in siTIMP1_p12 (SEQ ID NOS:854 and 935). In some embodiments the antisense and sense strands of a nucleic acid molecule (e.g., a siNA molecule) as disclosed herein includes the sequence pairs set forth in siTIMP1_p13 (SEQ ID NOS:855 and 936). In some embodiments the antisense and sense strands of a nucleic acid molecule (e.g., a siNA molecule) as disclosed herein includes the sequence pairs set forth in siTIMP1_p15 (SEQ ID NOS:856 and 937). In some embodiments the antisense and sense strands of a nucleic acid molecule (e.g., a siNA molecule) as disclosed herein includes the sequence pairs set forth in siTIMP1_p18 (SEQ ID NOS:857 and 938). In some embodiments the antisense and sense strands of a nucleic acid molecule (e.g., a siNA molecule) as disclosed herein includes the sequence pairs set forth in siTIMP1_p22 (SEQ ID NOS:858 and 939). In some embodiments the antisense and sense strands of a nucleic acid molecule (e.g., a siNA molecule) as disclosed herein includes the sequence pairs set forth in siTIMP1_p26 (SEQ ID NOS:860 and 941). In some embodiments the antisense and sense strands of a nucleic acid molecule (e.g., a siNA molecule) as disclosed herein includes the sequence pairs set forth in siTIMP1_p36 (SEQ ID NOS:866 and 947). In some embodiments the antisense and sense strands of a nucleic acid molecule (e.g., a siNA molecule) as disclosed herein includes the sequence pairs set forth in siTIMP1_p37 (SEQ ID NOS:867 and 948). In some embodiments the antisense and sense strands of a nucleic acid molecule (e.g., a siNA molecule) as disclosed herein includes the sequence pairs set forth in siTIMP1_p39 (SEQ ID NOS:868 and 949). In some embodiments the antisense and sense strands of a nucleic acid molecule (e.g., a siNA molecule) as disclosed herein includes the sequence pairs set forth in siTIMP1_p40 (SEQ ID NOS:869 and 950). In some embodiments the antisense and sense strands of a nucleic acid molecule (e.g., a siNA molecule) as disclosed herein includes the sequence pairs set forth in siTIMP1_p41 (SEQ ID NOS:870 and 951). In some embodiments the antisense and sense strands of a nucleic acid molecule (e.g., a siNA molecule) as disclosed herein includes the sequence pairs set forth in siTIMP1_p44 (SEQ ID NOS:871 and 952). In some embodiments the antisense and sense strands of a nucleic acid molecule (e.g., a siNA molecule) as disclosed herein includes the sequence pairs set forth in siTIMP1_p47 (SEQ ID NOS:873 and 954). In some embodiments the antisense and sense strands of a nucleic acid molecule (e.g., a siNA molecule) as disclosed herein includes the sequence pairs set forth in siTIMP1_p48 (SEQ ID NOS:874 and 955). In some embodiments the antisense and sense strands of a nucleic acid molecule (e.g., a siNA molecule) as disclosed herein includes the sequence pairs set forth in siTIMP1_p50 (SEQ ID NOS:875 and 956). In some embodiments the antisense and sense strands of a nucleic acid molecule (e.g., a siNA molecule) as disclosed herein includes the sequence pairs set forth in siTIMP1_p51 (SEQ ID NOS:876 and 957). In some embodiments the antisense and sense strands of a nucleic acid molecule (e.g., a siNA molecule) as disclosed herein includes the sequence pairs set forth in siTIMP1_p52 (SEQ ID NOS:877 and 958). In some embodiments the antisense and sense strands of a nucleic acid molecule (e.g., a siNA molecule) as disclosed herein includes the sequence pairs set forth in siTIMP1_p55 (SEQ ID NOS:880 and 961). In some embodiments the antisense and sense strands of a nucleic acid molecule (e.g., a siNA molecule) as disclosed herein includes the sequence pairs set forth in siTIMP1_p56 (SEQ ID NOS:881 and 962). In some embodiments the antisense and sense strands of a nucleic acid molecule (e.g., a siNA molecule) as disclosed herein includes the sequence pairs set forth in siTIMP1_p58 (SEQ ID NOS:883 and 964). In some embodiments the antisense and sense strands of a nucleic acid molecule (e.g., a siNA molecule) as disclosed herein includes the sequence pairs set forth in siTIMP1_p61 (SEQ ID NOS:885 and 966). In some embodiments the antisense and sense strands of a nucleic acid molecule (e.g., a siNA molecule) as disclosed herein includes the sequence pairs set forth in siTIMP1_p64 (SEQ ID NOS:888 and 969). In some embodiments the antisense and sense strands of a nucleic acid molecule (e.g., a siNA molecule) as disclosed herein includes the sequence pairs set forth in siTIMP1_p66 (SEQ ID NOS:890 and 971). In some embodiments the antisense and sense strands of a nucleic acid molecule (e.g., a siNA molecule) as disclosed herein includes the sequence pairs set forth in siTIMP1_p68 (SEQ ID NOS:892 and 973). In some embodiments the antisense and sense strands of a nucleic acid molecule (e.g., a siNA molecule) as disclosed herein includes the sequence pairs set forth in siTIMP1_p70 (SEQ ID NOS:894 and 975). In some embodiments the antisense and sense strands of a nucleic acid molecule (e.g., a siNA molecule) as disclosed herein includes the sequence pairs set forth in siTIMP1_p75 (SEQ ID NOS:897 and 978). In some embodiments the antisense and sense strands of a nucleic acid molecule (e.g., a siNA molecule) as disclosed herein includes the sequence pairs set forth in siTIMP1_p83 (SEQ ID NOS:902 and 983). In some embodiments the antisense and sense strands of a nucleic acid molecule (e.g., a siNA molecule) as disclosed herein includes the sequence pairs set forth in siTIMP1_p86 (SEQ ID NOS:904 and 985). In some embodiments the antisense and sense strands of a nucleic acid molecule (e.g., a siNA molecule) as disclosed herein includes the sequence pairs set forth in siTIMP1_p88 (SEQ ID NOS:906 and 987). In some embodiments the antisense and sense strands of a nucleic acid molecule (e.g., a siNA molecule) as disclosed herein includes the sequence pairs set forth in siTIMP1_p92 (SEQ ID NOS:908 and 989). In some embodiments the antisense and sense strands of a nucleic acid molecule (e.g., a siNA molecule) as disclosed herein includes the sequence pairs set forth in siTIMP1_p93 (SEQ ID NOS:909 and 990). In some embodiments the antisense and sense strands of a nucleic acid molecule (e.g., a siNA molecule) as disclosed herein includes the sequence pairs set forth in siTIMP1_p95 (SEQ ID NOS:911 and 992). In some embodiments the antisense and sense strands of a nucleic acid molecule (e.g., a siNA molecule) as disclosed herein includes the sequence pairs set forth in siTIMP1_p97 (SEQ ID NOS:912 and 993). In some embodiments the antisense and sense strands of a nucleic acid molecule (e.g., a siNA molecule) as disclosed herein includes the sequence pairs set forth in siTIMP1_p102 (SEQ ID NOS:915 and 996). In some embodiments the antisense and sense strands of a nucleic acid molecule (e.g., a siNA molecule) as disclosed herein includes the sequence pairs set forth in siTIMP1_p104 (SEQ ID NOS:917 and 998). In some embodiments the antisense and sense strands of a nucleic acid molecule (e.g., a siNA molecule) as disclosed herein includes the sequence pairs set forth in siTIMP1_p105 (SEQ ID NOS:918 and 999). In some embodiments the antisense and sense strands of a nucleic acid molecule (e.g., a siNA molecule) as disclosed herein includes the sequence pairs set forth in siTIMP1_p106 (SEQ ID NOS:919 and 1000). In some embodiments the antisense and sense strands of a nucleic acid molecule (e.g., a siNA molecule) as disclosed herein includes the sequence pairs set forth in siTIMP1_p110 (SEQ ID NOS:921 and 1002). In some embodiments the antisense and sense strands of a nucleic acid molecule (e.g., a siNA molecule) as disclosed herein includes the sequence pairs set forth in siTIMP1_p112 (SEQ ID NOS:923 and 1004).

In some preferred embodiments the nucleic acid molecule (e.g., a siNA molecule) as disclosed herein includes the antisense strand and the sense strand of a sequence pair set forth in siTIMP1_p1 (SEQ ID NOS:845 and 926). In some preferred embodiments the nucleic acid molecule (e.g., a siNA molecule) as disclosed herein includes the antisense strand and the sense strand of a sequence pair set forth in siTIMP1_p4 (SEQ ID NOS:847 and 928. In some preferred embodiments the nucleic acid molecule (e.g., a siNA molecule) as disclosed herein includes the antisense strand and the sense strand of a sequence pair set forth in siTIMP1_p5 (SEQ ID NOS:848 and 929). In some preferred embodiments the nucleic acid molecule (e.g., a siNA molecule) as disclosed herein includes the antisense strand and the sense strand of a sequence pair set forth in siTIMP1_p7 (SEQ ID NOS:849 and 930). In some preferred embodiments the nucleic acid molecule (e.g., a siNA molecule) as disclosed herein includes the antisense strand and the sense strand of a sequence pair set forth in siTIMP1_p9 (SEQ ID NOS:850 and 931). In some preferred embodiments the nucleic acid molecule (e.g., a siNA molecule) as disclosed herein includes the antisense strand and the sense strand of a sequence pair set forth in siTIMP1_p10 (SEQ ID NOS:852 and 933). In some preferred embodiments the nucleic acid molecule (e.g., a siNA molecule) as disclosed herein includes the antisense strand and the sense strand of a sequence pair set forth in siTIMP1_p11 (SEQ ID NOS:853 and 934). In some preferred embodiments the nucleic acid molecule (e.g., a siNA molecule) as disclosed herein includes the antisense strand and the sense strand of a sequence pair set forth in siTIMP1_p12 (SEQ ID NOS:854 and 935). In some preferred embodiments the nucleic acid molecule (e.g., a siNA molecule) as disclosed herein includes the antisense strand and the sense strand of a sequence pair set forth in siTIMP1_p13 (SEQ ID NOS:855 and 936). In some preferred embodiments the nucleic acid molecule (e.g., a siNA molecule) as disclosed herein includes the antisense strand and the sense strand of a sequence pair set forth in siTIMP1_p15 (SEQ ID NOS:856 and 937). In some preferred embodiments the nucleic acid molecule (e.g., a siNA molecule) as disclosed herein includes the antisense strand and the sense strand of a sequence pair set forth in siTIMP1_p18 (SEQ ID NOS:857 and 938). In some preferred embodiments the nucleic acid molecule (e.g., a siNA molecule) as disclosed herein includes the antisense strand and the sense strand of a sequence pair set forth in siTIMP1_p44 (SEQ ID NOS:871 and 952). In some preferred embodiments the nucleic acid molecule (e.g., a siNA molecule) as disclosed herein includes the antisense strand and the sense strand of a sequence pair set forth in siTIMP1_p48 (SEQ ID NOS:874 and 955). In some preferred embodiments the nucleic acid molecule (e.g., a siNA molecule) as disclosed herein includes the antisense strand and the sense strand of a sequence pair set forth in siTIMP1_p51 (SEQ ID NOS:876 and 957). In some preferred embodiments the nucleic acid molecule (e.g., a siNA molecule) as disclosed herein includes the antisense strand and the sense strand of a sequence pair set forth in siTIMP1_p52 (SEQ ID NOS:877 and 958).

In some embodiments (N)x has complementarity to a consecutive sequence in SEQ ID NO:2 (human TIMP2 mRNA). In some embodiments (N)x includes an antisense oligonucleotide present in any one of Tables B5, B6, B7, and B8. In some embodiments x=y=18 and N1-(N)x includes an antisense oligonucleotide present in any one of Tables B3 or B4. In some embodiments x=y=19 or x=y=20. In certain preferred embodiments x=y=18.

In certain preferred embodiments, the antisense strand of a nucleic acid molecule (e.g., a siNA molecule) as disclosed herein includes a sequence corresponding to any one of the antisense sequences shown on Table B5. In certain preferred embodiments the antisense strand and the strand are selected from the sequence pairs shown in Table B6. In certain preferred embodiments the antisense strand and the strand are active in more than one species (human and at least one other species) and are selected from the sequence pairs shown in Table B6. In certain preferred embodiments the antisense strand and the strand are selected from the sequence pairs shown in Table B7, and preferably from Table B8.

In some embodiments the antisense and sense strands are selected from the sequence pairs set forth in siTIMP2_p1; siTIMP2_p2; siTIMP2_p3; siTIMP2_p5; siTIMP2_p6; siTIMP2_p7; siTIMP2_p8; siTIMP2_p9; siTIMP2_p10; siTIMP2_p11; siTIMP2_p12; siTIMP2_p13; siTIMP2_p14; siTIMP2_p15; siTIMP2_p19; siTIMP2_p21; siTIMP2_p22; siTIMP2_p23; siTIMP2_p26; siTIMP2_p28; siTIMP2_p31; siTIMP2_p32; siTIMP2_p34; siTIMP2_p36; siTIMP2_p42; siTIMP2_p43; siTIMP2_p45; siTIMP2_p47; siTIMP2_p48; siTIMP2_p49; siTIMP2_p50; siTIMP2_p52; siTIMP2_p53; siTIMP2_p54; siTIMP2_p56; siTIMP2_p57; siTIMP2_p58; siTIMP2_p59; siTIMP2_p60; siTIMP2_p63; siTIMP2_p66; siTIMP2_p70; siTIMP2_p72; siTIMP2_p73; siTIMP2_p74; siTIMP2_p77; siTIMP2_p80 and siTIMP2_p81, shown in Table B7, infra.

In some embodiments the antisense and sense strands are selected from the sequence pairs set forth in siTIMP2_p6 (SEQ ID NOS:4771 and 4819); siTIMP2_p9 (SEQ ID NOS:4774 and 4822); siTIMP2_p15 (SEQ ID NOS:4780 and 4828); siTIMP2_p19 (SEQ ID NOS:4781 and 4829); siTIMP2_p21 (SEQ ID NOS:4782 and 4830); siTIMP2_p22 (SEQ ID NOS:4783 and 4831); siTIMP2_p23 (SEQ ID NOS:4784 and 4832); siTIMP2_p28 (SEQ ID NOS:4786 and 4834); siTIMP2_p31 (SEQ ID NOS:4787 and 4835); siTIMP2_p36 (SEQ ID NOS:4790 and 4838); siTIMP2_p42 (SEQ ID NOS:4791 and 4839); siTIMP2_p47 (SEQ ID NOS:4794 and 4842); siTIMP2_p50 (SEQ ID NOS:4797 and 4845); siTIMP2_p56 (SEQ ID NOS:4801 and 4849); siTIMP2_p57 (SEQ ID NOS:4802 and 4850); siTIMP2_p58 (SEQ ID NOS:4803 and 4851); siTIMP2_p60 (SEQ ID NOS:4805 and 4853); siTIMP2_p63 (SEQ ID NOS:4806 and 4854); siTIMP2_p70 (SEQ ID NOS:4808 and 4856); siTIMP2_p73 (SEQ ID NOS:4810 and 4858); siTIMP2_p74 (SEQ ID NOS:4811 and 4859); and siTIMP2_p81 (SEQ ID NOS:4814 and 4862), shown in Table B8, infra.

In some embodiments the antisense and sense strands of a nucleic acid molecule (e.g., a siNA molecule) as disclosed herein includes the sequence pairs set forth in siTIMP2_p6 (SEQ ID NOS:4771 and 4819). In some embodiments the antisense and sense strands of a nucleic acid molecule (e.g., a siNA molecule) as disclosed herein includes the sequence pairs set forth in siTIMP2_p9 (SEQ ID NOS:4774 and 4822). In some embodiments the antisense and sense strands of a nucleic acid molecule (e.g., a siNA molecule) as disclosed herein includes the sequence pairs set forth in siTIMP2_p15 (SEQ ID NOS:4780 and 4828). In some embodiments the antisense and sense strands of a nucleic acid molecule (e.g., a siNA molecule) as disclosed herein includes the sequence pairs set forth in siTIMP2_p19 (SEQ ID NOS:4781 and 4829). In some embodiments the antisense and sense strands of a nucleic acid molecule (e.g., a siNA molecule) as disclosed herein includes the sequence pairs set forth in siTIMP2_p21 (SEQ ID NOS:4782 and 4830). In some embodiments the antisense and sense strands of a nucleic acid molecule (e.g., a siNA molecule) as disclosed herein includes the sequence pairs set forth in siTIMP2_p22 (SEQ ID NOS:4783 and 4831). In some embodiments the antisense and sense strands of a nucleic acid molecule (e.g., a siNA molecule) as disclosed herein includes the sequence pairs set forth in siTIMP2_p23 (SEQ ID NOS:4784 and 4832). In some embodiments the antisense and sense strands of a nucleic acid molecule (e.g., a siNA molecule) as disclosed herein includes the sequence pairs set forth in siTIMP2_p28 (SEQ ID NOS:4786 and 4834). In some embodiments the antisense and sense strands of a nucleic acid molecule (e.g., a siNA molecule) as disclosed herein includes the sequence pairs set forth in siTIMP2_p31 (SEQ ID NOS:4787 and 4835). In some embodiments the antisense and sense strands of a nucleic acid molecule (e.g., a siNA molecule) as disclosed herein includes the sequence pairs set forth in siTIMP2_p36 (SEQ ID NOS:4790 and 4838). In some embodiments the antisense and sense strands of a nucleic acid molecule (e.g., a siNA molecule) as disclosed herein includes the sequence pairs set forth in siTIMP2_p42 (SEQ ID NOS:4791 and 4839). In some embodiments the antisense and sense strands of a nucleic acid molecule (e.g., a siNA molecule) as disclosed herein includes the sequence pairs set forth in siTIMP2_p47 (SEQ ID NOS:4794 and 4842). In some embodiments the antisense and sense strands of a nucleic acid molecule (e.g., a siNA molecule) as disclosed herein includes the sequence pairs set forth in siTIMP2_p50 (SEQ ID NOS:4797 and 4845). In some embodiments the antisense and sense strands of a nucleic acid molecule (e.g., a siNA molecule) as disclosed herein includes the sequence pairs set forth in siTIMP2_p56 (SEQ ID NOS:4801 and 4849). In some embodiments the antisense and sense strands of a nucleic acid molecule (e.g., a siNA molecule) as disclosed herein includes the sequence pairs set forth in siTIMP2_p57 (SEQ ID NOS:4802 and 4850). In some embodiments the antisense and sense strands of a nucleic acid molecule (e.g., a siNA molecule) as disclosed herein includes the sequence pairs set forth in siTIMP2_p58 (SEQ ID NOS:4803 and 4851). In some embodiments the antisense and sense strands of a nucleic acid molecule (e.g., a siNA molecule) as disclosed herein includes the sequence pairs set forth in siTIMP2_p60 (SEQ ID NOS:4805 and 4853). In some embodiments the antisense and sense strands of a nucleic acid molecule (e.g., a siNA molecule) as disclosed herein includes the sequence pairs set forth in siTIMP2_p63 (SEQ ID NOS:4806 and 4854); siTIMP2_p70 (SEQ ID NOS:4808 and 4856). In some embodiments the antisense and sense strands of a nucleic acid molecule (e.g., a siNA molecule) as disclosed herein includes the sequence pairs set forth in siTIMP2_p73 (SEQ ID NOS:4810 and 4858). In some embodiments the antisense and sense strands of a nucleic acid molecule (e.g., a siNA molecule) as disclosed herein includes the sequence pairs set forth in siTIMP2_p74 (SEQ ID NOS:4811 and 4859). In some embodiments the antisense and sense strands of a nucleic acid molecule (e.g., a siNA molecule) as disclosed herein includes the sequence pairs set forth in siTIMP2_p81 (SEQ ID NOS:4814 and 4862).

In some embodiments N1 and N2 form a Watson-Crick base pair. In other embodiments N1 and N2 form a non-Watson-Crick base pair. In some embodiments N1 is a modified riboadenosine or a modified ribouridine.

In certain embodiments N1 is selected from the group consisting of riboadenosine, modified riboadenosine, deoxyriboadenosine, modified deoxyriboadenosine. In other embodiments N1 is selected from the group consisting of ribouridine, deoxyribouridine, modified ribouridine, and modified deoxyribouridine.

In certain embodiments N1 is selected from the group consisting of riboadenosine, modified riboadenosine, deoxyriboadenosine, modified deoxyriboadenosine and N2 is selected from the group consisting of ribouridine, deoxyribouridine, modified ribouridine, and modified deoxyribouridine. In certain embodiments N1 is selected from the group consisting of riboadenosine and modified riboadenosine and N2 is selected from the group consisting of ribouridine and modified ribouridine.

In certain embodiments N2 is selected from the group consisting of riboadenosine, modified riboadenosine, deoxyriboadenosine, modified deoxyriboadenosine and N1 is selected from the group consisting of ribouridine, deoxyribouridine, modified ribouridine, and modified deoxyribouridine. In certain embodiments N1 is selected from the group consisting of ribouridine and modified ribouridine and N2 is selected from the group consisting of riboadenine and modified riboadenine. In certain embodiments N1 is ribouridine and N2 is riboadenine.

In some embodiments of Structure (A2), N1 includes 2′OMe sugar-modified ribouracil or 2′OMe sugar-modified riboadenosine. In certain embodiments of structure (A), N2 includes a 2′OMe sugar modified ribonucleotide or deoxyribonucleotide.

In some embodiments Z and Z′ are absent. In other embodiments one of Z or Z′ is present.

In some embodiments each of N and N′ is an unmodified nucleotide. In some embodiments at least one of N or N′ includes a chemically modified nucleotide or an unconventional moiety. In some embodiments the unconventional moiety is selected from a mirror nucleotide, an abasic ribose moiety and an abasic deoxyribose moiety. In some embodiments the unconventional moiety is a mirror nucleotide, preferably an L-DNA moiety. In some embodiments at least one of N or N′ includes a 2′ OMe sugar-modified ribonucleotide.

In some embodiments the sequence of (N′)y is fully complementary to the sequence of (N)x. In other embodiments the sequence of (N′)y is substantially complementary to the sequence of (N)x.

In some embodiments (N)x includes an antisense sequence that is fully complementary to about 17 to about 39 consecutive nucleotides in a target mRNA. In other embodiments (N)x includes an antisense that is substantially complementary to about 17 to about 39 consecutive nucleotides in a target mRNA. In some embodiments (N)x includes an antisense that is substantially complementary to about 17, 18, 19, 20, 21, 22, 23, 24, 25, 26, 27, 28, 29, 30, 31, 32, 33, 34, 35, 36, 37, 38, to about 39 consecutive nucleotides in a target mRNA. In other embodiments (N)x includes an antisense that is substantially complementary to about 17 to about 23, 18 to about 23, 18 to about 21, or 18 to about 19 consecutive nucleotides in a target mRNA.

In some embodiments of Structure A1 and Structure A2 the compound is blunt ended, for example wherein both Z and Z′ are absent. In an alternative embodiment, at least one of Z or Z′ is present. Z and Z′ independently include one or more covalently linked modified and or unmodified nucleotides, including deoxyribonucleotides and ribonucleotides, or an unconventional moiety for example inverted abasic deoxyribose moiety or abasic ribose moiety; a non-nucleotide C3, C4 or C5 moiety, an amino-6 moiety, a mirror nucleotide and the like. In some embodiments each of Z and Z′ independently includes a C3 moiety or an amino-C6 moiety. In some embodiments Z′ is absent and Z is present and includes a non-nucleotide C3 moiety. In some embodiments Z is absent and Z′ is present and includes a non-nucleotide C3 moiety.

In some preferred embodiments of Structures A1 and Structure A2 an asymmetrical siNA compound molecule has a 3′ terminal non-nucleotide overhang (for example C3-C3 3′-overhang) on one side of a duplex occurring on the antisense strand; and a blunt end on the other side of the molecule. In some preferred embodiments z′ is present and the dsNA molecule has a 5′ terminal non-nucleotide overhang (for example an abasic moiety) on one side of a duplex occurring on the sense strand; and a blunt end on the other side of the molecule.

In some embodiments of Structure A1 and Structure A2 each N consists of an unmodified ribonucleotide. In some embodiments of Structure A1 and Structure A2 each N′ consists of an unmodified nucleotide. In preferred embodiments, at least one of N and N′ is a modified ribonucleotide or an unconventional moiety.

In other embodiments the compound of Structure A1 or Structure A2 includes at least one ribonucleotide modified in the sugar residue. In some embodiments the compound includes a modification at the 2′ position of the sugar residue. In some embodiments the modification in the 2′ position includes the presence of an amino, a fluoro, an alkoxy or an alkyl moiety. In certain embodiments the 2′ modification includes an alkoxy moiety. In preferred embodiments the alkoxy moiety is a methoxy moiety (also known as 2′-O-methyl; 2′OMe; 2′-OCH3). In some embodiments the nucleic acid compound includes 2′OMe sugar modified alternating ribonucleotides in one or both of the antisense and the sense strands. In other embodiments the compound includes 2′OMe sugar modified ribonucleotides in the antisense strand, (N)x or N1-(N)x, only. In certain embodiments the middle ribonucleotide of the antisense strand; e.g. ribonucleotide in position 10 in a 19-mer strand is unmodified. In various embodiments the nucleic acid compound includes at least 5 alternating 2′OMe sugar modified and unmodified ribonucleotides.

In additional embodiments the compound of Structure A1 or Structure A2 includes modified ribonucleotides in alternating positions wherein each ribonucleotide at the 5′ and 3′ termini of (N)x or N1-(N)x are modified in their sugar residues, and each ribonucleotide at the 5′ and 3′ termini of (N′)y or N2-(N)y are unmodified in their sugar residues.

In some embodiments, (N)x or N1-(N)x includes 2′OMe modified ribonucleotides at positions 2, 4, 6, 8, 11, 13, 15, 17 and 19. In other embodiments (N)x (N)x or N1-(N)x includes 2′OMe modified ribonucleotides at positions 1, 3, 5, 7, 9, 11, 13, 15, 17 and 19. In some embodiments (N)x or N1-(N)x includes 2′OMe modified pyrimidines. In some embodiments all the pyrimidine nucleotides in (N)x or N1-(N)x are 2′OMe modified. In some embodiments (N′)y or N2-(N′)y includes 2′OMe modified pyrimidines.

In additional embodiments the compound of Structure A1 or Structure A2 includes modified ribonucleotides in alternating positions wherein each ribonucleotide at the 5′ and 3′ termini of (N)x or N1-(N)x are modified in their sugar residues, and each ribonucleotide at the 5′ and 3′ termini of (N′)y or N2-(N)y are unmodified in their sugar residues.

In some embodiments of Structure A1 and Structure A2, neither of the sense strand nor the antisense strand is phosphorylated at the 3′ and 5′ termini. In other embodiments one or both of the sense strand or the antisense strand are phosphorylated at the 3′ termini.

In some embodiments of Structure A1 and Structure A2 (N)y includes at least one unconventional moiety selected from a mirror nucleotide and a nucleotide joined to an adjacent nucleotide by a 2′-5′ internucleotide phosphate bond also known as 2′-5′ linked or 2′-5′ linkage. In some embodiments the unconventional moiety is a mirror nucleotide. In various embodiments the mirror nucleotide is selected from an L-ribonucleotide (L-RNA) and an L-deoxyribonucleotide (L-DNA). In preferred embodiments the mirror nucleotide is L-DNA.

In some embodiments of Structure A1 (N′)y includes at least one L-DNA moiety. In some embodiments x=y=19 and (N′)y, consists of unmodified ribonucleotides at positions 1-17 and 19 and one L-DNA at the 3′ penultimate position (position 18). In other embodiments x=y=19 and (N′)y consists of unmodified ribonucleotides at position 1-16 and 19 and two consecutive L-DNA at the 3′ penultimate position (positions 17 and 18). In various embodiments the unconventional moiety is a nucleotide joined to an adjacent nucleotide by a 2′-5′ internucleotide phosphate linkage. According to various embodiments (N′)y includes 2, 3, 4, 5, or 6 consecutive ribonucleotides at the 3′ terminus linked by 2′-5′ internucleotide linkages. In one embodiment, four consecutive nucleotides at the 3′ terminus of (N′)y are joined by three 2′-5′ phosphodiester bonds, wherein one or more of the 2′-5′ nucleotides which form the 2′-5′ phosphodiester bonds further includes a 3′-O-methyl (3′OMe) sugar modification. Preferably the 3′ terminal nucleotide of (N′)y includes a 2′OMe sugar modification. In certain embodiments x=y=19 and (N′)y includes two or more consecutive nucleotides at positions 15, 16, 17, 18 and 19 include a nucleotide joined to an adjacent nucleotide by a 2′-5′ internucleotide bond. In various embodiments the nucleotide forming the 2′-5′ internucleotide bond includes a 3′ deoxyribose nucleotide or a 3′ methoxy nucleotide. In some embodiments x=y=19 and (N′)y includes nucleotides joined to the adjacent nucleotide by a 2′-5′ internucleotide bond between positions 15-16, 16-17 and 17-18 or between positions 16-17, 17-18 and 18-19. In some embodiments x=y=19 and (N′)y includes nucleotides joined to the adjacent nucleotide by a 2′-5′ internucleotide bond between positions 16-17 and 17-18 or between positions 17-18 and 18-19 or between positions 15-16 and 17-18. In other embodiments the pyrimidine ribonucleotides (rU, rC) in (N′)y are substituted with nucleotides joined to the adjacent nucleotide by a 2′-5′ internucleotide bond.

In some embodiments of Structure A2, (N)y includes at least one L-DNA moiety. In some embodiments x=y=18 and (N′)y consists of unmodified ribonucleotides at positions 1-16 and 18 and one L-DNA at the 3′ penultimate position (position 17). In other embodiments x=y=18 and (N′)y consists of unmodified ribonucleotides at position 1-15 and 18 and two consecutive L-DNA at the 3′ penultimate position (positions 16 and 17). In various embodiments the unconventional moiety is a nucleotide joined to an adjacent nucleotide by a 2′-5′ internucleotide phosphate linkage. According to various embodiments (N′)y includes 2, 3, 4, 5, or 6 consecutive ribonucleotides at the 3′ terminus linked by 2′-5′ internucleotide linkages. In one embodiment, four consecutive nucleotides at the 3′ terminus of (N′)y are joined by three 2′-5′ phosphodiester bonds, wherein one or more of the 2′-5′ nucleotides which form the 2′-5′ phosphodiester bonds further includes a 3′-O-methyl (3′OMe) sugar modification. Preferably the 3′ terminal nucleotide of (N′)y includes a 2′OMe sugar modification. In certain embodiments x=y=18 and in (N′)y two or more consecutive nucleotides at positions 14, 15, 16, 17, and 18 include a nucleotide joined to an adjacent nucleotide by a 2′-5′ internucleotide bond. In various embodiments the nucleotide forming the 2′-5′ internucleotide bond includes a 3′ deoxyribose nucleotide or a 3′ methoxy nucleotide. In some embodiments x=y=18 and (N′)y includes nucleotides joined to the adjacent nucleotide by a 2′-5′ internucleotide bond between positions 15-16, 16-17 and 17-18 or between positions 16-17 and 17-18. In some embodiments x=y=18 and (N′)y includes nucleotides joined to the adjacent nucleotide by a 2′-5′ internucleotide bond between positions 14-15, 15-16, 16-17, and 17-18 or between positions 15-16, 16-17, and 17-18 or between positions 16-17 and 17-18 or between positions 17-18 or between positions 15-16 and 17-18. In other embodiments the pyrimidine ribonucleotides (rU, rC) in (N′)y are substituted with nucleotides joined to the adjacent nucleotide by a 2′-5′ internucleotide bond.

In some embodiments, x=y=19 and (N′)y comprises five consecutive nucleotides at the 3′ terminus joined by four 2′-5′ linkages, specifically the linkages between the nucleotides position 15-16, 16-17, 17-18 and 18-19.

In some embodiments the internucleotide linkages include phosphodiester bonds. In some embodiments x=y=19 and (N′)y comprises five consecutive nucleotides at the 3′ terminus joined by four 2′-5′ linkages and optionally further includes Z′ and z′ independently selected from an inverted abasic moiety and a C3 alkyl [C3; 1,3-propanediol mono(dihydrogen phosphate)] cap.

In some embodiments x=y=19 and (N′)y comprises an L-DNA position 18; and (N′)y optionally further includes Z′ and z′ independently selected from an inverted abasic moiety and a C3 alkyl [C3; 1,3-propanediol mono(dihydrogen phosphate)] cap.

In some embodiments (N′)y comprises a 3′ terminal phosphate. In some embodiments (N′)y comprises a 3′ terminal hydroxyl.

In some embodiments x=y=19 and (N)x includes 2′OMe sugar modified ribonucleotides at positions 1, 3, 5, 7, 9, 11, 13, 15, 17, 19 or at positions 2, 4, 6, 8, 11, 13, 15, 17, 19. In some embodiments x=y=19 and (N)x includes 2′OMe sugar modified pyrimidines. In some embodiments all pyrimidines in (N)x include the 2′OMe sugar modification.

In some embodiments x=y=18 and N2 is a riboadenine moiety.

In some embodiments in x=y=18, and N2-(N′)y comprises five consecutive nucleotides at the 3′ terminus joined by four 2′-5′ linkages, specifically the linkages between the nucleotides position 15-16, 16-17, 17-18 and 18-19. In some embodiments the linkages include phosphodiester bonds.

In some embodiments x=y=18 and N2-(N′)y comprises five consecutive nucleotides at the 3′ terminus joined by four 2′-5′ linkages and optionally further includes Z′ and z′ independently selected from an inverted abasic moiety and a C3 alkyl [C3; 1,3-propanediol mono(dihydrogen phosphate)] cap.

In some embodiments x=y=18 and N2-(N′)y comprises an L-DNA position 18; and (N′)y optionally further includes Z′ and z′ independently selected from an inverted abasic moiety and a C3 alkyl [C3; 1,3-propanediol mono(dihydrogen phosphate)] cap.

In some embodiments N2-(N′)y comprises a 3′ terminal phosphate. In some embodiments N2-(N′)y comprises a 3′ terminal hydroxyl.

In some embodiments x=y=18 and N1-(N)x includes 2′OMe sugar modified ribonucleotides at positions 1, 3, 5, 7, 9, 11, 13, 15, 17, 19 or at positions 2, 4, 6, 8, 11, 13, 15, 17, 19.

In some embodiments x=y=18 and N1-(N)x includes 2′OMe sugar modified pyrimidines. In some embodiments all pyrimidines in (N)x include the 2′OMe sugar modification. In some embodiments N1-(N)x further comprises an L-DNA at position 6 or 7 (5′>3′). In other embodiments N1-(N)x further comprises a ribonucleotide which generates a 2′5′ internucleotide linkage in between the ribonucleotides at positions 5-6 or 6-7 (5′>3′)

In additional embodiments N1-(N)x further includes Z wherein Z comprises a non-nucleotide overhang. In some embodiments the non-nucleotide overhang is C3-C3 [1,3-propanediol mono(dihydrogen phosphate)]2.

In some embodiments the double stranded molecules disclosed herein, in particular molecules set forth in Tables A3, A4, A7, A8 and B3, B4, B7 and B8, include one or more of the following modifications:
a) N in at least one of positions 5, 6, 7, 8, or 9 from the 5′ terminus of the antisense strand is selected from a DNA, TNA, a 2′5′ nucleotide or a mirror nucleotide;
b) N′ in at least one of positions 9 or 10 from the 5′ terminus of the sense strand is selected from a TNA, 2′5′ nucleotide and a pseudoUridine;
c) N′ in 4, 5, or 6 consecutive positions at the 3′ terminus positions of (N′)y comprises a 2′5′ nucleotide;
d) one or more pyrimidine ribonucleotides are 2′ modified in the sense strand, the antisense strand or both the sense strand and the antisense strand.

In some embodiments the double stranded molecules in particular molecules set forth in Tables A3, A4, A7, A8 and B3, B4, B7 and B8 include a combination of the following modifications
a) the antisense strand includes a DNA, TNA, a 2′5′ nucleotide or a mirror nucleotide in at least one of positions 5, 6, 7, 8, or 9 from the 5′ terminus;
b) the sense strand includes at least one of a TNA, a 2′5′ nucleotide and a pseudoUridine in positions 9 or 10 from the 5′ terminus; and
c) one or more pyrimidine ribonucleotides are 2′ modified in the sense strand, the antisense strand or both the sense strand and the antisense strand.

In some embodiments the double stranded molecules in particular molecules set forth in Tables A3, A4, A7, A8 and B3, B4, B7 and B8 include a combination of the following modifications
a) the antisense strand includes a DNA, 2′5′ nucleotide or a mirror nucleotide in at least one of positions 5, 6, 7, 8, or 9 from the 5′ terminus;
b) the sense strand includes 4, 5, or 6 consecutive 2′5′ nucleotides at the 3′ penultimate or 3′ terminal positions; and
c) one or more pyrimidine ribonucleotides are 2′ modified in the sense strand, the antisense strand or both the sense strand and the antisense strand.

In some embodiments of Structure A1 and/or Structure A2 (N)y includes at least one unconventional moiety selected from a mirror nucleotide, a 2′5′ nucleotide and a TNA. In some embodiments the unconventional moiety is a mirror nucleotide. In various embodiments the mirror nucleotide is selected from an L-ribonucleotide (L-RNA) and an L-deoxyribonucleotide (L-DNA). In preferred embodiments the mirror nucleotide is L-DNA. In certain embodiments the sense strand comprises an unconventional moiety in position 9 or 10 (from the 5′ terminus). In preferred embodiments the sense strand includes an unconventional moiety in position 9 (from the 5′ terminus). In some embodiments the sense strand is 19 nucleotides in length and comprises 4, 5, or 6 consecutive unconventional moieties in positions 15, (from the 5′ terminus). In some embodiments the sense strand includes 4 consecutive 2′5′ ribonucleotides in positions 15, 16, 17, and 18. In some embodiments the sense strand includes 5 consecutive 2′5′ ribonucleotides in positions 15, 16, 17, 18 and 19. In various embodiments the sense strand further comprises Z′. In some embodiments Z′ includes a C3OH moiety or a C3Pi moiety.

In some embodiments of Structure A1 and/or Structure A2 (N)y comprises at least one unconventional moiety selected from a mirror nucleotide and a nucleotide joined to an adjacent nucleotide by a 2′-5′ internucleotide phosphate bond. In some embodiments the unconventional moiety is a mirror nucleotide. In various embodiments the mirror nucleotide is selected from an L-ribonucleotide (L-RNA) and an L-deoxyribonucleotide (L-DNA). In preferred embodiments the mirror nucleotide is L-DNA.

In some embodiments of Structure A1 (N′)y comprises at least one L-DNA moiety. In some embodiments x=y=19 and (N′)y consists of unmodified ribonucleotides at positions 1-17 and 19 and one L-DNA at the 3′ penultimate position (position 18). In other embodiments x=y=19 and (N′)y consists of unmodified ribonucleotides at position 1-16 and 19 and two consecutive L-DNA at the 3′ penultimate position (positions 17 and 18). In various embodiments the unconventional moiety is a nucleotide joined to an adjacent nucleotide by a 2′-5′ internucleotide phosphate linkage. According to various embodiments (N′)y comprises 2, 3, 4, 5, or 6 consecutive ribonucleotides at the 3′ terminus linked by 2′-5′ internucleotide linkages. In one embodiment, four consecutive nucleotides at the 3′ terminus of (N′)y are joined by three 2′-5′ phosphodiester bonds. In one embodiment, five consecutive nucleotides at the 3′ terminus of (N′)y are joined by four 2′-5′ phosphodiester bonds. In some embodiments, wherein one or more of the 2′-5′ nucleotides form a 2′-5′ phosphodiester bonds the nucleotide further comprises a 3′-O-methyl (3′OMe) sugar modification. In some embodiments the 3′ terminal nucleotide of (N′)y comprises a 3′OMe sugar modification. In certain embodiments x=y=19 and (N′)y comprises two or more consecutive nucleotides at positions 15, 16, 17, 18 and 19 comprise a nucleotide joined to an adjacent nucleotide by a 2′-5′ internucleotide bond. In various embodiments the nucleotide forming the 2′-5′ internucleotide bond comprises a 3′ deoxyribose nucleotide or a 3′ methoxy nucleotide. In some embodiments x=y=19 and (N′)y comprises nucleotides joined to the adjacent nucleotide by a 2′-5′ internucleotide bond between positions 15-16, 16-17 and 17-18 or between positions 16-17, 17-18 and 18-19. In some embodiments x=y=19 and (N′)y comprises nucleotides joined to the adjacent nucleotide by a 2′-5′ internucleotide bond between positions 16-17 and 17-18 or between positions 17-18 and 18-19 or between positions 15-16 and 17-18. In other embodiments the pyrimidine ribonucleotides (rU, rC) in (N′)y are substituted with nucleotides joined to the adjacent nucleotide by a 2′-5′ internucleotide bond.

In some embodiments of Structure A2 (N)y comprises at least one L-DNA moiety. In some embodiments x=y=18 and N2-(N′)y, consists of unmodified ribonucleotides at positions 1-17 and 19 and one L-DNA at the 3′ penultimate position (position 18). In other embodiments x=y=18 and N2-(N′)y consists of unmodified ribonucleotides at position 1-16 and 19 and two consecutive L-DNA at the 3′ penultimate position (positions 17 and 18). In various embodiments the unconventional moiety is a nucleotide joined to an adjacent nucleotide by a 2′-5′ internucleotide phosphate linkage. According to various embodiments N2-(N′)y comprises 2, 3, 4, 5, or 6 consecutive ribonucleotides at the 3′ terminus linked by 2′-5′ internucleotide linkages. In one embodiment, four consecutive nucleotides at the 3′ terminus of N2-(N′)y are joined by three 2′-5′ phosphodiester bonds, wherein one or more of the 2′-5′ nucleotides which form the 2′-5′ phosphodiester bonds further comprises a 3′-O-methyl (3′OMe) sugar modification. In some embodiments the 3′ terminal nucleotide of N2-(N′)y comprises a 2′OMe sugar modification. In certain embodiments x=y=18 and N2-(N′)y comprises two or more consecutive nucleotides at positions 15, 16, 17, 18 and 19 comprise a nucleotide joined to an adjacent nucleotide by a 2′-5′ internucleotide bond. In various embodiments the nucleotide forming the 2′-5′ internucleotide bond comprises a 3′ deoxyribose nucleotide or a 3′ methoxy nucleotide. In some embodiments x=y=18 and N2-(N′)y comprises nucleotides joined to the adjacent nucleotide by a 2′-5′ internucleotide bond between positions 16-17 and 17-18 or between positions 17-18 and 18-19 or between positions 15-16 and 17-18. In other embodiments the pyrimidine ribonucleotides (rU, rC) in (N′)y comprise nucleotides joined to the adjacent nucleotide by a 2′-5′ internucleotide bond.

In further embodiments of Structures A1 and A2 (N′)y comprises 1-8 modified ribonucleotides wherein the modified ribonucleotide is a deoxyribose (DNA) nucleotide. In certain embodiments (N′)y comprises 1, 2, 3, 4, 5, 6, 7, or up to 8 DNA moieties. In further embodiments of Structures A1 and A2 (N′)y includes 1-8 modified ribonucleotides wherein the modified ribonucleotide is a DNA nucleotide. In certain embodiments (N′)y includes 1, 2, 3, 4, 5, 6, 7, or up to 8 DNA moieties.

In some embodiments either Z or Z′ is present and independently includes two non-nucleotide moieties.

In additional embodiments Z and Z′ are present and each independently includes two non-nucleotide moieties.

In some embodiments each of Z and Z′ includes an abasic moiety, for example a deoxyriboabasic moiety (referred to herein as “dAb”) or riboabasic moiety (referred to herein as “rAb”). In some embodiments each of Z and/or Z′ includes two covalently linked abasic moieties and is for example dAb-dAb or rAb-rAb or dAb-rAb or rAb-dAb, wherein each moiety is covalently attached to an adjacent moiety, preferably via a phospho-based bond. In some embodiments the phospho-based bond includes a phosphorothioate, a phosphonoacetate or a phosphodiester bond. In preferred embodiments the phospho-based bond includes a phosphodiester bond.

In some embodiments each of Z and/or Z′ independently includes an alkyl moiety, optionally propane [(CH2)3] moiety (C3) or a derivative thereof including propanol (C3-OH) and phospho derivative of propanediol (“C3-3′Pi”). In some embodiments each of Z and/or Z′ includes two alkyl moieties and in some examples is C3-C3-OH. The 3′ terminus of the antisense strand and/or the 3′ terminus of the sense strand is covalently attached to a C3 moiety via a phospho-based bond and the C3 moiety is covalently conjugated a C3-OH moiety via a phospho-based bond. In some embodiments the phospho-based bonds include a phosphorothioate, a phosphonoacetate or a phosphodiester bond. In preferred embodiments the phospho-based bond includes a phosphodiester bond.

In one specific embodiment of Structure A1 or Structure A2, Z includes C3-C3-OH (a propyl moiety covalently linked to a propanol moiety via a phosphodiester bond). In some embodiments Z includes a propanol moiety covalently attached to the 3′ terminus of the antisense strand via a phosphodiester bond. In some embodiments the C3-C3-OH overhang is covalently attached to the 3′ terminus of (N)x or (N′)y via covalent linkage, for example a phosphodiester linkage. In some embodiments the linkage between a first C3 and a second C3 is a phosphodiester linkage.

In various embodiments the alkyl moiety is a C3 alkyl (“C3”) to C6 alkyl (“C6”) (e.g. C3, C4, C5 or C6) moiety including a terminal hydroxyl, a terminal amino, terminal phosphate group.

In some embodiments the alkyl moiety is a C3 alkyl moiety. In some embodiments the C3 alkyl moiety includes propanol, propylphosphate, propylphosphorothioate or a combination thereof.

The C3 alkyl moiety may be covalently linked to the 3′ terminus of (N′)y and or the 3′ terminus of (N)x via a phosphodiester bond. In some embodiments the alkyl moiety includes propanol, propyl phosphate (trimethyl phosphate) or propyl phosphorothioate (trimethyl phosphorothioate).

In some embodiments each of Z and Z′ is independently selected from propanol, propyl phosphate (trimethyl phosphate), propyl phosphorothioate (trimethyl phosphorothioate), combinations thereof or multiples thereof.

In some embodiments each of Z and Z′ is independently selected from propyl phosphate (trimethyl phosphate), propyl phosphorothioate (trimethyl phosphorothioate), propyl phospho-propanol; propyl phospho-propyl phosphorothioate; propylphospho-propyl phosphate; (propyl phosphate)3, (propyl phosphate)-2-propanol, (propyl phosphate)2-propyl phosphorothioate. Any propane or propanol conjugated moiety can be included in Z or Z′.

In additional embodiments each of Z and/or Z′ includes a combination of an abasic moiety and an unmodified deoxyribonucleotide or ribonucleotide or a combination of a hydrocarbon moiety and an unmodified deoxyribonucleotide or ribonucleotide or a combination of an abasic moiety (deoxyribo or ribo) and a hydrocarbon moiety. In such embodiments, each of Z and/or Z′ includes C3-rAb or C3-dAb wherein each moiety is covalently bond to the adjacent moiety via a phospho-based bond, preferably a phosphodiester, phosphorothioate or phosphonoacetate bond.

In certain embodiments nucleic acid molecules as disclosed herein include a sense oligonucleotide sequence selected from any one of Tables A1-B8.

In some embodiments, provided is a tandem structure and a triple armed structure, also known as RNAstar. Such structures are disclosed in PCT patent publication WO 2007/091269. A tandem oligonucleotide comprises at least two siRNA compounds.

A triple-stranded oligonucleotide may be an oligoribonucleotide having the general structure:

| | | | | |
|---|---|---|---|---|
| 5′ | oligo1 (sense) | LINKER A | Oligo2 (sense) | 3′ |
| 3′ | oligo1 (antisense) | LINKER B | Oligo3 (sense) | 5′ |
| 3′ | oligo3 (antisense) | LINKER C | oligo2 (antisense) | 5′ |
| | or | | | |
| 5′ | oligo1 (sense) | LINKER A | Oligo2 (antisense) | 3′ |
| 3′ | oligo1 (antisense) | LINKER B | Oligo3 (sense) | 5′ |
| 3′ | oligo3 (antisense) | LINKER C | oligo2 (sense) | 5′ |
| | or | | | |
| 5′ | oligo1 (sense) | LINKER A | oligo3 (antisense) | 3′ |
| 3′ | oligo1 (antisense) | LINKER B | oligo2 (sense) | 5′ |
| 5′ | oligo3 (sense) | LINKER C | oligo2 (antisense) | 3′ |

wherein one or more of linker A, linker B or linker C is present; any combination of two or more oligonucleotides and one or more of linkers A-C is possible, so long as the polarity of the strands and the general structure of the molecule remains. Further, if two or more of linkers A-C are present, they may be identical or different.

In some embodiments a “gapped” RNAstar compound is preferred wherein the compound consists of four ribonucleotide strands forming three siRNA duplexes having the general structure as follows:

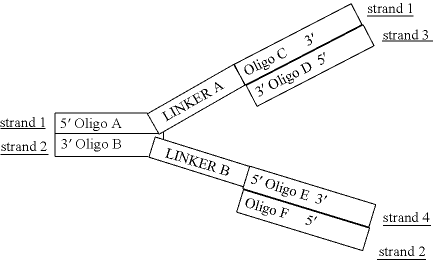

wherein each of oligo A, oligo B, oligo C, oligo D, oligo E and oligo F represents at least 19 consecutive ribonucleotides, wherein from 19 to 40 of such consecutive ribonucleotides, in each of oligo A, B, C, D, E and F comprise a strand of a siRNA duplex, wherein each ribonucleotide may be modified or unmodified;
wherein strand 1 comprises oligo A which is either a sense portion or an antisense portion of a first siRNA duplex of the compound, strand 2 comprises oligo B which is complementary to at least 19 nucleotides in oligo A, and oligo A and oligo B together form a first siRNA duplex that targets a first target mRNA;
wherein strand 1 further comprises oligo C which is either a sense portion or an antisense strand portion of a second siRNA duplex of the compound, strand 3 comprises oligo D which is complementary to at least 19 nucleotides in oligo C and oligo C and oligo D together form a second siRNA duplex that targets a second target mRNA;
wherein strand 4 comprises oligo E which is either a sense portion or an antisense strand portion of a third siRNA duplex of the compound, strand 2 further comprises oligo F which is complementary to at least 19 nucleotides in oligo E and oligo E and oligo F together form a third siRNA duplex that targets a third target mRNA; and
wherein linker A is a moiety that covalently links oligo A and oligo C; linker B is a moiety that covalently links oligo B and oligo F, and linker A and linker B can be the same or different.

In some embodiments the first, second and third siRNA duplex target the same gene, In other embodiments two of the first, second or third siRNA duplexes target the same mRNA and the third siRNA duplex targets a different mRNA. In some embodiments each of the first, second and third duplex targets a different mRNA.

In another aspect, provided are methods for reducing the expression of TIMP1 and TIMP2 in a cell by introducing into a cell a nucleic acid molecule as provided herein in an amount sufficient to reduce expression of TIMP1 and TIMP2. In one embodiment, the cell is hepatocellular stellate cell. In another embodiment, the cell is a stellate cell in renal or pulmonary tissue. In certain embodiments, the method is performed in vitro, in other embodiments, the method is performed in vivo.

In yet another aspect, provided are methods for treating an individual suffering from a disease associated with TIMP1 and/or TIMP2. The methods include administering to the individual a nucleic acid molecule such as provided herein in an amount sufficient to reduce expression of TIMP1 or TIMP2. In certain embodiments the disease associated with TIMP1 or TIMP2 is a disease selected from the group consisting of liver fibrosis, cirrhosis, pulmonary fibrosis including lung fibrosis (including ILF), any condition causing kidney fibrosis (e.g., CKD including ESRD), peritoneal fibrosis, chronic hepatic damage, fibrillogenesis, fibrotic diseases in other organs, abnormal scarring (keloids) associated with all possible types of skin injury accidental and jatrogenic (operations); scleroderma; cardiofibrosis, fibrosis in the brain; failure of glaucoma filtering operation; and intestinal adhesions. The compounds are useful in treating organ specific indications including those shown in Table I below:

TABLE I

| Organ | Indication |
|---|---|
| Skin | Pathologic scarring as keloid and hypertrophic scar |
| | Surgical scarring |
| | Injury scarring |
| | keloid, or nephrogenic fibrosing dermatopathy |
| Peritoneum | Peritoneal fibrosis |
| | Adhesions |
| | Peritoneal Sclerosis associated with continual ambulatory peritoneal dialysis |
| | (CAPD) |
| Liver | Cirrhosis including post-hepatitis C cirrhosis, primary biliary cirrhosis |
| | Liver fibrosis, e.g. Prevention of Liver Fibrosis in Hepatitis C carriers |
| | schistomasomiasis |
| | cholangitis |
| | Liver cirrhosis due to Hepatitis C post liver transplant or Non-Alcoholic |
| | Steatohepatitis (NASH) |
| Pancreas | inter(peri)lobular fibrosis (as in alcoholic chronic pancreatitis), periductal |
| | fibrosis (as in hereditary pancreatitis), periductal and interlobular fibrosis (as |
| | in autoimmune pancreatitis), diffuse inter- and intralobular fibrosis (as in |
| | obstructive chronic pancreatitis) |
| Kidney | Chronic Kidney Disease (CKD) of any etiology. Treatment of early stage |
| | CKD (elevated SCr) in diabetic patients (“prevent” further deterioration in |
| | renal function) |
| | kidney fibrosis associated with lupus glomeruloschelerosis |
| | Diabetic Nephropathy |
| Heart | Congestive heart failure, |
| | Endomyocardial fibrosis, |
| | cardiofibrosis |
| | fibrosis associated with myocardial infarction |
| Lung | Asthma, Idiopathic pulmonary fibrosis (IPF); Radiation fibrosis, a sequel of |
| | radiation pneumonitis (e.g. due to cancer treating radiation) |
| | Interstitial lung fibrosis (ILF) |
| | Radiation Pneumonitis leading to Pulmonary Fibrosis (e.g. due to cancer |
| | treating radiation) |
| Bone marrow | Myeloproliferative disorders: Myelofibrosis (MF), Polycythemia vera (PV), |
| | Essential thrombocythemia (ET) |
| | idiopathic myelofibrosis |
| | drug induced myelofibrosis. |
| Eye | Anterior segment: Corneal opacification e,g, following inherited dystrophies, |
| | herpetic keratitis or pterygia; Glaucoma |
| | Posterior segment fibrosis and traction retinal detachment, a complication of |
| | advanced diabetic retinopathy (DR); Fibrovascular scarring and gliosis in the |
| | retina; |
| | Under the retina fibrosis for example subsequent to subretinal hemorrhage |
| | associated with neovascular AMD |
| | Retro-orbital fibrosis, postcataract surgery, proliferative vitreoretinopathy. |
| | Ocular cicatricial pemphigoid |
| Intestine | Intestinal fibrosis, Crohn's disease |
| Vocal cord | Vocal cord scarring, vocal cord mucosal fibrosis, laryngeal fibrosis |
| Vasculature | Atherosclerosis, postangioplasty arterial restenosis |
| Brain | Fibrosis associated with brain (cerebral) infarction |
| Multisystemic | Scleroderma systemic sclerosis; multifocal fibrosclerosis; sclerodermatous |
| | graft-versus-host disease in bone marrow transplant recipients, and |
| | nephrogenic systemic fibrosis (exposure to gadolinium-based contrast agents |
| | (GBCAs), 30% of MRIs) |
| Malignancies | Metastatic and invasive cancer by inhibiting function of activated tumor |
| of various | associated myofibroblasts |
| origin | |

In some embodiments the preferred indications include, Liver cirrhosis due to Hepatitis C post liver transplant; Liver cirrhosis due to Non-Alcoholic Steatohepatitis (NASH); Idiopathic Pulmonary Fibrosis (IPF); Radiation Pneumonitis leading to Pulmonary Fibrosis; Diabetic Nephropathy; Peritoneal Sclerosis associated with continual ambulatory peritoneal dialysis (CAPD) and Ocular cicatricial pemphigoid.

Fibrotic Liver indications include Alcoholic Cirrhosis, Hepatitis B cirrhosis, Hepatitis C cirrhosis, Hepatitis C (Hep C) cirrhosis post orthotopic liver transplant (OLTX), NASH/NAFLD wherein NASH is an extreme form of nonalcoholic fatty liver disease (NAFLD), Primary biliary cirrhosis (PBC), Primary sclerosing cholangitis (PSC), Biliary atresia, alpha1 antitrypsin deficiency (A1AD), Copper storage diseases (Wilson's disease), Fructosemia, Galactosemia, Glycogen storage diseases (especially types III, IV, VI, IX, and X), Iron-overload syndromes (hemochromatosis), Lipid abnormalities (e.g., Gaucher's disease). Peroxisomal disorders (eg, Zellweger syndrome), Tyrosinemia, Congenital hepatic fibrosis, Bacterial Infections (eg, brucellosis), Parasitic (eg, echinococcosis), Budd-Chiari syndrome (hepatic veno-occlusive disease).

Pulmonary Indications indications include Idiopathic Pulmonary Fibrosis, Silicosis, Pneumoconiosis, Bronchopulmonary Dysplasia in newborn following neonatal respiratory distress syndrome, Bleomycin/chemotherapeutic induced lung injury, Brochiolitis Obliterans (BOS) post lung transplant, Chronic obstructive pulmonary disorder (COPD), Cystic Fibrosis, Asthma.

Cardiac indications include Cardiomyopathy, Atherosclerosis (Bergers disease, etc), Endomyocardial fibrosis, Atrial Fibrillation, Scarring post Myocardial Infarction (MI).

Other Thoracic indications include Radiation-induced capsule tissue reactions around textured breast implants and Oral submucosal fibrosis.

Renal indications include Autosomal Dominant Polycystic Kidney Disease (ADPKD), Diabetic nephropathy (diabetic glomerulosclerosis), FSGS (collapsing vs. other histologic variants), IgA Nephropathy (Berger Disease), Lupus Nephritis, Wegner's, Scleroderma, Goodpasture Syndrome, tubulointerstitial fibrosis: drug induced (protective) pencillins, cephalosporins, analgesic nephropathy, Membranoproliferative glomerulonephritis (MPGN), Henoch-Schonlein Purpura, Congenital nephropathies: Medullary Cystic Disease, Nail-Patella Syndrome and Alport Syndrome.

Bone Marrow indications include lympangiolyomyositosis (LAM), Chronic graft vs. host disease, Polycythemia vera, Essential thrombocythemia, Myelofibrosis.

Ocular indications include Retinopathy of Prematurity (RoP), Ocular cicatricial pemphigoid, Lacrimal gland fibrosis, Retinal attachment surgery, Corneal opacity, Herpetic keratitis, Pterygia, Glaucoma, Age-related macular degeneration (AMD/ARMD), Retinal fibrosis associated Diabetes mellitus (DM) retinopathy.

Brain indications include fibrosis associated with brain infarction.

Gynecological indications include Endometriosis add on to hormonal therapy for prevention of scarring, post STD fibrosis/salphingitis.

Systemic indications include Dupuytren's disease, palmar fibromatosis, Peyronie's disease, Ledderhose disease, keloids, multifocal fibrosclerosis, nephrogenic systemic fibrosis, nephrogenic myelofibrosis (anemia).

Injury Associated Fibrotic Diseases include Burn (chemical included) induced skin & soft tissue scarring and contraction, Radiation induce skin & organ scarring post cancer therapeutic radiation treatment, Keloid (skin).

Surgical indications include peritoneal fibrosis post peritoneal dialysis catheter, corneal implant, cochlear implant, other implants, silicone implants in breasts, chronic sinusitis; adhesions, pseudointimal hyperplasia of dialysis grafts.

Other indications include Chronic Pancreatitis.

In some embodiments the methods include administering to the individual a nucleic acid molecule such as provided herein in an amount sufficient to reduce expression of TIMP1. In some embodiments the methods include administering to the individual a nucleic acid molecule such as provided herein in an amount sufficient to reduce expression of TIMP2. In some embodiments the methods include administering to the individual nucleic acid molecules such as provided herein in an amount sufficient to reduce expression of TIMP1. In some embodiments the methods include administering to the individual nucleic acid molecules such as provided herein in an amount sufficient to reduce expression of TIMP2. In some embodiments provided is a nucleic acid disclosed herein for the treatment of a fibrotic disease selected from a disease or disorder set forth in Table I. In another embodiment provided is a nucleic acid molecule for use in therapy. In some embodiments therapy comprises treatment of a fibrotic disease or disorder set forth in Table I. In some embodiments provided is use of a nucleic acid molecule disclosed herein for the preparation of a medicament useful in treating a fibrotic disease or disorder set forth in Table I. In some embodiments the nucleic acid molecule is set forth in Table C, e.g. TIMP1-A, TIMP1-B, TIMP1-C. In some embodiments the nucleic acid molecule is set forth in Table D, e.g. TIMP2-A, TIMP2-B, TIMP2-C, TIMP2-D, TIMP2-E. In some embodiments the sense and antisense sequences of the nucleic acid molecule are selected from the sequence pairs set forth in any one of Table A3, Table A4, Table A7 or Table A8. In some embodiments the sense and antisense sequences of the nucleic acid molecule are selected from the sequence pairs set forth in any one of Table B3, Table B4, Table B7 or Table B8.

In one aspect, provided are pharmaceutical compositions that include a nucleic acid molecule (e.g., an siNA molecule) as described herein in a pharmaceutically acceptable carrier. In certain embodiments, the pharmaceutical formulation includes, or involves, a delivery system suitable for delivering nucleic acid molecules (e.g., siNA molecules) to an individual such as a patient; for example delivery systems described in more detail below.

In a related aspect, provided are compositions or kits that include a nucleic acid molecule (e.g., an siNA molecule) packaged for use by a patient. The package may be labeled or include a package label or insert that indicates the content of the package and provides certain information regarding how the nucleic acid molecule (e.g., an siNA molecule) should be or can be used by a patient, for example the label may include dosing information and/or indications for use. In certain embodiments the contents of the label will bear a notice in a form prescribed by a government agency, for example the United States Food and Drug Administration (FDA). In certain embodiments, the label may indicate that the nucleic acid molecule (e.g., an siNA molecule) is suitable for use in treating a patient suffering from a disease associated with TIMP1 or TIMP2; for example, the label may indicate that the nucleic acid molecule (e.g., an siNA molecule) is suitable for use in treating fibroids; or for example the label may indicate that the nucleic acid molecule (e.g., an siNA molecule) is suitable for use in treating a disease selected from the group consisting of fibrosis, liver fibrosis, cirrhosis, pulmonary fibrosis, kidney fibrosis, peritoneal fibrosis, chronic hepatic damage, and fibrillogenesis.

As used herein, the term “tissue inhibitor of metalloproteinases 1” or “TIMP1” are used interchangeably and refer to any tissue inhibitor of metalloproteinases 1 peptide, or polypeptide having any TIMP1 protein activity. Tissue inhibitor of metalloproteinases 1 is a natural inhibitor of matrix metalloproteinases. In certain preferred embodiments, “TIMP1” refers to human TIMP1. Tissue inhibitor of metalloproteinases 1 (or more particularly human TIMP1) may have an amino acid sequence that is the same, or substantially the same, as SEQ ID NO. 3 (FIG. 1C).

As used herein, the term “tissue inhibitor of metalloproteinases 2” or “TIMP2” are used interchangeably and refer to any tissue inhibitor of metalloproteinases 2 peptide, or polypeptide having any TIMP2 protein activity. Tissue inhibitor of metalloproteinases 2 (or more particularly human TIMP2) may have an amino acid sequence that is the same, or substantially the same, as SEQ ID NO. 4 (FIG. 1D).

As used herein the term “nucleotide sequence encoding TIMP1 and TIMP2” means a nucleotide sequence that codes for a TIMP1 and TIMP2 protein or portion thereof. The term “nucleotide sequence encoding TIMP1 and TIMP2” is also meant to include TIMP1 and TIMP2 coding sequences such as TIMP1 and TIMP2 isoforms, mutant TIMP1 and TIMP2 genes, splice variants of TIMP1 and TIMP2 genes, and TIMP1 and TIMP2 gene polymorphisms. A nucleic acid sequence encoding TIMP1 and TIMP2 includes mRNA sequences encoding TIMP1 and TIMP2, which can also be referred to as “TIMP1 and TIMP2 mRNA.” Exemplary sequences of human TIMP1 mRNA and TIMP2 mRNA are set forth as SEQ ID. NO. 1 and SEQ ID NO:2, respectively.

As used herein, the term “nucleic acid molecule” or “nucleic acid” are used interchangeably and refer to an oligonucleotide, nucleotide or polynucleotide. Variations of “nucleic acid molecule” are described in more detail herein. A nucleic acid molecule encompasses both modified nucleic acid molecules and unmodified nucleic acid molecules as described herein. A nucleic acid molecule may include deoxyribonucleotides, ribonucleotides, modified nucleotides or nucleotide analogs in any combination.

As used herein, the term “nucleotide” refers to a chemical moiety having a sugar (or an analog thereof, or a modified sugar), a nucleotide base (or an analog thereof, or a modified base), and a phosphate group (or analog thereof, or a modified phosphate group). A nucleotide encompasses both modified nucleotides or unmodified nucleotides as described herein. As used herein, nucleotides may include deoxyribonucleotides (e.g., unmodified deoxyribonucleotides), ribonucleotides (e.g., unmodified ribonucleotides), and modified nucleotide analogs including, inter alia, locked nucleic acids and unlocked nucleic acids, peptide nucleic acids, L-nucleotides (also referred to as mirror nucleotides), ethylene-bridged nucleic acid (ENA), arabinoside, PACE, nucleotides with a 6 carbon sugar, as well as nucleotide analogs (including abasic nucleotides) often considered to be non-nucleotides. In some embodiments, nucleotides may be modified in the sugar, nucleotide base and/or in the phosphate group with any modification known in the art, such as modifications described herein. A “polynucleotide” or “oligonucleotide” as used herein refer to a chain of linked nucleotides; polynucleotides and oligonucleotides may likewise have modifications in the nucleotide sugar, nucleotide bases and phosphate backbones as are well known in the art and/or are disclosed herein.

As used herein, the term “short interfering nucleic acid”, “siNA”, or “short interfering nucleic acid molecule” refers to any nucleic acid molecule capable of modulating gene expression or viral replication. Preferably siNA inhibits or down regulates gene expression or viral replication. siNA includes without limitation nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. As used herein, “short interfering nucleic acid”, “siNA”, or “short interfering nucleic acid molecule” has the meaning described in more detail elsewhere herein.

As used herein, the term “complementary” means that a nucleic acid can form hydrogen bond(s) with another nucleic acid sequence by either traditional Watson-Crick or other non-traditional types. In reference to the nucleic molecules disclosed herein, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). “Fully complementary” means that all the contiguous residues of a nucleic acid sequence will hydrogen bond with the same number of contiguous residues in a second nucleic acid sequence. In one embodiment, a nucleic acid molecule disclosed herein includes about 15 to about 35 or more (e.g., about 15, 16, 17, 18, 19, 20, 21, 22, 23, 24, 25, 26, 27, 28, 29, 30, 31, 32, 33, 34 or 35 or more) nucleotides that are complementary to one or more target nucleic acid molecules or a portion thereof.

As used herein, the term “sense region” refers to a nucleotide sequence of a siNA molecule complementary (partially or fully) to an antisense region of the siNA molecule. The sense strand of a siNA molecule can include a nucleic acid sequence having homology with a target nucleic acid sequence. As used herein, “sense strand” refers to nucleic acid molecule that includes a sense region and may also include additional nucleotides.

As used herein, the term “antisense region” refers to a nucleotide sequence of a siNA molecule complementary (partially or fully) to a target nucleic acid sequence. The antisense strand of a siNA molecule can optionally include a nucleic acid sequence complementary to a sense region of the siNA molecule. As used herein, “antisense strand” refers to nucleic acid molecule that includes an antisense region and may also include additional nucleotides.

As used herein, the term “RNA” refers to a molecule that includes at least one ribonucleotide residue.

As used herein, the term “duplex region” refers to the region in two complementary or substantially complementary oligonucleotides that form base pairs with one another, either by Watson-Crick base pairing or any other manner that allows for a duplex between oligonucleotide strands that are complementary or substantially complementary. For example, an oligonucleotide strand having 21 nucleotide units can base pair with another oligonucleotide of 21 nucleotide units, yet only 19 bases on each strand are complementary or substantially complementary, such that the “duplex region” consists of 19 base pairs. The remaining base pairs may, for example, exist as 5′ and 3′ overhangs. Further, within the duplex region, 100% complementarity is not required; substantial complementarity is allowable within a duplex region. Substantial complementarity refers to complementarity between the strands such that they are capable of annealing under biological conditions. Techniques to empirically determine if two strands are capable of annealing under biological conditions are well know in the art. Alternatively, two strands can be synthesized and added together under biological conditions to determine if they anneal to one another.

As used herein, the terms “non-pairing nucleotide analog” means a nucleotide analog which includes a non-base pairing moiety including but not limited to: 6 des amino adenosine (Nebularine), 4-Me-indole, 3-nitropyrrole, 5-nitroindole, Ds, Pa, N3-Me ribo U, N3-Me riboT, N3-Me dC, N3-Me-dT, N1-Me-dG, N1-Me-dA, N3-ethyl-dC, N3-Me dC. In some embodiments the non-base pairing nucleotide analog is a ribonucleotide. In other embodiments it is a deoxyribonucleotide.

As used herein, the term, “terminal functional group” includes without limitation a halogen, alcohol, amine, carboxylic, ester, amide, aldehyde, ketone, ether groups.

An “abasic nucleotide” or “abasic nucleotide analog” is as used herein may also be often referred to herein and in the art as a pseudo-nucleotide or an unconventional moiety. While a nucleotide is a monomeric unit of nucleic acid, generally consisting of a ribose or deoxyribose sugar, a phosphate, and a base (adenine, guanine, thymine, or cytosine in DNA; adenine, guanine, uracil, or cytosine in RNA). an abasic or pseudo-nucleotide lacks a base, and thus is not strictly a nucleotide as the term is generally used in the art. Abasic deoxyribose moieties include for example, abasic deoxyribose-3′-phosphate; 1,2-dideoxy-D-ribofuranose-3-phosphate; 1,4-anhydro-2-deoxy-D-ribitol-3-phosphate. Inverted abasic deoxyribose moieties include inverted deoxyriboabasic; 3′,5′ inverted deoxyabasic 5′-phosphate.

The term “capping moiety” (z″) as used herein includes a moiety which can be covalently linked to the 5′ terminus of (N′)y and includes abasic ribose moiety, abasic deoxyribose moiety, modifications abasic ribose and abasic deoxyribose moieties including 2′ O alkyl modifications; inverted abasic ribose and abasic deoxyribose moieties and modifications thereof; C6-imino-Pi; a mirror nucleotide including L-DNA and L-RNA; 5′OMe nucleotide; and nucleotide analogs including 4′,5′-methylene nucleotide; 1-(β-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non bridging methylphosphonate and 5′-mercapto moieties.

Certain capping moieties may be abasic ribose or abasic deoxyribose moieties; inverted abasic ribose or abasic deoxyribose moieties; C6-amino-Pi; a mirror nucleotide including L-DNA and L-RNA. The nucleic acid molecules as disclosed herein may be synthesized using one or more inverted nucleotides, for example inverted thymidine or inverted adenine (for example see Takei, et al., 2002. JBC 277(26):23800-06).

The term “unconventional moiety” as used herein refers to non-nucleotide moieties including an abasic moiety, an inverted abasic moiety, a hydrocarbon (alkyl) moiety and derivatives thereof, and further includes a deoxyribonucleotide, a modified deoxyribonucleotide, a mirror nucleotide (L-DNA or L-RNA), a non-base pairing nucleotide analog and a nucleotide joined to an adjacent nucleotide by a 2′-5′ internucleotide phosphate bond; bridged nucleic acids including LNA and ethylene bridged nucleic acids, linkage modified (e.g. PACE) and base modified nucleotides as well as additional moieties explicitly disclosed herein as unconventional moieties.

As used herein, the term “inhibit”, “down-regulate”, or “reduce” with respect to gene expression means the expression of the gene, or level of RNA molecules or equivalent RNA molecules encoding one or more proteins or protein subunits (e.g., mRNA), or activity of one or more proteins or protein subunits, is reduced below that observed in the absence of an inhibitory factor (such as a nucleic acid molecule, e.g., an siNA, for example having structural features as described herein); for example the expression may be reduced to 90%, 80%, 70%, 60%, 50%, 40%, 30%, 20%, 10%, 5% or less than that observed in the absence of an inhibitor.

### BRIEF DESCRIPTION OF THE FIGURES

FIGS. 1A-1D show exemplary polynucleotide and polypeptide sequences. FIG. 1A shows mRNA sequence of human TIMP1 (NM_{—}003254.2 GI:73858576; SEQ ID NO:1). FIG. 1B shows mRNA sequence of TIMP2 (NM_{—}003255.4 GI:738585774; SEQ ID NO:2). FIG. 1C shows polypeptide sequence of human TIMP1 (NP_{—}003245.1 GI:4507509; SEQ ID NO:3). FIG. 1D shows polypeptide sequence of human TIMP2 (NP_{—}003246.1 GI:4507511; SEQ ID NO:4).

FIG. 2 shows knock down efficacy as determined by qPCR of TIMP1-A, TIMP1-B or TIMP1-C siRNAs (Table C) for TIMP1. The siRNA compounds were capable of knocking down the target TIMP1 gene.

FIG. 3 shows knock down efficacy as determined by qPCR TIMP2-A, TIMP2-B, TIMP2-C, TIMP2-D and TIMP2-E siRNAs (Table D). The siRNA compounds were capable of knocking down the target TIMP2 gene.

FIG. 4 shows the results of an in vivo assay testing the efficacy of siTIMP1 and siTIMP2 in treating liver fibrosis. Analysis of the liver fibrosis area was performed using Sirius red staining. The fibrotic area was calculated as the mean of 4 liver sections. The bar graph summarizes the digital quantification of staining for each group.

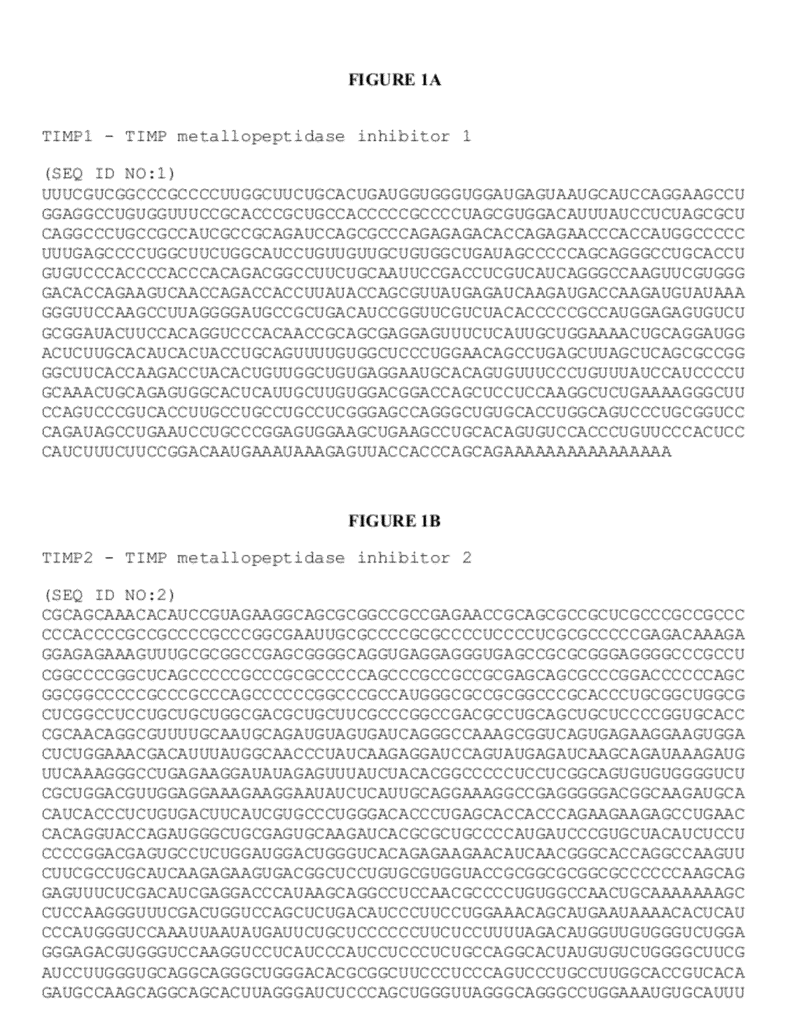

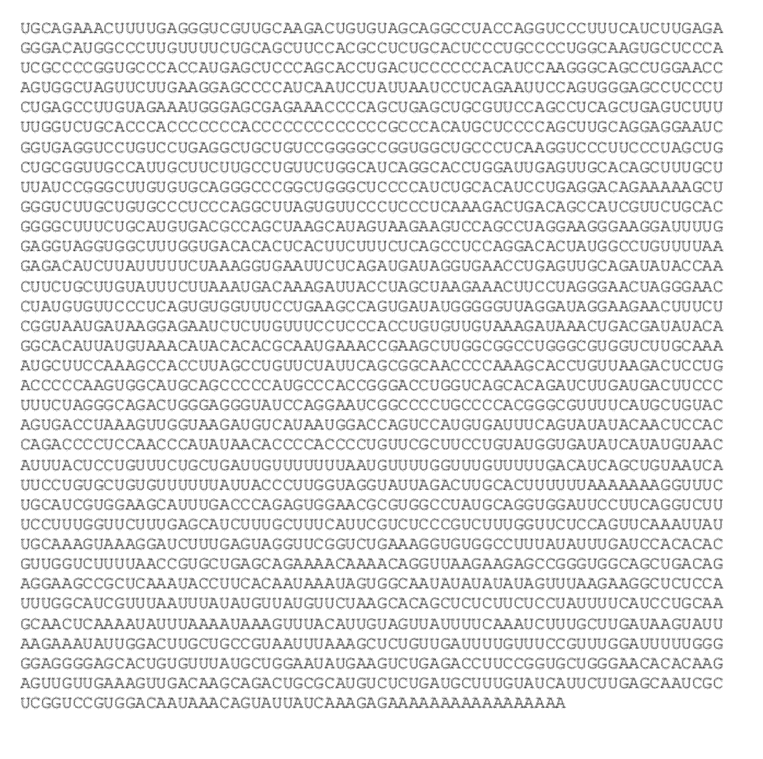

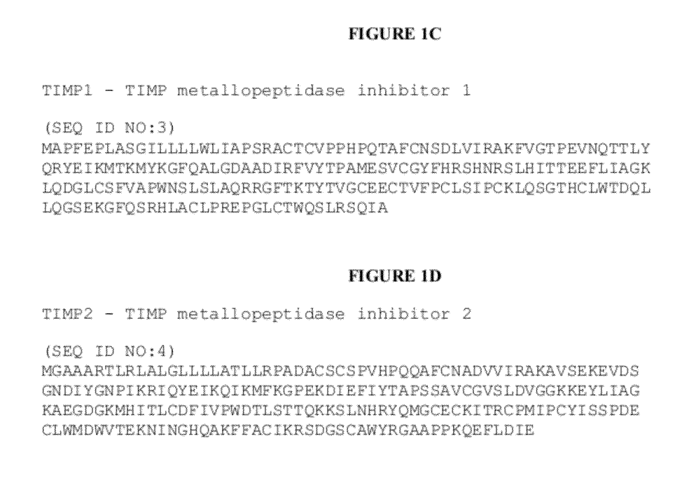

### DETAILED DESCRIPTION OF THE INVENTION

RNA Interference and siNA Nucleic Acid Molecules

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13:139-141; and Strauss, 1999, Science, 286, 886). The corresponding process in plants (Heifetz et al., International PCT Publication No. WO 99/61631) is often referred to as post-transcriptional gene silencing (PTGS) or RNA silencing. The process of post-transcriptional gene silencing is thought to be an evolutionarily-conserved cellular defense mechanism used to prevent the expression of foreign genes (Fire et al., 1999, Trends Genet., 15, 358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response through a mechanism that has yet to be fully characterized. This mechanism appears to be different from other known mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see for example U.S. Pat. Nos. 6,107,094; 5,898,031; Clemens et al., 1997, J. Interferon & Cytokine Res., 17, 503-524; Adah et al., 2001, Curr. Med. Chem., 8, 1189).

The presence of long dsRNAs in cells stimulates the activity of a ribonuclease III enzyme referred to as dicer (Bass, 2000, Cell, 101, 235; Zamore et al., 2000, Cell, 101, 25-33; Hammond et al., 2000, Nature, 404, 293). Dicer is involved in the processing of the dsRNA into short pieces of dsRNA known as short interfering RNAs (siRNAs) (Zamore et al., 2000, Cell, 101, 25-33; Bass, 2000, Cell, 101, 235; Berstein et al., 2001, Nature, 409, 363). Short interfering RNAs derived from dicer activity are typically about 21 to about 23 nucleotides in length and include about 19 base pair duplexes (Zamore et al., 2000, Cell, 101, 25-33; Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

RNAi has been studied in a variety of systems. Fire et al., 1998, Nature, 391, 806, were the first to observe RNAi in C. elegans. Bahramian and Zarbl, 1999, Molecular and Cellular Biology, 19, 274-283 and Wianny and Goetz, 1999, Nature Cell Biol., 2, 70, describe RNAi mediated by dsRNA in mammalian systems. Hammond et al., 2000, Nature, 404, 293, describe RNAi in Drosophila cells transfected with dsRNA. Elbashir et al., 2001, Nature, 411, 494 and Tuschl et al., International PCT Publication No. WO 01/75164, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J., 20, 6877 and Tuschl et al., International PCT Publication No. WO 01/75164) has revealed certain requirements for siRNA length, structure, chemical composition, and sequence that are essential to mediate efficient RNAi activity.

Nucleic acid molecules (for example having structural features as disclosed herein) may inhibit or down regulate gene expression or viral replication by mediating RNA interference “RNAi” or gene silencing in a sequence-specific manner; see e.g., Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831).

An siNA nucleic acid molecule can be assembled from two separate polynucleotide strands, where one strand is the sense strand and the other is the antisense strand in which the antisense and sense strands are self-complementary (i.e. each strand includes nucleotide sequence that is complementary to nucleotide sequence in the other strand); such as where the antisense strand and sense strand form a duplex or double stranded structure having any length and structure as described herein for nucleic acid molecules as provided, for example wherein the double stranded region (duplex region) is about 15 to about 49 (e.g., about 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 base pairs); the antisense strand includes nucleotide sequence that is complementary to nucleotide sequence in a target nucleic acid molecule (i.e., TIMP1 and TIMP2 mRNA) or a portion thereof and the sense strand includes nucleotide sequence corresponding to the target nucleic acid sequence or a portion thereof (e.g., about 17 to about 49 or more nucleotides of the nucleic acid molecules herein are complementary to the target nucleic acid or a portion thereof).

In certain aspects and embodiments a nucleic acid molecule (e.g., a siNA molecule) provided herein may be a “RISC length” molecule or may be a Dicer substrate as described in more detail below.

An siNA nucleic acid molecule may include separate sense and antisense sequences or regions, where the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions. Nucleic acid molecules may include a nucleotide sequence that is complementary to nucleotide sequence of a target gene. Nucleic acid molecules may interact with nucleotide sequence of a target gene in a manner that causes inhibition of expression of the target gene.

Alternatively, an siNA nucleic acid molecule is assembled from a single polynucleotide, where the self-complementary sense and antisense regions of the nucleic acid molecules are linked by means of a nucleic acid based or non-nucleic acid-based linker(s), i.e., the antisense strand and the sense strand are part of one single polynucleotide that having an antisense region and sense region that fold to form a duplex region (for example to form a “hairpin” structure as is well known in the art). Such siNA nucleic acid molecules can be a polynucleotide with a duplex, asymmetric duplex, hairpin or asymmetric hairpin secondary structure, having self-complementary sense and antisense regions, wherein the antisense region includes nucleotide sequence that is complementary to nucleotide sequence in a separate target nucleic acid molecule or a portion thereof and the sense region having nucleotide sequence corresponding to the target nucleic acid sequence (e.g., a sequence of TIMP1 and TIMP2 mRNA). Such siNA nucleic acid molecules can be a circular single-stranded polynucleotide having two or more loop structures and a stem comprising self-complementary sense and antisense regions, wherein the antisense region includes nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active nucleic acid molecule capable of mediating RNAi.

The following nomenclature is often used in the art to describe lengths and overhangs of siNA molecules and may be used throughout the specification and Examples. Names given to duplexes indicate the length of the oligomers and the presence or absence of overhangs. For example, a “21+2” duplex contains two nucleic acid strands both of which are 21 nucleotides in length, also termed a 21-mer siRNA duplex or a 21-mer nucleic acid and having a 2 nucleotides 3′-overhang. A “21-2” design refers to a 21-mer nucleic acid duplex with a 2 nucleotides 5′-overhang. A 21-0 design is a 21-mer nucleic acid duplex with no overhangs (blunt). A “21+2UU” is a 21-mer duplex with 2-nucleotides 3′-overhang and the terminal 2 nucleotides at the 3′-ends are both U residues (which may result in mismatch with target sequence). The aforementioned nomenclature can be applied to siNA molecules of various lengths of strands, duplexes and overhangs (such as 19-0, 21+2, 27+2, and the like). In an alternative but similar nomenclature, a “ 25/27” is an asymmetric duplex having a 25 base sense strand and a 27 base antisense strand with a 2-nucleotides 3′-overhang. A “ 27/25” is an asymmetric duplex having a 27 base sense strand and a 25 base antisense strand.

### Chemical Modifications

In certain aspects and embodiments, nucleic acid molecules (e.g., siNA molecules) as provided herein include one or more modifications (or chemical modifications). In certain embodiments, such modifications include any changes to a nucleic acid molecule or polynucleotide that would make the molecule different than a standard ribonucleotide or RNA molecule (i.e., that includes standard adenine, cytosine, uracil, or guanine moieties); which may be referred to as an “unmodified” ribonucleotide or unmodified ribonucleic acid. Traditional DNA bases and polynucleotides having a 2′-deoxy sugar represented by adenine, cytosine, thymine, or guanine moieties may be referred to as an “unmodified deoxyribonucleotide” or “unmodified deoxyribonucleic acid”; accordingly, the term “unmodified nucleotide” or “unmodified nucleic acid” as used herein refers to an “unmodified ribonucleotide” or “unmodified ribonucleic acid” unless there is a clear indication to the contrary. Such modifications can be in the nucleotide sugar, nucleotide base, nucleotide phosphate group and/or the phosphate backbone of a polynucleotide.

In certain embodiments modifications as disclosed herein may be used to increase RNAi activity of a molecule and/or to increase the in vivo stability of the molecules, particularly the stability in serum, and/or to increase bioavailability of the molecules. Non-limiting examples of modifications include without limitation internucleotide or internucleoside linkages; deoxyribonucleotides or dideoxyribonucleotides at any position and strand of the nucleic acid molecule; nucleic acid (e.g., ribonucleic acid) with a modification at the 2′-position preferably selected from an amino, fluoro, methoxy, alkoxy and alkyl; 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, biotin group, and terminal glyceryl and/or inverted deoxy abasic residue incorporation, sterically hindered molecules, such as fluorescent molecules and the like. Other nucleotides modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidi-ne (d4T) and the monophosphate nucleotides of 3′-azido-3′-deoxythymidine (AZT), 2′,3′-dideoxy-3′-thiacytidine (3TC) and 2′,3′-didehydro-2′,3′-dide-oxythymidine (d4T). Further details on various modifications are described in more detail below.

Modified nucleotides include those having a Northern conformation (e.g., Northern pseudorotation cycle, see for example Saenger, Principles of Nucleic Acid Structure, Springer-Verlag ed., 1984). Non-limiting examples of nucleotides having a northern configuration include locked nucleic acid (LNA) nucleotides (e.g., 2′-O, 4′-C-methylene-(D-ribofuranosyl)nucleotides); 2′-methoxyethoxy (MOE) nucleotides; 2′-methyl-thio-ethyl, 2′-deoxy-2′-fluoro nucleotides, 2′-deoxy-2′-chloro nucleotides, 2′-azido nucleotides, and 2′-O-methyl nucleotides. Locked nucleic acids, or LNA's are described, for example, in Elman et al., 2005; Kurreck et al., 2002; Crinelli et al., 2002; Braasch and Corey, 2001; Bondensgaard et al., 2000; Wahlestedt et al., 2000; and International Patent Publication Nos. WO 00/47599, WO 99/14226, and WO 98/39352 and WO 2004/083430. In one embodiment, an LNA is incorporated at the 5′ terminus of the sense strand.

Chemical modifications also include unlocked nucleic acids, or UNAs, which are non-nucleotide, acyclic analogues, in which the C2′-C3′ bond is not present (although UNAs are not truly nucleotides, they are expressly included in the scope of “modified” nucleotides or modified nucleic acids as contemplated herein). In particular embodiments, nucleic acid molecules with an overhang may be modified to have UNAs at the overhang positions (i.e., 2 nucleotide overhand). In other embodiments, UNAs are included at the 3′- or 5′-ends. A UNA may be located anywhere along a nucleic acid strand, i.e. at position 7. Nucleic acid molecules may contain one or more than UNA. Exemplary UNAs are disclosed in Nucleic Acids Symposium Series No. 52 p. 133-134 (2008). In certain embodiments a nucleic acid molecule (e.g., a siNA molecule) as described herein include one or more UNAs; or one UNA. In some embodiments, a nucleic acid molecule (e.g., a siNA molecule) as described herein that has a 3′-overhang include one or two UNAs in the 3′ overhang. In some embodiments a nucleic acid molecule (e.g., a siNA molecule) as described herein includes a UNA (for example one UNA) in the antisense strand; for example in position 6 or position 7 of the antisense strand. Chemical modifications also include non-pairing nucleotide analogs, for example as disclosed herein. Chemical modifications further include unconventional moieties as disclosed herein.

Chemical modifications also include terminal modifications on the 5′ and/or 3′ part of the oligonucleotides and are also known as capping moieties. Such terminal modifications are selected from a nucleotide, a modified nucleotide, a lipid, a peptide, and a sugar.

Chemical modifications also include six membered “six membered ring nucleotide analogs.” Examples of six-membered ring nucleotide analogs are disclosed in Allart, et al (Nucleosides & Nucleotides, 1998, 17:1523-1526; and Perez-Perez, et al., 1996, Bioorg. and Medicinal Chem Letters 6:1457-1460) Oligonucleotides including 6-membered ring nucleotide analogs including hexitol and altritol nucleotide monomers are disclosed in International patent application publication No. WO 2006/047842.

Chemical modifications also include “mirror” nucleotides which have a reversed chirality as compared to normal naturally occurring nucleotide; that is a mirror nucleotide may be an “L-nucleotide” analogue of naturally occurring D-nucleotide (see U.S. Pat. No. 6,602,858). Mirror nucleotides may further include at least one sugar or base modification and/or a backbone modification, for example, as described herein, such as a phosphorothioate or phosphonate moiety. U.S. Pat. No. 6,602,858 discloses nucleic acid catalysts including at least one L-nucleotide substitution. Mirror nucleotides include for example L-DNA (L-deoxyriboadenosine-3′-phosphate (mirror dA); L-deoxyribocytidine-3′-phosphate (mirror dC); L-deoxyriboguanosine-3′-phosphate (mirror dG); L-deoxyribothymidine-3′-phosphate (mirror image dT)) and L-RNA (L-riboadenosine-3′-phosphate (mirror rA); L-ribocytidine-3′-phosphate (mirror rC); L-riboguanosine-3′-phosphate (mirror rG); L-ribouracil-3′-phosphate (mirror dU).

In some embodiments, modified ribonucleotides include modified deoxyribonucleotides, for example 5′OMe DNA (5-methyl-deoxyriboguanosine-3′-phosphate) which may be useful as a nucleotide in the 5′ terminal position (position number 1); PACE (deoxyriboadenine 3′ phosphonoacetate, deoxyribocytidine 3′ phosphonoacetate, deoxyriboguanosine 3′ phosphonoacetate, deoxyribothymidine 3′ phosphonoacetate.

Modifications may be present in one or more strands of a nucleic acid molecule disclosed herein, e.g., in the sense strand, the antisense strand, or both strands. In certain embodiments, the antisense strand may include modifications and the sense strand my only include unmodified RNA.

### Nucleobases

Nucleobases of the nucleic acid disclosed herein may include unmodified ribonucleotides (purines and pyrimidines) such as adenine, guanine, cytosine, uridine. The nucleobases in one or both strands can be modified with natural and synthetic nucleobases such as thymine, xanthine, hypoxanthine, inosine, 2-aminoadenine, 6-methyl and other alkyl derivatives of adenine and guanine, any “universal base” nucleotides; 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 8-halo, amino, thiol, thioalkyl, hydroxyl and other 8-substituted adenines and guanines, 5-trifluoromethyl and other 5-substituted uracils and cytosines, 7-methylguanine, deazapurines, heterocyclic substituted analogs of purines and pyrimidines, e.g., aminoethyoxy phenoxazine, derivatives of purines and pyrimidines (e.g., 1-alkyl-, 1-alkenyl-, heteroaromatic- and 1-alkynyl derivatives) and tautomers thereof, 8-oxo-N6-methyladenine, 7-diazaxanthine, 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

### Sugar Moieties

Sugar moieties in nucleic acid disclosed herein may include 2′-hydroxyl-pentofuranosyl sugar moiety without any modification. Alternatively, sugar moieties can be modified such as, 2′-deoxy-pentofuranosyl sugar moiety, D-ribose, hexose, modification at the 2′ position of the pentofuranosyl sugar moiety such as 2′-O-alkyl (including 2′-O-methyl and 2′-O-ethyl), i.e., 2′-alkoxy, 2′-amino, 2′-O-allyl, 2′-S-alkyl, 2′-halogen (including 2′-fluoro, chloro, and bromo), 2′-methoxyethoxy, 2′-O-methoxyethyl, 2′-O-2-methoxyethyl, 2′-allyloxy (—OCH₂CH═CH₂), 2′-propargyl, 2′-propyl, ethynyl, propenyl, CF, cyano, imidazole, carboxylate, thioate, C₁ to C₁₀ lower alkyl, substituted lower alkyl, alkaryl or aralkyl, OCF₃, OCN, O-, S-, or N-alkyl; O-, S, or N-alkenyl; SOCH₃; SO₂CH₃; ONO₂; NO₂, N₃; heterozycloalkyl; heterozycloalkaryl; aminoalkylamino; polyalkylamino or substituted silyl, as, among others, for example as described in European patents EP 0 586 520 B1 or EP 0 618 925 B1.

Alkyl group includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls may have from 3-8 carbon atoms in their ring structure, and more preferably have 5 or 6 carbons in the ring structure. The term C₁-C₆ includes alkyl groups containing 1 to 6 carbon atoms. The alkyl group can be substituted alkyl group such as alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Alkoxy group includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

In some embodiments, the pentafuronosyl ring may be replaced with acyclic derivatives lacking the C2′-C3′-bond of the pentafuronosyl ring. For example, acyclonucleotides may substitute a 2-hydroxyethoxymethyl group for-the 2′-deoxyribofuranosyl sugar normally present in dNMPs.

Halogens include fluorine, bromine, chlorine, iodine.

### Backbone

The nucleoside subunits of the nucleic acid disclosed herein may be linked to each other by phosphodiester bond. The phosphodiester bond may be optionally substituted with other linkages. For example, phosphorothioate, thiophosphate-D-ribose entities, triester, thioate, 2′-5′ bridged backbone (may also be referred to as 5′-2′), PACE, 3′-(or -5′)deoxy-3′-(or -5′)thio-phosphorothioate, phosphorodithioate, phosphoroselenates, 3′-(or -5′)deoxy phosphinates, borano phosphates, 3′-(or -5′)deoxy-3′-(or 5′-)amino phosphoramidates, hydrogen phosphonates, phosphonates, borano phosphate esters, phosphoramidates, alkyl or aryl phosphonates and phosphotriester modifications such as alkylphosphotriesters, phosphotriester phosphorus linkages, 5′-ethoxyphosphodiester, P-alkyloxyphosphotriester, methylphosphonate, and nonphosphorus containing linkages for example, carbonate, carbamate, silyl, sulfur, sulfonate, sulfonamide, formacetal, thioformacetyl, oxime, methyleneimino, methylenemethylimino, methylenehydrazo, methylenedimethylhydrazo and methyleneoxymethylimino linkages.

Nucleic acid molecules disclosed herein may include a peptide nucleic acid (PNA) backbone. The PNA backbone is includes repeating N-(2-aminoethyl)-glycine units linked by peptide bonds. The various bases such as purine, pyrimidine, natural and synthetic bases are linked to the backbone by methylene carbonyl bonds.

### Terminal Phosphates

Modifications can be made at terminal phosphate groups. Non-limiting examples of different stabilization chemistries can be used, e.g., to stabilize the 3′-end of nucleic acid sequences, including (1) [3-3′]-inverted deoxyribose; (2) deoxyribonucleotide; (3) [5′-3′]-3′-deoxyribonucleotide; (4) [5′-3′]-ribonucleotide; (5) [5′-3′]-3′-O-methyl ribonucleotide; (6) 3′-glyceryl; (7) [3′-5′]-3′-deoxyribonucleotide; (8) [3′-3′]-deoxyribonucleotide; (9) [5′-2′]-deoxyribonucleotide; and (10) [5-3′]-dideoxyribonucleotide. In addition to unmodified backbone chemistries can be combined with one or more different backbone modifications described herein.

Exemplary chemically modified terminal phosphate groups include those shown below:

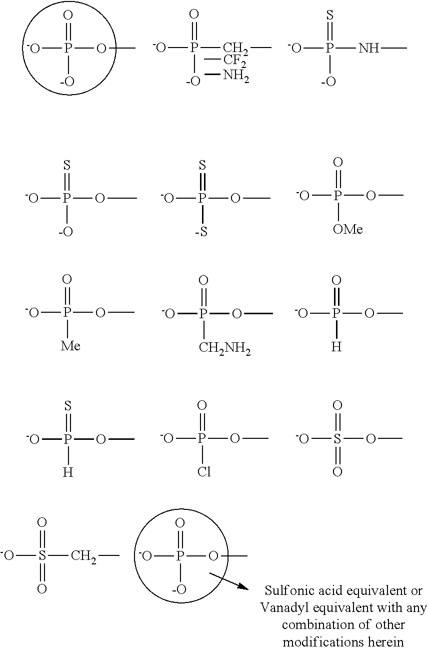

### Conjugates

Modified nucleotides and nucleic acid molecules (e.g., siNA molecules) as provided herein may include conjugates, for example, a conjugate covalently attached to the chemically-modified nucleic acid molecule. Non-limiting examples of conjugates include conjugates and ligands described in Vargeese et al., U.S. Ser. No. 10/427,160. The conjugate may be covalently attached to a nucleic acid molecule (such as an siNA molecule) via a biodegradable linker. The conjugate molecule may be attached at the 3′-end of either the sense strand, the antisense strand, or both strands of the chemically-modified nucleic acid molecule.

The conjugate molecule may be attached at the 5′-end of either the sense strand, the antisense strand, or both strands of the chemically-modified nucleic acid molecule. The conjugate molecule may be attached both the 3′-end and 5′-end of either the sense strand, the antisense strand, or both strands of the chemically-modified nucleic acid molecule, or any combination thereof. In one embodiment, a conjugate molecule may include a molecule that facilitates delivery of a chemically-modified nucleic acid molecule into a biological system, such as a cell. In another embodiment, the conjugate molecule attached to the chemically-modified nucleic acid molecule is a polyethylene glycol, human serum albumin, or a ligand for a cellular receptor that can mediate cellular uptake. Examples of specific conjugate molecules contemplated by herein that can be attached to chemically-modified nucleic acid molecules are described in Vargeese et al., U.S. Ser. No. 10/201,394.

### Linkers

A nucleic acid molecule provided herein (e.g., an siNA) molecule may include a nucleotide, non-nucleotide, or mixed nucleotide/non-nucleotide linker that joins the sense region of the nucleic acid to the antisense region of the nucleic acid. A nucleotide linker can be a linker of ≧2 nucleotides in length, for example about 3, 4, 5, 6, 7, 8, 9, or 10 nucleotides in length. The nucleotide linker can be a nucleic acid aptamer. By “aptamer” or “nucleic acid aptamer” as used herein refers to a nucleic acid molecule that binds specifically to a target molecule wherein the nucleic acid molecule has sequence that includes a sequence recognized by the target molecule in its natural setting. Alternately, an aptamer can be a nucleic acid molecule that binds to a target molecule (such as TIMP1 and TIMP2 mRNA) where the target molecule does not naturally bind to a nucleic acid. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art. See e.g., Gold et al.; 1995, Annu Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.

A non-nucleotide linker may include an abasic nucleotide, polyether, polyamine, polyamide, peptide, carbohydrate, lipid, polyhydrocarbon, or other polymeric compounds (e.g. polyethylene glycols such as those having between 2 and 100 ethylene glycol units). Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000.

### 5′ Ends, 3′ Ends and Overhangs

Nucleic acid molecules disclosed herein (e.g., siNA molecules) may be blunt-ended on both sides, have overhangs on both sides or a combination of blunt and overhang ends. Overhangs may occur on either the 5′- or 3′-end of the sense or antisense strand.

5′- and/or 3′-ends of double stranded nucleic acid molecules (e.g., siNA) may be blunt ended or have an overhang. The 5′-end may be blunt ended and the 3′-end has an overhang in either the sense strand or the antisense strand. In other embodiments, the 3′-end may be blunt ended and the 5′-end has an overhang in either the sense strand or the antisense strand. In yet other embodiments, both the 5′- and 3′-end are blunt ended or both the 5′- and 3′-ends have overhangs.

The 5′- and/or 3′-end of one or both strands of the nucleic acid may include a free hydroxyl group. The 5′- and/or 3′-end of any nucleic acid molecule strand may be modified to include a chemical modification. Such modification may stabilize nucleic acid molecules, e.g., the 3′-end may have increased stability due to the presence of the nucleic acid molecule modification. Examples of end modifications (e.g., terminal caps) include, but are not limited to, abasic, deoxy abasic, inverted (deoxy) abasic, glyceryl, dinucleotide, acyclic nucleotide, amino, fluoro, chloro, bromo, CN, CF, methoxy, imidazole, carboxylate, thioate, C₁ to C₁₀ lower alkyl, substituted lower alkyl, alkaryl or aralkyl, OCF₃, OCN, O-, S-, or N-alkyl; O-, S-, or N-alkenyl; SOCH₃; SO₂CH₃; ONO₂; NO₂, N₃; heterocycloalkyl; heterocycloalkaryl; aminoalkylamino; polyalkylamino or substituted silyl, as, among others, described in European patents EP 586,520 and EP 618,925 and other modifications disclosed herein.

Nucleic acid molecules include those with blunt ends, i.e., ends that do not include any overhanging nucleotides. A nucleic acid molecule can include one or more blunt ends. The blunt ended nucleic acid molecule has a number of base pairs equal to the number of nucleotides present in each strand of the nucleic acid molecule. The nucleic acid molecule can include one blunt end, for example where the 5′-end of the antisense strand and the 3′-end of the sense strand do not have any overhanging nucleotides. Nucleic acid molecule may include one blunt end, for example where the 3′-end of the antisense strand and the 5′-end of the sense strand do not have any overhanging nucleotides. A nucleic acid molecule may include two blunt ends, for example where the 3′-end of the antisense strand and the 5′-end of the sense strand as well as the 5′-end of the antisense strand and 3′-end of the sense strand do not have any overhanging nucleotides. Other nucleotides present in a blunt ended nucleic acid molecule can include, for example, mismatches, bulges, loops, or wobble base pairs to modulate the activity of the nucleic acid molecule to mediate RNA interference.

In certain embodiments of the nucleic acid molecules (e.g., siNA molecules) provided herein, at least one end of the molecule has an overhang of at least one nucleotide (for example 1 to 8 overhang nucleotides). For example, one or both strands of a double stranded nucleic acid molecule disclosed herein may have an overhang at the 5′-end or at the 3′-end or both. An overhang may be present at either or both the sense strand and antisense strand of the nucleic acid molecule. The length of the overhang may be as little as one nucleotide and as long as 1 to 8 or more nucleotides (e.g., 1, 2, 3, 4, 5, 6, 7 or 8 nucleotides; in some preferred embodiments an overhang is 2, 3, 4, 5, 6, 7 or 8 nucleotides; for example an overhang may be 2 nucleotides. The nucleotide(s) forming the overhang may be include deoxyribonucleotide(s), ribonucleotide(s), natural and non-natural nucleobases or any nucleotide modified in the sugar, base or phosphate group such as disclosed herein. A double stranded nucleic acid molecule may have both 5′- and 3′-overhangs. The overhangs at the 5′- and 3′-end may be of different lengths. An overhang may include at least one nucleic acid modification which may be deoxyribonucleotide. One or more deoxyribonucleotides may be at the 5′-terminal. The 3′-end of the respective counter-strand of the nucleic acid molecule may not have an overhang, more preferably not a deoxyribonucleotide overhang. The one or more deoxyribonucleotide may be at the 3′-terminal. The 5′-end of the respective counter-strand of the dsRNA may not have an overhang, more preferably not a deoxyribonucleotide overhang. The overhang in either the 5′- or the 3′-end of a strand may be 1 to 8 (e.g., about 1, 2, 3, 4, 5, 6, 7 or 8) unpaired nucleotides, preferably, the overhang is 2-3 unpaired nucleotides; more preferably 2 unpaired nucleotides. Nucleic acid molecules may include duplex nucleic acid molecules with overhanging ends of about 1 to about 20 (e.g., about 1, 2, 3, 4, 5, 6, 7, 8, 9, 10, 11, 12, 13, 1, 15, 16, 17, 18, 19 or 20); preferably 1-8 (e.g., about 1, 2, 3, 4, 5, 6, 7 or 8) nucleotides, for example, about 21-nucleotide duplexes with about 19 base pairs and 3′-terminal mononucleotide, dinucleotide, or trinucleotide overhangs. Nucleic acid molecules herein may include duplex nucleic acid molecules with blunt ends, where both ends are blunt, or alternatively, where one of the ends is blunt. Nucleic acid molecules disclosed herein can include one or more blunt ends, i.e. where a blunt end does not have any overhanging nucleotides. In one embodiment, the blunt ended nucleic acid molecule has a number of base pairs equal to the number of nucleotides present in each strand of the nucleic acid molecule. The nucleic acid molecule may include one blunt end, for example where the 5′-end of the antisense strand and the 3′-end of the sense strand do not have any overhanging nucleotides. The nucleic acid molecule may include one blunt end, for example where the 3′-end of the antisense strand and the 5′-end of the sense strand do not have any overhanging nucleotides. A nucleic acid molecule may include two blunt ends, for example where the 3′-end of the antisense strand and the 5′-end of the sense strand as well as the 5′-end of the antisense strand and 3′-end of the sense strand do not have any overhanging nucleotides. In certain preferred embodiments the nucleic acid compounds are blunt ended. Other nucleotides present in a blunt ended siNA molecule can include, for example, mismatches, bulges, loops, or wobble base pairs to modulate the activity of the nucleic acid molecule to mediate RNA interference.

In many embodiments one or more, or all, of the overhang nucleotides of a nucleic acid molecule (e.g., a siNA molecule) as described herein includes are modified such as described herein; for example one or more, or all, of the nucleotides may be 2′-deoxyribonucleotides.

### Amount, Location and Patterns of Modifications.

Nucleic acid molecules (e.g., siNA molecules) disclosed herein may include modified nucleotides as a percentage of the total number of nucleotides present in the nucleic acid molecule. As such, a nucleic acid molecule may include about 5% to about 100% modified nucleotides (e.g., about 5%, 10%, 15%, 20%, 25%, 30%, 35%, 40%, 45%, 50%, 55%, 60%, 65%, 70%, 75%, 80%, 85%, 90%, 95% or 100% modified nucleotides). The actual percentage of modified nucleotides present in a given nucleic acid molecule will depend on the total number of nucleotides present in the nucleic acid. If the nucleic acid molecule is single stranded, the percent modification can be based upon the total number of nucleotides present in the single stranded nucleic acid molecule. Likewise, if the nucleic acid molecule is double stranded, the percent modification can be based upon the total number of nucleotides present in the sense strand, antisense strand, or both the sense and antisense strands.

Nucleic acid molecules disclosed herein may include unmodified RNA as a percentage of the total nucleotides in the nucleic acid molecule. As such, a nucleic acid molecule may include about 5% to about 100% modified nucleotides (e.g., about 5%, 10%, 15%, 20%, 25%, 30%, 35%, 40%, 45%, 50%, 55%, 60%, 65%, 70%, 75%, 80%, 85%, 90%, 95% or 100% of total nucleotides present in a nucleic acid molecule.

A nucleic acid molecule (e.g., an siNA molecule) may include a sense strand that includes about 1 to about 5, specifically about 1, 2, 3, 4, or 5 phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5, or more) universal base modified nucleotides, and optionally a terminal cap molecule at the 3-end, the 5′-end, or both of the 3′- and 5′-ends of the sense strand; and wherein the antisense strand includes about 1 to about 5 or more, specifically about 1, 2, 3, 4, 5, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. A nucleic acid molecule may include about 1, 2, 3, 4, 5, 6, 7, 8, 9, 10, or more, pyrimidine nucleotides of the sense and/or antisense nucleic acid strand are chemically-modified with 2′-deoxy, 2′-O-methyl and/or 2′-deoxy-2′-fluoro nucleotides, with or without about 1 to about 5 or more, for example about 1, 2, 3, 4, 5, or more phosphorothioate internucleotide linkages and/or a terminal cap molecule at the 3′-end, the 5′-end, or both of the 3′- and 5′-ends, being present in the same or different strand.

A nucleic acid molecule may include about 1 to about 5 or more (specifically about 1, 2, 3, 4, 5 or more) phosphorothioate internucleotide linkages in each strand of the nucleic acid molecule.

A nucleic acid molecule may include 2′-5′ internucleotide linkages, for example at the 3′-end, the 5′-end, or both of the 3′- and 5′-ends of one or both nucleic acid sequence strands. In addition, the 2′-5′ internucleotide linkage(s) can be present at various other positions within one or both nucleic acid sequence strands, for example, about 1, 2, 3, 4, 5, 6, 7, 8, 9, 10, or more including every internucleotide linkage of a pyrimidine nucleotide in one or both strands of the siNA molecule can include a 2′-5′ internucleotide linkage, or about 1, 2, 3, 4, 5, 6, 7, 8, 9, 10, or more including every internucleotide linkage of a purine nucleotide in one or both strands of the siNA molecule can include a 2′-5′ internucleotide linkage.

A chemically-modified short interfering nucleic acid (siNA) molecule may include an antisense region, wherein any (e.g., one or more or all) pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any (e.g., one or more or all) purine nucleotides present in the antisense region are 2′-deoxy purine nucleotides (e.g., wherein all purine nucleotides are 2′-deoxy purine nucleotides or alternately a plurality of purine nucleotides are 2′-deoxy purine nucleotides).

A chemically-modified short interfering nucleic acid (siNA) molecule may include an antisense region, wherein any (e.g., one or more or all) pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any (e.g., one or more or all) purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides).

A chemically-modified short interfering nucleic acid (siNA) molecule capable of mediating RNA interference (RNAi) against TIMP1 and TIMP2 inside a cell or reconstituted in vitro system may include a sense region, wherein one or more pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and one or more purine nucleotides present in the sense region are 2′-deoxy purine nucleotides (e.g., wherein all purine nucleotides are 2′-deoxy purine nucleotides or alternately a plurality of purine nucleotides are 2′-deoxy purine nucleotides), and an antisense region, wherein one or more pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and one or more purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides). The sense region and/or the antisense region can have a terminal cap modification, such as any modification, that is optionally present at the 3′-end, the 5′-end, or both of the 3′ and 5′-ends of the sense and/or antisense sequence. The sense and/or antisense region can optionally further include a 3′-terminal nucleotide overhang having about 1 to about 4 (e.g., about 1, 2, 3, or 4) 2′-deoxyribonucleotides. The overhang nucleotides can further include one or more (e.g., about 1, 2, 3, 4 or more) phosphorothioate, phosphonoacetate, and/or thiophosphonoacetate internucleotide linkages. The purine nucleotides in the sense region may alternatively be 2′-O-methyl purine nucleotides (e.g., wherein all purine nucleotides are 2′-O-methyl purine nucleotides or alternately a plurality of purine nucleotides are 2′-O-methyl purine nucleotides) and one or more purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides). One or more purine nucleotides in the sense region may alternatively be purine ribonucleotides (e.g., wherein all purine nucleotides are purine ribonucleotides or alternately a plurality of purine nucleotides are purine ribonucleotides) and any purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides). One or more purine nucleotides in the sense region and/or present in the antisense region may alternatively selected from the group consisting of 2′-deoxy nucleotides, locked nucleic acid (LNA) nucleotides, 2′-methoxyethyl nucleotides, 4′-thionucleotides, and 2′-O-methyl nucleotides (e.g., wherein all purine nucleotides are selected from the group consisting of 2′-deoxy nucleotides, locked nucleic acid (LNA) nucleotides, 2′-methoxyethyl nucleotides, 4′-thionucleotides, and 2′-O-methyl nucleotides or alternately a plurality of purine nucleotides are selected from the group consisting of 2′-deoxy nucleotides, locked nucleic acid (LNA) nucleotides, 2′-methoxyethyl nucleotides, 4′-thionucleotides, and 2′-O-methyl nucleotides).

In some embodiments, a nucleic acid molecule (e.g., a siNA molecule) as described herein includes a modified nucleotide (for example one modified nucleotide) in the antisense strand; for example in position 6 or position 7 of the antisense strand.

### Modification Patterns and Alternating Modifications

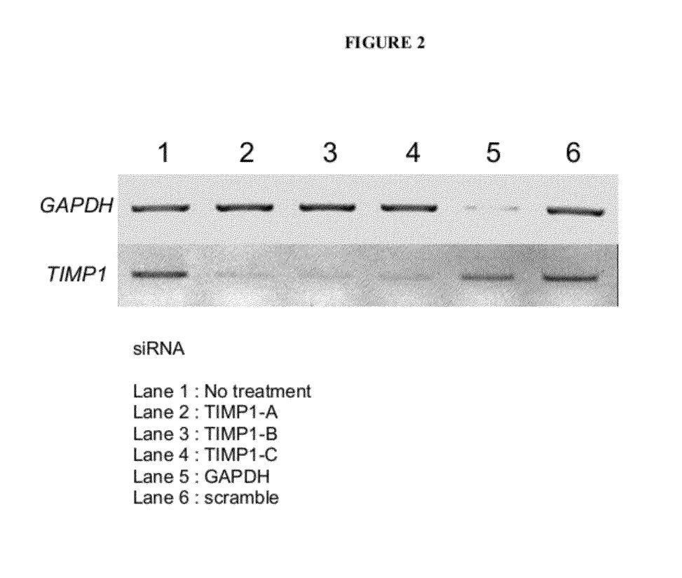

Nucleic acid molecules (e.g., siNA molecules) provided herein may have patterns of modified and unmodified nucleic acids. A pattern of modification of the nucleotides in a contiguous stretch of nucleotides may be a modification contained within a single nucleotide or group of nucleotides that are covalently linked to each other via standard phosphodiester bonds or, at least partially, through phosphorothioate bonds. Accordingly, a “pattern” as contemplated herein, does not necessarily need to involve repeating units, although it may. Examples of modification patterns that may be used in conjunction with the nucleic acid molecules (e.g., siNA molecules) provided herein include those disclosed in Giese, U.S. Pat. No. 7,452,987. For example, nucleic acid molecules (e.g., siNA molecules) provided herein include those having modification patters such as, similar to, or the same as, the patterns shown diagrammatically in FIG. 2 of the Giese U.S. Pat. No. 7,452,987.

A modified nucleotide or group of modified nucleotides may be at the 5′-end or 3′-end of the sense or antisense strand, a flanking nucleotide or group of nucleotides is arrayed on both sides of the modified nucleotide or group, where the flanking nucleotide or group either is unmodified or does not have the same modification of the preceding nucleotide or group of nucleotides. The flanking nucleotide or group of nucleotides may, however, have a different modification. This sequence of modified nucleotide or group of modified nucleotides, respectively, and unmodified or differently modified nucleotide or group of unmodified or differently modified nucleotides may be repeated one or more times.

In some patterns, the 5′-terminal nucleotide of a strand is a modified nucleotide while in other patterns the 5′-terminal nucleotide of a strand is an unmodified nucleotide. In some patterns, the 5′-end of a strand starts with a group of modified nucleotides while in other patterns, the 5′-terminal end is an unmodified group of nucleotides. This pattern may be either on the first stretch or the second stretch of the nucleic acid molecule or on both.

Modified nucleotides of one strand of the nucleic acid molecule may be complementary in position to the modified or unmodified nucleotides or groups of nucleotides of the other strand.

There may be a phase shift between modifications or patterns of modifications on one strand relative to the pattern of modification of the other strand such that the modification groups do not overlap. In one instance, the shift is such that the modified group of nucleotides of the sense strand corresponds to the unmodified group of nucleotides of the antisense strand and vice versa.

There may be a partial shift of the pattern of modification such that the modified groups overlap. The groups of modified nucleotides in any given strand may optionally be the same length, but may be of different lengths. Similarly, groups of unmodified nucleotides in any given strand may optionally be the same length, or of different lengths.

In some patterns, the second (penultimate) nucleotide at the terminus of the strand, is an unmodified nucleotide or the beginning of group of unmodified nucleotides. Preferably, this unmodified nucleotide or unmodified group of nucleotides is located at the 5′-end of the either or both the sense and antisense strands and even more preferably at the terminus of the sense strand. An unmodified nucleotide or unmodified group of nucleotide may be located at the 5′-end of the sense strand. In a preferred embodiment the pattern consists of alternating single modified and unmodified nucleotides.

In some double stranded nucleic acid molecules include a 2′-O-methyl modified nucleotide and a non-modified nucleotide, preferably a nucleotide which is not 2′-O-methyl modified, are incorporated on both strands in an alternating fashion, resulting in a pattern of alternating 2′-O-methyl modified nucleotides and nucleotides that are either unmodified or at least do not include a 2′-O-methyl modification. In certain embodiments, the same sequence of 2′-O-methyl modification and non-modification exists on the second strand; in other embodiments the alternating 2′-O-methyl modified nucleotides are only present in the sense strand and are not present in the antisense strand; and in yet other embodiments the alternating 2′-O-methyl modified nucleotides are only present in the sense strand and are not present in the antisense strand. In certain embodiments, there is a phase shift between the two strands such that the 2′-O-methyl modified nucleotide on the first strand base pairs with a non-modified nucleotide(s) on the second strand and vice versa. This particular arrangement, i.e. base pairing of 2′-O-methyl modified and non-modified nucleotide(s) on both strands is particularly preferred in certain embodiments. In certain embodiments, the pattern of alternating 2′-O-methyl modified nucleotides exists throughout the entire nucleic acid molecule; or the entire duplex region. In other embodiments the pattern of alternating 2′-O-methyl modified nucleotides exists only in a portion of the nucleic acid; or the entire duplex region.

In “phase shift” patterns, it may be preferred if the antisense strand starts with a 2′-O-methyl modified nucleotide at the 5′ end whereby consequently the second nucleotide is non-modified, the third, fifth, seventh and so on nucleotides are thus again 2′-O-methyl modified whereas the second, fourth, sixth, eighth and the like nucleotides are non-modified nucleotides.

Exemplary Modification Locations and Patterns

While exemplary patterns are provided in more detail below, all permutations of patterns with of all possible characteristics of the nucleic acid molecules disclosed herein and those known in the art are contemplated (e.g., characteristics include, but are not limited to, length of sense strand, length of antisense strand, length of duplex region, length of hangover, whether one or both ends of a double stranded nucleic acid molecule is blunt or has an overhang, location of modified nucleic acid, number of modified nucleic acids, types of modifications, whether a double overhang nucleic acid molecule has the same or different number of nucleotides on the overhang of each side, whether a one or more than one type of modification is used in a nucleic acid molecule, and number of contiguous modified/unmodified nucleotides). With respect to all detailed examples provided below, while the duplex region is shown to be 19 nucleotides, the nucleic acid molecules provided herein can have a duplex region ranging from 1 to 49 nucleotides in length as each strand of a duplex region can independently be 17-49 nucleotides in length Exemplary patterns are provided herein.

Nucleic acid molecules may have a blunt end (when n is 0) on both ends that include a single or contiguous set of modified nucleic acids. The modified nucleic acid may be located at any position along either the sense or antisense strand. Nucleic acid molecules may include a group of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 contiguous modified nucleotides. Modified nucleic acids may make up 1%, 2%, 3%, 5%, 10%, 15%, 20%, 25%, 30%, 35%, 40%, 45%, 50%, 55%, 60%, 65%, 70%, 75%, 80%, 85%, 90%, 95%, 97%, 98% or 100% of a nucleic acid strand. Modified nucleic acids of the examples immediately below may be in the sense strand only, the antisense strand only, or in both the sense and antisense strand.

General nucleic acid patters are shown below where X=sense strand nucleotide in the duplex region; Xₐ=5′-overhang nucleotide in the sense strand; X_{b}=3′-overhang nucleotide in the sense strand; Y=antisense strand nucleotide in the duplex region; Yₐ=3′-overhang nucleotide in the antisense strand; Y_{b}=5′-overhang nucleotide in the antisense strand; and M=a modified nucleotide in the duplex region. Each a and b are independently 0 to 8 (e.g., 0, 1, 2, 3, 4, 5, 6, 7 or 8). Each X, Y, a and b are independently modified or unmodified. The sense and antisense strands can are each independently 17-49 nucleotides in length. The examples provided below have a duplex region of 19 nucleotides; however, nucleic acid molecules disclosed herein can have a duplex region anywhere between 17 and 49 nucleotides and where each strand is independently between 17 and 49 nucleotides in length.

5′ XₐXXXXXXXXXXXXXXXXXXXX_{b}
3′ Y_{b}YYYYYYYYYYYYYYYYYYYYₐ

Further exemplary nucleic acid molecule patterns are shown below where X=unmodified sense strand nucleotides; x=an unmodified overhang nucleotide in the sense strand; Y=unmodified antisense strand nucleotides; y=an unmodified overhang nucleotide in the antisense strand; and M=a modified nucleotide. The sense and antisense strands can are each independently 17-49 nucleotides in length. The examples provided below have a duplex region of 19 nucleotides; however, nucleic acid molecules disclosed herein can have a duplex region anywhere between 17 and 49 nucleotides and where each strand is independently between 17 and 49 nucleotides in length.

5′ [symbol] XXXXXXXXXMXXXXXXXXX [symbol]
3′ [symbol] YYYYYYYYYYYYYYYYYYY [symbol]
5′ XXXXXXXXXXXXXXXXXXX
3′ YYYYYYYYYMYYYYYYYYY
5′ XXXXXXXXMMXXXXXXXXX
3′ YYYYYYYYYYYYYYYYYYY
5′ XXXXXXXXXXXXXXXXXXX
3′ YYYYYYYYMMYYYYYYYYY
5′ XXXXXXXXXMXXXXXXXXX
3′ YYYYYYYYYMYYYYYYYYY
5′ XXXXXMXXXXXXXXXXXXX
3′ YYYYYYYYYMYYYYYYYYY
5′ MXXXXXXXXXXXXXXXXXX
3′ YYYYYYYYYYYYMYYYYYY
5′ XXXXXXXXXXXXXXXXXXM
3′ YYYYYMYYYYYYYYYYYYY
5′ XXXXXXXXXMXXXXXXXX
3′ MYYYYYYYYYYYYYYYYY
5′ XXXXXXXMXXXXXXXXXX
3′ YYYYYYYYYYYYYYYYYM
5′ XXXXXXXXXXXXXMXXXX
3′ MYYYYYYYYYYYYYYYYY
5′ MMMMMMMMMMMMMMMMMM
3′ MMMMMMMMMMMMMMMMMM

Nucleic acid molecules may have blunt ends on both ends with alternating modified nucleic acids. The modified nucleic acids may be located at any position along either the sense or antisense strand.

5′ MXMXMXMXMXMXMXMXMXM
3′ YMYMYMYMYMYMYMYMYMY
5′ XMXMXMXMXMXMXMXMXMX
3′ MYMYMYMYMYMYMYMYMYM
5′ MMXMMXMMXMMXMMXMMXM
3′ YMMYMMYMMYMMYMMYMMY
5′ XMMXMMXMMXMMXMMXMMX
3′ MMYMMYMMYMMYMMYMMYM
5′ MMMXMMMXMMMXMMMXMMM
3′ YMMMYMMMYMMMYMMMYMM
5′ XMMMXMMMXMMMXMMMXMM
3′ MMMYMMMYMMMYMMMYMMM

Nucleic acid molecules with a blunt 5′-end and 3′-end overhang end with a single modified nucleic acid.

Nucleic acid molecules with a 5′-end overhang and a blunt 3′-end with a single modified nucleic acid.

Nucleic acid molecules with overhangs on both ends and all overhangs are modified nucleic acids. In the pattern immediately below, M is n number of modified nucleic acids, where n is an integer from 0 to 8 (i.e., 0, 1, 2, 3, 4, 5, 6, 7 and 8).

5′ XXXXXXXXXXXXXXXXXXXM
3′ MYYYYYYYYYYYYYYYYYYY

Nucleic acid molecules with overhangs on both ends and some overhang nucleotides are modified nucleotides. In the patterns immediately below, M is n number of modified nucleotides, x is n number of unmodified overhang nucleotides in the sense strand, y is n number of unmodified overhang nucleotides in the antisense strand, where each n is independently an integer from 0 to 8 (i.e., 0, 1, 2, 3, 4, 5, 6, 7 and 8), and where each overhang is maximum of 20 nucleotides; preferably a maximum of 8 nucleotides (modified and/or unmodified).

5′ XXXXXXXXXXXXXXXXXXXM
3′ yYYYYYYYYYYYYYYYYYYY
5′ XXXXXXXXXXXXXXXXXXXMx
3′ yYYYYYYYYYYYYYYYYYYY
5′ XXXXXXXXXXXXXXXXXXXMxM
3′ yYYYYYYYYYYYYYYYYYYY
5′ XXXXXXXXXXXXXXXXXXXMxMx
3′ yYYYYYYYYYYYYYYYYYYY
5′ XXXXXXXXXXXXXXXXXXXMxMxM
3′ yYYYYYYYYYYYYYYYYYYY
5′ XXXXXXXXXXXXXXXXXXXMxMxMx
3′ yYYYYYYYYYYYYYYYYYYY
5′ XXXXXXXXXXXXXXXXXXXMxMxMxM
3′ yYYYYYYYYYYYYYYYYYYY
5′ XXXXXXXXXXXXXXXXXXXMxMxMxMx
3′ yYYYYYYYYYYYYYYYYYYY
5′ MXXXXXXXXXXXXXXXXXXX
3′ YYYYYYYYYYYYYYYYYYYy
5′ xMXXXXXXXXXXXXXXXXXXX
3′ YYYYYYYYYYYYYYYYYYYy
5′ MxMXXXXXXXXXXXXXXXXXXX
3′ YYYYYYYYYYYYYYYYYYYy
5′ xMxMXXXXXXXXXXXXXXXXXXX
3′ YYYYYYYYYYYYYYYYYYYy
5′ MxMxMXXXXXXXXXXXXXXXXXXX
3′ YYYYYYYYYYYYYYYYYYYy
5′ xMxMxMXXXXXXXXXXXXXXXXXXX
3′ YYYYYYYYYYYYYYYYYYYy
5′ MxMxMxMXXXXXXXXXXXXXXXXXXX
3′ YYYYYYYYYYYYYYYYYYYy
5′ xMxMxMxMXXXXXXXXXXXXXXXXXXX
3′ YYYYYYYYYYYYYYYYYYYy
5′ xXXXXXXXXXXXXXXXXXXX
3′ YYYYYYYYYYYYYYYYYYYM
5′ xXXXXXXXXXXXXXXXXXXX
3′ YYYYYYYYYYYYYYYYYYYMy
5′ xXXXXXXXXXXXXXXXXXXX
3′ YYYYYYYYYYYYYYYYYYYMyM
5′ xXXXXXXXXXXXXXXXXXXX
3′ YYYYYYYYYYYYYYYYYYYMyMy
5′ xXXXXXXXXXXXXXXXXXXX
3′ YYYYYYYYYYYYYYYYYYYMyMyM
5′ xXXXXXXXXXXXXXXXXXXX
3′ YYYYYYYYYYYYYYYYYYYMyMyMy
5′ xXXXXXXXXXXXXXXXXXXX
3′ YYYYYYYYYYYYYYYYYYYMyMyMyM
5′ xXXXXXXXXXXXXXXXXXXX
3′ YYYYYYYYYYYYYYYYYYYMyMyMyMy
5′ XXXXXXXXXXXXXXXXXXXx
3′ MYYYYYYYYYYYYYYYYYYY
5′ XXXXXXXXXXXXXXXXXXXx
3′ yMYYYYYYYYYYYYYYYYYYY
5′ XXXXXXXXXXXXXXXXXXXx
3′ MyMYYYYYYYYYYYYYYYYYYY
5′ XXXXXXXXXXXXXXXXXXXx
3′ yMyMYYYYYYYYYYYYYYYYYYY
5′ XXXXXXXXXXXXXXXXXXXx
3′ MyMyMYYYYYYYYYYYYYYYYYYY
5′ XXXXXXXXXXXXXXXXXXXx
3′ yMyMyMYYYYYYYYYYYYYYYYYYY
5′ XXXXXXXXXXXXXXXXXXXx
3′ MyMyMyMYYYYYYYYYYYYYYYYYYY
5′ XXXXXXXXXXXXXXXXXXXx
3′ yMyMyMyMYYYYYYYYYYYYYYYYYYY

Modified nucleotides at the 3′ end of the sense region.

5′ XXXXXXXXXXXXXXXXXXXM
3′ YYYYYYYYYYYYYYYYYYY
5′ XXXXXXXXXXXXXXXXXXXMM
3′ YYYYYYYYYYYYYYYYYYY
5′ XXXXXXXXXXXXXXXXXXXMMM
3′ YYYYYYYYYYYYYYYYYYY
5′ XXXXXXXXXXXXXXXXXXXMMMM
3′ YYYYYYYYYYYYYYYYYYY
5′ XXXXXXXXXXXXXXXXXXXMMMMM
3′ YYYYYYYYYYYYYYYYYYY
5′ XXXXXXXXXXXXXXXXXXXMMMMMM
3′ YYYYYYYYYYYYYYYYYYY
5′ XXXXXXXXXXXXXXXXXXXMMMMMMMM
3′ YYYYYYYYYYYYYYYYYYY
5′ XXXXXXXXXXXXXXXXXXXMMMMMMMM
3′ YYYYYYYYYYYYYYYYYYY

Overhang at the 5′ end of the sense region.

5′ MXXXXXXXXXXXXXXXXXXX
3′ YYYYYYYYYYYYYYYYYYY
5′ MMXXXXXXXXXXXXXXXXXXX
3′ YYYYYYYYYYYYYYYYYYY
5′ MMMXXXXXXXXXXXXXXXXXXX
3′ YYYYYYYYYYYYYYYYYYY
5′ MMMMXXXXXXXXXXXXXXXXXXX
3′ YYYYYYYYYYYYYYYYYYY
5′ MMMMMXXXXXXXXXXXXXXXXXXX
3′ YYYYYYYYYYYYYYYYYYY
5′ MMMMMMXXXXXXXXXXXXXXXXXXX
3′ YYYYYYYYYYYYYYYYYYY
5′ MMMMMMMXXXXXXXXXXXXXXXXXXX
3′ YYYYYYYYYYYYYYYYYYY
5′ MMMMMMMMXXXXXXXXXXXXXXXXXXX
3′ YYYYYYYYYYYYYYYYYYY

Overhang at the 3′ end of the antisense region.

5′ XXXXXXXXXXXXXXXXXXX
3′ MYYYYYYYYYYYYYYYYYYY
5′ XXXXXXXXXXXXXXXXXXX
3′ MMYYYYYYYYYYYYYYYYYYY
5′ XXXXXXXXXXXXXXXXXXX
3′ MMMYYYYYYYYYYYYYYYYYYY
5′ XXXXXXXXXXXXXXXXXXX
3′ MMMMYYYYYYYYYYYYYYYYYYY
5′ XXXXXXXXXXXXXXXXXXX
3′ MMMMMYYYYYYYYYYYYYYYYYYY
5′ XXXXXXXXXXXXXXXXXXX
3′ MMMMMMYYYYYYYYYYYYYYYYYYY
5′ XXXXXXXXXXXXXXXXXXX
3′ MMMMMMMYYYYYYYYYYYYYYYYYYY
5′ XXXXXXXXXXXXXXXXXXX
3′ MMMMMMMMYYYYYYYYYYYYYYYYYYY

Modified nucleotide(s) within the sense region

5′ XXXXXXXXXMXXXXXXXXX
3′ YYYYYYYYYYYYYYYYYYY
5′ XXXXXXXXXXXXXXXXXXX
3′ YYYYYYYYYMYYYYYYYYY
5′ XXXXXXXXXXXXXXXXXXXMM
3′ YYYYYYYYYYYYYYYYYYY
5′ XXXXXXXXXXXXXXXXXXX
3′ MMYYYYYYYYYYYYYYYYYYY

Exemplary nucleic acid molecules are provided below with the equivalent general structure in line with the symbols used above. The following duplexes are in accordance with the pattern:

5′ XXXXXXXXXXXXXXXXXXXMM
3′ MMYYYYYYYYYYYYYYYYYYY

TIMP1-A siRNA to human, mouse, rat and rhesus TIMP1 having a 19 nucleotide (i.e., 19mer) duplex region and modified 2 nucleotide (i.e., deoxynucleotide) overhangs at the 3′-ends of the sense and antisense strands.

5′ CCACCUUAUACCAGCGUUATT 3′
3′ TTGGUGGAAUAUGGUCGCAAU 5′

TIMP1-B siRNA to human and rhesus TIMP1 having a 19 nucleotide (i.e., 19mer) duplex region and modified 2 nucleotide (i.e., deoxynucleotide) overhangs at the 3′-ends of the sense and antisense strands.

5′ CACUGUUGGCUGUGAGGAATT 3′
3′ TTGUGACAACCGACACUCCUU 5′

TIMP1-C siRNA to human, mouse, rat and rhesus TIMP1 having a 19 nucleotide (i.e., 19mer) duplex region and modified 2 nucleotide (i.e., deoxynucleotide) overhangs at the 3′-ends of the sense and antisense strands.

5′ GGAAUAUCUCAUUGCAGGATT 3′
3′ TTCCUUAUAGAGUAACGUCCU 5′

TIMP2-A siRNA to human TIMP2 having a 19 nucleotide (i.e., 19mer) duplex region and modified 2 nucleotide (i.e., deoxynucleotide) overhangs at the 3′-ends of the sense and antisense strands.

5′ UGCAGAUGUAGUGAUCAGGTT 3′
3′ TTACGUCUACAUCACUAGUCC 5′

TIMP2-B siRNA to human, rhesus and rabbit TIMP2 having a 19 nucleotide (i.e., 19mer) duplex region and modified 2 nucleotide (i.e., deoxynucleotide) overhangs at the 3′-ends of the sense and antisense strands.

5′ GAGGAUCCAGUAUGAGAUCTT 3′
3′ TTCUCCUAGGUCAUACUCUAG 5′

TIMP2-C siRNA to human, mouse, rat, cow, dog and pig TIMP2 having a 19 nucleotide (i.e., 19mer) duplex region and modified 2 nucleotide (i.e., deoxynucleotide) overhangs at the 3′-ends of the sense and antisense strands.

5′ GCAGAUAAAGAUGUUCAAATT 3′
3′ TTCGUCUAUUUCUACAAGUUU 5′

TIMP2-D siRNA to human TIMP2 having a 19 nucleotide (i.e., 19mer) duplex region and modified 2 nucleotide (i.e., deoxynucleotide) overhangs at the 3′-ends of the sense and antisense strands.

5′ UAUCUCAUUGCAGGAAAGGTT 3′
3′ TTAUAGAGUAACGUCCUUUCC 5′

TIMP2-E siRNA to human TIMP2 having a 19 nucleotide (i.e., 19mer) duplex region and modified 2 nucleotide (i.e., deoxynucleotide) overhangs at the 3′-ends of the sense and antisense strands.

5′ GCACAGUGUUUCCCUGUUUTT 3′
3′ TTCGUGUCACAAAGGGACAAA 5′

### Nicks and Gaps in Nucleic Acid Strands

Nucleic acid molecules (e.g., siNA molecules) provided herein may have a strand, preferably the sense strand, that is nicked or gapped. As such, nucleic acid molecules may have three or more strand, for example, such as a meroduplex RNA (mdRNA) disclosed in International Patent Application No. PCT/US07/081,836. Nucleic acid molecules with a nicked or gapped strand may be between about 1-49 nucleotides, or may be RISC length (e.g., about 15 to 25 nucleotides) or Dicer substrate length (e.g., about 25 to 30 nucleotides) such as disclosed herein.

Nucleic acid molecules with three or more strands include, for example, an ‘A’ (antisense) strand, ‘S1’ (second) strand, and ‘S2’ (third) strand in which the ‘S1’ and ‘S2’ strands are complementary to and form base pairs with non-overlapping regions of the ‘A’ strand (e.g., an mdRNA can have the form of A:S1S2). The S1, S2, or more strands together form what is substantially similar to a sense strand to the ‘A’ antisense strand. The double-stranded region formed by the annealing of the ‘S1’ and ‘A’ strands is distinct from and non-overlapping with the double-stranded region formed by the annealing of the ‘S2’ and ‘A’ strands. An nucleic acid molecule (e.g., an siNA molecule) may be a “gapped” molecule, meaning a “gap” ranging from 0 nucleotides up to about 10 nucleotides (e.g., 0, 1, 2, 3, 4, 5, 6, 7, 8, 9 or 10 nucleotides). Preferably, the sense strand is gapped. In some embodiments, the A:S1 duplex is separated from the A:S2 duplex by a gap resulting from at least one unpaired nucleotide (up to about 10 unpaired nucleotides) in the ‘A’ strand that is positioned between the A:S1 duplex and the A:S2 duplex and that is distinct from any one or more unpaired nucleotide at the 3′-end of one or more of the ‘A’, ‘S1’, or ‘S2 strands. The A:S1 duplex may be separated from the A:B2 duplex by a gap of zero nucleotides (i.e., a nick in which only a phosphodiester bond between two nucleotides is broken or missing in the polynucleotide molecule) between the A:S1 duplex and the A:S2 duplex-which can also be referred to as nicked dsRNA (ndsRNA). For example, A:S1S2 may be include a dsRNA having at least two double-stranded regions that combined total about 14 base pairs to about 40 base pairs and the double-stranded regions are separated by a gap of about 0 to about 10 nucleotides, optionally having blunt ends, or A:S1S2 may include a dsRNA having at least two double-stranded regions separated by a gap of up to 10 nucleotides wherein at least one of the double-stranded regions includes between about 5 base pairs and 13 base pairs.

### Dicer Substrates

In certain embodiments, the nucleic acid molecules (e.g., siNA molecules) provided herein may be a precursor “Dicer substrate” molecule, e.g., double stranded nucleic acid, processed in vivo to produce an active nucleic acid molecules, for example as described in Rossi, US Patent App. No. 20050244858. In certain conditions and situations, it has been found that these relatively longer dsRNA siNA species, e.g., of from about 25 to about 30 nucleotides, can give unexpectedly effective results in terms of potency and duration of action. Without wishing to be bound by any particular theory, it is thought that the longer dsRNA species serve as a substrate for the enzyme Dicer in the cytoplasm of a cell. In addition to cleaving double stranded nucleic acid into shorter segments, Dicer may facilitate the incorporation of a single-stranded cleavage product derived from the cleaved dsRNA into the RNA-induced silencing complex (RISC complex) that is responsible for the destruction of the cytoplasmic RNA derived from the target gene.

Dicer substrates may have certain properties which enhance its processing by Dicer. Dicer substrates are of a length sufficient such that it is processed by Dicer to produce an active nucleic acid molecule and may further include one or more of the following properties: (i) the dsRNA is asymmetric, e.g., has a 3′ overhang on the first strand (antisense strand) and (ii) the dsRNA has a modified 3′ end on the antisense strand (sense strand) to direct orientation of Dicer binding and processing of the dsRNA to an active siRNA. In certain embodiments, the longest strand in the Dicer substrate may be 24-30 nucleotides.

Dicer substrates may be symmetric or asymmetric. The Dicer substrate may have a sense strand includes 22-28 nucleotides and the antisense strand may include 24-30 nucleotides; thus, in some embodiments the resulting Dicer substrate may have an overhang on the 3′ end of the antisense strand. Dicer substrate may have a sense strand 25 nucleotides in length, and the antisense strand having 27 nucleotides in length with a 2 base 3′-overhang. The overhang may be 1-3 nucleotides, for example 2 nucleotides. The sense strand may also have a 5′ phosphate.

An asymmetric Dicer substrate may further contain two deoxyribonucleotides at the 3′-end of the sense strand in place of two of the ribonucleotides. Some exemplary Dicer substrates lengths and structures are 21+0, 21+2, 21−2, 22+0, 22+1, 22−1, 23+0, 23+2, 23−2, 24+0, 24+2, 24−2, 25+0, 25+2, 25−2, 26+0, 26+2, 26−2, 27+0, 27+2, and 27−2.

The sense strand of a Dicer substrate may be between about 22 to about 30 (e.g., about 22, 23, 24, 25, 26, 27, 28, 29 or 30); about 22 to about 28; about 24 to about 30; about 25 to about 30; about 26 to about 30; about 26 and 29; or about 27 to about 28 nucleotides in length. In certain preferred embodiments Dicer substrates contain sense and antisense strands, that are at least about 25 nucleotides in length and no longer than about 30 nucleotides; between about 26 and 29 nucleotides; or 27 nucleotides in length. The sense and antisense strands may be the same length (blunt ended), different lengths (have overhangs), or a combination. The sense and antisense strands may exist on the same polynucleotide or on different polynucleotides. A Dicer substrate may have a duplex region of about 19, 20, 21, 22, 23, 24, 25 or 27 nucleotides.

Like other siNA molecules provided herein, the antisense strand of a Dicer substrate may have any sequence that anneals to the antisense strand under biological conditions, such as within the cytoplasm of a eukaryotic cell.

Dicer substrates may have any modifications to the nucleotide base, sugar or phosphate backbone as known in the art and/or as described herein for other nucleic acid molecules (such as siNA molecules). In certain embodiments, Dicer substrates may have a sense strand is modified for Dicer processing by suitable modifiers located at the 3′ end of the sense strand, i.e., the dsRNA is designed to direct orientation of Dicer binding and processing. Suitable modifiers include nucleotides such as deoxyribonucleotides, dideoxyribonucleotides, acyclonucleotides and the like and sterically hindered molecules, such as fluorescent molecules and the like. Acyclonucleotides substitute a 2-hydroxyethoxymethyl group for-the 2′-deoxyribofuranosyl sugar normally present in dNMPs. Other nucleotides modifiers that could be used in Dicer substrate siNA molecules include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidi-ne (d4T) and the monophosphate nucleotides of 3′-azido-3′-deoxythymidine (AZT), 2′,3′-dideoxy-3′-thiacytidine (3TC) and 2′,3′-didehydro-2′,3′-dide-oxythymidine (d4T). In one embodiment, deoxyribonucleotides are used as the modifiers. When nucleotide modifiers are utilized, they may replace ribonucleotides (e.g., 1-3 nucleotide modifiers, or 2 nucleotide modifiers are substituted for the ribonucleotides on the 3′ end of the sense strand) such that the length of the Dicer substrate does not change. When sterically hindered molecules are utilized, they may be attached to the ribonucleotide at the 3′ end of the antisense strand. Thus, in certain embodiments the length of the strand does not change with the incorporation of the modifiers. In certain embodiments, two DNA bases in the dsRNA are substituted to direct the orientation of Dicer processing of the antisense strand. In a further embodiment of, two terminal DNA bases are substituted for two ribonucleotides on the 3′-end of the sense strand forming a blunt end of the duplex on the 3′ end of the sense strand and the 5′ end of the antisense strand, and a two-nucleotide RNA overhang is located on the 3′-end of the antisense strand. This is an asymmetric composition with DNA on the blunt end and RNA bases on the overhanging end.

In certain embodiments modifications are included in the Dicer substrate such that the modification does not prevent the nucleic acid molecule from serving as a substrate for Dicer. In one embodiment, one or more modifications are made that enhance Dicer processing of the Dicer substrate. One or more modifications may be made that result in more effective RNAi generation. One or more modifications may be made that support a greater RNAi effect. One or more modifications are made that result in greater potency per each Dicer substrate to be delivered to the cell. Modifications may be incorporated in the 3′-terminal region, the 5′-terminal region, in both the 3′-terminal and 5′-terminal region or at various positions within the sequence. Any number and combination of modifications can be incorporated into the Dicer substrate so long as the modification does not prevent the nucleic acid molecule from serving as a substrate for Dicer. Where multiple modifications are present, they may be the same or different. Modifications to bases, sugar moieties, the phosphate backbone, and their combinations are contemplated. Either 5′-terminus can be phosphorylated.

Examples of Dicer substrate phosphate backbone modifications include phosphonates, including methylphosphonate, phosphorothioate, and phosphotriester modifications such as alkylphosphotriesters, and the like. Examples of Dicer substrate sugar moiety modifications include 2′-alkyl pyrimidine, such as 2′-O-methyl, 2′-fluoro, amino, and deoxy modifications and the like (see, e.g., Amarzguioui et al., 2003). Examples of Dicer substrate base group modifications include abasic sugars, 2-O-alkyl modified pyrimidines, 4-thiouracil, 5-bromouracil, 5-iodouracil, and 5-(3-aminoallyl)-uracil and the like. Locked nucleic acids, or LNA's, could also be incorporated.

The sense strand may be modified for Dicer processing by suitable modifiers located at the 3′ end of the sense strand, i.e., the Dicer substrate is designed to direct orientation of Dicer binding and processing. Suitable modifiers include nucleotides such as deoxyribonucleotides, dideoxyribonucleotides, acyclonucleotides and the like and sterically hindered molecules, such as fluorescent molecules and the like. Acyclonucleotides substitute a 2-hydroxyethoxymethyl group for-the 2′-deoxyribofuranosyl sugar normally present in dNMPs. Other nucleotides modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidi-ne (d4T) and the monophosphate nucleotides of 3′-azido-3′-deoxythymidine (AZT), 2′,3′-dideoxy-3′-thiacytidine (3TC) and 2′,3′-didehydro-2′,3′-dide-oxythymidine (d4T). In one embodiment, deoxyribonucleotides are used as the modifiers. When nucleotide modifiers are utilized, 1-3 nucleotide modifiers, or 2 nucleotide modifiers are substituted for the ribonucleotides on the 3′ end of the sense strand. When sterically hindered molecules are utilized, they are attached to the ribonucleotide at the 3′ end of the antisense strand. Thus, the length of the strand does not change with the incorporation of the modifiers. In another embodiment, substituting two DNA bases in the Dicer substrate to direct the orientation of Dicer processing of the antisense strand is contemplated. In a further embodiment of the present invention, two terminal DNA bases are substituted for two ribonucleotides on the 3′-end of the sense strand forming a blunt end of the duplex on the 3′ end of the sense strand and the 5′ end of the antisense strand, and a two-nucleotide RNA overhang is located on the 3′-end of the antisense strand. This is an asymmetric composition with DNA on the blunt end and RNA bases on the overhanging end.

The antisense strand may be modified for Dicer processing by suitable modifiers located at the 3′ end of the antisense strand, i.e., the dsRNA is designed to direct orientation of Dicer binding and processing. Suitable modifiers include nucleotides such as deoxyribonucleotides, dideoxyribonucleotides, acyclonucleotides and the like and sterically hindered molecules, such as fluorescent molecules and the like. Acyclonucleotides substitute a 2-hydroxyethoxymethyl group for the 2′-deoxyribofuranosyl sugar normally present in dNMPs. Other nucleotide modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidi-ne (d4T) and the monophosphate nucleotides of 3′-azido-3′-deoxythymidine (AZT), 2′,3′-dideoxy-3′-thiacytidine (3TC) and 2′,3′-didehydro-2′,3′-dide-oxythymidine (d4T). In one embodiment, deoxyribonucleotides are used as the modifiers. When nucleotide modifiers are utilized, 1-3 nucleotide modifiers, or 2 nucleotide modifiers are substituted for the ribonucleotides on the 3′ end of the antisense strand. When sterically hindered molecules are utilized, they are attached to the ribonucleotide at the 3′ end of the antisense strand. Thus, the length of the strand does not change with the incorporation of the modifiers. In another embodiment, the two DNA bases in the dsRNA may be substituted to direct the orientation of Dicer processing. In a further embodiment, two terminal DNA bases are located on the 3′ end of the antisense strand in place of two ribonucleotides forming a blunt end of the duplex on the 5′ end of the sense strand and the 3′ end of the antisense strand, and a two-nucleotide RNA overhang is located on the 3′-end of the sense strand. This is an asymmetric composition with DNA on the blunt end and RNA bases on the overhanging end.

Dicer substrates with a sense and an antisense strand can be linked by a third structure. The third structure will not block Dicer activity on the Dicer substrate and will not interfere with the directed destruction of the RNA transcribed from the target gene. The third structure may be a chemical linking group. Suitable chemical linking groups are known in the art and can be used. Alternatively, the third structure may be an oligonucleotide that links the two oligonucleotides of the dsRNA is a manner such that a hairpin structure is produced upon annealing of the two oligonucleotides making up the Dicer substrate. The hairpin structure preferably does not block Dicer activity on the Dicer substrate or interfere with the directed destruction of the RNA transcribed from the target gene.

The sense and antisense strands of the Dicer substrate are not required to be completely complementary. They only need to be substantially complementary to anneal under biological conditions and to provide a substrate for Dicer that produces an siRNA sufficiently complementary to the target sequence.

Dicer substrate can have certain properties that enhance its processing by Dicer. The Dicer substrate can have a length sufficient such that it is processed by Dicer to produce an active nucleic acid molecules (e.g., siRNA) and may have one or more of the following properties: (i) the Dicer substrate is asymmetric, e.g., has a 3′ overhang on the first strand (antisense strand) and (ii) the Dicer substrate has a modified 3′ end on the second strand (sense strand) to direct orientation of Dicer binding and processing of the Dicer substrate to an active siRNA. The Dicer substrate can be asymmetric such that the sense strand includes 22-28 nucleotides and the antisense strand includes 24-30 nucleotides. Thus, the resulting Dicer substrate has an overhang on the 3′ end of the antisense strand. The overhang is 1-3 nucleotides, for example 2 nucleotides. The sense strand may also have a 5′ phosphate.

A Dicer substrate may have an overhang on the 3′ end of the antisense strand and the sense strand is modified for Dicer processing. The 5′ end of the sense strand may have a phosphate. The sense and antisense strands may anneal under biological conditions, such as the conditions found in the cytoplasm of a cell. A region of one of the strands, particularly the antisense strand, of the Dicer substrate may have a sequence length of at least 19 nucleotides, wherein these nucleotides are in the 21-nucleotide region adjacent to the 3′ end of the antisense strand and are sufficiently complementary to a nucleotide sequence of the RNA produced from the target gene. A Dicer substrate may also have one or more of the following additional properties: (a) the antisense strand has a right shift from a corresponding 21-mer (i.e., the antisense strand includes nucleotides on the right side of the molecule when compared to the corresponding 21-mer), (b) the strands may not be completely complementary, i.e., the strands may contain simple mismatch pairings and (c) base modifications such as locked nucleic acid(s) may be included in the 5′ end of the sense strand.

An antisense strand of a Dicer substrate nucleic acid molecule may be modified to include 1-9 ribonucleotides on the 5′-end to give a length of 22-28 nucleotides. When the antisense strand has a length of 21 nucleotides, then 1-7 ribonucleotides, or 2-5 ribonucleotides and or 4 ribonucleotides may be added on the 3′-end. The added ribonucleotides may have any sequence. Although the added ribonucleotides may be complementary to the target gene sequence, full complementarity between the target sequence and the antisense strands is not required. That is, the resultant antisense strand is sufficiently complementary with the target sequence. A sense strand may then have 24-30 nucleotides. The sense strand may be substantially complementary with the antisense strand to anneal to the antisense strand under biological conditions. In one embodiment, the antisense strand may be synthesized to contain a modified 3′-end to direct Dicer processing. The sense strand may have a 3′ overhang. The antisense strand may be synthesized to contain a modified 3′-end for Dicer binding and processing and the sense strand has a 3′ overhang.

### TIMP1 and TIMP2

Exemplary nucleic acid sequence of target tissue inhibitors of metalloproteinase-1 and -2 (human TIMP1 and TIMP2) cDNA is disclosed in GenBank accession numbers: NM_{—}003454 and NM_{—}003455 and the corresponding mRNA sequence, for example as listed as SEQ ID NO: 1 and SEQ ID NO:2. One of ordinary skill in the art would understand that a given sequence may change over time and to incorporate any changes needed in the nucleic acid molecules herein accordingly.

Expression of TIMP1 and TIMP2 was shown to be increased in fibrotic liver from rats with hepatic fibrosis (Nie, et al 2004. World J. Gastroenterol. 10:86-90). TIMP1 and TIMP2 are potential targets for the treatment of fibrosis.

### Methods and Compositions for Inhibiting TIMP1 and TIMP2

Provided are compositions and methods for inhibition of TIMP1 and TIMP2 expression by using small nucleic acid molecules, such as short interfering nucleic acid (siNA), interfering RNA (RNAi), short interfering RNA (siRNA), double-stranded RNA (dsRNA), micro-RNA (miRNA), and short hairpin RNA (shRNA) molecules capable of mediating or that mediate RNA interference against TIMP1 and TIMP2 gene expression. The composition and methods disclosed herein are also useful in treating various fibrosis such as liver fibrosis, lung fibrosis, kidney fibrosis and fibrotic conditions shown in Table I, supra.

Nucleic acid molecule(s) and/or methods as disclosed herein may be used to down regulate the expression of gene(s) that encode RNA referred to, by example, Genbank Accession NM_{—}003254.2 and NM_{—}004255.4.

Compositions, methods and kits provided herein may include one or more nucleic acid molecules (e.g., siNA) and methods that independently or in combination modulate (e.g., downregulate) the expression of TIMP1 and or TIMP2 protein and/or genes encoding TIMP1 and TIMP2 proteins, proteins and/or genes encoding TIMP1 and TIMP2 associated with the maintenance and/or development of diseases, conditions or disorders associated with TIMP1 and TIMP2, such as liver fibrosis, cirrhosis, pulmonary fibrosis, kidney fibrosis, peritoneal fibrosis, chronic hepatic damage, and fibrillogenesis (e.g., genes encoding sequences comprising those sequences referred to by GenBank Accession Nos. NM-003254 and NM_{—}003255), or a TIMP1 and TIMP2 gene family member where the genes or gene family sequences share sequence homology. The description of the various aspects and embodiments is provided with reference to exemplary genes TIMP1 and TIMP2. However, the various aspects and embodiments are also directed to other related TIMP1 and TIMP2 genes, such as homolog genes and transcript variants, and polymorphisms (e.g., single nucleotide polymorphism, (SNPs)) associated with certain TIMP1 and TIMP2 genes. As such, the various aspects and embodiments are also directed to other genes that are involved in TIMP1 and TIMP2 mediated pathways of signal transduction or gene expression that are involved, for example, in the maintenance or development of diseases, traits, or conditions described herein. These additional genes can be analyzed for target sites using the methods described for the TIMP1 and TIMP2 gene herein. Thus, the modulation of other genes and the effects of such modulation of the other genes can be performed, determined, and measured as described herein.

In one embodiment, compositions and methods provided herein include a double-stranded short interfering nucleic acid (siNA) molecule that down-regulates expression of a TIMP1 and TIMP2 gene (e.g., human TIMP1 and TIMP2 exemplified by SEQ ID NO:1 and SEQ ID NO:2, respectively), where the nucleic acid molecule includes about 15 to about 49 base pairs.

In one embodiment, a nucleic acid disclosed may be used to inhibit the expression of the TIMP1 and TIMP2 gene or a TIMP1 and TIMP2 gene family where the genes or gene family sequences share sequence homology. Such homologous sequences can be identified as is known in the art, for example using sequence alignments. Nucleic acid molecules can be designed to target such homologous sequences, for example using perfectly complementary sequences or by incorporating non-canonical base pairs, for example mismatches and/or wobble base pairs, that can provide additional target sequences. In instances where mismatches are identified, non-canonical base pairs (for example, mismatches and/or wobble bases) can be used to generate nucleic acid molecules that target more than one gene sequence. In a non-limiting example, non-canonical base pairs such as UU and CC base pairs are used to generate nucleic acid molecules that are capable of targeting sequences for differing TIMP1 and TIMP2 targets that share sequence homology. As such, one advantage of using siNAs disclosed herein is that a single nucleic acid can be designed to include nucleic acid sequence that is complementary to the nucleotide sequence that is conserved between the homologous genes. In this approach, a single nucleic acid can be used to inhibit expression of more than one gene instead of using more than one nucleic acid molecule to target the different genes.

Nucleic acid molecules may be used to target conserved sequences corresponding to a gene family or gene families such as TIMP1 and TIMP2 family genes. As such, nucleic acid molecules targeting multiple TIMP1 and TIMP2 targets can provide increased therapeutic effect. In addition, nucleic acid can be used to characterize pathways of gene function in a variety of applications. For example, nucleic acid molecules can be used to inhibit the activity of target gene(s) in a pathway to determine the function of uncharacterized gene(s) in gene function analysis, mRNA function analysis, or translational analysis. The nucleic acid molecules can be used to determine potential target gene pathways involved in various diseases and conditions toward pharmaceutical development. The nucleic acid molecules can be used to understand pathways of gene expression involved in, for example fibroses such as liver, kidney or pulmonary fibrosis, and/or inflammatory and proliferative traits, diseases, disorders, and/or conditions.

In one embodiment, the compositions and methods provided herein include a nucleic acid molecule having RNAi activity against TIMP1 RNA, where the nucleic acid molecule includes a sequence complementary to any RNA having TIMP1 encoding sequence. In another embodiment, a nucleic acid molecule may have RNAi activity against TIMP1 RNA, where the nucleic acid molecule includes a sequence complementary to an RNA having variant TIMP1 encoding sequence, for example other mutant TIMP1 genes known in the art to be associated with the maintenance and/or development of fibrosis. In another embodiment, a nucleic acid molecule disclosed herein includes a nucleotide sequence that can interact with nucleotide sequence of a TIMP1 gene and thereby mediate silencing of TIMP1 gene expression, for example, wherein the nucleic acid molecule mediates regulation of TIMP1 gene expression by cellular processes that modulate the chromatin structure or methylation patterns of the TIMP1 gene and prevent transcription of the TIMP1 gene.

In another embodiment the compositions and methods provided herein include a nucleic acid molecule having RNAi activity against TIMP2 RNA, where the nucleic acid molecule includes a sequence complementary to any RNA having TIMP2 encoding sequence, such as those sequences having GenBank Accession No. NM_{—}003455. Nucleic acid molecules may have RNAi activity against TIMP2 RNA, where the nucleic acid molecule includes a sequence complementary to an RNA having variant TIMP2 encoding sequence, for example other mutant TIMP2 genes known in the art to be associated with the maintenance and/or development of fibrosis. Nucleic acid molecules disclosed herein include a nucleotide sequence that can interact with nucleotide sequence of a TIMP2 gene and thereby mediate silencing of TIMP1 gene expression, e.g., where the nucleic acid molecule mediates regulation of TIMP2 gene expression by cellular processes that modulate the chromatin structure or methylation patterns of the TIMP2 gene and prevent transcription of the TIMP2 gene.

### Methods of Treatment

In one embodiment, nucleic acid molecules may be used to down regulate or inhibit the expression of TIMP1 and/or TIMP1 proteins arising from TIMP1 and/or TIMP1 haplotype polymorphisms that are associated with a disease or condition, (e.g., fibrosis). Analysis of TIMP1 and/or TIMP1 genes, or TIMP1 and/or TIMP1 protein or RNA levels can be used to identify subjects with such polymorphisms or those subjects who are at risk of developing traits, conditions, or diseases described herein. These subjects are amenable to treatment, for example, treatment with nucleic acid molecules disclosed herein and any other composition useful in treating diseases related to TIMP1 and/or TIMP1 gene expression. As such, analysis of TIMP1 and/or TIMP1 protein or RNA levels can be used to determine treatment type and the course of therapy in treating a subject. Monitoring of TIMP1 and/or TIMP1 protein or RNA levels can be used to predict treatment outcome and to determine the efficacy of compounds and compositions that modulate the level and/or activity of certain TIMP1 and/or TIMP1 proteins associated with a trait, condition, or disease.

In one embodiment, nucleic acid molecules may be used to down regulate or inhibit the expression of TIMP2 and/or TIMP2 proteins arising from TIMP2 and/or TIMP2 haplotype polymorphisms that are associated with a disease or condition, (e.g., fibrosis). Analysis of TIMP2 and/or TIMP2 genes, or TIMP2 and/or TIMP2 protein or RNA levels can be used to identify subjects with such polymorphisms or those subjects who are at risk of developing traits, conditions, or diseases described herein. These subjects are amenable to treatment, for example, treatment with nucleic acid molecules disclosed herein and any other composition useful in treating diseases related to TIMP2 and/or TIMP2 gene expression. As such, analysis of TIMP2 and/or TIMP2 protein or RNA levels can be used to determine treatment type and the course of therapy in treating a subject. Monitoring of TIMP2 and/or TIMP2 protein or RNA levels can be used to predict treatment outcome and to determine the efficacy of compounds and compositions that modulate the level and/or activity of certain TIMP2 and/or TIMP2 proteins associated with a trait, condition, or disease.

Provided are compositions and methods for inhibition of TIMP1 and TIMP2 expression by using small nucleic acid molecules as provided herein, such as short interfering nucleic acid (siNA), interfering RNA (RNAi), short interfering RNA (siRNA), double-stranded RNA (dsRNA), micro-RNA (miRNA), and short hairpin RNA (shRNA) molecules capable of mediating or that mediate RNA interference against TIMP1 and TIMP2 gene expression. The composition and methods disclosed herein are also useful in treating various fibrosis such as liver fibrosis, lung fibrosis, and kidney fibrosis.

The nucleic acid molecules disclosed herein individually, or in combination or in conjunction with other drugs, can be use for preventing or treating diseases, traits, conditions and/or disorders associated with TIMP1 and TIMP2, such as liver fibrosis, cirrhosis, pulmonary fibrosis, kidney fibrosis, peritoneal fibrosis, chronic hepatic damage, and fibrillogenesis.

The nucleic acid molecules disclosed herein are able to inhibit the expression of TIMP1 or TIMP2 in a sequence specific manner. The nucleic acid molecules may include a sense strand and an antisense strand which include contiguous nucleotides that are at least partially complementary (antisense) to a TIMP1 or TIMP2 mRNA.

In some embodiments, dsRNA specific for TIMP1 or TIMP2 can be used in conjunction with other dsRNA specific for other molecular chaperones that assist in the folding of newly synthesized proteins such as, calnexin, calreticulin, BiP (Bergeron et al. Trends Biochem. Sci. 1994; 19:124-128; Herbert et al. 1995; Cold Spring Harb. Symp. Quant. Biol. 60:405-415)

Fibrosis can be treated by RNA interference using nucleic acid molecules as disclosed herein. Exemplary fibrosis include liver fibrosis, peritoneal fibrosis, lung fibrosis, kidney fibrosis, vocal cord fibrosis, intestinal fibrosis. The nucleic acid molecules disclosed herein may inhibit the expression of TIMP1 or TIMP2 in a sequence specific manner.

Treatment of fibrosis can be monitored by determining the level of extracellular collagen using suitable techniques known in the art such as, using anti-collagen I antibodies. Treatment can also be monitored by determining the level of TIMP1 or TIMP2 mRNA or the level of TIMP1 or TIMP2 protein in the cells of the affected tissue. Treatment can also be monitored by non-invasive scanning of the affected organ or tissue such as by computer assisted tomography scan, magnetic resonance elastography scans.

A method for treating or preventing TIMP1 associated disease or condition in a subject or organism may include contacting the subject or organism with a nucleic acid molecule as provided herein under conditions suitable to modulate the expression of the TIMP1 gene in the subject or organism.

A method for treating or preventing TIMP2 associated disease or condition in a subject or organism may include contacting the subject or organism with a nucleic acid molecule as provided herein under conditions suitable to modulate the expression of the TIMP2 gene in the subject or organism.

A method for treating or preventing fibrosis in a subject or organism may include contacting the subject or organism with a nucleic acid molecule under conditions suitable to modulate the expression of the TIMP1 and/or TIMP2 gene in the subject or organism.

A method for treating or preventing one or more fibroses selected from the group consisting of liver fibrosis, kidney fibrosis, and pulmonary fibrosis in a subject or organism may include contacting the subject or organism with a nucleic acid molecule under conditions suitable to modulate the expression of the TIMP1 and/or TIMP2 gene in the subject or organism.

### Fibrotic Diseases

Fibrotic diseases are generally characterized by the excess deposition of a fibrous material within the extracellular matrix, which contributes to abnormal changes in tissue architecture and interferes with normal organ function.

All tissues damaged by trauma respond by the initiation of a wound-healing program. Fibrosis, a type of disorder characterized by excessive scarring, occurs when the normal self-limiting process of wound healing response is disturbed, and causes excessive production and deposition of collagen. As a result, normal organ tissue is replaced with scar tissue, which eventually leads to the functional failure of the organ.

Fibrosis may be initiated by diverse causes and in various organs. Liver cirrhosis, pulmonary fibrosis, sarcoidosis, keloids and kidney fibrosis are all chronic conditions associated with progressive fibrosis, thereby causing a continuous loss of normal tissue function.

Acute fibrosis (usually with a sudden and severe onset and of short duration) occurs as a common response to various forms of trauma including accidental injuries (particularly injuries to the spine and central nervous system), infections, surgery, ischemic illness (e.g. cardiac scarring following heart attack), burns, environmental pollutants, alcohol and other types of toxins, acute respiratory distress syndrome, radiation and chemotherapy treatments).

Fibrosis, a fibrosis related pathology or a pathology related to aberrant crosslinking of cellular proteins may all be treated by the siRNAs disclosed herein. Fibrotic diseases or diseases in which fibrosis is evident (fibrosis related pathology) include both acute and chronic forms of fibrosis of organs, including all etiological variants of the following: pulmonary fibrosis, including interstitial lung disease and fibrotic lung disease, liver fibrosis, cardiac fibrosis including myocardial fibrosis, kidney fibrosis including chronic renal failure, skin fibrosis including scleroderma, keloids and hypertrophic scars; myelofibrosis (bone marrow fibrosis); fibrosis in the brain associated with brain infarction; all types of ocular scarring including proliferative vitreoretinopathy (PVR) and scarring resulting from surgery to treat cataract or glaucoma; inflammatory bowel disease of variable etiology, macular degeneration, Grave's ophthalmopathy, drug induced ergotism, keloid scars, scleroderma, psoriasis, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease, fibrosis associated with brain infarction and collagenous colitis.

In various embodiments, the compounds (nucleic acid molecules) as disclosed herein may be used to treat fibrotic diseases, for example as disclosed herein, as well as many other diseases and conditions apart from fibrotic diseases, for example such as disclosed herein. Other conditions to be treated include fibrotic diseases in other organs—kidney fibrosis for any reason (CKD including ESRD); lung fibrosis (including ILF); myelofibrosis, abnormal scarring (keloids) associated with all possible types of skin injury accidental and jatrogenic (operations); scleroderma; cardiofibrosis, failure of glaucoma filtering operation; intestinal adhesions.

### Ocular Surgery and Fibrotic Complications

Contracture of scar tissue resulting from eye surgery may often occur. Glaucoma surgery to create new drainage channels often fails due to scarring and contraction of tissues and the generated drainage system may be blocked requiring additional surgical intervention. Current anti-scarring regimens (Mitomycin C or 5FU) are limited due to the complications involved (e.g. blindness) e.g. see Cordeiro M F, et al., Human anti-transforming growth factor-beta2 antibody: a new glaucoma anti-scarring agent Invest Ophthalmol Vis Sci. 1999 September; 40(10):2225-34. There may also be contraction of scar tissue formed after corneal trauma or corneal surgery, for example laser or surgical treatment for myopia or refractive error in which contraction of tissues may lead to inaccurate results. Scar tissue may be formed on/in the vitreous humor or the retina, for example, and may eventually causes blindness in some diabetics, and may be formed after detachment surgery, called proliferative vitreoretinopathy (PVR). PVR is the most common complication following retinal detachment and is associated with a retinal hole or break. PVR refers to the growth of cellular membranes within the vitreous cavity and on the front and back surfaces of the retina containing retinal pigment epithelial (RPE) cells. These membranes, which are essentially scar tissues, exert traction on the retina and may result in recurrences of retinal detachment, even after an initially successful retinal detachment procedure.

Scar tissue may be formed in the orbit or on eye and eyelid muscles after squint, orbital or eyelid surgery, or thyroid eye disease, and where scarring of the conjunctiva occurs as may happen after glaucoma surgery or in cicatricial disease, inflammatory disease, for example, pemphigoid, or infective disease, for example, trachoma. A further eye problem associated with the contraction of collagen-including tissues is the opacification and contracture of the lens capsule after cataract extraction. Important role for MMPs has been recognized in ocular diseases including wound healing, dry eye, sterile corneal ulceration, recurrent epithelial erosion, corneal neovascularization, pterygium, conjuctivochalasis, glaucoma, PVR, and ocular fibrosis.

### Liver Fibrosis

Liver fibrosis (LF) is a generally irreversible consequence of hepatic damage of several etiologies. In the Western world, the main etiologic categories are: alcoholic liver disease (30-50%), viral hepatitis (30%), biliary disease (5-10%), primary hemochromatosis (5%), and drug-related and cryptogenic cirrhosis of, unknown etiology, (10-15%). Wilson's disease, α₁-antitrypsin deficiency and other rare diseases also have liver fibrosis as one of the symptoms. Liver cirrhosis, the end stage of liver fibrosis, frequently requires liver transplantation and is among the top ten causes of death in the Western world.

### Kidney Fibrosis and Related Conditions.

### Chronic Renal Failure (CRF)

Chronic renal failure is a gradual and progressive loss of the ability of the kidneys to excrete wastes, concentrate urine, and conserve electrolytes. CRF is slowly progressive. It most often results from any disease that causes gradual loss of kidney function, and fibrosis is the main pathology that produces CRF.

### Diabetic Nephropathy

Diabetic nephropathy, hallmarks of which are glomerulosclerosis and tubulointerstitial fibrosis, is the single most prevalent cause of end-stage renal disease in the modern world, and diabetic patients constitute the largest population on dialysis. Such therapy is costly and far from optimal. Transplantation offers a better outcome but suffers from a severe shortage of donors.

### Chronic Kidney Disease

Chronic kidney disease (CKD) is a worldwide public health problem and is recognized as a common condition that is associated with an increased risk of cardiovascular disease and chronic renal failure (CRF).

The Kidney Disease Outcomes Quality Initiative (K/DOQI) of the National Kidney Foundation (NKF) defines chronic kidney disease as either kidney damage or a decreased kidney glomerular filtration rate (GFR) for three or more months. Other markers of CKD are also known and used for diagnosis. In general, the destruction of renal mass with irreversible sclerosis and loss of nephrons leads to a progressive decline in GFR. Recently, the K/DOQI published a classification of the stages of CKD, as follows:
Stage 1: Kidney damage with normal or increased GFR (>90 mL/min/1.73 m2)
Stage 2: Mild reduction in GFR (60-89 mL/min/1.73 m2)
Stage 3: Moderate reduction in GFR (30-59 mL/min/1.73 m2)
Stage 4: Severe reduction in GFR (15-29 mL/min/1.73 m2)
Stage 5: Kidney failure (GFR<15 mL/min/1.73 m2 or dialysis)

In stages 1 and 2 CKD, GFR alone does not confirm the diagnosis. Other markers of kidney damage, including abnormalities in the composition of blood or urine or abnormalities in imaging tests, may be relied upon.

### Pathophysiology of CKD

Approximately 1 million nephrons are present in each kidney, each contributing to the total GFR. Irrespective of the etiology of renal injury, with progressive destruction of nephrons, the kidney is able to maintain GFR by hyperfiltration and compensatory hypertrophy of the remaining healthy nephrons. This nephron adaptability allows for continued normal clearance of plasma solutes so that substances such as urea and creatinine start to show significant increases in plasma levels only after total GFR has decreased to 50%, when the renal reserve has been exhausted. The plasma creatinine value will approximately double with a 50% reduction in GFR. Therefore, a doubling in plasma creatinine from a baseline value of 0.6 mg/dL to 1.2 mg/dL in a patient actually represents a loss of 50% of functioning nephron mass.

The residual nephron hyperfiltration and hypertrophy, although beneficial for the reasons noted, is thought to represent a major cause of progressive renal dysfunction. This is believed to occur because of increased glomerular capillary pressure, which damages the capillaries and leads initially to focal and segmental glomerulosclerosis and eventually to global glomerulosclerosis. This hypothesis has been based on studies of five-sixths nephrectomized rats, which develop lesions that are identical to those observed in humans with CKD.

The two most common causes of chronic kidney disease are diabetes and hypertension. Other factors include acute insults from nephrotoxins, including contrasting agents, or decreased perfusion; Proteinuria; Increased renal ammoniagenesis with interstitial injury; Hyperlipidemia; Hyperphosphatemia with calcium phosphate deposition; Decreased levels of nitrous oxide and smoking

In the United States, the incidence and prevalence of CKD is rising, with poor outcomes and high cost to the health system. Kidney disease is the ninth leading cause of death in the US. The high rate of mortality has led the US Surgeon General's mandate for America's citizenry, Healthy People 2010, to contain a chapter focused on CKD. The objectives of this chapter are to articulate goals and to provide strategies to reduce the incidence, morbidity, mortality, and health costs of chronic kidney disease in the United States.

The incidence rates of end-stage renal disease (ESRD) have also increased steadily internationally since 1989. The United States has the highest incident rate of ESRD, followed by Japan. Japan has the highest prevalence per million population, followed by the US.

The mortality rates associated with hemodialysis are striking and indicate that the life expectancy of patients entering into hemodialysis is markedly shortened. At every age, patients with ESRD on dialysis have significantly increased mortality when compared with nondialysis patients and individuals without kidney disease. At age 60 years, a healthy person can expect to live for more than 20 years, whereas the life expectancy of a 60-year-old patient starting hemodialysis is closer to 4 years (Aurora and Verelli, May 21, 2009. Chronic Renal Failure: Treatment & Medication. Emedicine. http://emedicine.medscape.com/article/238798-treatment).

### Pulmonary Fibrosis

Interstitial pulmonary fibrosis (IPF) is scarring of the lung caused by a variety of inhaled agents including mineral particles, organic dusts, and oxidant gases, or by unknown reasons (idiopathic lung fibrosis). The disease afflicts millions of individuals worldwide, and there are no effective therapeutic approaches. A major reason for the lack of useful treatments is that few of the molecular mechanisms of disease have been defined sufficiently to design appropriate targets for therapy (Lasky J A., Brody A R. (2000), “Interstitial fibrosis and growth factors”, Environ Health Perspect.; 108 Suppl 4:751-62).

### Cardiac Fibrosis

Heart failure is unique among the major cardiovascular disorders in that it alone is increasing in prevalence while there has been a striking decrease in other conditions. Some of this can be attributed to the aging of the populations of the United States and Europe. The ability to salvage patients with myocardial damage is also a major factor, as these patients may develop progression of left ventricular dysfunction due to deleterious remodelling of the heart.

The normal myocardium is composed of a variety of cells, cardiac myocytes and noncardiomyocytes, which include endothelial and vascular smooth muscle cells and fibroblasts.

Structural remodeling of the ventricular wall is a key determinant of clinical outcome in heart disease. Such remodeling involves the production and destruction of extracellular matrix proteins, cell proliferation and migration, and apoptotic and necrotic cell death. Cardiac fibroblasts are crucially involved in these processes, producing growth factors and cytokines that act as autocrine and paracrine factors, as well as extracellular matrix proteins and proteinases. Recent studies have shown that the interactions between cardiac fibroblasts and cardiomyocytes are essential for the progression of cardiac remodeling of which the net effect is deterioration in cardiac function and the onset of heart failure (Manabe I, et al., (2002), “Gene expression in fibroblasts and fibrosis: involvement in cardiac hypertrophy”, Circ Res. 13; 91(12):1103-13).

### Burns and Scars

A particular problem which may arise, particularly in fibrotic disease, is contraction of tissues, for example contraction of scars. Contraction of tissues including extracellular matrix components, especially of collagen-including tissues, may occur in connection with many different pathological conditions and with surgical or cosmetic procedures. Contracture, for example, of scars, may cause physical problems, which may lead to the need for medical treatment, or it may cause problems of a purely cosmetic nature. Collagen is the major component of scar and other contracted tissue and as such is the most important structural component to consider. Nevertheless, scar and other contracted tissue also includes other structural components, especially other extracellular matrix components, for example, elastin, which may also contribute to contraction of the tissue.

Contraction of collagen-including tissue, which may also include other extracellular matrix components, frequently occurs in the healing of burns. The burns may be chemical, thermal or radiation burns and may be of the eye, the surface of the skin or the skin and the underlying tissues. It may also be the case that there are burns on internal tissues, for example, caused by radiation treatment. Contraction of burnt tissues is often a problem and may lead to physical and/or cosmetic problems, for example, loss of movement and/or disfigurement.

Skin grafts may be applied for a variety of reasons and may often undergo contraction after application. As with the healing of burnt tissues the contraction may lead to both physical and cosmetic problems. It is a particularly serious problem where many skin grafts are needed as, for example, in a serious burns case.

Contraction is also a problem in production of artificial skin. To make a true artificial skin it is necessary to have an epidermis made of epithelial cells (keratinocytes) and a dermis made of collagen populated with fibroblasts. It is important to have both types of cells because they signal and stimulate each other using growth factors. The collagen component of the artificial skin often contracts to less than one tenth of its original area when populated by fibroblasts.

Cicatricial contraction, contraction due to shrinkage of the fibrous tissue of a scar, is common. In some cases the scar may become a vicious cicatrix, a scar in which the contraction causes serious deformity. A patient's stomach may be effectively separated into two separate chambers in an hour-glass contracture by the contraction of scar tissue formed when a stomach ulcer heals. Obstruction of passages and ducts, cicatricial stenosis, may occur due to the contraction of scar tissue. Contraction of blood vessels may be due to primary obstruction or surgical trauma, for example, after surgery or angioplasty. Stenosis of other hollow visci, for examples, ureters, may also occur. Problems may occur where any form of scarring takes place, whether resulting from accidental wounds or from surgery. Conditions of the skin and tendons which involve contraction of collagen-including tissues include post-trauma conditions resulting from surgery or accidents, for example, hand or foot tendon injuries, post-graft conditions and pathological conditions, such as scleroderma, Dupuytren's contracture and epidermolysis bullosa. Scarring and contraction of tissues in the eye may occur in various conditions, for example, the sequelae of retinal detachment or diabetic eye disease (as mentioned above). Contraction of the sockets found in the skull for the eyeballs and associated structures, including extra-ocular muscles and eyelids, may occur if there is trauma or inflammatory damage. The tissues contract within the sockets causing a variety of problems including double vision and an unsightly appearance.

Other indications include Vocal cord fibrosis, Intestinal fibrosis and Fibrosis associated with brain infarction.

For further information on different types of fibrosis see: Molina V, et al., (2002), “Fibrotic diseases”, Harefuah, 141(11): 973-8, 1009; Yu L, et al., (2002), “Therapeutic strategies to halt renal fibrosis”, Curr Opin Pharmacol. 2(2):177-81; Keane W F and Lyle P A. (2003), “Recent advances in management of type 2 diabetes and nephropathy: lessons from the RENAAL study”, Am J Kidney Dis. 41(3 Suppl 2): S22-5; Bohle A, et al., (1989), “The pathogenesis of chronic renal failure”, Pathol Res Pract. 185(4):421-40; Kikkawa R, et al., (1997), “Mechanism of the progression of diabetic nephropathy to renal failure”, Kidney Int Suppl. 62:S39-40; Bataller R, and Brenner D A. (2001), “Hepatic stellate cells as a target for the treatment of liver fibrosis”, Semin Liver Dis. 21(3):437-51; Gross T J and Hunninghake G W, (2001) “Idiopathic pulmonary fibrosis”, N Engl J Med. 345(7):517-25; Frohlich E D. (2001) “Fibrosis and ischemia: the real risks in hypertensive heart disease”, Am J Hypertens; 14(6 Pt 2):194S-199S; Friedman S L. (2003), “Liver fibrosis—from bench to bedside”, J Hepatol. 38 Suppl 1:S38-53; Albanis E, et al., (2003), “Treatment of hepatic fibrosis: almost there”, Curr Gastroenterol Rep. 5(1):48-56; (Weber K T. (2000), “Fibrosis and hypertensive heart disease”, Curr Opin Cardiol. 15(4):264-72).

### Delivery of Nucleic Acid Molecules and Pharmaceutical Formulations

Nucleic acid molecules may be adapted for use to prevent or treat fibrotic (e.g., liver, kidney, peritoneal, and pulmonary) diseases, traits, conditions and/or disorders, and/or any other trait, disease, disorder or condition that is related to or will respond to the levels of TIMP1 and TIMP2 in a cell or tissue. A nucleic acid molecule may include a delivery vehicle, including liposomes, for administration to a subject, carriers and diluents and their salts, and/or can be present in pharmaceutically acceptable formulations.

Nucleic acid molecules of the present invention may be delivered to the target tissue by direct application of the naked molecules prepared with a carrier or a diluent.

The terms “naked nucleic acid” or “naked dsRNA” or “naked siRNA” refers to nucleic acid molecules that are free from any delivery vehicle that acts to assist, promote or facilitate entry into the cell, including viral sequences, viral particles, liposome formulations, lipofectin or precipitating agents and the like. For example, dsRNA in PBS is “naked dsRNA”.

Nucleic acid molecules disclosed herein may be delivered or administered directly with a carrier or diluent but not any delivery vehicle that acts to assist, promote or facilitate entry to the cell, including viral vectors, viral particles, liposome formulations, lipofectin or precipitating agents and the like.

Nucleic acid molecules may be delivered or administered to a subject by direct application of the nucleic acid molecules with a carrier or diluent or any other delivery vehicle that acts to assist, promote or facilitate entry into a cell, including viral sequences, viral particular, liposome formulations, lipofectin or precipitating agents and the like. Polypeptides that facilitate introduction of nucleic acid into a desired subject such as those described in US. Application Publication No. 20070155658 (e.g., a melamine derivative such as 2,4,6-Triguanidino Traizine and 2,4,6-Tramidosarcocyl Melamine, a polyarginine polypeptide, and a polypeptide including alternating glutamine and asparagine residues).

Methods for the delivery of nucleic acid molecules are described in Akhtar et al., Trends Cell Bio., 2: 139 (1992); Delivery Strategies for Antisense Oligonucleotide Therapeutics, ed. Akhtar, (1995), Maurer et al., Mol. Membr. Biol., 16: 129-140 (1999); Hofland and Huang, Handb. Exp. Pharmacol., 137: 165-192 (1999); and Lee et al., ACS Symp. Ser., 752: 184-192 (2000); U.S. Pat. Nos. 6,395,713; 6,235,310; 5,225,182; 5,169,383; 5,167,616; 4,959217; 4,925,678; 4,487,603; and 4,486,194 and Sullivan et al., PCT WO 94/02595; PCT WO 00/03683 and PCT WO 02/08754; and U.S. Patent Application Publication No. 2003077829. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see e.g., Gonzalez et al., Bioconjugate Chem., 10: 1068-1074 (1999); Wang et al., International PCT publication Nos. WO 03/47518 and WO 03/46185), poly(lactic-co-glycolic)acid (PLGA) and PLCA microspheres (see for example U.S. Pat. No. 6,447,796 and U.S. Application Publication No. 2002130430), biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules as disclosed herein, whether subcutaneous, intramuscular, or intradermal, can take place using standard needle and syringe methodologies, or by needle-free technologies such as those described in Conry et al., Clin. Cancer Res., 5: 2330-2337 (1999) and Barry et al., International PCT Publication No. WO 99/31262. The molecules of as described herein can be used as pharmaceutical agents. Pharmaceutical agents may prevent, modulate the occurrence, or treat (alleviate a symptom to some extent, preferably all of the symptoms) of a disease state in a subject.

Nucleic acid molecules may be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers.

Delivery systems include surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011).

Nucleic acid molecules may be formulated or complexed with polyethylenimine (e.g., linear or branched PEI) and/or polyethylenimine derivatives, including for example polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives, grafted PEIs such as galactose PEI, cholesterol PEI, antibody derivatized PEI, and polyethylene glycol PEI (PEG-PEI) derivatives thereof (see for example Ogris et al., 2001, AAPA PharmSci, 3, 1-11; Furgeson et al., 2003, Bioconjugate Chem., 14, 840-847; Kunath et al., 2002, Pharmaceutical Research, 19, 810-817; Choi et al., 2001, Bull. Korean Chem. Soc., 22, 46-52; Bettinger et al., 1999, Bioconjugate Chem., 10, 558-561; Peterson et al., 2002, Bioconjugate Chem., 13, 845-854; Erbacher et al., 1999, Journal of Gene Medicine Preprint, 1, 1-18; Godbey et al., 1999., PNAS USA, 96, 5177-5181; Godbey et al., 1999, Journal of Controlled Release, 60, 149-160; Diebold et al., 1999, Journal of Biological Chemistry, 274, 19087-19094; Thomas and Klibanov, 2002, PNAS USA, 99, 14640-14645; Sagara, U.S. Pat. No. 6,586,524 and United States Patent Application Publication No. 20030077829.

Nucleic acid molecules may be complexed with membrane disruptive agents such as those described in U.S. Patent Application Publication No. 20010007666. The membrane disruptive agent or agents and the nucleic acid molecule may also be complexed with a cationic lipid or helper lipid molecule, such as those lipids described in U.S. Pat. No. 6,235,310.

The nucleic acid molecules may be administered via pulmonary delivery, such as by inhalation of an aerosol or spray dried formulation administered by an inhalation device or nebulizer, providing rapid local uptake of the nucleic acid molecules into relevant pulmonary tissues. Solid particulate compositions containing respirable dry particles of micronized nucleic acid compositions can be prepared by grinding dried or lyophilized nucleic acid compositions, and then passing the micronized composition through, for example, a 400 mesh screen to break up or separate out large agglomerates. A solid particulate composition comprising the nucleic acid compositions of contemplated herein can optionally contain a dispersant which serves to facilitate the formation of an aerosol as well as other therapeutic compounds. A suitable dispersant is lactose, which can be blended with the nucleic acid compound in any suitable ratio, such as a 1 to 1 ratio by weight.

Aerosols of liquid particles may include a nucleic acid molecules disclosed herein and can be produced by any suitable means, such as with a nebulizer (see e.g., U.S. Pat. No. 4,501,729). Nebulizers are commercially available devices which transform solutions or suspensions of an active ingredient into a therapeutic aerosol mist either by means of acceleration of a compressed gas, typically air or oxygen, through a narrow venturi orifice or by means of ultrasonic agitation. Suitable formulations for use in nebulizers include the active ingredient in a liquid carrier in an amount of up to 40% w/w preferably less than 20% w/w of the formulation. The carrier is typically water or a dilute aqueous alcoholic solution, preferably made isotonic with body fluids by the addition of, e.g., sodium chloride or other suitable salts. Optional additives include preservatives if the formulation is not prepared sterile, e.g., methyl hydroxybenzoate, anti-oxidants, flavorings, volatile oils, buffering agents and emulsifiers and other formulation surfactants. The aerosols of solid particles including the active composition and surfactant can likewise be produced with any solid particulate aerosol generator. Aerosol generators for administering solid particulate therapeutics to a subject produce particles which are respirable, as explained above, and generate a volume of aerosol containing a predetermined metered dose of a therapeutic composition at a rate suitable for human administration. One illustrative type of solid particulate aerosol generator is an insufflator. Suitable formulations for administration by insufflation include finely comminuted powders which can be delivered by means of an insufflator. In the insufflator, the powder, e.g., a metered dose thereof effective to carry out the treatments described herein, is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically includes from 0.1 to 100 w/w of the formulation. A second type of illustrative aerosol generator includes a metered dose inhaler. Metered dose inhalers are pressurized aerosol dispensers, typically containing a suspension or solution formulation of the active ingredient in a liquefied propellant. During use these devices discharge the formulation through a valve adapted to deliver a metered volume to produce a fine particle spray containing the active ingredient. Suitable propellants include certain chlorofluorocarbon compounds, e.g., dichlorodifluoromethane, trichlorofluoromethane, dichlorotetrafluoroethane and mixtures thereof. The formulation can additionally contain one or more co-solvents, for example, ethanol, emulsifiers and other formulation surfactants, such as oleic acid or sorbitan trioleate, anti-oxidants and suitable flavoring agents. Other methods for pulmonary delivery are described in, e.g., US Patent Application No. 20040037780, and U.S. Pat. Nos. 6,592,904; 6,582,728; 6,565,885. PCT Patent Publiction No. WO2008/132723 discloses aerosol delivery of oligonucleotides in general, and of siRNA in particular, to the respiratory system.

Nucleic acid molecules may be administered to the central nervous system (CNS) or peripheral nervous system (PNS). Experiments have demonstrated the efficient in vivo uptake of nucleic acids by neurons. See e.g., Sommer et al., 1998, Antisense Nuc. Acid Drug Dev., 8, 75; Epa et al., 2000, Antisense Nuc. Acid Drug Dev., 10, 469; Broaddus et al., 1998, J. Neurosurg., 88(4), 734; Karle et al., 1997, Eur. J. Pharmocol., 340(2/3), 153; Bannai et al., 1998, Brain Research, 784(1,2), 304; Rajakumar et al., 1997, Synapse, 26(3), 199; Wu-pong et al., 1999, BioPharm, 12(1), 32; Bannai et al., 1998, Brain Res. Protoc., 3(1), 83; and Simantov et al., 1996, Neuroscience, 74(1), 39. Nucleic acid molecules are therefore amenable to delivery to and uptake by cells in the CNS and/or PNS.

Delivery of nucleic acid molecules to the CNS is provided by a variety of different strategies. Traditional approaches to CNS delivery that can be used include, but are not limited to, intrathecal and intracerebroventricular administration, implantation of catheters and pumps, direct injection or perfusion at the site of injury or lesion, injection into the brain arterial system, or by chemical or osmotic opening of the blood-brain barrier. Other approaches can include the use of various transport and carrier systems, for example though the use of conjugates and biodegradable polymers. Furthermore, gene therapy approaches, e.g., as described in Kaplitt et al., U.S. Pat. No. 6,180,613 and Davidson, WO 04/013280, can be used to express nucleic acid molecules in the CNS.

Delivery systems may include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Examples of liposomes which can be used include the following: (1) CellFectin, 1:1.5 (M/M) liposome formulation of the cationic lipid N,NI,NII,NIII-tetramethyl-N,NI,NII,NIII-tetrapalmit-y-spermine and dioleoyl phosphatidylethanolamine (DOPE) (GIBCO BRL); (2) Cytofectin GSV, 2:1 (M/M) liposome formulation of a cationic lipid and DOPE (Glen Research); (3) DOTAP(N-[1-(2,3-dioleoyloxy)-N,N,N-tri-methyl-ammoniummethylsulfate) (Boehringer Manheim); and (4) Lipofectamine, 3:1 (M/M) liposome formulation of the polycationic lipid DOSPA, the neutral lipid DOPE (GIBCO BRL) and Di-Alkylated Amino Acid (DiLA2).

Delivery systems may include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Nucleic acid molecules may be formulated or complexed with polyethylenimine (e.g., linear or branched PEI) and/or polyethylenimine derivatives, including for example grafted PEIs such as galactose PEI, cholesterol PEI, antibody derivatized PEI, and polyethylene glycol PEI (PEG-PEI) derivatives thereof (see for example Ogris et al., 2001, AAPA PharmSci, 3, 1-11; Furgeson et al., 2003, Bioconjugate Chem., 14, 840-847; Kunath et al., 2002, Pharmaceutical Research, 19, 810-817; Choi et al., 2001, Bull. Korean Chem. Soc., 22, 46-52; Bettinger et al., 1999, Bioconjugate Chem., 10, 558-561; Peterson et al., 2002, Bioconjugate Chem., 13, 845-854; Erbacher et al., 1999, Journal of Gene Medicine Preprint, 1, 1-18; Godbey et al., 1999., PNAS USA, 96, 5177-5181; Godbey et al., 1999, Journal of Controlled Release, 60, 149-160; Diebold et al., 1999, Journal of Biological Chemistry, 274, 19087-19094; Thomas and Klibanov, 2002, PNAS USA, 99, 14640-14645; and Sagara, U.S. Pat. No. 6,586,524.

Nucleic acid molecules may include a bioconjugate, for example a nucleic acid conjugate as described in Vargeese et al., U.S. Ser. No. 10/427,160; U.S. Pat. No. 6,528,631; U.S. Pat. No. 6,335,434; U.S. Pat. No. 6,235,886; U.S. Pat. No. 6,153,737; U.S. Pat. No. 5,214,136; U.S. Pat. No. 5,138,045.

Compositions, methods and kits disclosed herein may include an expression vector that includes a nucleic acid sequence encoding at least one nucleic acid molecule as provided herein in a manner that allows expression of the nucleic acid molecule. Methods of introducing nucleic acid molecules or one or more vectors capable of expressing the strands of dsRNA into the environment of the cell will depend on the type of cell and the make up of its environment. The nucleic acid molecule or the vector construct may be directly introduced into the cell (i.e., intracellularly); or introduced extracellularly into a cavity, interstitial space, into the circulation of an organism, introduced orally, or may be introduced by bathing an organism or a cell in a solution containing dsRNA. The cell is preferably a mammalian cell; more preferably a human cell. The nucleic acid molecule of the expression vector can include a sense region and an antisense region. The antisense region can include a sequence complementary to a RNA or DNA sequence encoding TIMP1 and TIMP2 and the sense region can include a sequence complementary to the antisense region. The nucleic acid molecule can include two distinct strands having complementary sense and antisense regions. The nucleic acid molecule can include a single strand having complementary sense and antisense regions.

Nucleic acid molecules that interact with target RNA molecules and down-regulate gene encoding target RNA molecules (e.g., target RNA molecules referred to by Genbank Accession numbers herein) may be expressed from transcription units inserted into DNA or RNA vectors. Recombinant vectors can be DNA plasmids or viral vectors. Nucleic acid molecule expressing viral vectors can be constructed based on, but not limited to, adeno-associated virus, retrovirus, adenovirus, or alphavirus. The recombinant vectors capable of expressing the nucleic acid molecules can be delivered as described herein, and persist in target cells. Alternatively, viral vectors can be used that provide for transient expression of nucleic acid molecules. Such vectors can be repeatedly administered as necessary. Once expressed, the nucleic acid molecules bind and down-regulate gene function or expression via RNA interference (RNAi). Delivery of nucleic acid molecule expressing vectors can be systemic, such as by intravenous or intramuscular administration, by administration to target cells ex-planted from a subject followed by reintroduction into the subject, or by any other means that would allow for introduction into the desired target cell.

Expression vectors may include a nucleic acid sequence encoding at least one nucleic acid molecule disclosed herein, in a manner which allows expression of the nucleic acid molecule. For example, the vector may contain sequence(s) encoding both strands of a nucleic acid molecule that include a duplex. The vector can also contain sequence(s) encoding a single nucleic acid molecule that is self-complementary and thus forms a nucleic acid molecule. Non-limiting examples of such expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725. Expression vectors may also be included in a mammalian (e.g., human) cell.

An expression vector may include a nucleic acid sequence encoding two or more nucleic acid molecules, which can be the same or different. Expression vectors may include a sequence for a nucleic acid molecule complementary to a nucleic acid molecule referred to by a Genbank Accession number NM_{—}003254 (TIMP1) or NM_{—}003255 (TIMP2).

An expression vector may encode one or both strands of a nucleic acid duplex, or a single self-complementary strand that self hybridizes into a nucleic acid duplex. The nucleic acid sequences encoding nucleic acid molecules can be operably linked in a manner that allows expression of the nucleic acid molecule (see for example Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725).

An expression vector may include one or more of the following: a) a transcription initiation region (e.g., eukaryotic pol I, II or III initiation region); b) a transcription termination region (e.g., eukaryotic pol I, II or III termination region); c) an intron and d) a nucleic acid sequence encoding at least one of the nucleic acid molecules, wherein said sequence is operably linked to the initiation region and the termination region in a manner that allows expression and/or delivery of the nucleic acid molecule. The vector can optionally include an open reading frame (ORF) for a protein operably linked on the 5′ side or the 3′-side of the sequence encoding the nucleic acid molecule; and/or an intron (intervening sequences).

Transcription of the nucleic acid molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as siNA in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736. The above nucleic acid transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (see Couture and Stinchcomb, 1996 supra).

Nucleic acid molecule may be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45. Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856.

A viral construct packaged into a viral particle would accomplish both efficient introduction of an expression construct into the cell and transcription of dsRNA construct encoded by the expression construct.

Methods for oral introduction include direct mixing of RNA with food of the organism, as well as engineered approaches in which a species that is used as food is engineered to express an RNA, then fed to the organism to be affected. Physical methods may be employed to introduce a nucleic acid molecule solution into the cell. Physical methods of introducing nucleic acids include injection of a solution containing the nucleic acid molecule, bombardment by particles covered by the nucleic acid molecule, soaking the cell or organism in a solution of the RNA, or electroporation of cell membranes in the presence of the nucleic acid molecule.

Other methods known in the art for introducing nucleic acids to cells may be used, such as lipid-mediated carrier transport, chemical mediated transport, such as calcium phosphate, and the like. Thus the nucleic acid molecules may be introduced along with components that perform one or more of the following activities: enhance RNA uptake by the cell, promote annealing of the duplex strands, stabilize the annealed strands, or other-wise increase inhibition of the target gene.

The nucleic acid molecules or the vector construct can be introduced into the cell using suitable formulations. One formulation comprises a lipid formulation such as in Lipofectamine™ 2000 (Invitrogen, CA, USA. Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate dsRNA in a buffer or saline solution and directly inject the formulated dsRNA into cells, as in studies with oocytes. The direct injection of dsRNA duplexes may also be done. For suitable methods of introducing dsRNA see U.S. published patent application No. 2004/0203145, 20070265220 which are incorporated herein by reference.

Polymeric nanocapsules or microcapsules facilitate transport and release of the encapsulated or bound dsRNA into the cell. They include polymeric and monomeric materials, especially including polybutylcyanoacrylate. A summary of materials and fabrication methods has been published (see Kreuter, 1991). The polymeric materials which are formed from monomeric and/or oligomeric precursors in the polymerization/nanoparticle generation step, are per se known from the prior art, as are the molecular weights and molecular weight distribution of the polymeric material which a person skilled in the field of manufacturing nanoparticles may suitably select in accordance with the usual skill.

Nucleic acid moles may be formulated as a microemulsion. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system.

Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

### Water Soluble Crosslinked Polymers

Delivery formulations can include water soluble degradable crosslinked polymers that include one or more degradable crosslinking lipid moiety, one or more PEI moiety, and/or one or more mPEG (methyl ether derivative of PEG (methoxypoly (ethylene glycol)).

Degradable lipid moieties preferably include compounds having the following structural motif:

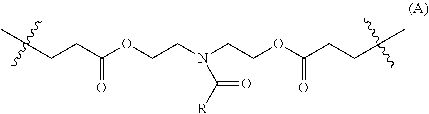

In the above formula, ester linkages are biodegradable groups, R represents a relatively hydrophobic “lipo” group, and the structural motif shown occurs m times where m is in the range of about 1 to about 30. For example, in certain embodiments R is selected from the group consisting of C₂-C₅₀ alkyl, C₂-C₅₀ heteroalkyl, C₂-C₅₀ alkenyl, C₂-C₅₀ heteroalkenyl, C₅-C₅₀ aryl; C₂-C₅₀ heteroaryl; C₂-C₅₀ alkynyl, C₂-C₅₀ heteroalkynyl, C₂-C₅₀ carboxyalkenyl, and C₂-C₅₀ carboxyheteroalkenyl. In preferred embodiments, R is a saturated or unsaturated alkyl having 4 to 30 carbons, more preferably 8 to 24 carbons or a sterol, preferably a cholesteryl moiety. In preferred embodiments, R is oleic, lauric, myristic, palmitic margaric, stearic, arachidic, behenic, or lignoceric. In a most preferred embodiment, R is oleic.

The N in formula (B) may have an electron pair or a bond to a hydrogen atom. When N has an electron pair, the recurring unit may be cationic at low pH.

The degradable crosslinking lipid moiety may be reacted with a polyethyleneimine (PEI) as shown in Scheme A below:

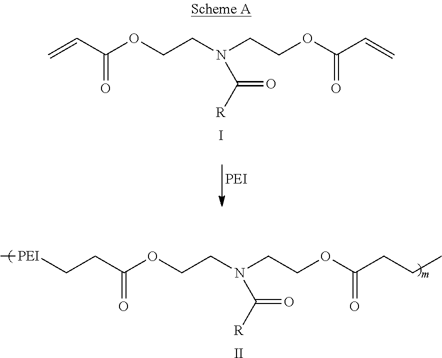

In formula (A), R has the same meanings as described above. The PEI may contain recurring units of formula (B) in which x is an integer in the range of about 1 to about 100 and y is an integer in the range of about 1 to about 100.

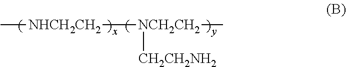

The reaction illustrated in Scheme A may be carried out by intermixing the PEI and the diacrylate (I) in a mutual solvent such as ethanol, methanol or dichloromethane with stirring, preferably at room temperature for several hours, then evaporating the solvent to recover the resulting polymer. While not wishing to be bound to any particular theory, it is believed that the reaction between the PEI and diacrylate (I) involves a Michael reaction between one or more amines of the PEI with double bond(s) of the diacrylate (see J. March, Advanced Organic Chemistry 3rd Ed., pp. 711-712 (1985)). The diacrylate shown in Scheme A may be prepared in the manner as described in U.S. application Ser. No. 11/216,986 (US Publication No. 2006/0258751).

The molecular weight of the PEI is preferably in the range of about 200 to 25,000 Daltons more preferably 400 to 5,000 Daltons, yet more preferably 600 to 2000 Daltons. PEI may be either branched or linear.

The molar ratio of PEI to diacrylate is preferably in the range of about 1:2 to about 1:20. The weight average molecular weight of the cationic lipopolymer may be in the range of about 500 Daltons to about 1,000,000 Daltons preferably in the range of about 2,000 Daltons to about 200,000 Daltons. Molecular weights may be determined by size exclusion chromatography using PEG standards or by agarose gel electrophoresis.

The cationic lipopolymer is preferably degradable, more preferably biodegradable, e.g., degradable by a mechanism selected from the group consisting of hydrolysis, enzyme cleavage, reduction, photo-cleavage, and sonication. While not wishing to be bound to any particular theory, but it is believed that degradation of the cationic lipopolymer of formula (II) within the cell proceeds by enzymatic cleavage and/or hydrolysis of the ester linkages.

Synthesis may be carried out by reacting the degradable lipid moiety with the PEI moiety as described above. Then the mPEG (methyl ether derivative of PEG (methoxypoly (ethylene glycol)), is added to form the degradable crosslinked polymer. In preferred embodiments, the reaction is carried out at room temperature. The reaction products may be isolated by any means known in the art including chromatographic techniques. In a preferred embodiment, the reaction product may be removed by precipitation followed by centrifugation.

### Dosages

The useful dosage to be administered and the particular mode of administration will vary depending upon such factors as the cell type, or for in vivo use, the age, weight and the particular animal and region thereof to be treated, the particular nucleic acid and delivery method used, the therapeutic or diagnostic use contemplated, and the form of the formulation, for example, suspension, emulsion, micelle or liposome, as will be readily apparent to those skilled in the art. Typically, dosage is administered at lower levels and increased until the desired effect is achieved.

When lipids are used to deliver the nucleic acid, the amount of lipid compound that is administered can vary and generally depends upon the amount of nucleic acid being administered. For example, the weight ratio of lipid compound to nucleic acid is preferably from about 1:1 to about 30:1, with a weight ratio of about 5:1 to about 10:1 being more preferred.

A suitable dosage unit of nucleic acid molecules may be in the range of 0.001 to 0.25 milligrams per kilogram body weight of the recipient per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day.

Suitable amounts of nucleic acid molecules may be introduced and these amounts can be empirically determined using standard methods. Effective concentrations of individual nucleic acid molecule species in the environment of a cell may be about 1 femtomolar, about 50 femtomolar, 100 femtomolar, 1 picomolar, 1.5 picomolar, 2.5 picomolar, 5 picomolar, 10 picomolar, 25 picomolar, 50 picomolar, 100 picomolar, 500 picomolar, 1 nanomolar, 2.5 nanomolar, 5 nanomolar, 10 nanomolar, 25 nanomolar, 50 nanomolar, 100 nanomolar, 500 nanomolar, 1 micromolar, 2.5 micromolar, 5 micromolar, 10 micromolar, 100 micromolar or more.

Dosage may be from 0.01 μg to 1 g per kg of body weight (e.g., 0.1 μg, 0.25 μg, 0.5 μg, 0.75 μg, 1 μg, 2.5 μg, 5 μg, 10 μg, 25 μg, 50 μg, 100 μg, 250 μg, 500 μg, 1 mg, 2.5 mg, 5 mg, 10 mg, 25 mg, 50 mg, 100 mg, 250 mg, or 500 mg per kg).

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

Pharmaceutical compositions that include the nucleic acid molecule disclosed herein may be administered once daily, qid, tid, bid, QD, or at any interval and for any duration that is medically appropriate. However, the therapeutic agent may also be dosed in dosage units containing two, three, four, five, six or more sub-doses administered at appropriate intervals throughout the day. In that case, the nucleic acid molecules contained in each sub-dose may be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. The dosage unit may contain a corresponding multiple of the daily dose. The composition can be compounded in such a way that the sum of the multiple units of a nucleic acid together contain a sufficient dose.

### Pharmaceutical Compositions, Kits, and Containers

Also provided are compositions, kits, containers and formulations that include a nucleic acid molecule (e.g., an siNA molecule) as provided herein for reducing expression of TIMP1 and TIMP2 for administering or distributing the nucleic acid molecule to a patient. A kit may include at least one container and at least one label. Suitable containers include, for example, bottles, vials, syringes, and test tubes. The containers can be formed from a variety of materials such as glass, metal or plastic. The container can hold amino acid sequence(s), small molecule(s), nucleic acid sequence(s), cell population(s) and/or antibody(s). In one embodiment, the container holds a polynucleotide for use in examining the mRNA expression profile of a cell, together with reagents used for this purpose. In another embodiment a container includes an antibody, binding fragment thereof or specific binding protein for use in evaluating TIMP1 and TIMP2 protein expression cells and tissues, or for relevant laboratory, prognostic, diagnostic, prophylactic and therapeutic purposes; indications and/or directions for such uses can be included on or with such container, as can reagents and other compositions or tools used for these purposes. Kits may further include associated indications and/or directions; reagents and other compositions or tools used for such purpose can also be included.

The container can alternatively hold a composition that is effective for treating, diagnosis, prognosing or prophylaxing a condition and can have a sterile access port (for example the container can be an intravenous solution bag or a vial having a stopper pierceable by a hypodermic injection needle). The active agents in the composition can be a nucleic acid molecule capable of specifically binding TIMP1 and TIMP2 and/or modulating the function of TIMP1 and TIMP2.

A kit may further include a second container that includes a pharmaceutically-acceptable buffer, such as phosphate-buffered saline, Ringer's solution and/or dextrose solution. It can further include other materials desirable from a commercial and user standpoint, including other buffers, diluents, filters, stirrers, needles, syringes, and/or package inserts with indications and/or instructions for use.

The units dosage ampoules or multidose containers, in which the nucleic acid molecules are packaged prior to use, may include an hermetically sealed container enclosing an amount of polynucleotide or solution containing a polynucleotide suitable for a pharmaceutically effective dose thereof, or multiples of an effective dose. The polynucleotide is packaged as a sterile formulation, and the hermetically sealed container is designed to preserve sterility of the formulation until use.

The container in which the polynucleotide including a sequence encoding a cellular immune response element or fragment thereof may include a package that is labeled, and the label may bear a notice in the form prescribed by a governmental agency, for example the Food and Drug Administration, which notice is reflective of approval by the agency under Federal law, of the manufacture, use, or sale of the polynucleotide material therein for human administration.

Federal law requires that the use of pharmaceutical compositions in the therapy of humans be approved by an agency of the Federal government. In the United States, enforcement is the responsibility of the Food and Drug Administration, which issues appropriate regulations for securing such approval, detailed in 21 U.S.C. §301-392. Regulation for biologic material, including products made from the tissues of animals is provided under 42 U.S.C. §262. Similar approval is required by most foreign countries. Regulations vary from country to country, but individual procedures are well known to those in the art and the compositions and methods provided herein preferably comply accordingly.

The dosage to be administered depends to a large extent on the condition and size of the subject being treated as well as the frequency of treatment and the route of administration. Regimens for continuing therapy, including dose and frequency may be guided by the initial response and clinical judgment. The parenteral route of injection into the interstitial space of tissues is preferred, although other parenteral routes, such as inhalation of an aerosol formulation, may be required in specific administration, as for example to the mucous membranes of the nose, throat, bronchial tissues or lungs.

As such, provided herein is a pharmaceutical product which may include a polynucleotide including a sequence encoding a cellular immune response element or fragment thereof in solution in a pharmaceutically acceptable injectable carrier and suitable for introduction interstitially into a tissue to cause cells of the tissue to express a cellular immune response element or fragment thereof, a container enclosing the solution, and a notice associated with the container in form prescribed by a governmental agency regulating the manufacture, use, or sale of pharmaceuticals, which notice is reflective of approval by the agency of manufacture, use, or sale of the solution of polynucleotide for human administration.

### Indications

The nucleic acid molecules disclosed herein can be used to treat diseases, conditions or disorders associated with TIMP1 and TIMP2, such as liver fibrosis, cirrhosis, pulmonary fibrosis, kidney fibrosis, peritoneal fibrosis, chronic hepatic damage, and fibrillogenesis and any other disease or conditions that are related to or will respond to the levels of TIMP1 and TIMP2 in a cell or tissue. As such, compositions, kits and methods disclosed herein may include packaging a nucleic acid molecule disclosed herein that includes a label or package insert. The label may include indications for use of the nucleic acid molecules such as use for treatment or prevention of liver fibrosis, peritoneal fibrosis, kidney fibrosis and pulmonary fibrosis, and any other disease or conditions that are related to or will respond to the levels of TIMP1 and TIMP2 in a cell or tissue. A label may include an indication for use in reducing expression of TIMP1 and TIMP2. A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

Those skilled in the art will recognize that two or more siTIMP1 and or siTIMP2 may be combined or that other anti-fibrosis treatments, drugs and therapies known in the art can be readily combined with the nucleic acid molecules herein (e.g. siNA molecules) and are hence contemplated herein.

The methods and compositions provided herein will now be described in greater detail by reference to the following non-limiting examples.

### EXAMPLES

### Example 1

### siRNA Sequences

siRNA sequences for TIMP-1, TIMP-2, positive control and negative control are listed in Tabls C and D. 100 μM siRNA stock solution was prepared by dissolving in nucrease free water (Ambion). In the “Sequence” columns in Tables C and D, lower case letters represent unmodified ribonucleotides, “T” represents deoxyribothymidine.

TABLE C

| | | | | | | |
|---|---|---|---|---|---|---|
| | | | | SEQ ID | | |
| Target | Name | Orientation | Sequence | NO: | Species | nucleotides |

| | | | | | | |
|---|---|---|---|---|---|---|
| TIMP1 | TIMP1-A | S (5′->3′) | ccaccuuauaccagcguuaTT | 5 | Human, | [355-373] |
| | | AS (3′->5′) | TTgguggaauauggucgcaau | 6 | mouse, rat, | ORF |
| | | | | | rhesus | |
| TIMP1 | TIMP1-B | S (5′->3′) | cacuguuggcugugaggaaTT | 7 | Human | [620-638] |
| | | AS (3′->5) | TTgugacaaccgacacuccuu | 8 | rhesus | ORF |
| TIMP1 | TIMP1-C | S (5′->3′) | gcacaguguuucccuguuuTT | 9 | Human mouse | [640-658] |
| | | AS (3′->5′) | TTcgugucacaaagggacaaa | 10 | rat rhesus | ORF |

TABLE D

| | | | | SEQ ID | | |
|---|---|---|---|---|---|---|
| Target | Name | Orientation | Sequence | NO | Species | nucleotides |
| TIMP2 | TIMP2-A | S (5′->3′) | ugcagauguagugaucaggTT | 11 | human | [421-439] |
| | | AS (3′->5′) | TTacgucuacaucacuagucc | 12 | | ORF |
| TIMP2 | TIMP2-B | S (5′->3′) | gaggauccaguaugagaucTT | 13 | human | [502-520] |
| | | AS (3′->5′) | TTcuccuaggucauacucuag | 14 | rhesus | ORF |
| | | | | | rabbit | |
| TIMP2 | TIMP2-C | S (5′->3′) | gcagauaaagauguucaaaTT | 15 | human | [523-541] |
| | | AS (3′->5′) | TTcgucuauuucuacaaguuu | 16 | mouse rat | ORF |
| | | | | | cow dog | |
| | | | | | pig | |
| TIMP2 | TIMP2-D | S (5′->3′) | ggaauaucucauugcaggaTT | 17 | human | [625-643] |
| | | AS (3′->5′) | TTccuuauagaguaacguccu | 18 | | ORF |
| TIMP2 | TIMP2-E | S (5′->3′) | uaucucauugcaggaaaggTT | 19 | human | [629-647] |
| | | AS (3′->5′) | TTauagaguaacguccuuucc | 20 | | ORF |

### Example 2

### siRNA Delivery

HT-1080 cell (Japanese Collection of Research Bioresources) was maintained incubated in DMEM (Sigma, Cat#D6546) with 10% fetal bovine serum (FBS; Hyclone, Cat.#SH30070.03) and 1% volume/volume L-Glutamine-penicillin-streptomycin solution (Sigma, Cat.#G1146) and 1% volume/volume L-Glutamine solution (Sigma, Cat.#G7513). Before delivering siRNA, cells were seeded in 6-well plate (Nunc. #140675) at the density of 5×10³ cells per well and incubated at 37° C. with 7.5% CO₂ for 2 days. siRNAs for TIMP1 were transfected to the cells with VA-coupled liposome (VA-liposome) as described by Sato et al. (Sato Y. et al. Nature Biotechnology 2008. Vol. 26, p431) and siRNAs for TIMP2 were delivered with VA-conjugated cationic polymer (VA-polymer), synthesized in-house at the ratio of 5:1 (VA-polymer:siRNA, weight per weight). The final concentration of siRNA was 50 nM. 2-hours after siRNA delivery, cell culture medium was replaced to fresh DMEM with 10% FBS and incubated for 2 overnight at 37° C. with 7.5% CO₂.

### Example 3

### Gene Knocking Down Assessment of siRNA by RT-PCR

After transfection as described in Example 2, total RNA was isolated with QIAshreader QIAGEN, 79654) and RNeasy Mini Kit (QIAGEN, 74104) by following manufacturer's protocol. 1 μg of the isolated total RNA was used for cDNA preparation with Hicapacity RNA-to-cDNA Master Mix (Applied Biosystems, 4390779) as indicated by manufacturer's protocol. Then, 0.05 μg of cDNA was employed for polymerase chain reaction (PCR) with ExTaq (TaKaRa, RR001B) polymerase by following supplied manual. PCR primers for detection of each gene are listed in excel file. PCR condition was as follows: 94° C. 4 min, then 4° C. 30 sec, 63° C. 30 sec, 72° C. 1 min for 23 cycles, 72° C. 5 min before termination. 15 μl of PCR products for TIMP-1 or TIMP-2 gene and 5 μl for GAPDH gene were identified by agarose gel electrophoresis.

FIG. 2 indicates knock down efficacy of siRNAs for TIMP1 as measured by qPCR. The amount of PCR product from cells transfected with TIMP-1 siRNA, e.g. TIMP1-A (SEQ ID NOS:5 and 6), TIMP1-B (SEQ ID NOS:7 and 8) or TIMP1-C (SEQ ID NOS:9 and 10), was less than that from untreated cells, therefore, those siRNA for TIMP1 gene are capable to knock down the target gene.

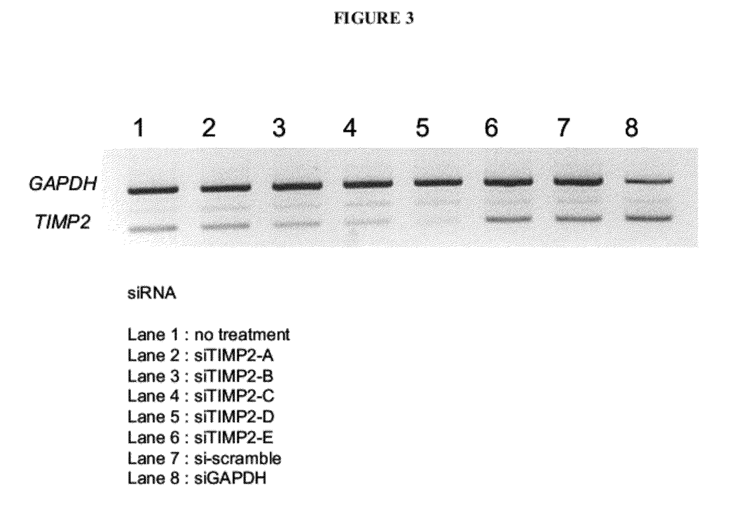

FIG. 3 represents knock down efficacy of siRNAs for TIMP2 as measured by qPCR: TIMP2-A (SEQ ID NOS:11 and 12), TIMP2-B (SEQ ID NOS:13 and 14), TIMP2-C (SEQ ID NOS:15 and 16), TIMP2-D (SEQ ID NOS:17 and 18) and TIMP2-E (SEQ ID NOS:19 and 20). TIMP2 siRNA showed target gene knock down and level of gene silencing was dependent on the sequence.

### Example 4

### Treatment of Liver Cirrhosis in Rats with siTIMP1 and siTIMP2

Liver cirrhosis animal model: Liver cirrhosis was induced in rats using the method described by Sato et al., (Sato Y. et al. Nat Biotech 2008. 26:431). Briefly, liver cirrhosis was induced in 4 week-old male SD rats by injecting them dimethylnitrosoamine (DMN) (Wako Chemicals, Japan) as follows: 0.5% DMN in phosphate-buffered saline (PBS) was administered to rats intraperitoneally at a dose of 2 ml/kg per body weight for 3 consecutive days per week. Specifically, DMN solution was injected on days 0 (start of the experiment), 2, 4, 7, 9, 11, 14, 16, 18, 21, 23, 25, 28, 30, 32, 34 and 36.

siRNA Sequence for TIMP1 (“siTIMP1-A”)

| | | |
|---|---|---|
| S (5′->3′) | ccaccuuauaccagcguuaTT | (SEQ ID NO: 5) |
| AS (3′->5′) | TTgguggaauauggucgcaau | (SEQ ID NO: 6) |

siRNA Sequence for TIMP2 (“siTIMP2-C”)

| | | |
|---|---|---|
| S (5′->3′) | gcagauaaagauguucaaaTT | (SEQ ID NO: 15) |
| AS (3′->5′) | TTcgucuauuucuacaaguuu | (SEQ ID NO: 16) |

A 10 μg/μl siRNA stock solution was prepared by dissolving siRNA duplexes (siTIMP1 or siTIMP2) in nuclease free water (Ambion). For treatment of rats, siRNA was formulated with vitamin A-coupled liposome as described by Sato et al (Sato Y. et al. Nature Biotech 2008. Vol. 26, p431). The vitamin A (VA)-liposome-siRNA formulation consisted of 0.33 μmol/ml of VA, 0.33 μmol/ml of liposome (Coatsome EL-01-D, NOF Corporation) and 0.5 μg/μl of siRNA in 5% glucose solution.

Injection Solution for siRNA Delivered at a Concentration of 0.75 mg/Kg

The liposomes were prepared at a concentration of 1 mM by addition of nuclease-free water, and left for 15 min at room temperature before use. To prepare VA-coupled liposomes, 100 nmol of vitamin A (dissolved in DMSO) was mixed with the liposomes (100 nmol) by vortex for 15 seconds at R.T.

The siRNA duplexes (150 μg) were prepared at a concentration of 10 μg/μl by addition of nuclease-free water. A 5% glucose (175 μl) solution was added to the liposomal suspension. (Total volume of 300 μl). The VA-liposome-siRNA solutions were injected to each rat to a final concentration of 0.75 ml/Kg body weight.

Injection solution for siRNA delivered at a concentration of 1.5 mg/Kg

The liposomes were prepared at a concentration of 1 mM by addition of nuclease-free water, and left to stand for 15 min before use. To prepare VA-coupled liposome, 200 nmol of vitamin A (dissolved in DMSO) was mixed with the liposome (200 nmol) by vortex for 15 seconds at R.T.

The siRNA duplexes (300 μg) were prepared at a concentration of 10 μg/μl by addition of nuclease-free water. A 5% glucose (50 μl) solution was added to the liposomal suspension. (Total volume 300 μl). The VA-liposome-siRNA solutions were injected to each rat to a final concentration of 1.5 ml/Kg body weight.

siRNA Treatment

The siRNA treatment was carried out from day 28 for 5 times by intravenous injection. In detail, rats were treated with siRNA on days 28, 30, 32, 34 and 36 post DMN treatment. Rats were sacrificed on day 38 or 39. Two different siRNA species (siTIMP1-A and siTIMP2-C) and 2 different doses (0.75 mg siRNA per kg body weight, 1.5 mg siRNA per kg body weight) were tested. Details of tested groups and number of animals in each group are as follows:
1) Control animals: Liver cirrhosis was induced by DMN injection, and a 5% glucose was administered (n=9)
2) VA-Lip-siTIMP1-A 0.75 mg/Kg (n=9)
3) VA-Lip-siTIMP1-A 1.5 mg/Kg (n=9)
4) VA-Lip-siTIMP2-C 0.75 mg/Kg (n=9)
5) VA-Lip-siTIMP2-C1.5 mg/Kg (n=9)
6) Sham (PBS was injected instead of DMN. 5% Glucose was administered instead of siRNA) (n=5)
7) Untreated control animals (Intact) (n=5)

Evaluation of Therapeutic Efficacy

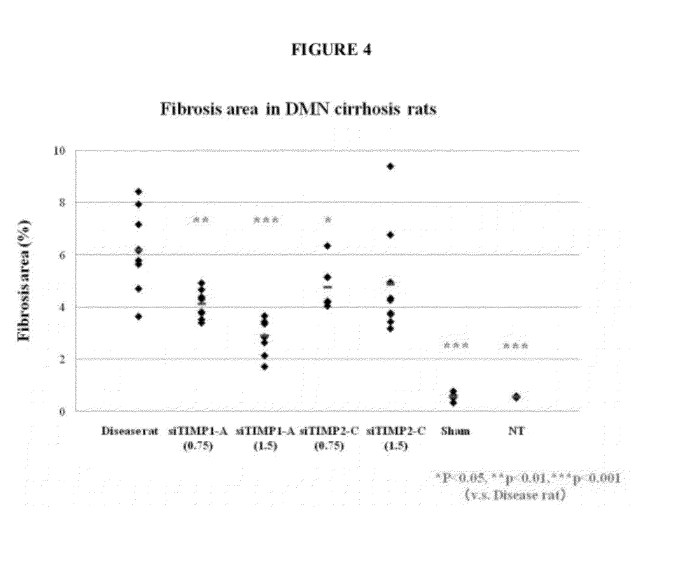

On day 38, 2 out of 10 animals in “siTIMP2-C” group died and were not analyzed further. However, other animals were survived before the sacrifice. After rats were sacrificed, liver tissues were fixed in 10% formalin. The left lobe of the liver was embedded in paraffin for tissue slide preparation. Tissue slides were stained with Sirius red as well as hematoxylin and eosin (HE). Sirius red staining was employed to visualize collagen-deposited area and determine the level of cirrhosis. HE staining was used for nuclei and cytoplasm as counter-staining Each slide was observed under microscope (BZ-9000, Keyence Corp. Japan) and the percentage of Sirius red-stained area per slide was determined by image analysis software attached to the microscope. At least 4 slides per each liver were prepared for image analysis, and whole area of each slide (slice of liver) was captured by camera and analyzed. Statistic analysis was carried out by t-test analysis. Results are shown in FIG. 4. Liver sections were photographed at ×32 magnification. The fibrotic areas were calculated as the mean of 4 liver sections. The bar graph summarizes the digital quantification of staining for each group. Statistical values are as follows: *=P<0.05, **=P<0.01, ***=P<0.001

FIG. 4 represents the fibrotic area in liver sections. The area of fibrosis in the “diseased rat” (group 1) was higher than “sham” (group 6) or “untreated” (group 7) groups. Therefore, DMN treatment induced collagen deposition in liver, typical of liver fibrosis. The area of fibrosis was significantly reduced by the treatment of siRNA targeting TIMP1 gene (groups 2 and 3), compared with “diseased rat” group, indicating that siRNA to TIMP1 has therapeutic efficacy in treating fibrotic diseases and disorders.

### Example 5

### Selecting TIMP1 and TIMP2 Nucleic Acid Molecule Sequences

Nucleic acid molecules (e.g., siNA≦25 nucleotides) against TIMP1 and TIMP2 were designed using a proprietary database. Candidate sequences are validated by in vitro knock down assays. Details of the nucleic acids set forth in the Tables are

The Tables (A1, A2, A5, A6, B1, B2, B5, B6) include
a. 19-mer and 18-mer siRNAs (sense and corresponding antisense sequences to form duplex siRNA) predicted to be active by a proprietary database and excludes known 19-mer siRNAs;
b. siRNAs which target human and at least two additional species (cross species) selected from dog, rat, mouse and rabbit and are predicted to be active;
   i. inclusion of cross-species siRNA compounds are siRNA with full match to the indicated target (“Sense”) and
   ii. inclusion of siRNAs with mismatches relative to the target, at positions 1, 19 (5′>3′) or both.

The Tables of “preferred” siRNA (A3, A7, B3, B7) include sense and corresponding antisense sequences that were selected as follows:
i. Selection for cross species to human (H) and rat (Rt) and inclusion of sequences with 1 MM (single mismatch (MM)) to rat target in positions other than 1/19 (5′>3′)
ii. Addition of predicted active siRNA compounds that don't target rat but target at least two other species selected from dog, mouse and rabbit.
iii. Addition of best siRNA targeting human or human+rhesus
iv. Exclusion of siRNAs that target miRNA seed sequence
v. Exclusion of siRNAs with high G/C content
vi. Exclusion of siRNAs targeting multiple SNPs

The Tables labeled as “lowest predicted OT effect” (Tables A4, A8, B4 and B8) relate to siRNA from the “preferred” Tables having best off-target (OT) features including
c. Column labeled “Crosses”—Indicates species specificity as follows:
   i. H/Rt=siRNA targeting at least human and rat
   ii. H/Rt (Rt cross—with 1MM)=siRNA targeting at least human and rat. Target match to rat is partial and there is one mismatch at a position other than 1 or 19
   iii. Other (w/o Rt)—siRNA targets human and other species but not rat
   iv. H+/−Rh=siRNA targeting only human or human and Rhesus but no other species

Column labeled “# in HTS list”—Indicates the siRNA number in the preceding “Preferred” Table (A3, A7, B3, B7).
2. Selection is done in the following manner:
   i. Mismatches (MM) are identified in positions 2-18 of the guide strand. MM in positions 1 and 19 are NOT considered as mismatches.
   ii. Exclusion of siRNAs having complete match (0 MM) to other genes
   iii. Exclusion of siRNAs having 1 MM in position 17 or 18 (of AS strand) to other genes
   iv. Preference (ranking of predicted OT activity):
      1—has 3MM within positions 2-16 of AS (5′>3′).
      2—has 2MM to 1-4 gene targets within positions 2-16
      3—has 2MM to 5-9 gene targets within (positions 2-16)
      4—targets 10-20 genes with 2MM (positions 2-16)

Sequences of sense and antisense oligonucleotides useful in the preparation of siRNA molecules are disclosed in Tables A1, A2, A3, A4, A5, A6, A7, A8, B1, B2, B3, B4, B5, B6, B7, B8 (Tables A1-B8) infra. Best OT refers to least number of matches to off-target genes.

The following abbreviations are used in the Tables A1-B8 (Tables A1, A2, A3, A4, A5, A6, A7 A8, B1, B2, B3, B4, B5, B6, B7 and B8) herein: “other spec or Sp.” refers to cross species identity with other animals: D or Dg-dog, Rt-rat, Rb-rabbit, Rh-rhesus monkey, Pg-Pig, M or Ms-Mouse, Ck-Chicken, Cw-Cow; ORF: open reading frame. 19-mers, and 18+1-mers refer to oligomers of 19 and 18+1 (U at position 1 of Antisense, A at position 19 of sense strand or A at position 1 of Antisense, U at position 19 of sense strand) ribonucleic acids in length, respectively.

TABLE A1
19-mer siTIMP1

| | | | | | | |
|---|---|---|---|---|---|---|
| | | SEQ | | SEQ | | |
| | | ID | | ID | | human-73858576 |
| No. | Sense (5′>3′) | NO. | Antisense (5′>3′) | NO. | Other Sp | ORF: 193-816 |

| | | | | | | |
|---|---|---|---|---|---|---|
| 1 | GUUUCUCAUUGCUGGAAAA | 21 | UUUUCCAGCAAUGAGAAAC | 129 | Rh | [506-524] ORF |
| 2 | CACAGUGUUUCCCUGUUUA | 22 | UAAACAGGGAAACACUGUG | 130 | Rh, M | [641-659] ORF |
| 3 | CUUUCUUCCGGACAAUGAA | 23 | UUCAUUGUCCGGAAGAAAG | 131 | | [874-892] 3′UTR |
| 4 | GCUGAAGCCUGCACAGUGU | 24 | ACACUGUGCAGGCUUCAGC | 132 | | [834-852] 3′UTR |
| 5 | GGAGUUUCUCAUUGCUGGA | 25 | UCCAGCAAUGAGAAACUCC | 133 | Rh | [503-521] ORF |
| 6 | GCACAGUGUUUCCCUGUUU | 26 | AAACAGGGAAACACUGUGC | 134 | Rh, Rt, M | [640-658] ORF |
| 7 | CAUCUUUCUUCCGGACAAU | 27 | AUUGUCCGGAAGAAAGAUG | 135 | | [871-889] 3′UTR |
| 8 | GGCUUCACCAAGACCUACA | 28 | UGUAGGUCUUGGUGAAGCC | 136 | Rh, Rb | [603-621] ORF |
| 9 | GGCACUCAUUGCUUGUGGA | 29 | UCCACAAGCAAUGAGUGCC | 137 | Rh | [684-702] ORF |
| 10 | CUCCCAUCUUUCUUCCGGA | 30 | UCCGGAAGAAAGAUGGGAG | 138 | | [867-885] 3′UTR |
| 11 | GUGUUUCCCUGUUUAUCCA | 31 | UGGAUAAACAGGGAAACAC | 139 | Rh | [645-663] ORF |
| 12 | AGUCAACCAGACCACCUUA | 32 | UAAGGUGGUCUGGUUGACU | 140 | Rh | [344-362] ORF |
| 13 | GCCCGGAGUGGAAGCUGAA | 33 | UUCAGCUUCCACUCCGGGC | 141 | | [821-839] 3′UTR |
| 14 | CCACCUUAUACCAGCGUUA | 34 | UAACGCUGGUAUAAGGUGG | 142 | Rh, Rt, M | [355-373] ORF |
| 15 | CAUGGAGAGUGUCUGCGGA | 35 | UCCGCAGACACUCUCCAUG | 143 | Rh | [455-473] ORF |
| 16 | CCAAGAUGUAUAAAGGGUU | 36 | AACCCUUUAUACAUCUUGG | 144 | Rh | [388-406] ORF |
| 17 | GAGAGUGUCUGCGGAUACU | 37 | AGUAUCCGCAGACACUCUC | 145 | Rh | [459-477] ORF |
| 18 | AGAUCAAGAUGACCAAGAU | 38 | AUCUUGGUCAUCUUGAUCU | 146 | Rh, Dg | [376-394] ORF |
| 19 | CUGCAGAGUGGCACUCAUU | 39 | AAUGAGUGCCACUCUGCAG | 147 | Rh | [675-693] ORF |
| 20 | CCUGGAACAGCCUGAGCUU | 40 | AAGCUCAGGCUGUUCCAGG | 148 | | [571-589] ORF |
| 21 | CACUGUUGGCUGUGAGGAA | 41 | UUCCUCACAGCCAACAGUG | 149 | Rh | [620-638] ORF |
| 22 | CCAGAAGUCAACCAGACCA | 42 | UGGUCUGGUUGACUUCUGG | 150 | Rh, Dg | [339-357] ORF |
| 23 | GAUGGACUCUUGCACAUCA | 43 | UGAUGUGCAAGAGUCCAUC | 151 | | [531-549] ORF |
| 24 | AGUUUCUCAUUGCUGGAAA | 44 | UUUCCAGCAAUGAGAAACU | 152 | Rh | [505-523] ORF |
| 25 | GCUCCUCCAAGGCUCUGAA | 45 | UUCAGAGCCUUGGAGGAGC | 153 | Rh | [710-728] ORF |
| 26 | ACAGUGUUUCCCUGUUUAU | 46 | AUAAACAGGGAAACACUGU | 154 | Rh, M | [642-660] ORF |
| 27 | GUCUGCGGAUACUUCCACA | 47 | UGUGGAAGUAUCCGCAGAC | 155 | Rh | [465-483] ORF |
| 28 | CUGGAACAGCCUGAGCUUA | 48 | UAAGCUCAGGCUGUUCCAG | 156 | | [572-590] ORF |
| 29 | GGGCUGUGCACCUGGCAGU | 49 | ACUGCCAGGUGCACAGCCC | 157 | Rh | [774-792] ORF |
| 30 | GUGUCUGCGGAUACUUCCA | 50 | UGGAAGUAUCCGCAGACAC | 158 | Rh | [463-481] ORF |
| 31 | AUGAGAUCAAGAUGACCAA | 51 | UUGGUCAUCUUGAUCUCAU | 159 | Rh, Dg | [373-391] ORF |
| 32 | GGAAGCUGAAGCCUGCACA | 52 | UGUGCAGGCUUCAGCUUCC | 160 | | [830-848] 3′UTR |
| 33 | GAGUUUCUCAUUGCUGGAA | 53 | UUCCAGCAAUGAGAAACUC | 161 | Rh | [504-522] ORF |
| 34 | CAGACGGCCUUCUGCAAUU | 54 | AAUUGCAGAAGGCCGUCUG | 162 | Rh | [285-303] ORF |
| 35 | GCACAUCACUACCUGCAGU | 55 | ACUGCAGGUAGUGAUGUGC | 163 | | [542-560] ORF |
| 36 | GGCAGUCCCUGCGGUCCCA | 56 | UGGGACCGCAGGGACUGCC | 164 | | [787-805] ORF |
| 37 | GAUGUAUAAAGGGUUCCAA | 57 | UUGGAACCCUUUAUACAUC | 165 | Rh | [392-410] ORF |
| 38 | CUGGCAUCCUGUUGUUGCU | 58 | AGCAACAACAGGAUGCCAG | 166 | | [217-235] ORF |
| 39 | GGACGGACCAGCUCCUCCA | 59 | UGGAGGAGCUGGUCCGUCC | 167 | Rh | [700-718] ORF |
| 40 | GAGUGGCACUCAUUGCUUG | 60 | CAAGCAAUGAGUGCCACUC | 168 | Rh | [680-698] ORF |
| 41 | AGAGUGUCUGCGGAUACUU | 61 | AAGUAUCCGCAGACACUCU | 169 | Rh | [460-478] ORF |
| 42 | ACCAGACCACCUUAUACCA | 62 | UGGUAUAAGGUGGUCUGGU | 170 | Rh | [349-367] ORF |
| 43 | CACCAAGACCUACACUGUU | 63 | AACAGUGUAGGUCUUGGUG | 171 | Rh | [608-626] ORF |
| 44 | GGCAUCCUGUUGUUGCUGU | 64 | ACAGCAACAACAGGAUGCC | 172 | Rh | [219-237] ORF |
| 45 | CCUGAAUCCUGCCCGGAGU | 65 | ACUCCGGGCAGGAUUCAGG | 173 | | [811-829] ORF + 3′UTR |
| 46 | GUCAACCAGACCACCUUAU | 66 | AUAAGGUGGUCUGGUUGAC | 174 | Rh | [345-363] ORF |
| 47 | GGACACCAGAAGUCAACCA | 67 | UGGUUGACUUCUGGUGUCC | 175 | Rh | [334-352] ORF |
| 48 | AGCGUUAUGAGAUCAAGAU | 68 | AUCUUGAUCUCAUAACGCU | 176 | Rh, Dg, Rt | [367-385] ORF |
| 49 | UGCACAGUGUUUCCCUGUU | 69 | AACAGGGAAACACUGUGCA | 177 | Rh, Rt, M | [639-657] ORF |
| 50 | ACUGCAGAGUGGCACUCAU | 70 | AUGAGUGCCACUCUGCAGU | 178 | Rh | [674-692] ORF |
| 51 | CCCAUCUUUCUUCCGGACA | 71 | UGUCCGGAAGAAAGAUGGG | 179 | | [869-887] 3′UTR |
| 52 | GUGAGGAAUGCACAGUGUU | 72 | AACACUGUGCAUUCCUCAC | 180 | Rh | [631-649] ORF |
| 53 | CCUCCAAGGCUCUGAAAAG | 73 | CUUUUCAGAGCCUUGGAGG | 181 | Rh | [713-731] ORF |
| 54 | CAUCACUACCUGCAGUUUU | 74 | AAAACUGCAGGUAGUGAUG | 182 | | [545-563] ORF |
| 55 | AGCUGAAGCCUGCACAGUG | 75 | CACUGUGCAGGCUUCAGCU | 183 | | [833-851] 3′UTR |
| 56 | GCACAGUGUCCACCCUGUU | 76 | AACAGGGUGGACACUGUGC | 184 | | [844-862] 3′UTR |
| 57 | CCAGCUCCUCCAAGGCUCU | 77 | AGAGCCUUGGAGGAGCUGG | 185 | Rh | [707-725] ORF |
| 58 | CUGUUGUUGCUGUGGCUGA | 78 | UCAGCCACAGCAACAACAG | 186 | Rh | [225-243] ORF |
| 59 | UCUUCCGGACAAUGAAAUA | 79 | UAUUUCAUUGUCCGGAAGA | 187 | | [877-895] 3′UTR |
| 60 | GCUCCCUGGAACAGCCUGA | 80 | UCAGGCUGUUCCAGGGAGC | 188 | | [567-585] ORF |
| 61 | AUAAAGGGUUCCAAGCCUU | 81 | AAGGCUUGGAACCCUUUAU | 189 | | [397-415] ORF |
| 62 | GAGUGGAAGCUGAAGCCUG | 82 | CAGGCUUCAGCUUCCACUC | 190 | Rh | [826-844] 3′UTR |
| 63 | CAAACUGCAGAGUGGCACU | 83 | AGUGCCACUCUGCAGUUUG | 191 | Rh | [671-689] ORF |
| 64 | CGGCCUUCUGCAAUUCCGA | 84 | UCGGAAUUGCAGAAGGCCG | 192 | Rh | [289-307] ORF |
| 65 | CUCCUCCAAGGCUCUGAAA | 85 | UUUCAGAGCCUUGGAGGAG | 193 | Rh | [711-729] ORF |
| 66 | CCUGCAAACUGCAGAGUGG | 86 | CCACUCUGCAGUUUGCAGG | 194 | Rh, Dg | [667-685] ORF |
| 67 | CACAUCACUACCUGCAGUU | 87 | AACUGCAGGUAGUGAUGUG | 195 | | [543-561] ORF |
| 68 | UGCCCGGAGUGGAAGCUGA | 88 | UCAGCUUCCACUCCGGGCA | 196 | | [820-838] 3′UTR |
| 69 | GCUUCUGGCAUCCUGUUGU | 89 | ACAACAGGAUGCCAGAAGC | 197 | | [213-231] ORF |
| 70 | UUUCUUCCGGACAAUGAAA | 90 | UUUCAUUGUCCGGAAGAAA | 198 | | [875-893] 3′UTR |
| 71 | UGCACAGUGUCCACCCUGU | 91 | ACAGGGUGGACACUGUGCA | 199 | | [843-861] 3′UTR |
| 72 | GACCUACACUGUUGGCUGU | 92 | ACAGCCAACAGUGUAGGUC | 200 | Rh | [614-632] ORF |
| 73 | CACAGACGGCCUUCUGCAA | 93 | UUGCAGAAGGCCGUCUGUG | 201 | Rh | [283-301] ORF |
| 74 | AGGGCUUCCAGUCCCGUCA | 94 | UGACGGGACUGGAAGCCCU | 202 | Rh | [730-748] ORF |
| 75 | CCCAGAUAGCCUGAAUCCU | 95 | AGGAUUCAGGCUAUCUGGG | 203 | | [802-820] ORF + 3′UTR |
| 76 | GUUGUUGCUGUGGCUGAUA | 96 | UAUCAGCCACAGCAACAAC | 204 | Rh | [227-245] ORF |
| 77 | GUCCCUGCGGUCCCAGAUA | 97 | UAUCUGGGACCGCAGGGAC | 205 | | [791-809] ORF |
| 78 | CUUCCAGUCCCGUCACCUU | 98 | AAGGUGACGGGACUGGAAG | 206 | Rh | [734-752] ORF |
| 79 | CCUACACUGUUGGCUGUGA | 99 | UCACAGCCAACAGUGUAGG | 207 | Rh | [616-634] ORF |
| 80 | ACAUCACUACCUGCAGUUU | 100 | AAACUGCAGGUAGUGAUGU | 208 | | [544-562] ORF |
| 81 | UCUUUCUUCCGGACAAUGA | 101 | UCAUUGUCCGGAAGAAAGA | 209 | | [873-891] 3′UTR |
| 82 | CCAGAUAGCCUGAAUCCUG | 102 | CAGGAUUCAGGCUAUCUGG | 210 | | [803-821] ORF + 3′UTR |
| 83 | CAGUGUUUCCCUGUUUAUC | 103 | GAUAAACAGGGAAACACUG | 211 | Rh, M | [643-661] ORF |
| 84 | CUGGCUUCUGGCAUCCUGU | 104 | ACAGGAUGCCAGAAGCCAG | 212 | | [210-228] ORF |
| 85 | GACGGACCAGCUCCUCCAA | 105 | UUGGAGGAGCUGGUCCGUC | 213 | Rh | [701-719] ORF |
| 86 | UGUUGUUGCUGUGGCUGAU | 106 | AUCAGCCACAGCAACAACA | 214 | Rh | [226-244] ORF |
| 87 | GACUCUUGCACAUCACUAC | 107 | GUAGUGAUGUGCAAGAGUC | 215 | | [535-553] ORF |
| 88 | CCCGCCAUGGAGAGUGUCU | 108 | AGACACUCUCCAUGGCGGG | 216 | Rh | [450-468] ORF |
| 89 | CGAGGAGUUUCUCAUUGCU | 109 | AGCAAUGAGAAACUCCUCG | 217 | Rh | [500-518] ORF |
| 90 | CACAGGUCCCACAACCGCA | 110 | UGCGGUUGUGGGACCUGUG | 218 | Rh | [480-498] ORF |
| 91 | ACACCAGAAGUCAACCAGA | 111 | UCUGGUUGACUUCUGGUGU | 219 | Rh | [336-354] ORF |
| 92 | UGUUCCCACUCCCAUCUUU | 112 | AAAGAUGGGAGUGGGAACA | 220 | Rh | [859-877] 3′UTR |
| 93 | CCCUGUUCCCACUCCCAUC | 113 | GAUGGGAGUGGGAACAGGG | 221 | Rh | [856-874] 3′UTR |
| 94 | CACCUUAUACCAGCGUUAU | 114 | AUAACGCUGGUAUAAGGUG | 222 | Rh, Rt, M | [356-374] ORF |
| 95 | CACCAGAAGUCAACCAGAC | 115 | GUCUGGUUGACUUCUGGUG | 223 | Rh | [337-355] ORF |
| 96 | UCCUCCAAGGCUCUGAAAA | 116 | UUUUCAGAGCCUUGGAGGA | 224 | Rh | [712-730] ORF |
| 97 | GAAGCCUGCACAGUGUCCA | 117 | UGGACACUGUGCAGGCUUC | 225 | | [837-855] 3′UTR |
| 98 | CCUGCACAGUGUCCACCCU | 118 | AGGGUGGACACUGUGCAGG | 226 | | [841-859] 3′UTR |
| 99 | CCGCCAUGGAGAGUGUCUG | 119 | CAGACACUCUCCAUGGCGG | 227 | Rh | [451-469] ORF |
| 100 | UAAAGGGUUCCAAGCCUUA | 120 | UAAGGCUUGGAACCCUUUA | 228 | | [398-416] ORF |
| 101 | CAAGAUGACCAAGAUGUAU | 121 | AUACAUCUUGGUCAUCUUG | 229 | Rh | [380-398] ORF |
| 102 | GUUUUGUGGCUCCCUGGAA | 122 | UUCCAGGGAGCCACAAAAC | 230 | | [559-577] ORF |
| 103 | GGAGUGGAAGCUGAAGCCU | 123 | AGGCUUCAGCUUCCACUCC | 231 | Rh | [825-843] 3′UTR |
| 104 | CUGACAUCCGGUUCGUCUA | 124 | UAGACGAACCGGAUGUCAG | 232 | Rh | [427-445] ORF |
| 105 | GCGUUAUGAGAUCAAGAUG | 125 | CAUCUUGAUCUCAUAACGC | 233 | Rh, Dg, Rt | [368-386] ORF |
| 106 | CGGACCAGCUCCUCCAAGG | 126 | CCUUGGAGGAGCUGGUCCG | 234 | Rh | [703-721] ORF |
| 107 | CAGGAUGGACUCUUGCACA | 127 | UGUGCAAGAGUCCAUCCUG | 235 | | [528-546] ORF |
| 108 | CAAGAUGUAUAAAGGGUUC | 128 | GAACCCUUUAUACAUCUUG | 236 | Rh | [389-407] ORF |

TABLE A2
19-mer Cross-Species siTIMP1

| | | | | | | |
|---|---|---|---|---|---|---|
| | | SEQ | | SEQ | | |
| | | ID | | ID | | human-73858576 |
| No. | Sense (5′>3′) | NO. | Antisense (5′>3′) | NO. | Other Sp | ORF: 193-816 |

| | | | | | | |
|---|---|---|---|---|---|---|
| 1 | ACCACCUUAUACCAGCGUU | 237 | AACGCUGGUAUAAGGUGGU | 252 | Rh, Rt, M | [354-372] ORF |
| 2 | ACCGCAGCGAGGAGUUUCU | 238 | AGAAACUCCUCGCUGCGGU | 253 | Rh, Rb, Dg, Rt | [493-511] ORF |
| 3 | AGACCACCUUAUACCAGCG | 239 | CGCUGGUAUAAGGUGGUCU | 254 | Rh, Rt, M | [352-370] ORF |
| 4 | GGGCUUCACCAAGACCUAC | 240 | GUAGGUCUUGGUGAAGCCC | 255 | Rh, Rb, Dg | [602-620] ORF |
| 5 | CAACCGCAGCGAGGAGUUU | 241 | AAACUCCUCGCUGCGGUUG | 256 | Rh, Rb, Dg, Rt | [491-509] ORF |
| 6 | CAGACCACCUUAUACCAGC | 242 | GCUGGUAUAAGGUGGUCUG | 257 | Rh, Rt, M | [351-369] ORF |
| 7 | ACCUUAUACCAGCGUUAUG | 243 | CAUAACGCUGGUAUAAGGU | 258 | Rh, Rt, M | [357-375] ORF |
| 8 | CGUCAUCAGGGCCAAGUUC | 244 | GAACUUGGCCCUGAUGACG | 259 | Rh, Rb, Dg | [311-329] ORF |
| 9 | AUGCACAGUGUUUCCCUGU | 245 | ACAGGGAAACACUGUGCAU | 260 | Rh, Rt, M | [638-656] ORF |
| 10 | ACCUGGCAGUCCCUGCGGU | 246 | ACCGCAGGGACUGCCAGGU | 261 | Rh, Rb, Dg | [783-801] ORF |
| 11 | GACCACCUUAUACCAGCGU | 247 | ACGCUGGUAUAAGGUGGUC | 262 | Rh, Rt, M | [353-371] ORF |
| 12 | CCGCAGCGAGGAGUUUCUC | 248 | GAGAAACUCCUCGCUGCGG | 263 | Rh, Rb, Dg, Rt | [494-512] ORF |
| 13 | AACCGCAGCGAGGAGUUUC | 249 | GAAACUCCUCGCUGCGGUU | 264 | Rh, Rb, Dg, Rt | [492-510] ORF |
| 14 | UUAUGAGAUCAAGAUGACC | 250 | GGUCAUCUUGAUCUCAUAA | 265 | Rh, Dg, Rt | [371-389] ORF |
| 15 | ACCAGCGUUAUGAGAUCAA | 251 | UUGAUCUCAUAACGCUGGU | 266 | Rh, Rt | [364-382] ORF |

TABLE A3
Preferred 19-mer siTIMP1

| | | SEQ | | SEQ | | human- |
|---|---|---|---|---|---|---|
| | | ID | | ID | | 73858576 |
| siTIMP1_pNo. | Sense (5′>3′) | NO. | Antisense (5′>3′) | NO. | length | ORF: 193-816 |
| siTIMP1_p2 | GCACAGUGUUUCCCUGUUU | 267 | AAACAGGGAAACACUGUGC | 299 | 19 | [640-658] ORF |
| siTIMP1_p6 | CCACCUUAUACCAGCGUUA | 268 | UAACGCUGGUAUAAGGUGG | 300 | 19 | [355-373] ORF |
| siTIMP1_p14 | UGCACAGUGUUUCCCUGUU | 269 | AACAGGGAAACACUGUGCA | 301 | 19 | [639-657] ORF |
| siTIMP1_p16 | CACCUUAUACCAGCGUUAU | 270 | AUAACGCUGGUAUAAGGUG | 302 | 19 | [356-374] ORF |
| siTIMP1_p17 | GCGUUAUGAGAUCAAGAUG | 271 | CAUCUUGAUCUCAUAACGC | 303 | 19 | [368-386] ORF |
| siTIMP1_p19 | ACCACCUUAUACCAGCGUU | 272 | AACGCUGGUAUAAGGUGGU | 304 | 19 | [354-372] ORF |
| siTIMP1_p20 | ACCGCAGCGAGGAGUUUCU | 273 | AGAAACUCCUCGCUGCGGU | 305 | 19 | [493-511] ORF |
| siTIMP1_p21 | ACCAGCGUUAUGAGAUCAA | 274 | UUGAUCUCAUAACGCUGGU | 306 | 19 | [364-382] ORF |
| siTIMP1_p23 | CAACCGCAGCGAGGAGUUU | 275 | AAACUCCUCGCUGCGGUUG | 307 | 19 | [491-509] ORF |
| siTIMP1_p24 | CAGACCACCUUAUACCAGC | 276 | GCUGGUAUAAGGUGGUCUG | 308 | 19 | [351-369] ORF |
| siTIMP1_p27 | AGAUCAAGAUGACCAAGAU | 277 | AUCUUGGUCAUCUUGAUCU | 309 | 19 | [376-394] ORF |
| siTIMP1_p29 | CCAGAAGUCAACCAGACCA | 278 | UGGUCUGGUUGACUUCUGG | 310 | 19 | [339-357] ORF |
| siTIMP1_p31 | AUGAGAUCAAGAUGACCAA | 279 | UUGGUCAUCUUGAUCUCAU | 311 | 19 | [373-391] ORF |
| siTIMP1_p33 | CCUGCAAACUGCAGAGUGG | 280 | CCACUCUGCAGUUUGCAGG | 312 | 19 | [667-685] ORF |
| siTIMP1_p38 | CACAGUGUUUCCCUGUUUA | 281 | UAAACAGGGAAACACUGUG | 313 | 19 | [641-659] ORF |
| siTIMP1_p42 | ACAGUGUUUCCCUGUUUAU | 282 | AUAAACAGGGAAACACUGU | 314 | 19 | [642-660] ORF |
| siTIMP1_p43 | CAGUGUUUCCCUGUUUAUC | 283 | GAUAAACAGGGAAACACUG | 315 | 19 | [643-661] ORF |
| siTIMP1_p45 | CUUUCUUCCGGACAAUGAA | 284 | UUCAUUGUCCGGAAGAAAG | 316 | 19 | [874-892] 3′UTR |
| siTIMP1_p49 | GUUUCUCAUUGCUGGAAAA | 285 | UUUUCCAGCAAUGAGAAAC | 317 | 19 | [506-524] ORF |
| siTIMP1_p60 | CAUCUUUCUUCCGGACAAU | 286 | AUUGUCCGGAAGAAAGAUG | 318 | 19 | [871-889] 3′UTR |
| siTIMP1_p71 | CUCCCAUCUUUCUUCCGGA | 287 | UCCGGAAGAAAGAUGGGAG | 319 | 19 | [867-885] 3′UTR |
| siTIMP1_p73 | GUGUUUCCCUGUUUAUCCA | 288 | UGGAUAAACAGGGAAACAC | 320 | 19 | [645-663] ORF |
| siTIMP1_p77 | GCCCGGAGUGGAAGCUGAA | 289 | UUCAGCUUCCACUCCGGGC | 321 | 19 | [821-839] 3′UTR |
| siTIMP1_p78 | CAUGGAGAGUGUCUGCGGA | 290 | UCCGCAGACACUCUCCAUG | 322 | 19 | [455-473] ORF |
| siTIMP1_p79 | CCAAGAUGUAUAAAGGGUU | 291 | AACCCUUUAUACAUCUUGG | 323 | 19 | [388-406] ORF |
| siTIMP1_p85 | GAGAGUGUCUGCGGAUACU | 292 | AGUAUCCGCAGACACUCUC | 324 | 19 | [459-477] ORF |
| siTIMP1_p89 | CUGCAGAGUGGCACUCAUU | 293 | AAUGAGUGCCACUCUGCAG | 325 | 19 | [675-693] ORF |
| siTIMP1_p91 | CCUGGAACAGCCUGAGCUU | 294 | AAGCUCAGGCUGUUCCAGG | 326 | 19 | [571-589] ORF |
| siTIMP1_p96 | CACUGUUGGCUGUGAGGAA | 295 | UUCCUCACAGCCAACAGUG | 327 | 19 | [620-638] ORF |
| siTIMP1_p98 | GAUGGACUCUUGCACAUCA | 296 | UGAUGUGCAAGAGUCCAUC | 328 | 19 | [531-549] ORF |
| siTIMP1_p99 | AGUUUCUCAUUGCUGGAAA | 297 | UUUCCAGCAAUGAGAAACU | 329 | 19 | [505-523] ORF |
| siTIMP1_p108 | GUCUGCGGAUACUUCCACA | 298 | UGUGGAAGUAUCCGCAGAC | 330 | 19 | [465-483] ORF |

TABLE A4
19-mer siTIMP1 with lowest predicted OT effect

| | | | | SEQ | | SEQ |
|---|---|---|---|---|---|---|
| No. in TABLE | | | | ID | | ID |
| A3 | Cross species | Ranking | Sense (5′>3′) | NO. | Antisense (5′>3′) | NO. |
| siTIMP1_p2 | H/Rt | 3 | GCACAGUGUUUCCCUGUUU | 267 | AAACAGGGAAACACUGUGC | 299 |
| siTIMP1_p6 | H/Rt | 2 | CCACCUUAUACCAGCGUUA | 268 | UAACGCUGGUAUAAGGUGG | 300 |
| siTIMP1_p14 | H/Rt | 4 | UGCACAGUGUUUCCCUGUU | 269 | AACAGGGAAACACUGUGCA | 301 |
| siTIMP1_p16 | H/Rt | 1 | CACCUUAUACCAGCGUUAU | 270 | AUAACGCUGGUAUAAGGUG | 302 |
| siTIMP1_p17 | H/Rt | 2 | GCGUUAUGAGAUCAAGAUG | 271 | CAUCUUGAUCUCAUAACGC | 303 |
| siTIMP1_p19 | H/Rt | 2 | ACCACCUUAUACCAGCGUU | 272 | AACGCUGGUAUAAGGUGGU | 304 |
| siTIMP1_p20 | H/Rt | 3 | ACCGCAGCGAGGAGUUUCU | 273 | AGAAACUCCUCGCUGCGGU | 305 |
| siTIMP1_p21 | H/Rt | 3 | ACCAGCGUUAUGAGAUCAA | 274 | UUGAUCUCAUAACGCUGGU | 306 |
| siTIMP1_p23 | H/Rt | 3 | CAACCGCAGCGAGGAGUUU | 275 | AAACUCCUCGCUGCGGUUG | 307 |
| siTIMP1_p29 | Other (w/o Rt) | 3 | CCAGAAGUCAACCAGACCA | 278 | UGGUCUGGUUGACUUCUGG | 310 |
| siTIMP1_p33 | Other (w/o Rt) | 4 | CCUGCAAACUGCAGAGUGG | 280 | CCACUCUGCAGUUUGCAGG | 312 |
| siTIMP1_p38 | H/Rt (Rt Cross- | 3 | CACAGUGUUUCCCUGUUUA | 281 | UAAACAGGGAAACACUGUG | 313 |
| | with 1MM) | | | | | |
| siTIMP1_p42 | Other (w/o Rt) | 3 | ACAGUGUUUCCCUGUUUAU | 282 | AUAAACAGGGAAACACUGU | 314 |
| siTIMP1_p43 | Other (w/o Rt) | 3 | CAGUGUUUCCCUGUUUAUC | 283 | GAUAAACAGGGAAACACUG | 315 |
| siTIMP1_p45 | H +/− Rh | 4 | CUUUCUUCCGGACAAUGAA | 284 | UUCAUUGUCCGGAAGAAAG | 316 |
| siTIMP1_p60 | H +/− Rh | 2 | CAUCUUUCUUCCGGACAAU | 286 | AUUGUCCGGAAGAAAGAUG | 318 |
| siTIMP1_p71 | H +/− Rh | 4 | CUCCCAUCUUUCUUCCGGA | 287 | UCCGGAAGAAAGAUGGGAG | 319 |
| siTIMP1_p73 | H +/− Rh | 3 | GUGUUUCCCUGUUUAUCCA | 288 | UGGAUAAACAGGGAAACAC | 320 |
| siTIMP1_p78 | H +/− Rh | 3 | CAUGGAGAGUGUCUGCGGA | 290 | UCCGCAGACACUCUCCAUG | 322 |
| siTIMP1_p79 | H +/− Rh | 3 | CCAAGAUGUAUAAAGGGUU | 291 | AACCCUUUAUACAUCUUGG | 323 |
| siTIMP1_p85 | H +/− Rh | 2 | GAGAGUGUCUGCGGAUACU | 292 | AGUAUCCGCAGACACUCUC | 324 |
| siTIMP1_p89 | H +/− Rh | 3 | CUGCAGAGUGGCACUCAUU | 293 | AAUGAGUGCCACUCUGCAG | 325 |
| siTIMP1_p91 | H +/− Rh | 4 | CCUGGAACAGCCUGAGCUU | 294 | AAGCUCAGGCUGUUCCAGG | 326 |
| siTIMP1_p96 | H +/− Rh | 4 | CACUGUUGGCUGUGAGGAA | 295 | UUCCUCACAGCCAACAGUG | 327 |
| siTIMP1_p98 | H +/− Rh | 3 | GAUGGACUCUUGCACAUCA | 296 | UGAUGUGCAAGAGUCCAUC | 328 |
| siTIMP1_p99 | H +/− Rh | 4 | AGUUUCUCAUUGCUGGAAA | 297 | UUUCCAGCAAUGAGAAACU | 329 |
| siTIMP1_p108 | H +/− Rh | 2 | GUCUGCGGAUACUUCCACA | 298 | UGUGGAAGUAUCCGCAGAC | 330 |

TABLE A5
18-mer siTIMP1

| | | | | | | |
|---|---|---|---|---|---|---|
| | | SEQ | | SEQ | | |
| | | ID | | ID | | human-73858576 |
| No. | Sense (5′>3′) | NO. | Antisense (5′>3′) | NO. | Other Sp | ORF: 193-816 |

| | | | | | | |
|---|---|---|---|---|---|---|
| 1 | GGAGAGUGUCUGCGGAUA | 331 | UAUCCGCAGACACUCUCC | 582 | Rh | [458-475] ORF |
| 2 | GCUGAAGCCUGCACAGUG | 332 | CACUGUGCAGGCUUCAGC | 583 | | [834-851] 3′UTR |
| 3 | CAUCUUUCUUCCGGACAA | 333 | UUGUCCGGAAGAAAGAUG | 584 | | [871-888] 3′UTR |
| 4 | CCUCCAAGGCUCUGAAAA | 334 | UUUUCAGAGCCUUGGAGG | 585 | Rh | [713-730] ORF |
| 5 | CCGCCAUGGAGAGUGUCU | 335 | AGACACUCUCCAUGGCGG | 586 | Rh | [451-468] ORF |
| 6 | GAGUGUCUGCGGAUACUU | 336 | AAGUAUCCGCAGACACUC | 587 | Rh | [461-478] ORF |
| 7 | GAGUGGCACUCAUUGCUU | 337 | AAGCAAUGAGUGCCACUC | 588 | Rh | [680-697] ORF |
| 8 | AGCUGAAGCCUGCACAGU | 338 | ACUGUGCAGGCUUCAGCU | 589 | | [833-850] 3′UTR |
| 9 | AGAUCAAGAUGACCAAGA | 339 | UCUUGGUCAUCUUGAUCU | 590 | Rh, Dg | [376-393] ORF |
| 10 | GACUCUUGCACAUCACUA | 340 | UAGUGAUGUGCAAGAGUC | 591 | | [535-552] ORF |
| 11 | GAGUGGAAGCUGAAGCCU | 341 | AGGCUUCAGCUUCCACUC | 592 | Rh | [826-843] 3′UTR |
| 12 | CCUGCAAACUGCAGAGUG | 342 | CACUCUGCAGUUUGCAGG | 593 | Rh, Dg | [667-684] ORF |
| 13 | CAGUGUUUCCCUGUUUAU | 343 | AUAAACAGGGAAACACUG | 594 | Rh, Ms | [643-660] ORF |
| 14 | CCCUGUUCCCACUCCCAU | 344 | AUGGGAGUGGGAACAGGG | 595 | Rh | [856-873] 3′UTR |
| 15 | CCAGAUAGCCUGAAUCCU | 345 | AGGAUUCAGGCUAUCUGG | 596 | | [803-820] ORF + 3′UTR |
| 16 | GGUCCCAGAUAGCCUGAA | 346 | UUCAGGCUAUCUGGGACC | 597 | | [799-816] ORF |
| 17 | GGGCUUCACCAAGACCUA | 347 | UAGGUCUUGGUGAAGCCC | 598 | Rh, Rb, Dg | [602-619] ORF |
| 18 | GCGGAUACUUCCACAGGU | 348 | ACCUGUGGAAGUAUCCGC | 599 | Rh | [469-486] ORF |
| 19 | GAGAGUGUCUGCGGAUAC | 349 | GUAUCCGCAGACACUCUC | 600 | Rh | [459-476] ORF |
| 20 | GCGUUAUGAGAUCAAGAU | 350 | AUCUUGAUCUCAUAACGC | 601 | Rh, Dg, Rt | [368-385] ORF |
| 21 | GGAACAGCCUGAGCUUAG | 351 | CUAAGCUCAGGCUGUUCC | 602 | | [574-591] ORF |
| 22 | CUGAAAAGGGCUUCCAGU | 352 | ACUGGAAGCCCUUUUCAG | 603 | Rh | [724-741] ORF |
| 23 | CCAGCGUUAUGAGAUCAA | 353 | UUGAUCUCAUAACGCUGG | 604 | Rh, Rt | [365-382] ORF |
| 24 | CAACCAGACCACCUUAUA | 354 | UAUAAGGUGGUCUGGUUG | 605 | Rh | [347-364] ORF |
| 25 | GAGGAAUGCACAGUGUUU | 355 | AAACACUGUGCAUUCCUC | 606 | Rh | [633-650] ORF |
| 26 | CCAAGAUGUAUAAAGGGU | 356 | ACCCUUUAUACAUCUUGG | 607 | Rh | [388-405] ORF |
| 27 | CAGACCACCUUAUACCAG | 357 | CUGGUAUAAGGUGGUCUG | 608 | Rh, Rt, Ms | [351-368] ORF |
| 28 | AGUGGAAGCUGAAGCCUG | 358 | CAGGCUUCAGCUUCCACU | 609 | Rh | [827-844] 3′UTR |
| 29 | ACAGUGUUUCCCUGUUUA | 359 | UAAACAGGGAAACACUGU | 610 | Rh, Ms | [642-659] ORF |
| 30 | CGCCAUGGAGAGUGUCUG | 360 | CAGACACUCUCCAUGGCG | 611 | Rh | [452-469] ORF |
| 31 | CACCAGAAGUCAACCAGA | 361 | UCUGGUUGACUUCUGGUG | 612 | Rh | [337-354] ORF |
| 32 | CAGAAGUCAACCAGACCA | 362 | UGGUCUGGUUGACUUCUG | 613 | Rh | [340-357] ORF |
| 33 | CCCACUCCCAUCUUUCUU | 363 | AAGAAAGAUGGGAGUGGG | 614 | Rh | [863-880] 3′UTR |
| 34 | GCGAGGAGUUUCUCAUUG | 364 | CAAUGAGAAACUCCUCGC | 615 | Rh | [499-516] ORF |
| 35 | GCUUCACCAAGACCUACA | 365 | UGUAGGUCUUGGUGAAGC | 616 | Rh | [604-621] ORF |
| 36 | CAUCACUACCUGCAGUUU | 366 | AAACUGCAGGUAGUGAUG | 617 | | [545-562] ORF |
| 37 | AGCGUUAUGAGAUCAAGA | 367 | UCUUGAUCUCAUAACGCU | 618 | Rh, Dg, Rt | [367-384] ORF |
| 38 | CUGCAGAGUGGCACUCAU | 368 | AUGAGUGCCACUCUGCAG | 619 | Rh | [675-692] ORF |
| 39 | GAAGCUGAAGCCUGCACA | 369 | UGUGCAGGCUUCAGCUUC | 620 | | [831-848] 3′UTR |
| 40 | AUCACUACCUGCAGUUUU | 370 | AAAACUGCAGGUAGUGAU | 621 | | [546-563] ORF |
| 41 | GCACAGUGUUUCCCUGUU | 371 | AACAGGGAAACACUGUGC | 622 | Rh, Rt, Ms | [640-657] ORF |
| 42 | CCUGGAACAGCCUGAGCU | 372 | AGCUCAGGCUGUUCCAGG | 623 | | [571-588] ORF |
| 43 | GCAUCCUGUUGUUGCUGU | 373 | ACAGCAACAACAGGAUGC | 624 | Rh | [220-237] ORF |
| 44 | GUCCCAGAUAGCCUGAAU | 374 | AUUCAGGCUAUCUGGGAC | 625 | | [800-817] ORF + 3′UTR |
| 45 | AGUGUUUCCCUGUUUAUC | 375 | GAUAAACAGGGAAACACU | 626 | Rh, Ms | [644-661] ORF |
| 46 | GGCUGUGAGGAAUGCACA | 376 | UGUGCAUUCCUCACAGCC | 627 | Rh | [627-644] ORF |
| 47 | AGACCACCUUAUACCAGC | 377 | GCUGGUAUAAGGUGGUCU | 628 | Rh, Rt, Ms | [352-369] ORF |
| 48 | GGAUGGACUCUUGCACAU | 378 | AUGUGCAAGAGUCCAUCC | 629 | | [530-547] ORF |
| 49 | CGGACCAGCUCCUCCAAG | 379 | CUUGGAGGAGCUGGUCCG | 630 | Rh | [703-720] ORF |
| 50 | GGCCUUCUGCAAUUCCGA | 380 | UCGGAAUUGCAGAAGGCC | 631 | Rh | [290-307] ORF |
| 51 | GGGCUUCCAGUCCCGUCA | 381 | UGACGGGACUGGAAGCCC | 632 | Rh | [731-748] ORF |
| 52 | GCAGAGUGGCACUCAUUG | 382 | CAAUGAGUGCCACUCUGC | 633 | Rh | [677-694] ORF |
| 53 | CAGCGAGGAGUUUCUCAU | 383 | AUGAGAAACUCCUCGCUG | 634 | Rh, Rb, Rt | [497-514] ORF |
| 54 | CAGAUAGCCUGAAUCCUG | 384 | CAGGAUUCAGGCUAUCUG | 635 | | [804-821] ORF + 3′UTR |
| 55 | GCAGCGAGGAGUUUCUCA | 385 | UGAGAAACUCCUCGCUGC | 636 | Rh, Rb, Rt | [496-513] ORF |
| 56 | CCUGCAGUUUUGUGGCUC | 386 | GAGCCACAAAACUGCAGG | 637 | | [553-570] ORF |
| 57 | GUUAUGAGAUCAAGAUGA | 387 | UCAUCUUGAUCUCAUAAC | 638 | Rh, Dg, Rt | [370-387] ORF |
| 58 | CAAGAUGUAUAAAGGGUU | 388 | AACCCUUUAUACAUCUUG | 639 | Rh | [389-406] ORF |
| 59 | CCGGAGUGGAAGCUGAAG | 389 | CUUCAGCUUCCACUCCGG | 640 | Rh | [823-840] 3′UTR |
| 60 | AGGAGUUUCUCAUUGCUG | 390 | CAGCAAUGAGAAACUCCU | 641 | Rh | [502-519] ORF |
| 61 | GGCUGUGCACCUGGCAGU | 391 | ACUGCCAGGUGCACAGCC | 642 | Rh | [775-792] ORF |
| 62 | GUUUCCCUGUUUAUCCAU | 392 | AUGGAUAAACAGGGAAAC | 643 | Rh | [647-664] ORF |
| 63 | GCAGUUUUGUGGCUCCCU | 393 | AGGGAGCCACAAAACUGC | 644 | | [556-573] ORF |
| 64 | GUCAACCAGACCACCUUA | 394 | UAAGGUGGUCUGGUUGAC | 645 | Rh | [345-362] ORF |
| 65 | CCAUGGAGAGUGUCUGCG | 395 | CGCAGACACUCUCCAUGG | 646 | Rh | [454-471] ORF |
| 66 | CUGGCAUCCUGUUGUUGC | 396 | GCAACAACAGGAUGCCAG | 647 | | [217-234] ORF |
| 67 | CAGACGGCCUUCUGCAAU | 397 | AUUGCAGAAGGCCGUCUG | 648 | Rh | [285-302] ORF |
| 68 | ACUGCAGAGUGGCACUCA | 398 | UGAGUGCCACUCUGCAGU | 649 | Rh | [674-691] ORF |
| 69 | CGGAGUGGAAGCUGAAGC | 399 | GCUUCAGCUUCCACUCCG | 650 | Rh | [824-841] 3′UTR |
| 70 | GCCUCGGGAGCCAGGGCU | 400 | AGCCCUGGCUCCCGAGGC | 651 | Rh | [761-778] ORF |
| 71 | CCAGACCACCUUAUACCA | 401 | UGGUAUAAGGUGGUCUGG | 652 | Rh | [350-367] ORF |
| 72 | GGCUCUGAAAAGGGCUUC | 402 | GAAGCCCUUUUCAGAGCC | 653 | Rh | [720-737] ORF |
| 73 | GCUGGAAAACUGCAGGAU | 403 | AUCCUGCAGUUUUCCAGC | 654 | | [516-533] ORF |
| 74 | CCUGAAUCCUGCCCGGAG | 404 | CUCCGGGCAGGAUUCAGG | 655 | | [811-828] ORF + 3′UTR |
| 75 | CUGAAGCCUGCACAGUGU | 405 | ACACUGUGCAGGCUUCAG | 656 | | [835-852] 3′UTR |
| 76 | GGCAUCCUGUUGUUGCUG | 406 | CAGCAACAACAGGAUGCC | 657 | Rh | [219-236] ORF |
| 77 | CCCUGCAAACUGCAGAGU | 407 | ACUCUGCAGUUUGCAGGG | 658 | Rh, Dg | [666-683] ORF |
| 78 | CUGGAAAACUGCAGGAUG | 408 | CAUCCUGCAGUUUUCCAG | 659 | | [517-534] ORF |
| 79 | UCUCAUUGCUGGAAAACU | 409 | AGUUUUCCAGCAAUGAGA | 660 | Rh | [509-526] ORF |
| 80 | GUGGCUCCCUGGAACAGC | 410 | GCUGUUCCAGGGAGCCAC | 661 | | [564-581] ORF |
| 81 | CCAGCUCCUCCAAGGCUC | 411 | GAGCCUUGGAGGAGCUGG | 662 | Rh | [707-724] ORF |
| 82 | AGACCUACACUGUUGGCU | 412 | AGCCAACAGUGUAGGUCU | 663 | Rh | [613-630] ORF |
| 83 | GGGACACCAGAAGUCAAC | 413 | GUUGACUUCUGGUGUCCC | 664 | Rh | [333-350] ORF |
| 84 | GGCUCCCUGGAACAGCCU | 414 | AGGCUGUUCCAGGGAGCC | 665 | | [566-583] ORF |
| 85 | GUUCCCACUCCCAUCUUU | 415 | AAAGAUGGGAGUGGGAAC | 666 | Rh | [860-877] 3′UTR |
| 86 | CUCUGAAAAGGGCUUCCA | 416 | UGGAAGCCCUUUUCAGAG | 667 | Rh | [722-739] ORF |
| 87 | GGCUUCUGGCAUCCUGUU | 417 | AACAGGAUGCCAGAAGCC | 668 | | [212-229] ORF |
| 88 | AGGAAUGCACAGUGUUUC | 418 | GAAACACUGUGCAUUCCU | 669 | Rh | [634-651] ORF |
| 89 | CUUCUGGCAUCCUGUUGU | 419 | ACAACAGGAUGCCAGAAG | 670 | | [214-231] ORF |
| 90 | CAAACUGCAGAGUGGCAC | 420 | GUGCCACUCUGCAGUUUG | 671 | Rh | [671-688] ORF |
| 91 | AUACCAGCGUUAUGAGAU | 421 | AUCUCAUAACGCUGGUAU | 672 | Rh, Rt | [362-379] ORF |
| 92 | AGAGUGUCUGCGGAUACU | 422 | AGUAUCCGCAGACACUCU | 673 | Rh | [460-477] ORF |
| 93 | CACCAAGACCUACACUGU | 423 | ACAGUGUAGGUCUUGGUG | 674 | Rh | [608-625] ORF |
| 94 | GAUCAAGAUGACCAAGAU | 424 | AUCUUGGUCAUCUUGAUC | 675 | Rh, Dg | [377-394] ORF |
| 95 | AUGUAUAAAGGGUUCCAA | 425 | UUGGAACCCUUUAUACAU | 676 | | [393-410] ORF |
| 96 | ACCAAGACCUACACUGUU | 426 | AACAGUGUAGGUCUUGGU | 677 | Rh | [609-626] ORF |
| 97 | CCGUCACCUUGCCUGCCU | 427 | AGGCAGGCAAGGUGACGG | 678 | Rh | [743-760] ORF |
| 98 | GGGAGCCAGGGCUGUGCA | 428 | UGCACAGCCCUGGCUCCC | 679 | Rh | [766-783] ORF |
| 99 | UGCACAGUGUUUCCCUGU | 429 | ACAGGGAAACACUGUGCA | 680 | Rh, Rt, Ms | [639-656] ORF |
| 100 | UGCAGAGUGGCACUCAUU | 430 | AAUGAGUGCCACUCUGCA | 681 | Rh | [676-693] ORF |
| 101 | GUGAGGAAUGCACAGUGU | 431 | ACACUGUGCAUUCCUCAC | 682 | Rh | [631-648] ORF |
| 102 | AGCGAGGAGUUUCUCAUU | 432 | AAUGAGAAACUCCUCGCU | 683 | Rh | [498-515] ORF |
| 103 | GGGCUGUGCACCUGGCAG | 433 | CUGCCAGGUGCACAGCCC | 684 | Rh | [774-791] ORF |
| 104 | ACUCAUUGCUUGUGGACG | 434 | CGUCCACAAGCAAUGAGU | 685 | Rh | [687-704] ORF |
| 105 | UGUUGUUGCUGUGGCUGA | 435 | UCAGCCACAGCAACAACA | 686 | Rh | [226-243] ORF |
| 106 | UGAGGAAUGCACAGUGUU | 436 | AACACUGUGCAUUCCUCA | 687 | Rh | [632-649] ORF |
| 107 | CCUGGCUUCUGGCAUCCU | 437 | AGGAUGCCAGAAGCCAGG | 688 | | [209-226] ORF |
| 108 | GCACAGUGUCCACCCUGU | 438 | ACAGGGUGGACACUGUGC | 689 | | [844-861] 3′UTR |
| 109 | AAAGGGUUCCAAGCCUUA | 439 | UAAGGCUUGGAACCCUUU | 690 | | [399-416] ORF |
| 110 | GCUUCUGGCAUCCUGUUG | 440 | CAACAGGAUGCCAGAAGC | 691 | | [213-230] ORF |
| 111 | CCAAGACCUACACUGUUG | 441 | CAACAGUGUAGGUCUUGG | 692 | Rh | [610-627] ORF |
| 112 | AAGGGUUCCAAGCCUUAG | 442 | CUAAGGCUUGGAACCCUU | 693 | | [400-417] ORF |
| 113 | UGCACAGUGUCCACCCUG | 443 | CAGGGUGGACACUGUGCA | 694 | | [843-860] 3′UTR |
| 114 | GACCUACACUGUUGGCUG | 444 | CAGCCAACAGUGUAGGUC | 695 | Rh | [614-631] ORF |
| 115 | CCCAGAUAGCCUGAAUCC | 445 | GGAUUCAGGCUAUCUGGG | 696 | | [802-819] ORF + 3′UTR |
| 116 | GGGUUCCAAGCCUUAGGG | 446 | CCCUAAGGCUUGGAACCC | 697 | | [402-419] ORF |
| 117 | GGCUUCCAGUCCCGUCAC | 447 | GUGACGGGACUGGAAGCC | 698 | Rh | [732-749] ORF |
| 118 | AGUGUCUGCGGAUACUUC | 448 | GAAGUAUCCGCAGACACU | 699 | Rh | [462-479] ORF |
| 119 | UGACCAAGAUGUAUAAAG | 449 | CUUUAUACAUCUUGGUCA | 700 | Rh | [385-402] ORF |
| 120 | CAGCCUGAGCUUAGCUCA | 450 | UGAGCUAAGCUCAGGCUG | 701 | | [578-595] ORF |
| 121 | CUUCCGGACAAUGAAAUA | 451 | UAUUUCAUUGUCCGGAAG | 702 | | [878-895] 3′UTR |
| 122 | CUGUGAGGAAUGCACAGU | 452 | ACUGUGCAUUCCUCACAG | 703 | Rh | [629-646] ORF |
| 123 | GCCUGAAUCCUGCCCGGA | 453 | UCCGGGCAGGAUUCAGGC | 704 | | [810-827] ORF + 3′UTR |
| 124 | ACUGCAGGAUGGACUCUU | 454 | AAGAGUCCAUCCUGCAGU | 705 | | [524-541] ORF |
| 125 | GUCCCACAACCGCAGCGA | 455 | UCGCUGCGGUUGUGGGAC | 706 | Rh | [485-502] ORF |
| 126 | AUCUUUCUUCCGGACAAU | 456 | AUUGUCCGGAAGAAAGAU | 707 | | [872-889] 3′UTR |
| 127 | AUAAAGGGUUCCAAGCCU | 457 | AGGCUUGGAACCCUUUAU | 708 | | [397-414] ORF |
| 128 | UCCCAUCUUUCUUCCGGA | 458 | UCCGGAAGAAAGAUGGGA | 709 | | [868-885] 3′UTR |
| 129 | GAAAAGGGCUUCCAGUCC | 459 | GGACUGGAAGCCCUUUUC | 710 | Rh | [726-743] ORF |
| 130 | UGGAACAGCCUGAGCUUA | 460 | UAAGCUCAGGCUGUUCCA | 711 | | [573-590] ORF |
| 131 | CACCUUAUACCAGCGUUA | 461 | UAACGCUGGUAUAAGGUG | 712 | Rh, Rt, Ms | [356-373] ORF |
| 132 | CUGUUGGCUGUGAGGAAU | 462 | AUUCCUCACAGCCAACAG | 713 | Rh | [622-639] ORF |
| 133 | GCACAUCACUACCUGCAG | 463 | CUGCAGGUAGUGAUGUGC | 714 | | [542-559] ORF |
| 134 | UGCUGUGGCUGAUAGCCC | 464 | GGGCUAUCAGCCACAGCA | 715 | Rh | [232-249] ORF |
| 135 | CCACUCCCAUCUUUCUUC | 465 | GAAGAAAGAUGGGAGUGG | 716 | Rh | [864-881] 3′UTR |
| 136 | CUGGCUUCUGGCAUCCUG | 466 | CAGGAUGCCAGAAGCCAG | 717 | | [210-227] ORF |
| 137 | CUUCCACAGGUCCCACAA | 467 | UUGUGGGACCUGUGGAAG | 718 | Rh | [476-493] ORF |
| 138 | ACCAGCUCCUCCAAGGCU | 468 | AGCCUUGGAGGAGCUGGU | 719 | Rh | [706-723] ORF |
| 139 | CAAGAUGACCAAGAUGUA | 469 | UACAUCUUGGUCAUCUUG | 720 | Rh | [380-397] ORF |
| 140 | CCCGCCAUGGAGAGUGUC | 470 | GACACUCUCCAUGGCGGG | 721 | Rh | [450-467] ORF |
| 141 | CCCGGAGUGGAAGCUGAA | 471 | UUCAGCUUCCACUCCGGG | 722 | Rh | [822-839] 3′UTR |
| 142 | CGAGGAGUUUCUCAUUGC | 472 | GCAAUGAGAAACUCCUCG | 723 | Rh | [500-517] ORF |
| 143 | CACAUCACUACCUGCAGU | 473 | ACUGCAGGUAGUGAUGUG | 724 | | [543-560] ORF |
| 144 | CUCCAAGGCUCUGAAAAG | 474 | CUUUUCAGAGCCUUGGAG | 725 | Rh | [714-731] ORF |
| 145 | UGAGAUCAAGAUGACCAA | 475 | UUGGUCAUCUUGAUCUCA | 726 | Rh, Dg | [374-391] ORF |
| 146 | GCACUCAUUGCUUGUGGA | 476 | UCCACAAGCAAUGAGUGC | 727 | Rh, Rb | [685-702] ORF |
| 147 | CAUUGCUUGUGGACGGAC | 477 | GUCCGUCCACAAGCAAUG | 728 | Rh | [690-707] ORF |
| 148 | GGACCAGCUCCUCCAAGG | 478 | CCUUGGAGGAGCUGGUCC | 729 | Rh | [704-721] ORF |
| 149 | GCCUGCACAGUGUCCACC | 479 | GGUGGACACUGUGCAGGC | 730 | | [840-857] 3′UTR |
| 150 | UGUAUAAAGGGUUCCAAG | 480 | CUUGGAACCCUUUAUACA | 731 | | [394-411] ORF |
| 151 | CCUGCACAGUGUCCACCC | 481 | GGGUGGACACUGUGCAGG | 732 | | [841-858] 3′UTR |
| 152 | ACCUUAUACCAGCGUUAU | 482 | AUAACGCUGGUAUAAGGU | 733 | Rh, Rt, Ms | [357-374] ORF |
| 153 | CUUCCAGUCCCGUCACCU | 483 | AGGUGACGGGACUGGAAG | 734 | Rh | [734-751] ORF |
| 154 | CAGUGUCCACCCUGUUCC | 484 | GGAACAGGGUGGACACUG | 735 | | [847-864] 3′UTR |
| 155 | GGAGUGGAAGCUGAAGCC | 485 | GGCUUCAGCUUCCACUCC | 736 | Rh | [825-842] 3′UTR |
| 156 | CUUAUACCAGCGUUAUGA | 486 | UCAUAACGCUGGUAUAAG | 737 | Rh, Rt | [359-376] ORF |
| 157 | CCGCAGCGAGGAGUUUCU | 487 | AGAAACUCCUCGCUGCGG | 738 | Rh, Rb, Dg, Rt | [494-511] ORF |
| 158 | CAGUUUUGUGGCUCCCUG | 488 | CAGGGAGCCACAAAACUG | 739 | | [557-574] ORF |
| 159 | GUAUAAAGGGUUCCAAGC | 489 | GCUUGGAACCCUUUAUAC | 740 | | [395-412] ORF |
| 160 | AAGCCUGCACAGUGUCCA | 490 | UGGACACUGUGCAGGCUU | 741 | | [838-855] 3′UTR |
| 161 | AGGAUGGACUCUUGCACA | 491 | UGUGCAAGAGUCCAUCCU | 742 | | [529-546] ORF |
| 162 | AUGGAGAGUGUCUGCGGA | 492 | UCCGCAGACACUCUCCAU | 743 | Rh | [456-473] ORF |
| 163 | GCCUUCUGCAAUUCCGAC | 493 | GUCGGAAUUGCAGAAGGC | 744 | Rh | [291-308] ORF |
| 164 | CUUCUGCAAUUCCGACCU | 494 | AGGUCGGAAUUGCAGAAG | 745 | Rh | [293-310] ORF |
| 165 | AGACGGCCUUCUGCAAUU | 495 | AAUUGCAGAAGGCCGUCU | 746 | Rh | [286-303] ORF |
| 166 | GCAGGAUGGACUCUUGCA | 496 | UGCAAGAGUCCAUCCUGC | 747 | | [527-544] ORF |
| 167 | GCUUGUGGACGGACCAGC | 497 | GCUGGUCCGUCCACAAGC | 748 | Rh | [694-711] ORF |
| 168 | AGAAGUCAACCAGACCAC | 498 | GUGGUCUGGUUGACUUCU | 749 | Rh | [341-358] ORF |
| 169 | CUGUGCACCUGGCAGUCC | 499 | GGACUGCCAGGUGCACAG | 750 | Rh | [777-794] ORF |
| 170 | GAGGAGUUUCUCAUUGCU | 500 | AGCAAUGAGAAACUCCUC | 751 | Rh | [501-518] ORF |
| 171 | UGUUCCCACUCCCAUCUU | 501 | AAGAUGGGAGUGGGAACA | 752 | Rh | [859-876] 3′UTR |
| 172 | ACCGCAGCGAGGAGUUUC | 502 | GAAACUCCUCGCUGCGGU | 753 | Rh, Rb, Dg, Rt | [493-510] ORF |
| 173 | ACCAGAAGUCAACCAGAC | 503 | GUCUGGUUGACUUCUGGU | 754 | Rh | [338-355] ORF |
| 174 | GCAAUUCCGACCUCGUCA | 504 | UGACGAGGUCGGAAUUGC | 755 | Rh | [298-315] ORF |
| 175 | CUGCAAACUGCAGAGUGG | 505 | CCACUCUGCAGUUUGCAG | 756 | Rh | [668-685] ORF |
| 176 | GGAGCCAGGGCUGUGCAC | 506 | GUGCACAGCCCUGGCUCC | 757 | Rh | [767-784] ORF |
| 177 | CCUUCUGCAAUUCCGACC | 507 | GGUCGGAAUUGCAGAAGG | 758 | Rh | [292-309] ORF |
| 178 | CUUGCACAUCACUACCUG | 508 | CAGGUAGUGAUGUGCAAG | 759 | | [539-556] ORF |
| 179 | GGAAAACUGCAGGAUGGA | 509 | UCCAUCCUGCAGUUUUCC | 760 | | [519-536] ORF |
| 180 | AAUGCACAGUGUUUCCCU | 510 | AGGGAAACACUGUGCAUU | 761 | Rh | [637-654] ORF |
| 181 | CCCUGGAACAGCCUGAGC | 511 | GCUCAGGCUGUUCCAGGG | 762 | | [570-587] ORF |
| 182 | GGAUGCCGCUGACAUCCG | 512 | CGGAUGUCAGCGGCAUCC | 763 | Rh | [419-436] ORF |
| 183 | GGAUACUUCCACAGGUCC | 513 | GGACCUGUGGAAGUAUCC | 764 | Rh | [471-488] ORF |
| 184 | CACUCAUUGCUUGUGGAC | 514 | GUCCACAAGCAAUGAGUG | 765 | Rh, Rb | [686-703] ORF |
| 185 | GACACCAGAAGUCAACCA | 515 | UGGUUGACUUCUGGUGUC | 766 | Rh | [335-352] ORF |
| 186 | CUACACUGUUGGCUGUGA | 516 | UCACAGCCAACAGUGUAG | 767 | Rh | [617-634] ORF |
| 187 | ACCACCUUAUACCAGCGU | 517 | ACGCUGGUAUAAGGUGGU | 768 | Rh, Rt, Ms | [354-371] ORF |
| 188 | AGAGUGGCACUCAUUGCU | 518 | AGCAAUGAGUGCCACUCU | 769 | Rh | [679-696] ORF |
| 189 | GCAAACUGCAGAGUGGCA | 519 | UGCCACUCUGCAGUUUGC | 770 | Rh | [670-687] ORF |
| 190 | GCCGCUGACAUCCGGUUC | 520 | GAACCGGAUGUCAGCGGC | 771 | Rh | [423-440] ORF |
| 191 | UGUUUCCCUGUUUAUCCA | 521 | UGGAUAAACAGGGAAACA | 772 | Rh | [646-663] ORF |
| 192 | AAGUCAACCAGACCACCU | 522 | AGGUGGUCUGGUUGACUU | 773 | Rh | [343-360] ORF |
| 193 | CCACAACCGCAGCGAGGA | 523 | UCCUCGCUGCGGUUGUGG | 774 | Rh | [488-505] ORF |
| 194 | GCCUGAGCUUAGCUCAGC | 524 | GCUGAGCUAAGCUCAGGC | 775 | | [580-597] ORF |
| 195 | GUCAUCAGGGCCAAGUUC | 525 | GAACUUGGCCCUGAUGAC | 776 | Rh, Dg | [312-329] ORF |
| 196 | AACCAGACCACCUUAUAC | 526 | GUAUAAGGUGGUCUGGUU | 777 | Rh | [348-365] ORF |
| 197 | CUCCCUGGAACAGCCUGA | 527 | UCAGGCUGUUCCAGGGAG | 778 | | [568-585] ORF |
| 198 | CCAAGGCUCUGAAAAGGG | 528 | CCCUUUUCAGAGCCUUGG | 779 | Rh | [716-733] ORF |
| 199 | CUCAUUGCUGGAAAACUG | 529 | CAGUUUUCCAGCAAUGAG | 780 | Rh | [510-527] ORF |
| 200 | GUGUCCACCCUGUUCCCA | 530 | UGGGAACAGGGUGGACAC | 781 | | [849-866] 3′UTR |
| 201 | CCUGUUCCCACUCCCAUC | 531 | GAUGGGAGUGGGAACAGG | 782 | Rh | [857-874] 3′UTR |
| 202 | CACCUUGCCUGCCUGCCU | 532 | AGGCAGGCAGGCAAGGUG | 783 | Rh | [747-764] ORF |
| 203 | UCACCAAGACCUACACUG | 533 | CAGUGUAGGUCUUGGUGA | 784 | Rh | [607-624] ORF |
| 204 | GAAUGCACAGUGUUUCCC | 534 | GGGAAACACUGUGCAUUC | 785 | Rh | [636-653] ORF |
| 205 | AUGGACUCUUGCACAUCA | 535 | UGAUGUGCAAGAGUCCAU | 786 | | [532-549] ORF |
| 206 | UGUGAGGAAUGCACAGUG | 536 | CACUGUGCAUUCCUCACA | 787 | Rh | [630-647] ORF |
| 207 | GAGCCAGGGCUGUGCACC | 537 | GGUGCACAGCCCUGGCUC | 788 | Rh | [768-785] ORF |
| 208 | UGCGGUCCCAGAUAGCCU | 538 | AGGCUAUCUGGGACCGCA | 789 | | [796-813] ORF |
| 209 | CUGUUCCCACUCCCAUCU | 539 | AGAUGGGAGUGGGAACAG | 790 | Rh | [858-875] 3′UTR |
| 210 | UGGCUUCUGGCAUCCUGU | 540 | ACAGGAUGCCAGAAGCCA | 791 | | [211-228] ORF |
| 211 | CGGAUACUUCCACAGGUC | 541 | GACCUGUGGAAGUAUCCG | 792 | Rh | [470-487] ORF |
| 212 | CACAGUGUCCACCCUGUU | 542 | AACAGGGUGGACACUGUG | 793 | | [845-862] 3′UTR |
| 213 | GGAAUGCACAGUGUUUCC | 543 | GGAAACACUGUGCAUUCC | 794 | Rh | [635-652] ORF |
| 214 | CCAGGGCUGUGCACCUGG | 544 | CCAGGUGCACAGCCCUGG | 795 | Rh | [771-788] ORF |
| 215 | CCCUGCGGUCCCAGAUAG | 545 | CUAUCUGGGACCGCAGGG | 796 | | [793-810] ORF |
| 216 | GCCUGCACCUGUGUCCCA | 546 | UGGGACACAGGUGCAGGC | 797 | Rb | [258-275] ORF |
| 217 | CAGAGUGGCACUCAUUGC | 547 | GCAAUGAGUGCCACUCUG | 798 | Rh | [678-695] ORF |
| 218 | UGCACAUCACUACCUGCA | 548 | UGCAGGUAGUGAUGUGCA | 799 | | [541-558] ORF |
| 219 | GAAGUCAACCAGACCACC | 549 | GGUGGUCUGGUUGACUUC | 800 | Rh | [342-359] ORF |
| 220 | UCCCUGCGGUCCCAGAUA | 550 | UAUCUGGGACCGCAGGGA | 801 | | [792-809] ORF |
| 221 | AGUGGCACUCAUUGCUUG | 551 | CAAGCAAUGAGUGCCACU | 802 | Rh | [681-698] ORF |
| 222 | GUGGCACUCAUUGCUUGU | 552 | ACAAGCAAUGAGUGCCAC | 803 | Rh | [682-699] ORF |
| 223 | CUGAAUCCUGCCCGGAGU | 553 | ACUCCGGGCAGGAUUCAG | 804 | | [812-829] ORF + 3′UTR |
| 224 | CCUGCACCUGUGUCCCAC | 554 | GUGGGACACAGGUGCAGG | 805 | Rb | [259-276] ORF |
| 225 | GCCUGCCUCGGGAGCCAG | 555 | CUGGCUCCCGAGGCAGGC | 806 | Rh | [757-774] ORF |
| 226 | GGGAUGCCGCUGACAUCC | 556 | GGAUGUCAGCGGCAUCCC | 807 | Rh | [418-435] ORF |
| 227 | GCACCUGGCAGUCCCUGC | 557 | GCAGGGACUGCCAGGUGC | 808 | Rh, Dg | [781-798] ORF |
| 228 | UCCUGUUGUUGCUGUGGC | 558 | GCCACAGCAACAACAGGA | 809 | Rh | [223-240] ORF |
| 229 | UGCGGAUACUUCCACAGG | 559 | CCUGUGGAAGUAUCCGCA | 810 | Rh | [468-485] ORF |
| 230 | GACCAAGAUGUAUAAAGG | 560 | CCUUUAUACAUCUUGGUC | 811 | Rh | [386-403] ORF |
| 231 | ACUGUUGGCUGUGAGGAA | 561 | UUCCUCACAGCCAACAGU | 812 | Rh | [621-638] ORF |
| 232 | CAUUGCUGGAAAACUGCA | 562 | UGCAGUUUUCCAGCAAUG | 813 | Rh | [512-529] ORF |
| 233 | UGAAAAGGGCUUCCAGUC | 563 | GACUGGAAGCCCUUUUCA | 814 | Rh | [725-742] ORF |
| 234 | CGUCAUCAGGGCCAAGUU | 564 | AACUUGGCCCUGAUGACG | 815 | Rh, Rb, Dg | [311-328] ORF |
| 235 | ACAACCGCAGCGAGGAGU | 565 | ACUCCUCGCUGCGGUUGU | 816 | Rh | [490-507] ORF |
| 236 | UGUCCACCCUGUUCCCAC | 566 | GUGGGAACAGGGUGGACA | 817 | Rh | [850-867] 3′UTR |
| 237 | CCUGGCAGUCCCUGCGGU | 567 | ACCGCAGGGACUGCCAGG | 818 | | [784-801] ORF |
| 238 | UGAAGCCUGCACAGUGUC | 568 | GACACUGUGCAGGCUUCA | 819 | | [836-853] 3′UTR |
| 239 | UCCCAGAUAGCCUGAAUC | 569 | GAUUCAGGCUAUCUGGGA | 820 | | [801-818] ORF + 3′UTR |
| 240 | AGCCUGAGCUUAGCUCAG | 570 | CUGAGCUAAGCUCAGGCU | 821 | | [579-596] ORF |
| 241 | CACUACCUGCAGUUUUGU | 571 | ACAAAACUGCAGGUAGUG | 822 | | [548-565] ORF |
| 242 | CUGCACAGUGUCCACCCU | 572 | AGGGUGGACACUGUGCAG | 823 | | [842-859] 3′UTR |
| 243 | CUCGGGAGCCAGGGCUGU | 573 | ACAGCCCUGGCUCCCGAG | 824 | Rh | [763-780] ORF |
| 244 | AGCCAGGGCUGUGCACCU | 574 | AGGUGCACAGCCCUGGCU | 825 | Rh | [769-786] ORF |
| 245 | GCCAUGGAGAGUGUCUGC | 575 | GCAGACACUCUCCAUGGC | 826 | Rh | [453-470] ORF |
| 246 | AGGGCUGUGCACCUGGCA | 576 | UGCCAGGUGCACAGCCCU | 827 | Rh | [773-790] ORF |
| 247 | GAGCUUAGCUCAGCGCCG | 577 | CGGCGCUGAGCUAAGCUC | 828 | Rh | [584-601] ORF |
| 248 | AGGCUCUGAAAAGGGCUU | 578 | AAGCCCUUUUCAGAGCCU | 829 | Rh | [719-736] ORF |
| 249 | ACAUCACUACCUGCAGUU | 579 | AACUGCAGGUAGUGAUGU | 830 | | [544-561] ORF |
| 250 | AACCGCAGCGAGGAGUUU | 580 | AAACUCCUCGCUGCGGUU | 831 | Rh, Rb, Dg, Rt | [492-509] ORF |
| 251 | CAGCUCCUCCAAGGCUCU | 581 | AGAGCCUUGGAGGAGCUG | 832 | Rh | [708-725] ORF |

TABLE A6
18-mer Cross-Species siTIMP1

| | | SEQ | | SEQ | | |
|---|---|---|---|---|---|---|
| | | ID | | ID | | human-73858576 |
| No. | Sense (5′>3′) | NO. | Antisense (5′>3′) | NO. | Other Sp | ORF: 193-816 |
| 1 | ACCUGGCAGUCCCUGCGG | 833 | CCGCAGGGACUGCCAGGU | 839 | Rh, Rb, Dg | [783-800] ORF |
| 2 | CAACCGCAGCGAGGAGUU | 834 | AACUCCUCGCUGCGGUUG | 840 | Rh, Rb, Dg, Rt | [491-508] ORF |
| 3 | AUGCACAGUGUUUCCCUG | 835 | CAGGGAAACACUGUGCAU | 841 | Rh, Rt, Ms | [638-655] ORF |
| 4 | GACCACCUUAUACCAGCG | 836 | CGCUGGUAUAAGGUGGUC | 842 | Rh, Rt, Ms | [353-370] ORF |
| 5 | UUAUGAGAUCAAGAUGAC | 837 | GUCAUCUUGAUCUCAUAA | 843 | Rh, Dg, Rt | [371-388] ORF |
| 6 | UAUACCAGCGUUAUGAGA | 838 | UCUCAUAACGCUGGUAUA | 844 | Rh, Rt | [361-378] ORF |

TABLE A7
Preferred 18 + A-mer siTIMP1

| | | | | | | |
|---|---|---|---|---|---|---|
| | | SEQ | | SEQ | | |
| | | ID | | ID | | human-73858576 |
| siTIMP1_pNo. | Sense (5′>3′) | NO. | Antisense (5′>3′) | NO. | length | ORF: 193-816 |

| | | | | | | |
|---|---|---|---|---|---|---|
| siTIMP1_p1 | GCGUUAUGAGAUCAAGAUA | 845 | UAUCUUGAUCUCAUAACGC | 926 | 18 + 1 | [368-385] ORF |
| siTIMP1_p3 | CAGACCACCUUAUACCAGA | 846 | UCUGGUAUAAGGUGGUCUG | 927 | 18 + 1 | [351-368] ORF |
| siTIMP1_p4 | GCACAGUGUUUCCCUGUUA | 847 | UAACAGGGAAACACUGUGC | 928 | 18 + 1 | [640-657] ORF |
| siTIMP1_p5 | CAGCGAGGAGUUUCUCAUA | 848 | UAUGAGAAACUCCUCGCUG | 929 | 18 + 1 | [497-514] ORF |
| siTIMP1_p7 | AUACCAGCGUUAUGAGAUA | 849 | UAUCUCAUAACGCUGGUAU | 930 | 18 + 1 | [362-379] ORF |
| siTIMP1_p8 | UGCACAGUGUUUCCCUGUA | 850 | UACAGGGAAACACUGUGCA | 931 | 18 + 1 | [639-656] ORF |
| siTIMP1_p9 | CACCUUAUACCAGCGUUAA | 851 | UUAACGCUGGUAUAAGGUG | 932 | 18 + 1 | [356-373] ORF |
| siTIMP1_p10 | ACCUUAUACCAGCGUUAUA | 852 | UAUAACGCUGGUAUAAGGU | 933 | 18 + 1 | [357-374] ORF |
| siTIMP1_p11 | CUUAUACCAGCGUUAUGAA | 853 | UUCAUAACGCUGGUAUAAG | 934 | 18 + 1 | [359-376] ORF |
| siTIMP1_p12 | CCGCAGCGAGGAGUUUCUA | 854 | UAGAAACUCCUCGCUGCGG | 935 | 18 + 1 | [494-511] ORF |
| siTIMP1_p13 | ACCGCAGCGAGGAGUUUCA | 855 | UGAAACUCCUCGCUGCGGU | 936 | 18 + 1 | [493-510] ORF |
| siTIMP1_p15 | ACCACCUUAUACCAGCGUA | 856 | UACGCUGGUAUAAGGUGGU | 937 | 18 + 1 | [354-371] ORF |
| siTIMP1_p18 | AACCGCAGCGAGGAGUUUA | 857 | UAAACUCCUCGCUGCGGUU | 938 | 18 + 1 | [492-509] ORF |
| siTIMP1_p22 | UAUACCAGCGUUAUGAGAA | 858 | UUCUCAUAACGCUGGUAUA | 939 | 18 + 1 | [361-378] ORF |
| siTIMP1_p25 | AGAUCAAGAUGACCAAGAA | 859 | UUCUUGGUCAUCUUGAUCU | 940 | 18 + 1 | [376-393] ORF |
| siTIMP1_p26 | CCUGCAAACUGCAGAGUGA | 860 | UCACUCUGCAGUUUGCAGG | 941 | 18 + 1 | [667-684] ORF |
| siTIMP1_p28 | CCCUGCAAACUGCAGAGUA | 861 | UACUCUGCAGUUUGCAGGG | 942 | 18 + 1 | [666-683] ORF |
| siTIMP1_p30 | GAUCAAGAUGACCAAGAUA | 862 | UAUCUUGGUCAUCUUGAUC | 943 | 18 + 1 | [377-394] ORF |
| siTIMP1_p32 | GUCAUCAGGGCCAAGUUCA | 863 | UGAACUUGGCCCUGAUGAC | 944 | 18 + 1 | [312-329] ORF |
| siTIMP1_p34 | CCUGCACCUGUGUCCCACA | 864 | UGUGGGACACAGGUGCAGG | 945 | 18 + 1 | [259-276] ORF |
| siTIMP1_p35 | GGGCUUCACCAAGACCUAA | 865 | UUAGGUCUUGGUGAAGCCC | 946 | 18 + 1 | [602-619] ORF |
| siTIMP1_p36 | CGUCAUCAGGGCCAAGUUA | 866 | UAACUUGGCCCUGAUGACG | 947 | 18 + 1 | [311-328] ORF |
| siTIMP1_p37 | CACUCAUUGCUUGUGGACA | 867 | UGUCCACAAGCAAUGAGUG | 948 | 18 + 1 | [686-703] ORF |
| siTIMP1_p39 | CAGUGUUUCCCUGUUUAUA | 868 | UAUAAACAGGGAAACACUG | 949 | 18 + 1 | [643-660] ORF |
| siTIMP1_p40 | ACAGUGUUUCCCUGUUUAA | 869 | UUAAACAGGGAAACACUGU | 950 | 18 + 1 | [642-659] ORF |
| siTIMP1_p41 | AGUGUUUCCCUGUUUAUCA | 870 | UGAUAAACAGGGAAACACU | 951 | 18 + 1 | [644-661] ORF |
| siTIMP1_p44 | CAUCUUUCUUCCGGACAAA | 871 | UUUGUCCGGAAGAAAGAUG | 952 | 18 + 1 | [871-888] 3′UTR |
| siTIMP1_p46 | CCAGAUAGCCUGAAUCCUA | 872 | UAGGAUUCAGGCUAUCUGG | 953 | 18 + 1 | [803-820] |
| | | | | | | ORF + 3′UTR |
| siTIMP1_p47 | GGUCCCAGAUAGCCUGAAA | 873 | UUUCAGGCUAUCUGGGACC | 954 | 18 + 1 | [799-816] ORF |
| siTIMP1_p48 | GGAGAGUGUCUGCGGAUAA | 874 | UUAUCCGCAGACACUCUCC | 955 | 18 + 1 | [458-475] ORF |
| siTIMP1_p50 | CCGCCAUGGAGAGUGUCUA | 875 | UAGACACUCUCCAUGGCGG | 956 | 18 + 1 | [458-475] ORF |
| siTIMP1_p51 | GAGUGUCUGCGGAUACUUA | 876 | UAAGUAUCCGCAGACACUC | 957 | 18 + 1 | [458-475] ORF |
| siTIMP1_p52 | GAGUGGCACUCAUUGCUUA | 877 | UAAGCAAUGAGUGCCACUC | 958 | 18 + 1 | [680-697] ORF |
| siTIMP1_p53 | GAGUGGAAGCUGAAGCCUA | 878 | UAGGCUUCAGCUUCCACUC | 959 | 18 + 1 | [826-843] 3′UTR |
| siTIMP1_p54 | CCCUGUUCCCACUCCCAUA | 879 | UAUGGGAGUGGGAACAGGG | 960 | 18 + 1 | [856-873] 3′UTR |
| siTIMP1_p55 | GCGGAUACUUCCACAGGUA | 880 | UACCUGUGGAAGUAUCCGC | 961 | 18 + 1 | [469-486] ORF |
| siTIMP1_p56 | GAGAGUGUCUGCGGAUACA | 881 | UGUAUCCGCAGACACUCUC | 962 | 18 + 1 | [459-476] ORF |
| siTIMP1_p57 | GGAACAGCCUGAGCUUAGA | 882 | UCUAAGCUCAGGCUGUUCC | 963 | 18 + 1 | [574-591] ORF |
| siTIMP1_p58 | CAACCAGACCACCUUAUAA | 883 | UUAUAAGGUGGUCUGGUUG | 964 | 18 + 1 | [347-364] ORF |
| siTIMP1_p59 | GAGGAAUGCACAGUGUUUA | 884 | UAAACACUGUGCAUUCCUC | 965 | 18 + 1 | [633-650] ORF |
| siTIMP1_p61 | CCAAGAUGUAUAAAGGGUA | 885 | UACCCUUUAUACAUCUUGG | 966 | 18 + 1 | [388-405] ORF |
| siTIMP1_p62 | CACCAGAAGUCAACCAGAA | 886 | UUCUGGUUGACUUCUGGUG | 967 | 18 + 1 | [337-354] ORF |
| siTIMP1_p63 | CAGAAGUCAACCAGACCAA | 887 | UUGGUCUGGUUGACUUCUG | 968 | 18 + 1 | [340-357] ORF |
| siTIMP1_p64 | CCCACUCCCAUCUUUCUUA | 888 | UAAGAAAGAUGGGAGUGGG | 969 | 18 + 1 | [863-880] 3′UTR |
| siTIMP1_p65 | GCGAGGAGUUUCUCAUUGA | 889 | UCAAUGAGAAACUCCUCGC | 970 | 18 + 1 | [499-516] ORF |
| siTIMP1_p66 | CUGCAGAGUGGCACUCAUA | 890 | UAUGAGUGCCACUCUGCAG | 971 | 18 + 1 | [675-692] ORF |
| siTIMP1_p67 | GAAGCUGAAGCCUGCACAA | 891 | UUGUGCAGGCUUCAGCUUC | 972 | 18 + 1 | [831-848] 3′UTR |
| siTIMP1_p68 | CCUGGAACAGCCUGAGCUA | 892 | UAGCUCAGGCUGUUCCAGG | 973 | 18 + 1 | [571-588] ORF |
| siTIMP1_p69 | GCAUCCUGUUGUUGCUGUA | 893 | UACAGCAACAACAGGAUGC | 974 | 18 + 1 | [220-237] ORF |
| siTIMP1_p70 | GUCCCAGAUAGCCUGAAUA | 894 | UAUUCAGGCUAUCUGGGAC | 975 | 18 + 1 | [800-817] |
| | | | | | | ORF + 3′UTR |
| siTIMP1_p72 | GGCUGUGAGGAAUGCACAA | 895 | UUGUGCAUUCCUCACAGCC | 976 | 18 + 1 | [627-644] ORF |
| siTIMP1_p74 | GGCCUUCUGCAAUUCCGAA | 896 | UUCGGAAUUGCAGAAGGCC | 977 | 18 + 1 | [290-307] ORF |
| siTIMP1_p75 | GCAGAGUGGCACUCAUUGA | 897 | UCAAUGAGUGCCACUCUGC | 978 | 18 + 1 | [677-694] ORF |
| siTIMP1_p76 | CAGAUAGCCUGAAUCCUGA | 898 | UCAGGAUUCAGGCUAUCUG | 979 | 18 + 1 | [804-821] |
| | | | | | | ORF + 3′UTR |
| siTIMP1_p80 | CCGGAGUGGAAGCUGAAGA | 899 | UCUUCAGCUUCCACUCCGG | 980 | 18 + 1 | [823-840] 3′UTR |
| siTIMP1_p81 | GGCUGUGCACCUGGCAGUA | 900 | UACUGCCAGGUGCACAGCC | 981 | 18 + 1 | [775-792] ORF |
| siTIMP1_p82 | GUCAACCAGACCACCUUAA | 901 | UUAAGGUGGUCUGGUUGAC | 982 | 18 + 1 | [345-362] ORF |
| siTIMP1_p83 | CCAUGGAGAGUGUCUGCGA | 902 | UCGCAGACACUCUCCAUGG | 983 | 18 + 1 | [454-471] ORF |
| siTIMP1_p84 | CUGGCAUCCUGUUGUUGCA | 903 | UGCAACAACAGGAUGCCAG | 984 | 18 + 1 | [217-234] ORF |
| siTIMP1_p86 | ACUGCAGAGUGGCACUCAA | 904 | UUGAGUGCCACUCUGCAGU | 985 | 18 + 1 | [674-691] ORF |
| siTIMP1_p87 | CGGAGUGGAAGCUGAAGCA | 905 | UGCUUCAGCUUCCACUCCG | 986 | 18 + 1 | [824-841] 3′UTR |
| siTIMP1_p88 | CCAGACCACCUUAUACCAA | 906 | UUGGUAUAAGGUGGUCUGG | 987 | 18 + 1 | [350-367] ORF |
| siTIMP1_p90 | GCUGGAAAACUGCAGGAUA | 907 | UAUCCUGCAGUUUUCCAGC | 988 | 18 + 1 | [516-533] ORF |
| siTIMP1_p92 | CCUGAAUCCUGCCCGGAGA | 908 | UCUCCGGGCAGGAUUCAGG | 989 | 18 + 1 | [811-828] |
| | | | | | | ORF + 3′UTR |
| siTIMP1_p93 | CUGAAGCCUGCACAGUGUA | 909 | UACACUGUGCAGGCUUCAG | 990 | 18 + 1 | [835-852] 3′UTR |
| siTIMP1_p94 | CUGGAAAACUGCAGGAUGA | 910 | UCAUCCUGCAGUUUUCCAG | 991 | 18 + 1 | [517-534] ORF |
| siTIMP1_p95 | UCUCAUUGCUGGAAAACUA | 911 | UAGUUUUCCAGCAAUGAGA | 992 | 18 + 1 | [509-526] ORF |
| siTIMP1_p97 | AGACCUACACUGUUGGCUA | 912 | UAGCCAACAGUGUAGGUCU | 993 | 18 + 1 | [613-630] ORF |
| siTIMP1_p100 | GGGACACCAGAAGUCAACA | 913 | UGUUGACUUCUGGUGUCCC | 994 | 18 + 1 | [333-350] ORF |
| siTIMP1_p101 | GGCUCCCUGGAACAGCCUA | 914 | UAGGCUGUUCCAGGGAGCC | 995 | 18 + 1 | [566-583] ORF |
| siTIMP1_p102 | GUUCCCACUCCCAUCUUUA | 915 | UAAAGAUGGGAGUGGGAAC | 996 | 18 + 1 | [860-877] 3′UTR |
| siTIMP1_p103 | GGCUUCUGGCAUCCUGUUA | 916 | UAACAGGAUGCCAGAAGCC | 997 | 18 + 1 | [212-229] ORF |
| siTIMP1_p104 | CUUCUGGCAUCCUGUUGUA | 917 | UACAACAGGAUGCCAGAAG | 998 | 18 + 1 | [214-231] ORF |
| siTIMP1_p105 | AGAGUGUCUGCGGAUACUA | 918 | UAGUAUCCGCAGACACUCU | 999 | 18 + 1 | [460-477] ORF |
| siTIMP1_p106 | CACCAAGACCUACACUGUA | 919 | UACAGUGUAGGUCUUGGUG | 1000 | 18 + 1 | [608-625] ORF |
| siTIMP1_p109 | GGGAGCCAGGGCUGUGCAA | 920 | UUGCACAGCCCUGGCUCCC | 1001 | 18 + 1 | [766-783] ORF |
| siTIMP1_p110 | UGCAGAGUGGCACUCAUUA | 921 | UAAUGAGUGCCACUCUGCA | 1002 | 18 + 1 | [676-693] ORF |
| siTIMP1_p111 | GUGAGGAAUGCACAGUGUA | 922 | UACACUGUGCAUUCCUCAC | 1003 | 18 + 1 | [631-648] ORF |
| siTIMP1_p112 | AGCGAGGAGUUUCUCAUUA | 923 | UAAUGAGAAACUCCUCGCU | 1004 | 18 + 1 | [498-515] ORF |
| siTIMP1_p113 | GGGCUGUGCACCUGGCAGA | 924 | UCUGCCAGGUGCACAGCCC | 1005 | 18 + 1 | [774-791] ORF |
| siTIMP1_p114 | UGUUGUUGCUGUGGCUGAA | 925 | UUCAGCCACAGCAACAACA | 1006 | 18 + 1 | [226-243] ORF |

TABLE A8
18 + 1-mer siTIMP1 with lowest predicted Off Target (OT) effect

| | | | | | | |
|---|---|---|---|---|---|---|
| | | | SEQ | | SEQ | |
| | | | ID | | ID | No. in Table |
| Cross species | Ranking | Sense (5′ > 3′) | NO. | Antisense (5′ > 3′) | NO. | A7 |

| | | | | | | |
|---|---|---|---|---|---|---|
| H/Rt | 2 | GCGUUAUGAGAUCAAGAUA | 845 | UAUCUUGAUCUCAUAACGC | 926 | siTIMP1_p1 |
| H/Rt | 3 | GCACAGUGUUUCCCUGUUA | 847 | UAACAGGGAAACACUGUGC | 928 | siTIMP1_p4 |
| H/Rt | 3 | CAGCGAGGAGUUUCUCAUA | 848 | UAUGAGAAACUCCUCGCUG | 929 | siTIMP1_p5 |
| H/Rt | 2 | AUACCAGCGUUAUGAGAUA | 849 | UAUCUCAUAACGCUGGUAU | 930 | siTIMP1_p7 |
| H/Rt | 4 | UGCACAGUGUUUCCCUGUA | 850 | UACAGGGAAACACUGUGCA | 931 | siTIMP1_p8 |
| H/Rt | 1 | CACCUUAUACCAGCGUUAA | 851 | UUAACGCUGGUAUAAGGUG | 932 | siTIMP1_p9 |
| H/Rt | 1 | ACCUUAUACCAGCGUUAUA | 852 | UAUAACGCUGGUAUAAGGU | 933 | siTIMP1_p10 |
| H/Rt | 1 | CUUAUACCAGCGUUAUGAA | 853 | UUCAUAACGCUGGUAUAAG | 934 | siTIMP1_p11 |
| H/Rt | 2 | CCGCAGCGAGGAGUUUCUA | 854 | UAGAAACUCCUCGCUGCGG | 935 | siTIMP1_p12 |
| H/Rt | 3 | ACCGCAGCGAGGAGUUUCA | 855 | UGAAACUCCUCGCUGCGGU | 936 | siTIMP1_p13 |
| H/Rt | 2 | ACCACCUUAUACCAGCGUA | 856 | UACGCUGGUAUAAGGUGGU | 937 | siTIMP1_p15 |
| H/Rt | 2 | AACCGCAGCGAGGAGUUUA | 857 | UAAACUCCUCGCUGCGGUU | 938 | siTIMP1_p18 |
| H/Rt | 2 | UAUACCAGCGUUAUGAGAA | 858 | UUCUCAUAACGCUGGUAUA | 939 | siTIMP1_p22 |
| Other (w/o Rt) | 4 | CCUGCAAACUGCAGAGUGA | 860 | UCACUCUGCAGUUUGCAGG | 941 | siTIMP1_p26 |
| Other (w/o Rt) | 4 | CGUCAUCAGGGCCAAGUUA | 866 | UAACUUGGCCCUGAUGACG | 947 | siTIMP1_p36 |
| H/Rt (Rt with | 1 | CACUCAUUGCUUGUGGACA | 867 | UGUCCACAAGCAAUGAGUG | 948 | siTIMP1_p37 |
| 1MM) | | | | | | |
| Other (w/o Rt) | 3 | CAGUGUUUCCCUGUUUAUA | 868 | UAUAAACAGGGAAACACUG | 949 | siTIMP1_p39 |
| Other (w/o Rt) | 3 | ACAGUGUUUCCCUGUUUAA | 869 | UUAAACAGGGAAACACUGU | 950 | siTIMP1_p40 |
| Other (w/o Rt) | 2 | AGUGUUUCCCUGUUUAUCA | 870 | UGAUAAACAGGGAAACACU | 951 | siTIMP1_p41 |
| H +/− Rh | 2 | CAUCUUUCUUCCGGACAAA | 871 | UUUGUCCGGAAGAAAGAUG | 952 | siTIMP1_p44 |
| H +/− Rh | 3 | GGUCCCAGAUAGCCUGAAA | 873 | UUUCAGGCUAUCUGGGACC | 954 | siTIMP1_p47 |
| H +/− Rh | 2 | GGAGAGUGUCUGCGGAUAA | 874 | UUAUCCGCAGACACUCUCC | 955 | siTIMP1_p48 |
| H +/− Rh | 3 | CCGCCAUGGAGAGUGUCUA | 875 | UAGACACUCUCCAUGGCGG | 956 | siTIMP1_p50 |
| H +/− Rh | 3 | GAGUGUCUGCGGAUACUUA | 876 | UAAGUAUCCGCAGACACUC | 957 | siTIMP1_p51 |
| H +/− Rh | 3 | GAGUGGCACUCAUUGCUUA | 877 | UAAGCAAUGAGUGCCACUC | 958 | siTIMP1_p52 |
| H +/− Rh | 2 | GCGGAUACUUCCACAGGUA | 880 | UACCUGUGGAAGUAUCCGC | 961 | siTIMP1_p55 |
| H +/− Rh | 2 | GAGAGUGUCUGCGGAUACA | 881 | UGUAUCCGCAGACACUCUC | 962 | siTIMP1_p56 |
| H +/− Rh | 3 | CAACCAGACCACCUUAUAA | 883 | UUAUAAGGUGGUCUGGUUG | 964 | siTIMP1_p58 |
| H +/− Rh | 3 | CCAAGAUGUAUAAAGGGUA | 885 | UACCCUUUAUACAUCUUGG | 966 | siTIMP1_p61 |
| H +/− Rh | 4 | CCCACUCCCAUCUUUCUUA | 888 | UAAGAAAGAUGGGAGUGGG | 969 | siTIMP1_p64 |
| H +/− Rh | 3 | CUGCAGAGUGGCACUCAUA | 890 | UAUGAGUGCCACUCUGCAG | 971 | siTIMP1_p66 |
| H +/− Rh | 4 | CCUGGAACAGCCUGAGCUA | 892 | UAGCUCAGGCUGUUCCAGG | 973 | siTIMP1_p68 |
| H +/− Rh | 3 | GUCCCAGAUAGCCUGAAUA | 894 | UAUUCAGGCUAUCUGGGAC | 975 | siTIMP1_p70 |
| H +/− Rh | 4 | GCAGAGUGGCACUCAUUGA | 897 | UCAAUGAGUGCCACUCUGC | 978 | siTIMP1_p75 |
| H +/− Rh | 3 | CCAUGGAGAGUGUCUGCGA | 903 | UCGCAGACACUCUCCAUGG | 983 | siTIMP1_p83 |
| H +/− Rh | 4 | ACUGCAGAGUGGCACUCAA | 904 | UUGAGUGCCACUCUGCAGU | 985 | siTIMP1_p86 |
| H +/− Rh | 2 | CCAGACCACCUUAUACCAA | 906 | UUGGUAUAAGGUGGUCUGG | 987 | siTIMP1_p88 |
| H +/− Rh | 4 | CCUGAAUCCUGCCCGGAGA | 908 | UCUCCGGGCAGGAUUCAGG | 989 | siTIMP1_p92 |
| H +/− Rh | 3 | CUGAAGCCUGCACAGUGUA | 909 | UACACUGUGCAGGCUUCAG | 990 | siTIMP1_p93 |
| H +/− Rh | 4 | UCUCAUUGCUGGAAAACUA | 911 | UAGUUUUCCAGCAAUGAGA | 992 | siTIMP1_p95 |
| H +/− Rh | 2 | AGACCUACACUGUUGGCUA | 912 | UAGCCAACAGUGUAGGUCU | 993 | siTIMP1_p97 |
| H +/− Rh | 4 | GUUCCCACUCCCAUCUUUA | 915 | UAAAGAUGGGAGUGGGAAC | 996 | siTIMP1_p102 |
| H +/− Rh | 4 | CUUCUGGCAUCCUGUUGUA | 917 | UACAACAGGAUGCCAGAAG | 998 | siTIMP1_p104 |
| H +/− Rh | 2 | AGAGUGUCUGCGGAUACUA | 918 | UAGUAUCCGCAGACACUCU | 999 | siTIMP1_p105 |
| H +/− Rh | 3 | CACCAAGACCUACACUGUA | 919 | UACAGUGUAGGUCUUGGUG | 1000 | siTIMP1_p106 |
| H +/− Rh | 2 | UGCAGAGUGGCACUCAUUA | 921 | UAAUGAGUGCCACUCUGCA | 1002 | siTIMP1_p110 |
| H +/− Rh | 4 | AGCGAGGAGUUUCUCAUUA | 923 | UAAUGAGAAACUCCUCGCU | 1004 | siTIMP1_p112 |

### TIMP2—TIMP Metallopeptidase Inhibitor 2

TABLE B1
siTIMP2 19-mers

| | | | | | |
|---|---|---|---|---|---|
| | SEQ | | SEQ | | |
| | ID | | ID | | human-73858577 |
| Sense (5′ > 3′) | NO: | Antisense (5′ > 3′) | NO: | Other Sp | ORF: 303-965 |

| | | | | | |
|---|---|---|---|---|---|
| GGAAGAACUUUCUCGGUAA | 1007 | UUACCGAGAAAGUUCUUCC | 1622 | Rh | [2332-2350] 3′UTR |
| GGUUCUCCAGUUCAAAUUA | 1008 | UAAUUUGAACUGGAGAACC | 1623 | | [3062-3080] 3′UTR |
| CUGUGUUUAUGCUGGAAUA | 1009 | UAUUCCAGCAUAAACACAG | 1624 | | [3495-3513] 3′UTR |
| CCUGUAUGGUGAUAUCAUA | 1010 | UAUGAUAUCACCAUACAGG | 1625 | | [2789-2807] 3′UTR |
| GGCACAUUAUGUAAACAUA | 1011 | UAUGUUUACAUAAUGUGCC | 1626 | Rh | [2412-2430] 3′UTR |
| GGUGAAUUCUCAGAUGAUA | 1012 | UAUCAUCUGAGAAUUCACC | 1627 | | [2165-2183] 3′UTR |
| CCAUGUGAUUUCAGUAUAU | 1013 | AUAUACUGAAAUCACAUGG | 1628 | Rh | [2718-2736] 3′UTR |
| CUCUGAGCCUUGUAGAAAU | 1014 | AUUUCUACAAGGCUCAGAG | 1629 | Rh | [1606-1624] 3′UTR |
| GGCUGCGAGUGCAAGAUCA | 1015 | UGAUCUUGCACUCGCAGCC | 1630 | Ck, Rb, Rt | [752-770] ORF |
| AGAGGAAGCCGCUCAAAUA | 1016 | UAUUUGAGCGGCUUCCUCU | 1631 | | [3214-3232] 3′UTR |
| CUUUGGUUCUCCAGUUCAA | 1017 | UUGAACUGGAGAACCAAAG | 1632 | | [3058-3076] 3′UTR |
| CAGUAUGAGAUCAAGCAGA | 1018 | UCUGCUUGAUCUCAUACUG | 1633 | Rh, Rb, Cw, Dg, Ms | [509-527] ORF |
| CUUGCAAAAUGCUUCCAAA | 1019 | UUUGGAAGCAUUUUGCAAG | 1634 | | [2471-2489] 3′UTR |
| CUUGGUAGGUAUUAGACUU | 1020 | AAGUCUAAUACCUACCAAG | 1635 | | [2906-2924] 3′UTR |
| CCCUCUGAGCCUUGUAGAA | 1021 | UUCUACAAGGCUCAGAGGG | 1636 | Rh | [1604-1622] 3′UTR |
| GGUAGGUAUUAGACUUGCA | 1022 | UGCAAGUCUAAUACCUACC | 1637 | | [2909-2927] 3′UTR |
| GGGUCACAGAGAAGAACAU | 1023 | AUGUUCUUCUCUGUGACCC | 1638 | Rh | [831-849] ORF |
| GAACCUGAGUUGCAGAUAU | 1024 | AUAUCUGCAACUCAGGUUC | 1639 | Rh | [2187-2205] 3′UTR |
| GGAUUGAGUUGCACAGCUU | 1025 | AAGCUGUGCAACUCAAUCC | 1640 | | [1853-1871] 3′UTR |
| GGUGAUAUCAUAUGUAACA | 1026 | UGUUACAUAUGAUAUCACC | 1641 | Rh | [2796-2814] 3′UTR |
| CCUGCAAGCAACUCAAAAU | 1027 | AUUUUGAGUUGCUUGCAGG | 1642 | | [3343-3361] 3′UTR |
| GGAUAUAGAGUUUAUCUAC | 1028 | GUAGAUAAACUCUAUAUCC | 1643 | | [553-571] ORF |
| GAUGCUUUGUAUCAUUCUU | 1029 | AAGAAUGAUACAAAGCAUC | 1644 | | [3589-3607] 3′UTR |
| GCAAGCAACUCAAAAUAUU | 1030 | AAUAUUUUGAGUUGCUUGC | 1645 | | [3346-3364] 3′UTR |
| CGUCUUUGGUUCUCCAGUU | 1031 | AACUGGAGAACCAAAGACG | 1646 | | [3055-3073] 3′UTR |
| CCUUUAUAUUUGAUCCACA | 1032 | UGUGGAUCAAAUAUAAAGG | 1647 | | [3127-3145] 3′UTR |
| GUGCUGAGCAGAAAACAAA | 1033 | UUUGUUUUCUGCUCAGCAC | 1648 | | [3164-3182] 3′UTR |
| CCAACUUCUGCUUGUAUUU | 1034 | AAAUACAAGCAGAAGUUGG | 1649 | Rh | [2207-2225] 3′UTR |
| CCUAUUAAUCCUCAGAAUU | 1035 | AAUUCUGAGGAUUAAUAGG | 1650 | Rh | [1572-1590] 3′UTR |
| CGGUAAUGAUAAGGAGAAU | 1036 | AUUCUCCUUAUCAUUACCG | 1651 | | [2345-2363] 3′UTR |
| CGCUCAAAUACCUUCACAA | 1037 | UUGUGAAGGUAUUUGAGCG | 1652 | | [3223-3241] 3′UTR |
| GGGCAGACUGGGAGGGUAU | 1038 | AUACCCUCCCAGUCUGCCC | 1653 | Rh | [2619-2637] 3′UTR |
| UGCUGAGCAGAAAACAAAA | 1039 | UUUUGUUUUCUGCUCAGCA | 1654 | | [3165-3183] 3′UTR |
| AGCGGUCAGUGAGAAGGAA | 1040 | UUCCUUCUCACUGACCGCU | 1655 | | [445-463] ORF |
| GGUAUUAGACUUGCACUUU | 1041 | AAAGUGCAAGUCUAAUACC | 1656 | | [2913-2931] 3′UTR |
| GCUGGAAUAUGAAGUCUGA | 1042 | UCAGACUUCAUAUUCCAGC | 1657 | Ms | [3505-3523] 3′UTR |
| CCUGUGUUGUAAAGAUAAA | 1043 | UUUAUCUUUACAACACAGG | 1658 | Rh | [2380-2398] 3′UTR |
| GGUAAGAUGUCAUAAUGGA | 1044 | UCCAUUAUGACAUCUUACC | 1659 | Rh | [2694-2712] 3′UTR |
| GUGGUUUCCUGAAGCCAGU | 1045 | ACUGGCUUCAGGAAACCAC | 1660 | | [2295-2313] 3′UTR |
| GGGUCCAAAUUAAUAUGAU | 1046 | AUCAUAUUAAUUUGGACCC | 1661 | | [1077-1095] 3′UTR |
| GGAACACACAAGAGUUGUU | 1047 | AACAACUCUUGUGUGUUCC | 1662 | | [3539-3557] 3′UTR |
| AGAUUACCUAGCUAAGAAA | 1048 | UUUCUUAGCUAGGUAAUCU | 1663 | | [2238-2256] 3′UTR |
| CUGGGAACACACAAGAGUU | 1049 | AACUCUUGUGUGUUCCCAG | 1664 | | [3536-3554] 3′UTR |
| UCCCAUGGGUCCAAAUUAA | 1050 | UUAAUUUGGACCCAUGGGA | 1665 | | [1071-1089] 3′UTR |
| GUUCUCCAGUUCAAAUUAU | 1051 | AUAAUUUGAACUGGAGAAC | 1666 | | [3063-3081] 3′UTR |
| CCAUGGGUCCAAAUUAAUA | 1052 | UAUUAAUUUGGACCCAUGG | 1667 | | [1073-1091] 3′UTR |
| UGGGCGUGGUCUUGCAAAA | 1053 | UUUUGCAAGACCACGCCCA | 1668 | | [2461-2479] 3′UTR |
| CGUGCUGAGCAGAAAACAA | 1054 | UUGUUUUCUGCUCAGCACG | 1669 | | [3163-3181] 3′UTR |
| CGGUCAGUGAGAAGGAAGU | 1055 | ACUUCCUUCUCACUGACCG | 1670 | | [447-465] ORF |
| CGAUAUACAGGCACAUUAU | 1056 | AUAAUGUGCCUGUAUAUCG | 1671 | | [2403-2421] 3′UTR |
| GCAUUUUGCAGAAACUUUU | 1057 | AAAAGUUUCUGCAAAAUGC | 1672 | Rh | [1334-1352] 3′UTR |
| GGACCAGUCCAUGUGAUUU | 1058 | AAAUCACAUGGACUGGUCC | 1673 | Rh | [2710-2728] 3′UTR |
| GCUCAAAUACCUUCACAAU | 1059 | AUUGUGAAGGUAUUUGAGC | 1674 | | [3224-3242] 3′UTR |
| CACCUUAGCCUGUUCUAUU | 1060 | AAUAGAACAGGCUAAGGUG | 1675 | Rh | [2492-2510] 3′UTR |
| GGAUCUCCCAGCUGGGUUA | 1061 | UAACCCAGCUGGGAGAUCC | 1676 | | [1296-1314] 3′UTR |
| GUAUUAGACUUGCACUUUU | 1062 | AAAAGUGCAAGUCUAAUAC | 1677 | | [2914-2932] 3′UTR |
| AGAGGAUCCAGUAUGAGAU | 1063 | AUCUCAUACUGGAUCCUCU | 1678 | Rh, Rb | [501-519] ORF |
| GAACCUAUGUGUUCCCUCA | 1064 | UGAGGGAACACAUAGGUUC | 1679 | | [2274-2292] 3′UTR |
| CUGAGUUGCAGAUAUACCA | 1065 | UGGUAUAUCUGCAACUCAG | 1680 | Rh | [2191-2209] 3′UTR |
| CUCAAAUACCUUCACAAUA | 1066 | UAUUGUGAAGGUAUUUGAG | 1681 | | [3225-3243] 3′UTR |
| UCCUAUUAAUCCUCAGAAU | 1067 | AUUCUGAGGAUUAAUAGGA | 1682 | Rh | [1571-1589] 3′UTR |
| CCAGUUCAAAUUAUUGCAA | 1068 | UUGCAAUAAUUUGAACUGG | 1683 | | [3068-3086] 3′UTR |
| UGUUUAUGCUGGAAUAUGA | 1069 | UCAUAUUCCAGCAUAAACA | 1684 | | [3498-3516] 3′UTR |
| GCACAGAUCUUGAUGACUU | 1070 | AAGUCAUCAAGAUCUGUGC | 1685 | Rh | [2591-2609] 3′UTR |
| AACCUGAGUUGCAGAUAUA | 1071 | UAUAUCUGCAACUCAGGUU | 1686 | Rh | [2188-2206] 3 ′UTR |
| CUGCAAGCAACUCAAAAUA | 1072 | UAUUUUGAGUUGCUUGCAG | 1687 | | [3344-3362] 3′UTR |
| GGCUUUGGUGACACACUCA | 1073 | UGAGUGUGUCACCAAAGCC | 1688 | | [2086-2104] 3′UTR |
| GUUGCAAGACUGUGUAGCA | 1074 | UGCUACACAGUCUUGCAAC | 1689 | Rh | [1360-1378] 3′UTR |
| GACAUUUAUGGCAACCCUA | 1075 | UAGGGUUGCCAUAAAUGUC | 1690 | | [479-497] ORF |
| GUAUGAGAUCAAGCAGAUA | 1076 | UAUCUGCUUGAUCUCAUAC | 1691 | Rh, Rb, Cw, Dg, Rt, Ms | [511-529] ORF |
| GACUUGCUGCCGUAAUUUA | 1077 | UAAAUUACGGCAGCAAGUC | 1692 | | [3428-3446] 3′UTR |
| GAAAGAAGGAAUAUCUCAU | 1078 | AUGAGAUAUUCCUUCUUUC | 1693 | | [618-636] ORF |
| GAGGAAAGAAGGAAUAUCU | 1079 | AGAUAUUCCUUCUUUCCUC | 1694 | Rt | [615-633] ORF |
| CGUGGACAAUAAACAGUAU | 1080 | AUACUGUUUAUUGUCCACG | 1695 | | [3624-3642] 3 ′UTR |
| GGUGAACCUGAGUUGCAGA | 1081 | UCUGCAACUCAGGUUCACC | 1696 | Rh | [2184-2202] 3′UTR |
| CCUGCAUCAAGAGAAGUGA | 1082 | UCACUUCUCUUGAUGCAGG | 1697 | Rt, Ms | [876-894] ORF |
| AGUCCAUGUGAUUUCAGUA | 1083 | UACUGAAAUCACAUGGACU | 1698 | Rh | [2715-2733] 3′UTR |
| AGUAAAGGAUCUUUGAGUA | 1084 | UACUCAAAGAUCCUUUACU | 1699 | | [3087-3105] 3′UTR |
| CCCAGAAGAAGAGCCUGAA | 1085 | UUCAGGCUCUUCUUCUGGG | 1700 | Rh, Cw, Ms, Pg | [717-735] ORF |
| GACAUCAGCUGUAAUCAUU | 1086 | AAUGAUUACAGCUGAUGUC | 1701 | | [2864-2882] 3′UTR |
| CCUCAAAGACUGACAGCCA | 1087 | UGGCUGUCAGUCUUUGAGG | 1702 | Rh | [1980-1998] 3′UTR |
| CUCGGUCCGUGGACAAUAA | 1088 | UUAUUGUCCACGGACCGAG | 1703 | | [3617-3635] 3′UTR |
| AGGGCAGCCUGGAACCAGU | 1089 | ACUGGUUCCAGGCUGCCCU | 1704 | | [1525-1543] 3′UTR |
| CCGUGGACAAUAAACAGUA | 1090 | UACUGUUUAUUGUCCACGG | 1705 | | [3623-3641] 3′UTR |
| GAAACGACAUUUAUGGCAA | 1091 | UUGCCAUAAAUGUCGUUUC | 1706 | | [474-492] ORF |
| CCUCAGAAUUCCAGUGGGA | 1092 | UCCCACUGGAAUUCUGAGG | 1707 | Rh | [1581-1599] 3′UTR |
| GUCACAGAGAAGAACAUCA | 1093 | UGAUGUUCUUCUCUGUGAC | 1708 | Rh | [833-851] ORF |
| CCAGUGGCUAGUUCUUGAA | 1094 | UUCAAGAACUAGCCACUGG | 1709 | | [1539-1557] 3′UTR |
| GGAACCAGUGGCUAGUUCU | 1095 | AGAACUAGCCACUGGUUCC | 1710 | | [1535-1553] 3′UTR |
| UCCAUGUGAUUUCAGUAUA | 1096 | UAUACUGAAAUCACAUGGA | 1711 | Rh | [2717-2735] 3′UTR |
| AGGUAUUAGACUUGCACUU | 1097 | AAGUGCAAGUCUAAUACCU | 1712 | | [2912-2930] 3′UTR |
| CAUUUGACCCAGAGUGGAA | 1098 | UUCCACUCUGGGUCAAAUG | 1713 | | [2961-2979] 3′UTR |
| GCACCUGGAUUGAGUUGCA | 1099 | UGCAACUCAAUCCAGGUGC | 1714 | | [1847-1865] 3′UTR |
| AGUUGUUGAAAGUUGACAA | 1100 | UUGUCAACUUUCAACAACU | 1715 | | [3551-3569] 3′UTR |
| GUGGCCAACUGCAAAAAAA | 1101 | UUUUUUUGCAGUUGGCCAC | 1716 | | [984-1002] 3′UTR |
| CUCAAAGACUGACAGCCAU | 1102 | AUGGCUGUCAGUCUUUGAG | 1717 | Rh | [1981-1999] 3′UTR |
| GCCUCAGCUGAGUCUUUUU | 1103 | AAAAAGACUCAGCUGAGGC | 1718 | Rh | [1658-1676] 3′UTR |
| UGCUUUGUAUCAUUCUUGA | 1104 | UCAAGAAUGAUACAAAGCA | 1719 | | [3591-3609] 3′UTR |
| GUUUAAGAAGGCUCUCCAU | 1105 | AUGGAGAGCCUUCUUAAAC | 1720 | | [3265-3283] 3′UTR |
| CCAGCUAAGCAUAGUAAGA | 1106 | UCUUACUAUGCUUAGCUGG | 1721 | | [2030-2048] 3′UTR |
| GUUGGUAAGAUGUCAUAAU | 1107 | AUUAUGACAUCUUACCAAC | 1722 | Rh | [2691-2709] 3′UTR |
| CACCUGUGUUGUAAAGAUA | 1108 | UAUCUUUACAACACAGGUG | 1723 | Rh | [2378-2396] 3′UTR |
| CAGCCUCAGCUGAGUCUUU | 1109 | AAAGACUCAGCUGAGGCUG | 1724 | Rh | [1656-1674] 3′UTR |
| AUGAGAUCAAGCAGAUAAA | 1110 | UUUAUCUGCUUGAUCUCAU | 1725 | Rh, Cw, Dg, Rt, Ms | [513-531] ORF |
| GUUGCACAGCUUUGCUUUA | 1111 | UAAAGCAAAGCUGUGCAAC | 1726 | | [1860-1878] 3′UTR |
| GUGGCUAGUUCUUGAAGGA | 1112 | UCCUUCAAGAACUAGCCAC | 1727 | | [1542-1560] 3′UTR |
| GAUUGAGUUGCACAGCUUU | 1113 | AAAGCUGUGCAACUCAAUC | 1728 | | [1854-1872] 3′UTR |
| GGAUCUUUGAGUAGGUUCG | 1114 | CGAACCUACUCAAAGAUCC | 1729 | | [3093-3111] 3′UTR |
| CGCUGGACGUUGGAGGAAA | 1115 | UUUCCUCCAACGUCCAGCG | 1730 | Rt, Ms | [603-621] ORF |
| CACACACGUUGGUCUUUUA | 1116 | UAAAAGACCAACGUGUGUG | 1731 | | [3142-3160] 3′UTR |
| CUCAGUGUGGUUUCCUGAA | 1117 | UUCAGGAAACCACACUGAG | 1732 | | [2289-2307] 3′UTR |
| AUGUUAUGUUCUAAGCACA | 1118 | UGUGCUUAGAACAUAACAU | 1733 | | [3303-3321] 3′UTR |
| GCCACCUUAGCCUGUUCUA | 1119 | UAGAACAGGCUAAGGUGGC | 1734 | Rh | [2490-2508] 3′UTR |
| AAGAGUUGUUGAAAGUUGA | 1120 | UCAACUUUCAACAACUCUU | 1735 | | [3548-3566] 3′UTR |
| CCUGAGAAGGAUAUAGAGU | 1121 | ACUCUAUAUCCUUCUCAGG | 1736 | | [545-563] ORF |
| ACCAGUGGCUAGUUCUUGA | 1122 | UCAAGAACUAGCCACUGGU | 1737 | | [1538-1556] 3′UTR |
| CAUCCUGCAAGCAACUCAA | 1123 | UUGAGUUGCUUGCAGGAUG | 1738 | | [3340-3358] 3′UTR |
| GUAAUGAUAAGGAGAAUCU | 1124 | AGAUUCUCCUUAUCAUUAC | 1739 | | [2347-2365] 3′UTR |
| GGAAUAUCUCAUUGCAGGA | 1125 | UCCUGCAAUGAGAUAUUCC | 1740 | | [625-643] ORF |
| GGGCGUGGUCUUGCAAAAU | 1126 | AUUUUGCAAGACCACGCCC | 1741 | | [2462-2480] 3′UTR |
| CAUCCUGAGGACAGAAAAA | 1127 | UUUUUCUGUCCUCAGGAUG | 1742 | Rh | [1921-1939] 3′UTR |
| UGGACUUGCUGCCGUAAUU | 1128 | AAUUACGGCAGCAAGUCCA | 1743 | | [3426-3444] 3′UTR |
| GUGACACACUCACUUCUUU | 1129 | AAAGAAGUGAGUGUGUCAC | 1744 | | [2093-2111] 3 ′UTR |
| CUGUUUUAAGAGACAUCUU | 1130 | AAGAUGUCUCUUAAAACAG | 1745 | Rh | [2135-2153] 3′UTR |
| GUUUAUGCUGGAAUAUGAA | 1131 | UUCAUAUUCCAGCAUAAAC | 1746 | | [3499-3517] 3′UTR |
| GUCCAUGUGAUUUCAGUAU | 1132 | AUACUGAAAUCACAUGGAC | 1747 | Rh | [2716-2734] 3′UTR |
| AGGAGUUUCUCGACAUCGA | 1133 | UCGAUGUCGAGAAACUCCU | 1748 | Ck, Dg | [936-954] ORF |
| GAAGAACUUUCUCGGUAAU | 1134 | AUUACCGAGAAAGUUCUUC | 1749 | Rh | [2333-2351] 3′UTR |
| GGGUCUGGAGGGAGACGUG | 1135 | CACGUCUCCCUCCAGACCC | 1750 | | [1130-1148] 3′UTR |
| GGAAGCCGCUCAAAUACCU | 1136 | AGGUAUUUGAGCGGCUUCC | 1751 | | [3217-3235] 3′UTR |
| GGUCCGUGGACAAUAAACA | 1137 | UGUUUAUUGUCCACGGACC | 1752 | | [3620-3638] 3′UTR |
| CCCUCCAACCCAUAUAACA | 1138 | UGUUAUAUGGGUUGGAGGG | 1753 | | [2752-2770] 3′UTR |
| CCCAUGGGUCCAAAUUAAU | 1139 | AUUAAUUUGGACCCAUGGG | 1754 | | [1072-1090] 3′UTR |
| CACACUCACUUCUUUCUCA | 1140 | UGAGAAAGAAGUGAGUGUG | 1755 | | [2097-2115] 3′UTR |
| GCAGAAAACAAAACAGGUU | 1141 | AACCUGUUUUGUUUUCUGC | 1756 | | [3171-3189] 3′UTR |
| CAUCAAUCCUAUUAAUCCU | 1142 | AGGAUUAAUAGGAUUGAUG | 1757 | Rh | [1565-1583] 3′UTR |
| CACAAUAAAUAGUGGCAAU | 1143 | AUUGCCACUAUUUAUUGUG | 1758 | | [3237-3255] 3′UTR |
| GUUGGAGGAAAGAAGGAAU | 1144 | AUUCCUUCUUUCCUCCAAC | 1759 | Rt | [611-629] ORF |
| GGCCUUUAUAUUUGAUCCA | 1145 | UGGAUCAAAUAUAAAGGCC | 1760 | | [3125-3143] 3′UTR |
| UGUUCAAAGGGCCUGAGAA | 1146 | UUCUCAGGCCCUUUGAACA | 1761 | | [534-552] ORF |
| ACUGGGUCACAGAGAAGAA | 1147 | UUCUUCUCUGUGACCCAGU | 1762 | Rh, Rt, Ms | [828-846] ORF |
| GGUAAUGAUAAGGAGAAUC | 1148 | GAUUCUCCUUAUCAUUACC | 1763 | | [2346-2364] 3′UTR |
| CACACAAGAGUUGUUGAAA | 1149 | UUUCAACAACUCUUGUGUG | 1764 | | [3543-3561] 3′UTR |
| CUCUGGAUGGACUGGGUCA | 1150 | UGACCCAGUCCAUCCAGAG | 1765 | Rh, Rb, Cw, Dg, Rt, Ms, | [818-836] ORF |
| | | | | Pg | |
| GGAACUAGGGAACCUAUGU | 1151 | ACAUAGGUUCCCUAGUUCC | 1766 | Rh | [2265-2283] 3′UTR |
| CUCGGUAAUGAUAAGGAGA | 1152 | UCUCCUUAUCAUUACCGAG | 1767 | | [2343-2361] 3′UTR |
| AGGUGAAUUCUCAGAUGAU | 1153 | AUCAUCUGAGAAUUCACCU | 1768 | | [2164-2182] 3′UTR |
| UCGGUAAUGAUAAGGAGAA | 1154 | UUCUCCUUAUCAUUACCGA | 1769 | | [2344-2362] 3′UTR |
| GCCAAAGCGGUCAGUGAGA | 1155 | UCUCACUGACCGCUUUGGC | 1770 | | [440-458] ORF |
| GAACCAGUGGCUAGUUCUU | 1156 | AAGAACUAGCCACUGGUUC | 1771 | | [1536-1554] 3′UTR |
| CCCUUCUCCUUUUAGACAU | 1157 | AUGUCUAAAAGGAGAAGGG | 1772 | | [1105-1123] 3′UTR |
| CCACCUUAGCCUGUUCUAU | 1158 | AUAGAACAGGCUAAGGUGG | 1773 | Rh | [2491-2509] 3′UTR |
| CCCUGAGCACCACCCAGAA | 1159 | UUCUGGGUGGUGCUCAGGG | 1774 | Rh, Pg | [705-723] ORF |
| UGCUGUACAGUGACCUAAA | 1160 | UUUAGGUCACUGUACAGCA | 1775 | | [2672-2690] 3′UTR |
| CCUUAGCCUGUUCUAUUCA | 1161 | UGAAUAGAACAGGCUAAGG | 1776 | Rh | [2494-2512] 3′UTR |
| GAACUUUCUCGGUAAUGAU | 1162 | AUCAUUACCGAGAAAGUUC | 1777 | | [2336-2354] 3′UTR |
| CCUAGGAAGGGAAGGAUUU | 1163 | AAAUCCUUCCCUUCCUAGG | 1778 | Rh | [2056-2074] 3′UTR |
| UCAGUGAGAAGGAAGUGGA | 1164 | UCCACUUCCUUCUCACUGA | 1779 | | [450-468] ORF |
| AUAUGAAGUCUGAGACCUU | 1165 | AAGGUCUCAGACUUCAUAU | 1780 | | [3511-3529] 3′UTR |
| GGACUCUGGAAACGACAUU | 1166 | AAUGUCGUUUCCAGAGUCC | 1781 | Rh | [466-484] ORF |
| CCUCUGAGCCUUGUAGAAA | 1167 | UUUCUACAAGGCUCAGAGG | 1782 | Rh | [1605-1623] 3′UTR |
| AGUUUAAGAAGGCUCUCCA | 1168 | UGGAGAGCCUUCUUAAACU | 1783 | | [3264-3282] 3′UTR |
| AGGGCAGACUGGGAGGGUA | 1169 | UACCCUCCCAGUCUGCCCU | 1784 | Rh | [2618-2636] 3′UTR |
| GUAGAAAUGGGAGCGAGAA | 1170 | UUCUCGCUCCCAUUUCUAC | 1785 | | [1617-1635] 3′UTR |
| GGACUUGCUGCCGUAAUUU | 1171 | AAAUUACGGCAGCAAGUCC | 1786 | | [3427-3445] 3′UTR |
| AGAACUUUCUCGGUAAUGA | 1172 | UCAUUACCGAGAAAGUUCU | 1787 | | [2335-2353] 3′UTR |
| GUAUCAUUCUUGAGCAAUC | 1173 | GAUUGCUCAAGAAUGAUAC | 1788 | | [3597-3615] 3′UTR |
| CAGCUAAGCAUAGUAAGAA | 1174 | UUCUUACUAUGCUUAGCUG | 1789 | | [2031-2049] 3′UTR |
| GGCCUGUUUUAAGAGACAU | 1175 | AUGUCUCUUAAAACAGGCC | 1790 | Rh | [2132-2150] 3′UTR |
| GACUGGGUCACAGAGAAGA | 1176 | UCUUCUCUGUGACCCAGUC | 1791 | Rh, Rt, Ms | [827-845] ORF |
| CUCUGAUGCUUUGUAUCAU | 1177 | AUGAUACAAAGCAUCAGAG | 1792 | | [3585-3603] 3′UTR |
| GUAACAUUUACUCCUGUUU | 1178 | AAACAGGAGUAAAUGUUAC | 1793 | Rh | [2809-2827] 3′UTR |
| UGAGUUGCAGAUAUACCAA | 1179 | UUGGUAUAUCUGCAACUCA | 1794 | Rh | [2192-2210] 3′UTR |
| AUCCCAUGGGUCCAAAUUA | 1180 | UAAUUUGGACCCAUGGGAU | 1795 | | [1070-1088] 3′UTR |
| CUCUGGAAACGACAUUUAU | 1181 | AUAAAUGUCGUUUCCAGAG | 1796 | Rh | [469-487] ORF |
| GAUCCAGUAUGAGAUCAAG | 1182 | CUUGAUCUCAUACUGGAUC | 1797 | Rh, Rb | [505-523] ORF |
| AGGUGUGGCCUUUAUAUUU | 1183 | AAAUAUAAAGGCCACACCU | 1798 | | [3119-3137] 3′UTR |
| CCCUGUUCGCUUCCUGUAU | 1184 | AUACAGGAAGCGAACAGGG | 1799 | | [2777-2795] 3′UTR |
| GGGAGACGUGGGUCCAAGG | 1185 | CCUUGGACCCACGUCUCCC | 1800 | | [1139-1157] 3′UTR |
| CAUGGGUCCAAAUUAAUAU | 1186 | AUAUUAAUUUGGACCCAUG | 1801 | | [1074-1092] 3′UTR |
| UGGGUCACAGAGAAGAACA | 1187 | UGUUCUUCUCUGUGACCCA | 1802 | Rh | [830-848] ORF |
| CCUCAAGGUCCCUUCCCUA | 1188 | UAGGGAAGGGACCUUGAGG | 1803 | | [1786-1804] 3′UTR |
| UGGUUCUCCAGUUCAAAUU | 1189 | AAUUUGAACUGGAGAACCA | 1804 | | [3061-3079] 3′UTR |
| GGACCUGGUCAGCACAGAU | 1190 | AUCUGUGCUGACCAGGUCC | 1805 | Rh | [2580-2598] 3′UTR |
| GGAGGGAGACGUGGGUCCA | 1191 | UGGACCCACGUCUCCCUCC | 1806 | | [1136-1154] 3′UTR |
| UCUGAUGCUUUGUAUCAUU | 1192 | AAUGAUACAAAGCAUCAGA | 1807 | | [3586-3604] 3′UTR |
| GGGACAUGGCCCUUGUUUU | 1193 | AAAACAAGGGCCAUGUCCC | 1808 | | [1407-1425] 3′UTR |
| GCCUGGGCGUGGUCUUGCA | 1194 | UGCAAGACCACGCCCAGGC | 1809 | | [2458-2476] 3′UTR |
| GGCGUUUUGCAAUGCAGAU | 1195 | AUCUGCAUUGCAAAACGCC | 1810 | Cw, Rt, Ms | [409-427] ORF |
| GAGUAGGUUCGGUCUGAAA | 1196 | UUUCAGACCGAACCUACUC | 1811 | | [3101-3119] 3′UTR |
| AGUUCUUCGCCUGCAUCAA | 1197 | UUGAUGCAGGCGAAGAACU | 1812 | Rh, Rb, Cw, Dg, Ms | [867-885] ORF |
| ACAAAGAUUACCUAGCUAA | 1198 | UUAGCUAGGUAAUCUUUGU | 1813 | | [2234-2252] 3′UTR |
| GAGGGAGACGUGGGUCCAA | 1199 | UUGGACCCACGUCUCCCUC | 1814 | | [1137-1155] 3′UTR |
| CUGUUUCUGCUGAUUGUUU | 1200 | AAACAAUCAGCAGAAACAG | 1815 | | [2822-2840] 3′UTR |
| CUGACGAUAUACAGGCACA | 1201 | UGUGCCUGUAUAUCGUCAG | 1816 | | [2399-2417] 3′UTR |
| UGUUGAAAGUUGACAAGCA | 1202 | UGCUUGUCAACUUUCAACA | 1817 | | [3554-3572] 3′UTR |
| GCCUAGGAAGGGAAGGAUU | 1203 | AAUCCUUCCCUUCCUAGGC | 1818 | Rh | [2055-2073] 3′UTR |
| GGUGACACACUCACUUCUU | 1204 | AAGAAGUGAGUGUGUCACC | 1819 | | [2092-2110] 3′UTR |
| GAGCCUUGUAGAAAUGGGA | 1205 | UCCCAUUUCUACAAGGCUC | 1820 | Rh | [1610-1628] 3′UTR |
| CAGAAAACAAAACAGGUUA | 1206 | UAACCUGUUUUGUUUUCUG | 1821 | | [3172-3190] 3′UTR |
| CGCAUGUCUCUGAUGCUUU | 1207 | AAAGCAUCAGAGACAUGCG | 1822 | | [3578-3596] 3′UTR |
| GACAAAGAUUACCUAGCUA | 1208 | UAGCUAGGUAAUCUUUGUC | 1823 | | [2233-2251] 3′UTR |
| CUGUAUGGUGAUAUCAUAU | 1209 | AUAUGAUAUCACCAUACAG | 1824 | | [2790-2808] 3′UTR |
| GACUCUGGAAACGACAUUU | 1210 | AAAUGUCGUUUCCAGAGUC | 1825 | Rh | [467-485] ORF |
| GGUUCGGUCUGAAAGGUGU | 1211 | ACACCUUUCAGACCGAACC | 1826 | | [3106-3124] 3′UTR |
| AGAUGAUAGGUGAACCUGA | 1212 | UCAGGUUCACCUAUCAUCU | 1827 | | [2176-2194] 3′UTR |
| GACACUAUGGCCUGUUUUA | 1213 | UAAAACAGGCCAUAGUGUC | 1828 | | [2124-2142] 3′UTR |
| CUGCAAAAAAAGCCUCCAA | 1214 | UUGGAGGCUUUUUUUGCAG | 1829 | | [992-1010] 3′UTR |
| CUGUUCUAUUCAGCGGCAA | 1215 | UUGCCGCUGAAUAGAACAG | 1830 | | [2501-2519] 3′UTR |
| GUGGGUCCAAGGUCCUCAU | 1216 | AUGAGGACCUUGGACCCAC | 1831 | | [1146-1164] 3′UTR |
| GCCUGAGAAGGAUAUAGAG | 1217 | CUCUAUAUCCUUCUCAGGC | 1832 | | [544-562] ORF |
| GAAACUUCCUAGGGAACUA | 1218 | UAGUUCCCUAGGAAGUUUC | 1833 | | [2253-2271] 3′UTR |
| CGCCAGCUAAGCAUAGUAA | 1219 | UUACUAUGCUUAGCUGGCG | 1834 | | [2028-2046] 3′UTR |
| GCCUCUGGAUGGACUGGGU | 1220 | ACCCAGUCCAUCCAGAGGC | 1835 | Rh, Rb, Cw, Dg, Rt, Ms | [816-834] ORF |
| ACGAUAUACAGGCACAUUA | 1221 | UAAUGUGCCUGUAUAUCGU | 1836 | | [2402-2420] 3′UTR |
| AGCACCACCCAGAAGAAGA | 1222 | UCUUCUUCUGGGUGGUGCU | 1837 | Rh, Pg | [710-728] ORF |
| CCUCCCUCAAAGACUGACA | 1223 | UGUCAGUCUUUGAGGGAGG | 1838 | Rh | [1976-1994] 3′UTR |
| GGAGCACUGUGUUUAUGCU | 1224 | AGCAUAAACACAGUGCUCC | 1839 | | [3489-3507] 3′UTR |
| ACUUGCUGCCGUAAUUUAA | 1225 | UUAAAUUACGGCAGCAAGU | 1840 | | [3429-3447] 3′UTR |
| GGUUUCCUGAAGCCAGUGA | 1226 | UCACUGGCUUCAGGAAACC | 1841 | | [2297-2315] 3′UTR |
| GGUCAGUGAGAAGGAAGUG | 1227 | CACUUCCUUCUCACUGACC | 1842 | | [448-466] ORF |
| GUGACGCCAGCUAAGCAUA | 1228 | UAUGCUUAGCUGGCGUCAC | 1843 | | [2024-2042] 3′UTR |
| AUGAUAAGGAGAAUCUCUU | 1229 | AAGAGAUUCUCCUUAUCAU | 1844 | | [2350-2368] 3′UTR |
| UAGUGUUCCCUCCCUCAAA | 1230 | UUUGAGGGAGGGAACACUA | 1845 | | [1968-1986] 3′UTR |
| GGAGACGUGGGUCCAAGGU | 1231 | ACCUUGGACCCACGUCUCC | 1846 | | [1140-1158] 3′UTR |
| CCUGUUCUAUUCAGCGGCA | 1232 | UGCCGCUGAAUAGAACAGG | 1847 | | [2500-2518] 3′UTR |
| UCCAGUAUGAGAUCAAGCA | 1233 | UGCUUGAUCUCAUACUGGA | 1848 | Rh, Rb | [507-525] ORF |
| CAAAAUGCUUCCAAAGCCA | 1234 | UGGCUUUGGAAGCAUUUUG | 1849 | Rh | [2475-2493] 3′UTR |
| CACACGCAAUGAAACCGAA | 1235 | UUCGGUUUCAUUGCGUGUG | 1850 | | [2431-2449] 3′UTR |
| CUCCAUUUGGCAUCGUUUA | 1236 | UAAACGAUGCCAAAUGGAG | 1851 | | [3278-3296] 3′UTR |
| AGCAGGAGUUUCUCGACAU | 1237 | AUGUCGAGAAACUCCUGCU | 1852 | Ck, Dg | [933-951] ORF |
| GUGUGGCCUUUAUAUUUGA | 1238 | UCAAAUAUAAAGGCCACAC | 1853 | | [3121-3139] 3′UTR |
| GGGACCUGGUCAGCACAGA | 1239 | UCUGUGCUGACCAGGUCCC | 1854 | Rh | [2579-2597] 3′UTR |
| CCUCAGUGUGGUUUCCUGA | 1240 | UCAGGAAACCACACUGAGG | 1855 | | [2288-2306] 3′UTR |
| GACCCAGAGUGGAACGCGU | 1241 | ACGCGUUCCACUCUGGGUC | 1856 | | [2966-2984] 3′UTR |
| CCACCUGUGUUGUAAAGAU | 1242 | AUCUUUACAACACAGGUGG | 1857 | Rh | [2377-2395] 3′UTR |
| ACCUGUGUUGUAAAGAUAA | 1243 | UUAUCUUUACAACACAGGU | 1858 | Rh | [2379-2397] 3′UTR |
| GUUUUGCAAUGCAGAUGUA | 1244 | UACAUCUGCAUUGCAAAAC | 1859 | | [412-430] ORF |
| AAAAAAGCCUCCAAGGGUU | 1245 | AACCCUUGGAGGCUUUUUU | 1860 | | [997-1015] 3′UTR |
| UAAGAAACUUCCUAGGGAA | 1246 | UUCCCUAGGAAGUUUCUUA | 1861 | | [2250-2268] 3′UTR |
| GCAUUUGACCCAGAGUGGA | 1247 | UCCACUCUGGGUCAAAUGC | 1862 | | [2960-2978] 3′UTR |
| CCCUCAAGGUCCCUUCCCU | 1248 | AGGGAAGGGACCUUGAGGG | 1863 | | [1785-1803] 3′UTR |
| AGGAUCCAGUAUGAGAUCA | 1249 | UGAUCUCAUACUGGAUCCU | 1864 | Rh, Rb | [503-521] ORF |
| AAGAUUACCUAGCUAAGAA | 1250 | UUCUUAGCUAGGUAAUCUU | 1865 | | [2237-2255] 3′UTR |
| CUAUGUGUUCCCUCAGUGU | 1251 | ACACUGAGGGAACACAUAG | 1866 | | [2278-2296] 3′UTR |
| GACAGAGGAAGCCGCUCAA | 1252 | UUGAGCGGCUUCCUCUGUC | 1867 | | [3211-3229] 3′UTR |
| UUAAGAAGGCUCUCCAUUU | 1253 | AAAUGGAGAGCCUUCUUAA | 1868 | | [3267-3285] 3′UTR |
| UAAGGAGAAUCUCUUGUUU | 1254 | AAACAAGAGAUUCUCCUUA | 1869 | | [2354-2372] 3′UTR |
| GUUUCCUGAAGCCAGUGAU | 1255 | AUCACUGGCUUCAGGAAAC | 1870 | | [2298-2316] 3′UTR |
| UGAGCACCACCCAGAAGAA | 1256 | UUCUUCUGGGUGGUGCUCA | 1871 | Rh, Pg | [708-726] ORF |
| CUAUUAAUCCUCAGAAUUC | 1257 | GAAUUCUGAGGAUUAAUAG | 1872 | Rh | [1573-1591] 3′UTR |
| CUGGGCGUGGUCUUGCAAA | 1258 | UUUGCAAGACCACGCCCAG | 1873 | | [2460-2478] 3′UTR |
| GGAGGAAAGAAGGAAUAUC | 1259 | GAUAUUCCUUCUUUCCUCC | 1874 | Rt | [614-632] ORF |
| CCAAGUUCUUCGCCUGCAU | 1260 | AUGCAGGCGAAGAACUUGG | 1875 | Rh, Rb, Cw, Dg, Ms | [864-882] ORF |
| GUUUCUGCUGAUUGUUUUU | 1261 | AAAAACAAUCAGCAGAAAC | 1876 | | [2824-2842] 3′UTR |
| GGUCCAAGGUCCUCAUCCC | 1262 | GGGAUGAGGACCUUGGACC | 1877 | | [1149-1167] 3′UTR |
| AGUUGGUAAGAUGUCAUAA | 1263 | UUAUGACAUCUUACCAACU | 1878 | Rh | [2690-2708] 3′UTR |
| GGAAUAUGAAGUCUGAGAC | 1264 | GUCUCAGACUUCAUAUUCC | 1879 | Ms | [3508-3526] 3′UTR |
| GAGUGGAACGCGUGGCCUA | 1265 | UAGGCCACGCGUUCCACUC | 1880 | | [2972-2990] 3′UTR |
| GGUUGUGGGUCUGGAGGGA | 1266 | UCCCUCCAGACCCACAACC | 1881 | Rh | [1124-1142] 3′UTR |
| GUUGAUUUUGUUUCCGUUU | 1267 | AAACGGAAACAAAAUCAAC | 1882 | | [3454-3472] 3′UTR |
| CACUGUGUUUAUGCUGGAA | 1268 | UUCCAGCAUAAACACAGUG | 1883 | | [3493-3511] 3′UTR |
| GAGCUGCGUUCCAGCCUCA | 1269 | UGAGGCUGGAACGCAGCUC | 1884 | | [1645-1663] 3′UTR |
| GGACUGGGUCACAGAGAAG | 1270 | CUUCUCUGUGACCCAGUCC | 1885 | Rh, Rt, Ms, Pg | [826-844] ORF |
| AGCUAAGCAUAGUAAGAAG | 1271 | CUUCUUACUAUGCUUAGCU | 1886 | | [2032-2050] 3′UTR |
| CACAAGAGUUGUUGAAAGU | 1272 | ACUUUCAACAACUCUUGUG | 1887 | | [3545-3563] 3′UTR |
| GGUCAGCACAGAUCUUGAU | 1273 | AUCAAGAUCUGUGCUGACC | 1888 | Rh | [2586-2604] 3′UTR |
| UUCUAAAGGUGAAUUCUCA | 1274 | UGAGAAUUCACCUUUAGAA | 1889 | | [2158-2176] 3′UTR |
| AGGGAACUAGGGAACCUAU | 1275 | AUAGGUUCCCUAGUUCCCU | 1890 | Rh | [2263-2281] 3′UTR |
| GGAAGUGGACUCUGGAAAC | 1276 | GUUUCCAGAGUCCACUUCC | 1891 | | [460-478] ORF |
| CCUCCCACCUGUGUUGUAA | 1277 | UUACAACACAGGUGGGAGG | 1892 | Rh | [2373-2391] 3′UTR |
| CGGACGAGUGCCUCUGGAU | 1278 | AUCCAGAGGCACUCGUCCG | 1893 | Rh, Rb, Cw | [807-825] ORF |
| CGUGGAAGCAUUUGACCCA | 1279 | UGGGUCAAAUGCUUCCACG | 1894 | Rh | [2953-2971] 3′UTR |
| GCACUGUGUUUAUGCUGGA | 1280 | UCCAGCAUAAACACAGUGC | 1895 | | [3492-3510] 3′UTR |
| AGUUGCAGAUAUACCAACU | 1281 | AGUUGGUAUAUCUGCAACU | 1896 | Rh | [2194-2212] 3′UTR |
| AAUGAUAAGGAGAAUCUCU | 1282 | AGAGAUUCUCCUUAUCAUU | 1897 | | [2349-2367] 3′UTR |
| CUUGCUGCCGUAAUUUAAA | 1283 | UUUAAAUUACGGCAGCAAG | 1898 | | [3430-3448] 3′UTR |
| GGAGAAUCUCUUGUUUCCU | 1284 | AGGAAACAAGAGAUUCUCC | 1899 | | [2357-2375] 3′UTR |
| CCUUGGUAGGUAUUAGACU | 1285 | AGUCUAAUACCUACCAAGG | 1900 | | [2905-2923] 3′UTR |
| GGACGUUGGAGGAAAGAAG | 1286 | CUUCUUUCCUCCAACGUCC | 1901 | Rt, Ms | [607-625] ORF |
| CGUUGGAGGAAAGAAGGAA | 1287 | UUCCUUCUUUCCUCCAACG | 1902 | Rt, Ms | [610-628] ORF |
| CUGACAUCCCUUCCUGGAA | 1288 | UUCCAGGAAGGGAUGUCAG | 1903 | Rh, Rt, Ms | [1031-1049] 3′UTR |
| UGACAUCCCUUCCUGGAAA | 1289 | UUUCCAGGAAGGGAUGUCA | 1904 | Rh, Rt, Ms | [1032-1050] 3′UTR |
| GAUAUACCAACUUCUGCUU | 1290 | AAGCAGAAGUUGGUAUAUC | 1905 | Rh | [2201-2219] 3′UTR |
| AGAUGGGCUGCGAGUGCAA | 1291 | UUGCACUCGCAGCCCAUCU | 1906 | Ck, Rb, Rt | [747-765] ORF |
| GGCUUAGUGUUCCCUCCCU | 1292 | AGGGAGGGAACACUAAGCC | 1907 | | [1964-1982] 3′UTR |
| GUAUGGUGAUAUCAUAUGU | 1293 | ACAUAUGAUAUCACCAUAC | 1908 | | [2792-2810] 3′UTR |
| ACCAACUUCUGCUUGUAUU | 1294 | AAUACAAGCAGAAGUUGGU | 1909 | Rh | [2206-2224] 3′UTR |
| CUCACUUCUUUCUCAGCCU | 1295 | AGGCUGAGAAAGAAGUGAG | 1910 | | [2101-2119] 3′UTR |
| CUCCCACCUGUGUUGUAAA | 1296 | UUUACAACACAGGUGGGAG | 1911 | Rh | [2374-2392] 3′UTR |
| GGGUCUCGCUGGACGUUGG | 1297 | CCAACGUCCAGCGAGACCC | 1912 | Rt, Ms | [597-615] ORF |
| GAGCCUCCCUCUGAGCCUU | 1298 | AAGGCUCAGAGGGAGGCUC | 1913 | Rh | [1598-1616] 3′UTR |
| GCAUGUCUCUGAUGCUUUG | 1299 | CAAAGCAUCAGAGACAUGC | 1914 | | [3579-3597] 3′UTR |
| GGCGUUUUCAUGCUGUACA | 1300 | UGUACAGCAUGAAAACGCC | 1915 | Rh | [2662-2680] 3′UTR |
| AUACCAACUUCUGCUUGUA | 1301 | UACAAGCAGAAGUUGGUAU | 1916 | Rh | [2204-2222] 3′UTR |
| GCAAUGCAGAUGUAGUGAU | 1302 | AUCACUACAUCUGCAUUGC | 1917 | | [417-435] ORF |
| ACUUCUGCUUGUAUUUCUU | 1303 | AAGAAAUACAAGCAGAAGU | 1918 | Rh | [2210-2228] 3′UTR |
| UCCAGUUCAAAUUAUUGCA | 1304 | UGCAAUAAUUUGAACUGGA | 1919 | | [3067-3085] 3′UTR |
| CCUGGUCAGCACAGAUCUU | 1305 | AAGAUCUGUGCUGACCAGG | 1920 | Rh | [2583-2601] 3′UTR |
| UGUUGAUUUUGUUUCCGUU | 1306 | AACGGAAACAAAAUCAACA | 1921 | | [3453-3471] 3′UTR |
| UGCAGAUAUACCAACUUCU | 1307 | AGAAGUUGGUAUAUCUGCA | 1922 | Rh | [2197-2215] 3′UTR |
| GCGGUCAGUGAGAAGGAAG | 1308 | CUUCCUUCUCACUGACCGC | 1923 | | [446-464] ORF |
| GGCGUGGUCUUGCAAAAUG | 1309 | CAUUUUGCAAGACCACGCC | 1924 | | [2463-2481] 3′UTR |
| GUCCAGCCUAGGAAGGGAA | 1310 | UUCCCUUCCUAGGCUGGAC | 1925 | Rh | [2050-2068] 3′UTR |
| ACUUUCUCGGUAAUGAUAA | 1311 | UUAUCAUUACCGAGAAAGU | 1926 | | [2338-2356] 3′UTR |
| CUUCUGCUUGUAUUUCUUA | 1312 | UAAGAAAUACAAGCAGAAG | 1927 | | [2211-2229] 3′UTR |
| CAGAGGAAGCCGCUCAAAU | 1313 | AUUUGAGCGGCUUCCUCUG | 1928 | | [3213-3231] 3′UTR |
| GAAGGAAGUGGACUCUGGA | 1314 | UCCAGAGUCCACUUCCUUC | 1929 | | [457-475] ORF |
| CAGUGAGAAGGAAGUGGAC | 1315 | GUCCACUUCCUUCUCACUG | 1930 | | [451-469] ORF |
| GACUUCCCUUUCUAGGGCA | 1316 | UGCCCUAGAAAGGGAAGUC | 1931 | Rh | [2605-2623] 3′UTR |
| CCUCCCUCUGAGCCUUGUA | 1317 | UACAAGGCUCAGAGGGAGG | 1932 | Rh | [1601-1619] 3′UTR |
| CAUGCUGUACAGUGACCUA | 1318 | UAGGUCACUGUACAGCAUG | 1933 | | [2670-2688] 3′UTR |
| GAGUGCCUCUGGAUGGACU | 1319 | AGUCCAUCCAGAGGCACUC | 1934 | Rh, Rb, Cw, Dg, Rt, Ms | [812-830] ORF |
| CUGGGAGGGUAUCCAGGAA | 1320 | UUCCUGGAUACCCUCCCAG | 1935 | Rh | [2626-2644] 3′UTR |
| AACCGUGCUGAGCAGAAAA | 1321 | UUUUCUGCUCAGCACGGUU | 1936 | | [3160-3178] 3′UTR |
| CAGUCCAUGUGAUUUCAGU | 1322 | ACUGAAAUCACAUGGACUG | 1937 | Rh | [2714-2732] 3′UTR |
| UAGACAUGGUUGUGGGUCU | 1323 | AGACCCACAACCAUGUCUA | 1938 | | [1117-1135] 3′UTR |
| GCGCAUGUCUCUGAUGCUU | 1324 | AAGCAUCAGAGACAUGCGC | 1939 | | [3577-3595] 3′UTR |
| AAGGUGAAUUCUCAGAUGA | 1325 | UCAUCUGAGAAUUCACCUU | 1940 | | [2163-2181] 3′UTR |
| AGAAGAACAUCAACGGGCA | 1326 | UGCCCGUUGAUGUUCUUCU | 1941 | Rh, Rb | [840-858] ORF |
| ACAUACACACGCAAUGAAA | 1327 | UUUCAUUGCGUGUGUAUGU | 1942 | Rh | [2426-2444] 3′UTR |
| CACAGAUCUUGAUGACUUC | 1328 | GAAGUCAUCAAGAUCUGUG | 1943 | Rh | [2592-2610] 3′UTR |
| AGCCGCUCAAAUACCUUCA | 1329 | UGAAGGUAUUUGAGCGGCU | 1944 | | [3220-3238] 3′UTR |
| CCAGUAUGAGAUCAAGCAG | 1330 | CUGCUUGAUCUCAUACUGG | 1945 | Rh, Rb, Cw, Dg, Ms | [508-526] ORF |
| GUGAGAAGGAAGUGGACUC | 1331 | GAGUCCACUUCCUUCUCAC | 1946 | | [453-471] ORF |
| ACCUUAGCCUGUUCUAUUC | 1332 | GAAUAGAACAGGCUAAGGU | 1947 | Rh | [2493-2511] 3′UTR |
| CCUGUUUCUGCUGAUUGUU | 1333 | AACAAUCAGCAGAAACAGG | 1948 | | [2821-2839] 3′UTR |
| GCCAUUGCUUCUUGCCUGU | 1334 | ACAGGCAAGAAGCAAUGGC | 1949 | | [1817-1835] 3′UTR |
| GCCUGGAAAUGUGCAUUUU | 1335 | AAAAUGCACAUUUCCAGGC | 1950 | Rh | [1322-1340] 3′UTR |
| GCACAGCUCUCUUCUCCUA | 1336 | UAGGAGAAGAGAGCUGUGC | 1951 | | [3317-3335] 3′UTR |
| CGACAUUUAUGGCAACCCU | 1337 | AGGGUUGCCAUAAAUGUCG | 1952 | | [478-496] ORF |
| CCUGUGCUGUGUUUUUUAU | 1338 | AUAAAAAACACAGCACAGG | 1953 | Rh | [2883-2901] 3′UTR |
| AGGAAGUGGACUCUGGAAA | 1339 | UUUCCAGAGUCCACUUCCU | 1954 | | [459-477] ORF |
| GCUAAGCAUAGUAAGAAGU | 1340 | ACUUCUUACUAUGCUUAGC | 1955 | | [2033-2051] 3′UTR |
| CCGUCUUUGGUUCUCCAGU | 1341 | ACUGGAGAACCAAAGACGG | 1956 | | [3054-3072] 3′UTR |
| UUUCCUGAAGCCAGUGAUA | 1342 | UAUCACUGGCUUCAGGAAA | 1957 | | [2299-2317] 3′UTR |
| AGACGUGGGUCCAAGGUCC | 1343 | GGACCUUGGACCCACGUCU | 1958 | | [1142-1160] 3′UTR |
| ACAUUUAUGGCAACCCUAU | 1344 | AUAGGGUUGCCAUAAAUGU | 1959 | | [480-498] ORF |
| GUGGACAAUAAACAGUAUU | 1345 | AAUACUGUUUAUUGUCCAC | 1960 | | [3625-3643] 3′UTR |
| GGGAACACACAAGAGUUGU | 1346 | ACAACUCUUGUGUGUUCCC | 1961 | | [3538-3556] 3′UTR |
| GCUCGGUCCGUGGACAAUA | 1347 | UAUUGUCCACGGACCGAGC | 1962 | | [3616-3634] 3′UTR |
| CCGUGCUGAGCAGAAAACA | 1348 | UGUUUUCUGCUCAGCACGG | 1963 | | [3162-3180] 3′UTR |
| CCGCUCAAAUACCUUCACA | 1349 | UGUGAAGGUAUUUGAGCGG | 1964 | | [3222-3240] 3′UTR |
| GUUCCCUCCCUCAAAGACU | 1350 | AGUCUUUGAGGGAGGGAAC | 1965 | | [1972-1990] 3′UTR |
| GGUCGUUGCAAGACUGUGU | 1351 | ACACAGUCUUGCAACGACC | 1966 | | [1356-1374] 3′UTR |
| GGUGCUGGGAACACACAAG | 1352 | CUUGUGUGUUCCCAGCACC | 1967 | | [3532-3550] 3′UTR |
| AGUAUAUACAACUCCACCA | 1353 | UGGUGGAGUUGUAUAUACU | 1968 | Rh | [2730-2748] 3′UTR |
| GGCAUCAGGCACCUGGAUU | 1354 | AAUCCAGGUGCCUGAUGCC | 1969 | | [1839-1857] 3′UTR |
| AGCAGAUAAAGAUGUUCAA | 1355 | UUGAACAUCUUUAUCUGCU | 1970 | Cw, Dg, Rt, Ms, Pg | [522-540] ORF |
| UGGAAUAUGAAGUCUGAGA | 1356 | UCUCAGACUUCAUAUUCCA | 1971 | Ms | [3507-3525] 3′UTR |
| CAGGCACCUGGAUUGAGUU | 1357 | AACUCAAUCCAGGUGCCUG | 1972 | Rh | [1844-1862] 3′UTR |
| AUAAGGAGAAUCUCUUGUU | 1358 | AACAAGAGAUUCUCCUUAU | 1973 | | [2353-2371] 3′UTR |
| GCCUGUUUUAAGAGACAUC | 1359 | GAUGUCUCUUAAAACAGGC | 1974 | Rh | [2133-2151] 3′UTR |
| CGCUUCCUGUAUGGUGAUA | 1360 | UAUCACCAUACAGGAAGCG | 1975 | | [2784-2802] 3′UTR |
| GCACCGUCACAGAUGCCAA | 1361 | UUGGCAUCUGUGACGGUGC | 1976 | | [1262-1280] 3′UTR |
| GUUCCAGCCUCAGCUGAGU | 1362 | ACUCAGCUGAGGCUGGAAC | 1977 | | [1652-1670] 3′UTR |
| GGAGGUAGGUGGCUUUGGU | 1363 | ACCAAAGCCACCUACCUCC | 1978 | Rh | [2076-2094] 3′UTR |
| GGAAACGACAUUUAUGGCA | 1364 | UGCCAUAAAUGUCGUUUCC | 1979 | | [473-491] ORF |
| GCAAGAUGCACAUCACCCU | 1365 | AGGGUGAUGUGCAUCUUGC | 1980 | Rh, Dg | [660-678] ORF |
| UGUAGAAAUGGGAGCGAGA | 1366 | UCUCGCUCCCAUUUCUACA | 1981 | Rh | [1616-1634] 3′UTR |
| GGCCUAUGCAGGUGGAUUC | 1367 | GAAUCCACCUGCAUAGGCC | 1982 | Rh | [2985-3003] 3′UTR |
| AAGAAGAGCCUGAACCACA | 1368 | UGUGGUUCAGGCUCUUCUU | 1983 | Rh, Rb, Cw, Ms, Pg | [722-740] ORF |
| GGGAGGGUAUCCAGGAAUC | 1369 | GAUUCCUGGAUACCCUCCC | 1984 | Rh | [2628-2646] 3′UTR |
| GUCAUAAUGGACCAGUCCA | 1370 | UGGACUGGUCCAUUAUGAC | 1985 | Rh | [2702-2720] 3′UTR |
| CCAAGGUCCUCAUCCCAUC | 1371 | GAUGGGAUGAGGACCUUGG | 1986 | | [1152-1170] 3′UTR |
| AGGUGGCUUUGGUGACACA | 1372 | UGUGUCACCAAAGCCACCU | 1987 | Rh | [2082-2100] 3′UTR |
| AGACUGUGUAGCAGGCCUA | 1373 | UAGGCCUGCUACACAGUCU | 1988 | Rh | [1366-1384] 3′UTR |
| GGCCUGGAAAUGUGCAUUU | 1374 | AAAUGCACAUUUCCAGGCC | 1989 | Rh | [1321-1339] 3′UTR |
| GGUUAGGAUAGGAAGAACU | 1375 | AGUUCUUCCUAUCCUAACC | 1990 | | [2322-2340] 3′UTR |
| GGCUAGUUCUUGAAGGAGC | 1376 | GCUCCUUCAAGAACUAGCC | 1991 | | [1544-1562] 3′UTR |
| AGCUCUGUUGAUUUUGUUU | 1377 | AAACAAAAUCAACAGAGCU | 1992 | | [3448-3466] 3′UTR |
| UGCAUUUUGCAGAAACUUU | 1378 | AAAGUUUCUGCAAAAUGCA | 1993 | Rh | [1333-1351] 3′UTR |
| GUCUGAAAGGUGUGGCCUU | 1379 | AAGGCCACACCUUUCAGAC | 1994 | | [3112-3130] 3′UTR |
| CAUCCAAGGGCAGCCUGGA | 1380 | UCCAGGCUGCCCUUGGAUG | 1995 | | [1519-1537] 3′UTR |
| UGUUUCUGCUGAUUGUUUU | 1381 | AAAACAAUCAGCAGAAACA | 1996 | | [2823-2841] 3′UTR |
| UGCAAGCAACUCAAAAUAU | 1382 | AUAUUUUGAGUUGCUUGCA | 1997 | | [3345-3363] 3′UTR |
| AACAUUUACUCCUGUUUCU | 1383 | AGAAACAGGAGUAAAUGUU | 1998 | Rh | [2811-2829] 3′UTR |
| GAAAGGUGUGGCCUUUAUA | 1384 | UAUAAAGGCCACACCUUUC | 1999 | | [3116-3134] 3′UTR |
| UCCUGUUUCUGCUGAUUGU | 1385 | ACAAUCAGCAGAAACAGGA | 2000 | | [2820-2838] 3′UTR |
| AUCCUAUUAAUCCUCAGAA | 1386 | UUCUGAGGAUUAAUAGGAU | 2001 | Rh | [1570-1588] 3′UTR |
| UCCUGAAGCCAGUGAUAUG | 1387 | CAUAUCACUGGCUUCAGGA | 2002 | | [2301-2319] 3′UTR |
| AGGGCCUGAGAAGGAUAUA | 1388 | UAUAUCCUUCUCAGGCCCU | 2003 | | [541-559] ORF |
| GUUGCAGAUAUACCAACUU | 1389 | AAGUUGGUAUAUCUGCAAC | 2004 | Rh | [2195-2213] 3′UTR |
| GGGCCUGAGAAGGAUAUAG | 1390 | CUAUAUCCUUCUCAGGCCC | 2005 | | [542-560] ORF |
| CGGGCGUUUUCAUGCUGUA | 1391 | UACAGCAUGAAAACGCCCG | 2006 | Rh | [2660-2678] 3′UTR |
| ACCAGUCCAUGUGAUUUCA | 1392 | UGAAAUCACAUGGACUGGU | 2007 | Rh | [2712-2730] 3′UTR |
| CGUGGGUCCAAGGUCCUCA | 1393 | UGAGGACCUUGGACCCACG | 2008 | | [1145-1163] 3′UTR |
| GCCUGGAACCAGUGGCUAG | 1394 | CUAGCCACUGGUUCCAGGC | 2009 | | [1531-1549] 3′UTR |
| CCUUUCAUCUUGAGAGGGA | 1395 | UCCCUCUCAAGAUGAAAGG | 2010 | Rh | [1392-1410] 3′UTR |
| CCAGUGGGAGCCUCCCUCU | 1396 | AGAGGGAGGCUCCCACUGG | 2011 | Rh | [1591-1609] 3′UTR |
| AAAUGUGCAUUUUGCAGAA | 1397 | UUCUGCAAAAUGCACAUUU | 2012 | Rh, Ms | [1328-1346] 3′UTR |
| UGGUCAGCACAGAUCUUGA | 1398 | UCAAGAUCUGUGCUGACCA | 2013 | Rh | [2585-2603] 3′UTR |
| CUAUGCAGGUGGAUUCCUU | 1399 | AAGGAAUCCACCUGCAUAG | 2014 | Rh | [2988-3006] 3′UTR |
| AGUAAGAAGUCCAGCCUAG | 1400 | CUAGGCUGGACUUCUUACU | 2015 | Rh | [2042-2060] 3′UTR |
| CUCAUCCCAUGGGUCCAAA | 1401 | UUUGGACCCAUGGGAUGAG | 2016 | | [1067-1085] 3′UTR |
| AGAGCCGGGUGGCAGCUGA | 1402 | UCAGCUGCCACCCGGCUCU | 2017 | | [3194-3212] 3′UTR |
| GCUGGGAACACACAAGAGU | 1403 | ACUCUUGUGUGUUCCCAGC | 2018 | | [3535-3553] 3′UTR |
| CCGGACGAGUGCCUCUGGA | 1404 | UCCAGAGGCACUCGUCCGG | 2019 | Rh, Rb, Cw | [806-824] ORF |
| CCCUCAGUGUGGUUUCCUG | 1405 | CAGGAAACCACACUGAGGG | 2020 | | [2287-2305] 3′UTR |
| AGUUGCACAGCUUUGCUUU | 1406 | AAAGCAAAGCUGUGCAACU | 2021 | | [1859-1877] 3′UTR |
| CCCAUAAGCAGGCCUCCAA | 1407 | UUGGAGGCCUGCUUAUGGG | 2022 | | [958-976] |
| | | | | | ORF + 3′UTR |
| CGGUGCUGGGAACACACAA | 1408 | UUGUGUGUUCCCAGCACCG | 2023 | | [3531-3549] 3′UTR |
| GGUUUGUUUUUGACAUCAG | 1409 | CUGAUGUCAAAAACAAACC | 2024 | Rh | [2853-2871] 3′UTR |
| GACGAUAUACAGGCACAUU | 1410 | AAUGUGCCUGUAUAUCGUC | 2025 | | [2401-2419] 3′UTR |
| CUUAGUGUUCCCUCCCUCA | 1411 | UGAGGGAGGGAACACUAAG | 2026 | | [1966-1984] 3′UTR |
| GACACACUCACUUCUUUCU | 1412 | AGAAAGAAGUGAGUGUGUC | 2027 | | [2095-2113] 3′UTR |
| GAAGCCGCUCAAAUACCUU | 1413 | AAGGUAUUUGAGCGGCUUC | 2028 | | [3218-3236] 3′UTR |
| GGUCCCUUUCAUCUUGAGA | 1414 | UCUCAAGAUGAAAGGGACC | 2029 | Rh | [1388-1406] 3′UTR |
| CCUGGAACCAGUGGCUAGU | 1415 | ACUAGCCACUGGUUCCAGG | 2030 | | [1532-1550] 3′UTR |
| UUCAGUAUAUACAACUCCA | 1416 | UGGAGUUGUAUAUACUGAA | 2031 | Rh | [2727-2745] 3′UTR |
| ACCUAUGUGUUCCCUCAGU | 1417 | ACUGAGGGAACACAUAGGU | 2032 | | [2276-2294] 3′UTR |
| CUCCUAUUUUCAUCCUGCA | 1418 | UGCAGGAUGAAAAUAGGAG | 2033 | | [3330-3348] 3′UTR |
| ACACACAAGAGUUGUUGAA | 1419 | UUCAACAACUCUUGUGUGU | 2034 | | [3542-3560] 3′UTR |
| UGUCUCUGAUGCUUUGUAU | 1420 | AUACAAAGCAUCAGAGACA | 2035 | | [3582-3600] 3′UTR |
| UAGAAAUGGGAGCGAGAAA | 1421 | UUUCUCGCUCCCAUUUCUA | 2036 | | [1618-1636] 3′UTR |
| GAAGCAUUUGACCCAGAGU | 1422 | ACUCUGGGUCAAAUGCUUC | 2037 | | [2957-2975] 3′UTR |
| GUUUUUGACAUCAGCUGUA | 1423 | UACAGCUGAUGUCAAAAAC | 2038 | | [2858-2876] 3′UTR |
| GGAGUUUCUCGACAUCGAG | 1424 | CUCGAUGUCGAGAAACUCC | 2039 | Ck, Dg | [937-955] ORF |
| GAUAAACUGACGAUAUACA | 1425 | UGUAUAUCGUCAGUUUAUC | 2040 | | [2393-2411] 3′UTR |
| GUGUUCCCUCCCUCAAAGA | 1426 | UCUUUGAGGGAGGGAACAC | 2041 | | [1970-1988] 3′UTR |
| UUUGUUUCCGUUUGGAUUU | 1427 | AAAUCCAAACGGAAACAAA | 2042 | | [3460-3478] 3′UTR |
| AGCUGAGUCUUUUUGGUCU | 1428 | AGACCAAAAAGACUCAGCU | 2043 | Rh | [1663-1681] 3′UTR |
| AACUUUCUCGGUAAUGAUA | 1429 | UAUCAUUACCGAGAAAGUU | 2044 | | [2337-2355] 3′UTR |
| CCGGGUGGCAGCUGACAGA | 1430 | UCUGUCAGCUGCCACCCGG | 2045 | | [3198-3216] 3′UTR |
| GAGUUGCACAGCUUUGCUU | 1431 | AAGCAAAGCUGUGCAACUC | 2046 | | [1858-1876] 3′UTR |
| CCGGGACCUGGUCAGCACA | 1432 | UGUGCUGACCAGGUCCCGG | 2047 | Rh | [2577-2595] 3′UTR |
| CAAAGUAAAGGAUCUUUGA | 1433 | UCAAAGAUCCUUUACUUUG | 2048 | | [3084-3102] 3′UTR |
| CAGCUCUCUUCUCCUAUUU | 1434 | AAAUAGGAGAAGAGAGCUG | 2049 | | [3320-3338] 3′UTR |
| GACAGAAAAAGCUGGGUCU | 1435 | AGACCCAGCUUUUUCUGUC | 2050 | Rh | [1930-1948] 3′UTR |
| UGCAUGUGACGCCAGCUAA | 1436 | UUAGCUGGCGUCACAUGCA | 2051 | | [2019-2037] 3′UTR |
| CCAGCCUCAGCUGAGUCUU | 1437 | AAGACUCAGCUGAGGCUGG | 2052 | Rh | [1655-1673] 3′UTR |
| GACCUAAAGUUGGUAAGAU | 1438 | AUCUUACCAACUUUAGGUC | 2053 | | [2683-2701] 3′UTR |
| GGUGUGGCCUUUAUAUUUG | 1439 | CAAAUAUAAAGGCCACACC | 2054 | | [3120-3138] 3′UTR |
| GUCCAAGGUCCUCAUCCCA | 1440 | UGGGAUGAGGACCUUGGAC | 2055 | | [1150-1168] 3′UTR |
| UAGUAAGAAGUCCAGCCUA | 1441 | UAGGCUGGACUUCUUACUA | 2056 | Rh | [2041-2059] 3′UTR |
| AAGCAUAGUAAGAAGUCCA | 1442 | UGGACUUCUUACUAUGCUU | 2057 | | [2036-2054] 3′UTR |
| AGGAUAGGAAGAACUUUCU | 1443 | AGAAAGUUCUUCCUAUCCU | 2058 | | [2326-2344] 3′UTR |
| AGGAGAAUCUCUUGUUUCC | 1444 | GGAAACAAGAGAUUCUCCU | 2059 | | [2356-2374] 3′UTR |
| CAAGAGUUGUUGAAAGUUG | 1445 | CAACUUUCAACAACUCUUG | 2060 | | [3547-3565] 3′UTR |
| CCCAUGAUCCCGUGCUACA | 1446 | UGUAGCACGGGAUCAUGGG | 2061 | Rh, Rb | [779-797] ORF |
| CAUCCCAUGGGUCCAAAUU | 1447 | AAUUUGGACCCAUGGGAUG | 2062 | | [1069-1087] 3′UTR |
| CUGAGCAGAAAACAAAACA | 1448 | UGUUUUGUUUUCUGCUCAG | 2063 | | [3167-3185] 3′UTR |
| AGCAGAAAACAAAACAGGU | 1449 | ACCUGUUUUGUUUUCUGCU | 2064 | | [3170-3188] 3′UTR |
| CUUGUUUCCUCCCACCUGU | 1450 | ACAGGUGGGAGGAAACAAG | 2065 | | [2366-2384] 3′UTR |
| GUGGACUCUGGAAACGACA | 1451 | UGUCGUUUCCAGAGUCCAC | 2066 | Rh | [464-482] ORF |
| UGAUAAGGAGAAUCUCUUG | 1452 | CAAGAGAUUCUCCUUAUCA | 2067 | Rh | [2351-2369] 3′UTR |
| UGAGUAGGUUCGGUCUGAA | 1453 | UUCAGACCGAACCUACUCA | 2068 | | [3100-3118] 3′UTR |
| CAUUUGGCAUCGUUUAAUU | 1454 | AAUUAAACGAUGCCAAAUG | 2069 | | [3281-3299] 3′UTR |
| GCUUCCUGUAUGGUGAUAU | 1455 | AUAUCACCAUACAGGAAGC | 2070 | | [2785-2803] 3′UTR |
| GAGGAUCCAGUAUGAGAUC | 1456 | GAUCUCAUACUGGAUCCUC | 2071 | Rh, Rb | [502-520] ORF |
| GCACAUCCUGAGGACAGAA | 1457 | UUCUGUCCUCAGGAUGUGC | 2072 | Rh | [1918-1936] 3′UTR |
| GCAUGAAUAAAACACUCAU | 1458 | AUGAGUGUUUUAUUCAUGC | 2073 | Rh | [1053-1071] 3′UTR |
| GCAACAGGCGUUUUGCAAU | 1459 | AUUGCAAAACGCCUGUUGC | 2074 | Cw, Rt, Ms | [403-421] ORF |
| GGGACGGCAAGAUGCACAU | 1460 | AUGUGCAUCUUGCCGUCCC | 2075 | | [654-672] ORF |
| CUGUAAUCAUUCCUGUGCU | 1461 | AGCACAGGAAUGAUUACAG | 2076 | Rh | [2872-2890] 3′UTR |
| GGUCUUUUAACCGUGCUGA | 1462 | UCAGCACGGUUAAAAGACC | 2077 | | [3152-3170] 3′UTR |
| ACAGCUCUCUUCUCCUAUU | 1463 | AAUAGGAGAAGAGAGCUGU | 2078 | | [3319-3337] 3′UTR |
| ACCCUUGGUAGGUAUUAGA | 1464 | UCUAAUACCUACCAAGGGU | 2079 | | [2903-2921] 3′UTR |
| GACUGGUCCAGCUCUGACA | 1465 | UGUCAGAGCUGGACCAGUC | 2080 | Rh | [1018-1036] 3′UTR |
| AUCCUGCAAGCAACUCAAA | 1466 | UUUGAGUUGCUUGCAGGAU | 2081 | | [3341-3359] 3′UTR |
| CCAUGAUCCCGUGCUACAU | 1467 | AUGUAGCACGGGAUCAUGG | 2082 | Rh, Rb | [780-798] ORF |
| UUGUAUCAUUCUUGAGCAA | 1468 | UUGCUCAAGAAUGAUACAA | 2083 | | [3595-3613] 3′UTR |
| GAGUCUUUUUGGUCUGCAC | 1469 | GUGCAGACCAAAAAGACUC | 2084 | | [1667-1685] 3′UTR |
| GUUCAAAGGGCCUGAGAAG | 1470 | CUUCUCAGGCCCUUUGAAC | 2085 | | [535-553] ORF |
| AGCCUCAGCUGAGUCUUUU | 1471 | AAAAGACUCAGCUGAGGCU | 2086 | Rh | [1657-1675] 3′UTR |
| GAUAAGGAGAAUCUCUUGU | 1472 | ACAAGAGAUUCUCCUUAUC | 2087 | | [2352-2370] 3′UTR |
| ACAUCACCCUCUGUGACUU | 1473 | AAGUCACAGAGGGUGAUGU | 2088 | Rh | [669-687] ORF |
| GCGUGGUCUUGCAAAAUGC | 1474 | GCAUUUUGCAAGACCACGC | 2089 | | [2464-2482] 3′UTR |
| GCCUUGGCACCGUCACAGA | 1475 | UCUGUGACGGUGCCAAGGC | 2090 | Rh | [1256-1274] 3′UTR |
| AGAAGAGCCUGAACCACAG | 1476 | CUGUGGUUCAGGCUCUUCU | 2091 | Rh, Rb, Cw, Ms, Pg | [723-741] ORF |
| UGGCCUGUUUUAAGAGACA | 1477 | UGUCUCUUAAAACAGGCCA | 2092 | Rh | [2131-2149] 3′UTR |
| GGGAAGGAUUUUGGAGGUA | 1478 | UACCUCCAAAAUCCUUCCC | 2093 | Rh | [2064-2082] 3′UTR |
| CCCUGUGGCCAACUGCAAA | 1479 | UUUGCAGUUGGCCACAGGG | 2094 | Rh | [980-998] 3′UTR |
| CUUUCUCGGUAAUGAUAAG | 1480 | CUUAUCAUUACCGAGAAAG | 2095 | | [2339-2357] 3′UTR |
| CACUCAUCCCAUGGGUCCA | 1481 | UGGACCCAUGGGAUGAGUG | 2096 | | [1065-1083] 3′UTR |
| GGUGGCAGCUGACAGAGGA | 1482 | UCCUCUGUCAGCUGCCACC | 2097 | | [3201-3219] 3′UTR |
| GUGAAUUCUCAGAUGAUAG | 1483 | CUAUCAUCUGAGAAUUCAC | 2098 | | [2166-2184] 3′UTR |
| UCCUGCAAGCAACUCAAAA | 1484 | UUUUGAGUUGCUUGCAGGA | 2099 | | [3342-3360] 3′UTR |
| CCUGUUUUAAGAGACAUCU | 1485 | AGAUGUCUCUUAAAACAGG | 2100 | Rh | [2134-2152] 3′UTR |
| GGGCAGCCUGGAACCAGUG | 1486 | CACUGGUUCCAGGCUGCCC | 2101 | | [1526-1544] 3′UTR |
| GCAUCAGGCACCUGGAUUG | 1487 | CAAUCCAGGUGCCUGAUGC | 2102 | | [1840-1858] 3′UTR |
| AGCCUGGAACCAGUGGCUA | 1488 | UAGCCACUGGUUCCAGGCU | 2103 | | [1530-1548] 3′UTR |
| UGCACAUCACCCUCUGUGA | 1489 | UCACAGAGGGUGAUGUGCA | 2104 | Rh | [666-684] ORF |
| AGAUAUACCAACUUCUGCU | 1490 | AGCAGAAGUUGGUAUAUCU | 2105 | Rh | [2200-2218] 3′UTR |
| CUAGCUAAGAAACUUCCUA | 1491 | UAGGAAGUUUCUUAGCUAG | 2106 | | [2245-2263] 3′UTR |
| CUCUCUUCUCCUAUUUUCA | 1492 | UGAAAAUAGGAGAAGAGAG | 2107 | | [3323-3341] 3′UTR |
| AUCCAAGGGCAGCCUGGAA | 1493 | UUCCAGGCUGCCCUUGGAU | 2108 | | [1520-1538] 3′UTR |
| AAAGGAUCUUUGAGUAGGU | 1494 | ACCUACUCAAAGAUCCUUU | 2109 | | [3090-3108] 3′UTR |
| CUGAGCACCACCCAGAAGA | 1495 | UCUUCUGGGUGGUGCUCAG | 2110 | Rh, Pg | [707-725] ORF |
| CCUGUUCUGGCAUCAGGCA | 1496 | UGCCUGAUGCCAGAACAGG | 2111 | | [1831-1849] 3′UTR |
| UGUGUUUAUGCUGGAAUAU | 1497 | AUAUUCCAGCAUAAACACA | 2112 | | [3496-3514] 3′UTR |
| AGGAAGAACUUUCUCGGUA | 1498 | UACCGAGAAAGUUCUUCCU | 2113 | Rh | [2331-2349] 3′UTR |
| AGAGCCUGAACCACAGGUA | 1499 | UACCUGUGGUUCAGGCUCU | 2114 | Rh, Rb, Cw, Ms, Pg | [726-744] ORF |
| GCCAAGCAGGCAGCACUUA | 1500 | UAAGUGCUGCCUGCUUGGC | 2115 | | [1276-1294] 3′UTR |
| GGGCUUUCUGCAUGUGACG | 1501 | CGUCACAUGCAGAAAGCCC | 2116 | | [2011-2029] 3′UTR |
| ACCCAGAAGAAGAGCCUGA | 1502 | UCAGGCUCUUCUUCUGGGU | 2117 | Rh, Cw, Ms, Pg | [716-734] ORF |
| CGCCUGCAUCAAGAGAAGU | 1503 | ACUUCUCUUGAUGCAGGCG | 2118 | Rh, Cw, Dg, Rt, Ms | [874-892] ORF |
| GCAGAUAUACCAACUUCUG | 1504 | CAGAAGUUGGUAUAUCUGC | 2119 | Rh | [2198-2216] 3′UTR |
| UGGACCAGUCCAUGUGAUU | 1505 | AAUCACAUGGACUGGUCCA | 2120 | Rh | [2709-2727] 3′UTR |
| UGUAACAUUUACUCCUGUU | 1506 | AACAGGAGUAAAUGUUACA | 2121 | Rh | [2808-2826] 3′UTR |
| CAGCUGUAAUCAUUCCUGU | 1507 | ACAGGAAUGAUUACAGCUG | 2122 | | [2869-2887] 3′UTR |
| GAGUUGUUGAAAGUUGACA | 1508 | UGUCAACUUUCAACAACUC | 2123 | | [3550-3568] 3′UTR |
| AGACUGCGCAUGUCUCUGA | 1509 | UCAGAGACAUGCGCAGUCU | 2124 | | [3572-3590] 3′UTR |
| UCCUGUGCUGUGUUUUUUA | 1510 | UAAAAAACACAGCACAGGA | 2125 | Rh | [2882-2900] 3′UTR |
| CCUUCUCCUUUUAGACAUG | 1511 | CAUGUCUAAAAGGAGAAGG | 2126 | | [1106-1124] 3′UTR |
| CUAAGAAACUUCCUAGGGA | 1512 | UCCCUAGGAAGUUUCUUAG | 2127 | | [2249-2267] 3′UTR |
| GCCAAGUUCUUCGCCUGCA | 1513 | UGCAGGCGAAGAACUUGGC | 2128 | Rh, Rb, Cw, Dg, Ms | [863-881] ORF |
| GCUGGACGUUGGAGGAAAG | 1514 | CUUUCCUCCAACGUCCAGC | 2129 | Rt, Ms | [604-622] ORF |
| AGGCGUUUUGCAAUGCAGA | 1515 | UCUGCAUUGCAAAACGCCU | 2130 | Cw, Rt, Ms | [408-426] ORF |
| UGUGCAUUUUGCAGAAACU | 1516 | AGUUUCUGCAAAAUGCACA | 2131 | Rh, Rt, Ms | [1331-1349] 3′UTR |
| CAGCCUGGAACCAGUGGCU | 1517 | AGCCACUGGUUCCAGGCUG | 2132 | | [1529-1547] 3′UTR |
| GCAAAAAAAGCCUCCAAGG | 1518 | CCUUGGAGGCUUUUUUUGC | 2133 | | [994-1012] 3′UTR |
| ACCUGGAUUGAGUUGCACA | 1519 | UGUGCAACUCAAUCCAGGU | 2134 | | [1849-1867] 3′UTR |
| CGUAAUUUAAAGCUCUGUU | 1520 | AACAGAGCUUUAAAUUACG | 2135 | | [3438-3456] 3′UTR |
| CUGUGCUGUGUUUUUUAUU | 1521 | AAUAAAAAACACAGCACAG | 2136 | Rh | [2884-2902] 3′UTR |
| GGCACCAGGCCAAGUUCUU | 1522 | AAGAACUUGGCCUGGUGCC | 2137 | Rh, Rb, Rt, Ms | [855-873] ORF |
| CCUGUGGCCAACUGCAAAA | 1523 | UUUUGCAGUUGGCCACAGG | 2138 | Rh | [981-999] 3′UTR |
| GGUUUCGACUGGUCCAGCU | 1524 | AGCUGGACCAGUCGAAACC | 2139 | Rh | [1012-1030] 3′UTR |
| GAAUAAAACACUCAUCCCA | 1525 | UGGGAUGAGUGUUUUAUUC | 2140 | Rh | [1057-1075] 3′UTR |
| GAGUUGCAGAUAUACCAAC | 1526 | GUUGGUAUAUCUGCAACUC | 2141 | Rh | [2193-2211] 3′UTR |
| CUUCCUGUAUGGUGAUAUC | 1527 | GAUAUCACCAUACAGGAAG | 2142 | | [2786-2804] 3′UTR |
| UUUGGUUCUCCAGUUCAAA | 1528 | UUUGAACUGGAGAACCAAA | 2143 | | [3059-3077] 3′UTR |
| GCCUGCAUCAAGAGAAGUG | 1529 | CACUUCUCUUGAUGCAGGC | 2144 | Rt, Ms | [875-893] ORF |
| GGCAACCCUAUCAAGAGGA | 1530 | UCCUCUUGAUAGGGUUGCC | 2145 | | [488-506] ORF |
| AAGCGGUCAGUGAGAAGGA | 1531 | UCCUUCUCACUGACCGCUU | 2146 | | [444-462] ORF |
| GUGGCUUUGGUGACACACU | 1532 | AGUGUGUCACCAAAGCCAC | 2147 | | [2084-2102] 3′UTR |
| AGAUCUUGAUGACUUCCCU | 1533 | AGGGAAGUCAUCAAGAUCU | 2148 | Rh | [2595-2613] 3′UTR |
| GAGACGUGGGUCCAAGGUC | 1534 | GACCUUGGACCCACGUCUC | 2149 | | [1141-1159] 3′UTR |
| UGACAUCAGCUGUAAUCAU | 1535 | AUGAUUACAGCUGAUGUCA | 2150 | | [2863-2881] 3′UTR |
| GGGAUCUCCCAGCUGGGUU | 1536 | AACCCAGCUGGGAGAUCCC | 2151 | | [1295-1313] 3′UTR |
| AGCACUUAGGGAUCUCCCA | 1537 | UGGGAGAUCCCUAAGUGCU | 2152 | Rh | [1287-1305] 3′UTR |
| GUCUUUGGUUCUCCAGUUC | 1538 | GAACUGGAGAACCAAAGAC | 2153 | | [3056-3074] 3′UTR |
| UAACCGUGCUGAGCAGAAA | 1539 | UUUCUGCUCAGCACGGUUA | 2154 | | [3159-3177] 3′UTR |
| CUGGAUGGACUGGGUCACA | 1540 | UGUGACCCAGUCCAUCCAG | 2155 | Rh, Rb, Cw, Dg, | [820-838] ORF |
| | | | | Rt, Ms, Pg | |
| GUGCUGGGAACACACAAGA | 1541 | UCUUGUGUGUUCCCAGCAC | 2156 | | [3533-3551] 3′UTR |
| AGUGGGAGCCUCCCUCUGA | 1542 | UCAGAGGGAGGCUCCCACU | 2157 | Rh | [1593-1611] 3′UTR |
| GCAGGCAGCACUUAGGGAU | 1543 | AUCCCUAAGUGCUGCCUGC | 2158 | Rh | [1281-1299] 3′UTR |
| UAUUGGACUUGCUGCCGUA | 1544 | UACGGCAGCAAGUCCAAUA | 2159 | | [3423-3441] 3′UTR |
| GAUCUUGAUGACUUCCCUU | 1545 | AAGGGAAGUCAUCAAGAUC | 2160 | Rh | [2596-2614] 3′UTR |
| ACGCCAGCUAAGCAUAGUA | 1546 | UACUAUGCUUAGCUGGCGU | 2161 | | [2027-2045] 3′UTR |
| AGGACACUAUGGCCUGUUU | 1547 | AAACAGGCCAUAGUGUCCU | 2162 | | [2122-2140] 3′UTR |
| GCCUGAACCACAGGUACCA | 1548 | UGGUACCUGUGGUUCAGGC | 2163 | Rh, Rb, Cw, Ms, Pg | [729-747] ORF |
| UGGUUUGUUUUUGACAUCA | 1549 | UGAUGUCAAAAACAAACCA | 2164 | Rh | [2852-2870] 3′UTR |
| GUGGCAGCUGACAGAGGAA | 1550 | UUCCUCUGUCAGCUGCCAC | 2165 | | [3202-3220] 3′UTR |
| CCAUCAAUCCUAUUAAUCC | 1551 | GGAUUAAUAGGAUUGAUGG | 2166 | Rh | [1564-1582] 3′UTR |
| GGACACUAUGGCCUGUUUU | 1552 | AAAACAGGCCAUAGUGUCC | 2167 | | [2123-2141] 3′UTR |
| GACCUUCCGGUGCUGGGAA | 1553 | UUCCCAGCACCGGAAGGUC | 2168 | | [3524-3542] 3′UTR |
| ACAUCCUGAGGACAGAAAA | 1554 | UUUUCUGUCCUCAGGAUGU | 2169 | Rh | [1920-1938] 3′UTR |
| AUGUAAACAUACACACGCA | 1555 | UGCGUGUGUAUGUUUACAU | 2170 | Rh | [2420-2438] 3 ′UTR |
| GCCUCCAAGGGUUUCGACU | 1556 | AGUCGAAACCCUUGGAGGC | 2171 | Rh | [1003-1021] 3′UTR |
| CUGCAUCAAGAGAAGUGAC | 1557 | GUCACUUCUCUUGAUGCAG | 2172 | Rt, Ms | [877-895] ORF |
| UGGAAGCAUUUGACCCAGA | 1558 | UCUGGGUCAAAUGCUUCCA | 2173 | | [2955-2973] 3′UTR |
| AACACACAAGAGUUGUUGA | 1559 | UCAACAACUCUUGUGUGUU | 2174 | | [3541-3559] 3′UTR |
| GGGAACUAGGGAACCUAUG | 1560 | CAUAGGUUCCCUAGUUCCC | 2175 | Rh | [2264-2282] 3′UTR |
| AUACAGGCACAUUAUGUAA | 1561 | UUACAUAAUGUGCCUGUAU | 2176 | | [2407-2425] 3′UTR |
| GACGUGGGUCCAAGGUCCU | 1562 | AGGACCUUGGACCCACGUC | 2177 | | [1143-1161] 3′UTR |
| GGGUUAGGAUAGGAAGAAC | 1563 | GUUCUUCCUAUCCUAACCC | 2178 | | [2321-2339] 3′UTR |
| GGACGAGUGCCUCUGGAUG | 1564 | CAUCCAGAGGCACUCGUCC | 2179 | Rh, Rb, Cw | [808-826] ORF |
| AAAAAGCCUCCAAGGGUUU | 1565 | AAACCCUUGGAGGCUUUUU | 2180 | | [998-1016] 3′UTR |
| CUUUGAGUAGGUUCGGUCU | 1566 | AGACCGAACCUACUCAAAG | 2181 | | [3097-3115] 3′UTR |
| GGCAGACUGGGAGGGUAUC | 1567 | GAUACCCUCCCAGUCUGCC | 2182 | Rh | [2620-2638] 3′UTR |
| GAAGGCUCUCCAUUUGGCA | 1568 | UGCCAAAUGGAGAGCCUUC | 2183 | | [3271-3289] 3′UTR |
| GCCUCCCUCUGAGCCUUGU | 1569 | ACAAGGCUCAGAGGGAGGC | 2184 | Rh | [1600-1618] 3′UTR |
| ACAAAACAGGUUAAGAAGA | 1570 | UCUUCUUAACCUGUUUUGU | 2185 | | [3178-3196] 3′UTR |
| CCUAUGUGUUCCCUCAGUG | 1571 | CACUGAGGGAACACAUAGG | 2186 | | [2277-2295] 3′UTR |
| AAAGGGCCUGAGAAGGAUA | 1572 | UAUCCUUCUCAGGCCCUUU | 2187 | | [539-557] ORF |
| GCAGACUGCGCAUGUCUCU | 1573 | AGAGACAUGCGCAGUCUGC | 2188 | | [3570-3588] 3′UTR |
| CCCUCCCAGGCUUAGUGUU | 1574 | AACACUAAGCCUGGGAGGG | 2189 | | [1956-1974] 3′UTR |
| CCUCCAAGGGUUUCGACUG | 1575 | CAGUCGAAACCCUUGGAGG | 2190 | Rh | [1004-1022] 3′UTR |
| GCUAGUUCUUGAAGGAGCC | 1576 | GGCUCCUUCAAGAACUAGC | 2191 | | [1545-1563] 3′UTR |
| CUUGAUGACUUCCCUUUCU | 1577 | AGAAAGGGAAGUCAUCAAG | 2192 | Rh | [2599-2617] 3′UTR |
| AUUAAUCCUCAGAAUUCCA | 1578 | UGGAAUUCUGAGGAUUAAU | 2193 | Rh | [1575-1593] 3′UTR |
| GACCAGUCCAUGUGAUUUC | 1579 | GAAAUCACAUGGACUGGUC | 2194 | Rh | [2711-2729] 3′UTR |
| AGUGAGAAGGAAGUGGACU | 1580 | AGUCCACUUCCUUCUCACU | 2195 | | [452-470] ORF |
| GAGAAUCUCUUGUUUCCUC | 1581 | GAGGAAACAAGAGAUUCUC | 2196 | | [2358-2376] 3′UTR |
| GGCUCUCCAUUUGGCAUCG | 1582 | CGAUGCCAAAUGGAGAGCC | 2197 | | [3274-3292] 3′UTR |
| CUUCUUGCCUGUUCUGGCA | 1583 | UGCCAGAACAGGCAAGAAG | 2198 | | [1824-1842] 3′UTR |
| CUUUAUAUUUGAUCCACAC | 1584 | GUGUGGAUCAAAUAUAAAG | 2199 | | [3128-3146] 3′UTR |
| GGGCCUGGAAAUGUGCAUU | 1585 | AAUGCACAUUUCCAGGCCC | 2200 | Rh | [1320-1338] 3′UTR |
| CUUUGGUGACACACUCACU | 1586 | AGUGAGUGUGUCACCAAAG | 2201 | | [2088-2106] 3′UTR |
| CAGCCUAGGAAGGGAAGGA | 1587 | UCCUUCCCUUCCUAGGCUG | 2202 | Rh | [2053-2071] 3′UTR |
| CUCGCUGGACGUUGGAGGA | 1588 | UCCUCCAACGUCCAGCGAG | 2203 | Rt, Ms | [601-619] ORF |
| GCUUUAUCCGGGCUUGUGU | 1589 | ACACAAGCCCGGAUAAAGC | 2204 | | [1873-1891] 3′UTR |
| UGGACUCUGGAAACGACAU | 1590 | AUGUCGUUUCCAGAGUCCA | 2205 | Rh | [465-483] ORF |
| GCAUCGUGGAAGCAUUUGA | 1591 | UCAAAUGCUUCCACGAUGC | 2206 | Rh | [2949-2967] 3′UTR |
| CCUAAAGUUGGUAAGAUGU | 1592 | ACAUCUUACCAACUUUAGG | 2207 | Rh | [2685-2703] 3′UTR |
| GUGGUCUUGCAAAAUGCUU | 1593 | AAGCAUUUUGCAAGACCAC | 2208 | | [2466-2484] 3′UTR |
| CAAGGUCCCUUCCCUAGCU | 1594 | AGCUAGGGAAGGGACCUUG | 2209 | | [1789-1807] 3′UTR |
| GCUUUGUAUCAUUCUUGAG | 1595 | CUCAAGAAUGAUACAAAGC | 2210 | | [3592-3610] 3′UTR |
| GGGCUUCGAUCCUUGGGUG | 1596 | CACCCAAGGAUCGAAGCCC | 2211 | Rh | [1198-1216] 3′UTR |
| UCAUAAUGGACCAGUCCAU | 1597 | AUGGACUGGUCCAUUAUGA | 2212 | Rh | [2703-2721] 3′UTR |
| UAUAGUUUAAGAAGGCUCU | 1598 | AGAGCCUUCUUAAACUAUA | 2213 | | [3261-3279] 3′UTR |
| UCGACAUCGAGGACCCAUA | 1599 | UAUGGGUCCUCGAUGUCGA | 2214 | Dg | [945-963] ORF |
| ACUUCUUUCUCAGCCUCCA | 1600 | UGGAGGCUGAGAAAGAAGU | 2215 | | [2104-2122] 3′UTR |
| AUUCUCAGAUGAUAGGUGA | 1601 | UCACCUAUCAUCUGAGAAU | 2216 | | [2170-2188] 3′UTR |
| UGAGAAGGAAGUGGACUCU | 1602 | AGAGUCCACUUCCUUCUCA | 2217 | | [454-472] ORF |
| CAAAGCACCUGUUAAGACU | 1603 | AGUCUUAACAGGUGCUUUG | 2218 | Rh | [2523-2541] 3′UTR |
| GCGUUCCAGCCUCAGCUGA | 1604 | UCAGCUGAGGCUGGAACGC | 2219 | | [1650-1668] 3′UTR |
| CCUGGGCGUGGUCUUGCAA | 1605 | UUGCAAGACCACGCCCAGG | 2220 | | [2459-2477] 3′UTR |
| GUCUCUGAUGCUUUGUAUC | 1606 | GAUACAAAGCAUCAGAGAC | 2221 | | [3583-3601] 3′UTR |
| CUGUGCCCUCCCAGGCUUA | 1607 | UAAGCCUGGGAGGGCACAG | 2222 | | [1951-1969] 3′UTR |
| CCUCCAACCCAUAUAACAC | 1608 | GUGUUAUAUGGGUUGGAGG | 2223 | | [2753-2771] 3′UTR |
| UUUGUAUCAUUCUUGAGCA | 1609 | UGCUCAAGAAUGAUACAAA | 2224 | | [3594-3612] 3′UTR |
| GCCUUUAUAUUUGAUCCAC | 1610 | GUGGAUCAAAUAUAAAGGC | 2225 | | [3126-3144] 3′UTR |
| AGGCCUACCAGGUCCCUUU | 1611 | AAAGGGACCUGGUAGGCCU | 2226 | Rh | [1378-1396] 3′UTR |
| GUUCUAAGCACAGCUCUCU | 1612 | AGAGAGCUGUGCUUAGAAC | 2227 | | [3310-3328] 3′UTR |
| CUGUGUUUUUUAUUACCCU | 1613 | AGGGUAAUAAAAAACACAG | 2228 | Rh | [2889-2907] 3′UTR |
| CUAGGAAGGGAAGGAUUUU | 1614 | AAAAUCCUUCCCUUCCUAG | 2229 | Rh | [2057-2075] 3′UTR |
| CAGAUGGGCUGCGAGUGCA | 1615 | UGCACUCGCAGCCCAUCUG | 2230 | Ck, Rb, Rt | [746-764] ORF |
| CUGGCAUCAGGCACCUGGA | 1616 | UCCAGGUGCCUGAUGCCAG | 2231 | | [1837-1855] 3′UTR |
| UGAUGCUUUGUAUCAUUCU | 1617 | AGAAUGAUACAAAGCAUCA | 2232 | | [3588-3606] 3′UTR |
| AGUCCAGCCUAGGAAGGGA | 1618 | UCCCUUCCUAGGCUGGACU | 2233 | Rh | [2049-2067] 3′UTR |
| CAAAGAUUACCUAGCUAAG | 1619 | CUUAGCUAGGUAAUCUUUG | 2234 | | [2235-2253] 3′UTR |
| AGAAGGAAGUGGACUCUGG | 1620 | CCAGAGUCCACUUCCUUCU | 2235 | | [456-474] ORF |
| UGUACAGUGACCUAAAGUU | 1621 | AACUUUAGGUCACUGUACA | 2236 | | [2675-2693] 3′UTR |

TABLE B2
19-mer siTIMP2 Cross-Species

| | | | | | | |
|---|---|---|---|---|---|---|
| | | SEQ | | SEQ | | |
| | | ID | | ID | | human-73858577 |
| No. | Sense (5′ > 3′) | NO: | Antisense (5′ > 3′) | NO: | Other Sp | ORF: 303-965 |

| | | | | | | |
|---|---|---|---|---|---|---|
| 1 | UUCGCCUGCAUCAAGAGAA | 2237 | UUCUCUUGAUGCAGGCGAA | 2354 | Rh, Cw, Dg, Ms | [872-890] ORF |
| 2 | GUGCAUUUUGCAGAAACUU | 2238 | AAGUUUCUGCAAAAUGCAC | 2355 | Rh, Rt, Ms | [1332-1350] 3′UTR |
| 3 | GGUACCAGAUGGGCUGCGA | 2239 | UCGCAGCCCAUCUGGUACC | 2356 | Rh, Ck, Rb, Rt | [741-759] ORF |
| 4 | GGUCUCGCUGGACGUUGGA | 2240 | UCCAACGUCCAGCGAGACC | 2357 | Rt, Ms | [598-616] ORF |
| 5 | UCGCCUGCAUCAAGAGAAG | 2241 | CUUCUCUUGAUGCAGGCGA | 2358 | Rh, Cw, Dg, Ms | [873-891] ORF |
| 6 | CUUCCUGGAAACAGCAUGA | 2242 | UCAUGCUGUUUCCAGGAAG | 2359 | Rh, Rb, Rt, Ms | [1040-1058] 3′UTR |
| 7 | GGGCACCAGGCCAAGUUCU | 2243 | AGAACUUGGCCUGGUGCCC | 2360 | Rh, Rb, Rt, Ms | [854-872] ORF |
| 8 | CCAGAAGAAGAGCCUGAAC | 2244 | GUUCAGGCUCUUCUUCUGG | 2361 | Rh, Rb, Cw, Ms, Pg | [718-736] ORF |
| 9 | UGGACGUUGGAGGAAAGAA | 2245 | UUCUUUCCUCCAACGUCCA | 2362 | Rt, Ms | [606-624] ORF |
| 10 | GGGCUGCGAGUGCAAGAUC | 2246 | GAUCUUGCACUCGCAGCCC | 2363 | Ck, Rb, Rt | [751-769] ORF |
| 11 | CCACCCAGAAGAAGAGCCU | 2247 | AGGCUCUUCUUCUGGGUGG | 2364 | Rh, Ck, Cw, Rt, Ms, Pg | [714-732] ORF |
| 12 | AUGGGCUGCGAGUGCAAGA | 2248 | UCUUGCACUCGCAGCCCAU | 2365 | Ck, Rb, Rt | [749-767] ORF |
| 13 | AUGGACUGGGUCACAGAGA | 2249 | UCUCUGUGACCCAGUCCAU | 2366 | Rh, Rt, Ms, Pg | [824-842] ORF |
| 14 | UGGGCUGCGAGUGCAAGAU | 2250 | AUCUUGCACUCGCAGCCCA | 2367 | Ck, Rb, Rt | [750-768] ORF |
| 15 | ACGUUGGAGGAAAGAAGGA | 2251 | UCCUUCUUUCCUCCAACGU | 2368 | Rt, Ms | [609-627] ORF |
| 16 | GACGUUGGAGGAAAGAAGG | 2252 | CCUUCUUUCCUCCAACGUC | 2369 | Rt, Ms | [608-626] ORF |
| 17 | AGGCCAAGUUCUUCGCCUG | 2253 | CAGGCGAAGAACUUGGCCU | 2370 | Rh, Rb, Cw, Dg, Ms | [861-879] ORF |
| 18 | GAAGAAGAGCCUGAACCAC | 2254 | GUGGUUCAGGCUCUUCUUC | 2371 | Rh, Rb, Cw, Ms, Pg | [721-739] ORF |
| 19 | GCACCCGCAACAGGCGUUU | 2255 | AAACGCCUGUUGCGGGUGC | 2372 | Cw, Dg, Rt, Ms | [397-415] ORF |
| 20 | UUCUUCGCCUGCAUCAAGA | 2256 | UCUUGAUGCAGGCGAAGAA | 2373 | Rh, Rb, Cw, Dg, Ms | [869-887] ORF |
| 21 | GAGCCUGAACCACAGGUAC | 2257 | GUACCUGUGGUUCAGGCUC | 2374 | Rh, Rb, Cw, Ms, Pg | [727-745] ORF |
| 22 | CUUCGCCUGCAUCAAGAGA | 2258 | UCUCUUGAUGCAGGCGAAG | 2375 | Rh, Rb, Cw, Dg, Ms | [871-889] ORF |
| 23 | UGGAAACAGCAUGAAUAAA | 2259 | UUUAUUCAUGCUGUUUCCA | 2376 | Rh, Rb, Rt, Ms | [1045-1063] 3′UTR |
| 24 | GACAUCCCUUCCUGGAAAC | 2260 | GUUUCCAGGAAGGGAUGUC | 2377 | Rh, Rt, Ms | [1033-1051] 3′UTR |
| 25 | GUUCUUCGCCUGCAUCAAG | 2261 | CUUGAUGCAGGCGAAGAAC | 2378 | Rh, Rb, Cw, Dg, Ms | [868-886] ORF |
| 26 | AAGUUCUUCGCCUGCAUCA | 2262 | UGAUGCAGGCGAAGAACUU | 2379 | Rh, Rb, Cw, Dg, Ms | [866-884] ORF |
| 27 | CCCUUCCUGGAAACAGCAU | 2263 | AUGCUGUUUCCAGGAAGGG | 2380 | Rh, Rb, Rt, Ms | [1038-1056] 3′UTR |
| 28 | CGCUCGGCCUCCUGCUGCU | 2264 | AGCAGCAGGAGGCCGAGCG | 2381 | Dg, Rt, Ms | [333-351] ORF |
| 29 | UGAACCACAGGUACCAGAU | 2265 | AUCUGGUACCUGUGGUUCA | 2382 | Rh, Rb, Cw, Ms, Pg | [732-750] ORF |
| 30 | CUGAACCACAGGUACCAGA | 2266 | UCUGGUACCUGUGGUUCAG | 2383 | Rh, Rb, Cw, Ms, Pg | [731-749] ORF |
| 31 | CAGAAGAAGAGCCUGAACC | 2267 | GGUUCAGGCUCUUCUUCUG | 2384 | Rh, Rb, Cw, Ms, Pg | [719-737] ORF |
| 32 | UCCUGGAAACAGCAUGAAU | 2268 | AUUCAUGCUGUUUCCAGGA | 2385 | Rh, Rb, Rt, Ms | [1042-1060] 3′UTR |
| 33 | GAUGGACUGGGUCACAGAG | 2269 | CUCUGUGACCCAGUCCAUC | 2386 | Rh, Rt, Ms, Pg | [823-841] ORF |
| 34 | GCCGACGCCUGCAGCUGCU | 2270 | AGCAGCUGCAGGCGUCGGC | 2387 | Cw, Dg, Rt, Ms | [371-389] ORF |
| 35 | CAGGCGUUUUGCAAUGCAG | 2271 | CUGCAUUGCAAAACGCCUG | 2388 | Cw, Rt, Ms | [407-425] ORF |
| 36 | UCAAGCAGAUAAAGAUGUU | 2272 | AACAUCUUUAUCUGCUUGA | 2389 | Cw, Dg, Rt, Ms, Pg | [519-537] ORF |
| 37 | GAACCACAGGUACCAGAUG | 2273 | CAUCUGGUACCUGUGGUUC | 2390 | Rh, Rb, Cw, Rt, Ms, Pg | [733-751] ORF |
| 38 | CACCCAGAAGAAGAGCCUG | 2274 | CAGGCUCUUCUUCUGGGUG | 2391 | Rh, Ck, Cw, Rt, Ms, Pg | [715-733] ORF |
| 39 | CCUUCCUGGAAACAGCAUG | 2275 | CAUGCUGUUUCCAGGAAGG | 2392 | Rh, Rb, Rt, Ms | [1039-1057] 3′UTR |
| 40 | CAACAGGCGUUUUGCAAUG | 2276 | CAUUGCAAAACGCCUGUUG | 2393 | Cw, Rt, Ms | [404-422] ORF |
| 41 | CACAGGUACCAGAUGGGCU | 2277 | AGCCCAUCUGGUACCUGUG | 2394 | Rh, Rb, Cw, Rt, Ms, Pg | [737-755] ORF |
| 42 | UCCCUUCCUGGAAACAGCA | 2278 | UGCUGUUUCCAGGAAGGGA | 2395 | Rh, Rb, Rt, Ms | [1037-1055] 3′UTR |
| 43 | UGAGAUCAAGCAGAUAAAG | 2279 | CUUUAUCUGCUUGAUCUCA | 2396 | Rh, Cw, Dg, Rt, Ms | [514-532] ORF |
| 44 | GGUGCACCCGCAACAGGCG | 2280 | CGCCUGUUGCGGGUGCACC | 2397 | Cw, Dg, Rt, Ms | [394-412] ORF |
| 45 | AGUGCCUCUGGAUGGACUG | 2281 | CAGUCCAUCCAGAGGCACU | 2398 | Rh, Rb, Cw, Dg, Rt, Ms | [813-831] ORF |
| 46 | AAGCAGAUAAAGAUGUUCA | 2282 | UGAACAUCUUUAUCUGCUU | 2399 | Cw, Dg, Rt, Ms, Pg | [521-539] ORF |
| 47 | UGCACCCGCAACAGGCGUU | 2283 | AACGCCUGUUGCGGGUGCA | 2400 | Cw, Dg, Rt, Ms | [396-414] ORF |
| 48 | CACCCGCAACAGGCGUUUU | 2284 | AAAACGCCUGUUGCGGGUG | 2401 | Cw, Rt, Ms | [398-416] ORF |
| 49 | CUGGACGUUGGAGGAAAGA | 2285 | UCUUUCCUCCAACGUCCAG | 2402 | Rt, Ms | [605-623] ORF |
| 50 | AUCCCUUCCUGGAAACAGC | 2286 | GCUGUUUCCAGGAAGGGAU | 2403 | Rh, Rb, Rt, Ms | [1036-1054] 3′UTR |
| 51 | ACAGGCGUUUUGCAAUGCA | 2287 | UGCAUUGCAAAACGCCUGU | 2404 | Cw, Rt, Ms | [406-424] ORF |
| 52 | GAUGGGCUGCGAGUGCAAG | 2288 | CUUGCACUCGCAGCCCAUC | 2405 | Ck, Rb, Rt | [748-766] ORF |
| 53 | AGCCUGAACCACAGGUACC | 2289 | GGUACCUGUGGUUCAGGCU | 2406 | Rh, Rb, Cw, Ms, Pg | [728-746] ORF |
| 54 | CCUCUGGAUGGACUGGGUC | 2290 | GACCCAGUCCAUCCAGAGG | 2407 | Rh, Rb, Cw, Dg, Rt, Ms, | [817-835] ORF |
| | | | | | Pg | |
| 55 | CGGGCACCAGGCCAAGUUC | 2291 | GAACUUGGCCUGGUGCCCG | 2408 | Rh, Rb, Rt, Ms | [853-871] ORF |
| 56 | CAGGCCAAGUUCUUCGCCU | 2292 | AGGCGAAGAACUUGGCCUG | 2409 | Rh, Rb, Cw, Dg, Ms | [860-878] ORF |
| 57 | CAAGUUCUUCGCCUGCAUC | 2293 | GAUGCAGGCGAAGAACUUG | 2410 | Rh, Rb, Cw, Dg, Ms | [865-883] ORF |
| 58 | ACUUCAUCGUGCCCUGGGA | 2294 | UCCCAGGGCACGAUGAAGU | 2411 | Rh, Rb, Cw, Dg, Pg | [684-702] ORF |
| 59 | ACAUCCCUUCCUGGAAACA | 2295 | UGUUUCCAGGAAGGGAUGU | 2412 | Rh, Rt, Ms | [1034-1052] 3′UTR |
| 60 | UACCAGAUGGGCUGCGAGU | 2296 | ACUCGCAGCCCAUCUGGUA | 2413 | Rh, Ck, Rb, Rt | [743-761] ORF |
| 61 | GGCCAAGUUCUUCGCCUGC | 2297 | GCAGGCGAAGAACUUGGCC | 2414 | Rh, Rb, Cw, Dg, Ms | [862-880] ORF |
| 62 | CCAGAUGGGCUGCGAGUGC | 2298 | GCACUCGCAGCCCAUCUGG | 2415 | Rh, Ck, Rb, Rt | [745-763] ORF |
| 63 | GUGACUUCAUCGUGCCCUG | 2299 | CAGGGCACGAUGAAGUCAC | 2416 | Rh, Rb, Cw, Dg, Pg | [681-699] ORF |
| 64 | UCGCUGGACGUUGGAGGAA | 2300 | UUCCUCCAACGUCCAGCGA | 2417 | Rt, Ms | [602-620] ORF |
| 65 | AUCAAGCAGAUAAAGAUGU | 2301 | ACAUCUUUAUCUGCUUGAU | 2418 | Cw, Dg, Rt, Ms, Pg | [518-536] ORF |
| 66 | GUACCAGAUGGGCUGCGAG | 2302 | CUCGCAGCCCAUCUGGUAC | 2419 | Rh, Ck, Rb, Rt | [742-760] ORF |
| 67 | CGAGUGCCUCUGGAUGGAC | 2303 | GUCCAUCCAGAGGCACUCG | 2420 | Rh, Rb, Cw, Dg, Rt, Ms | [811-829] ORF |
| 68 | UCUGGAUGGACUGGGUCAC | 2304 | GUGACCCAGUCCAUCCAGA | 2421 | Rh, Rb, Cw, Dg, Rt, Ms, | [819-837] ORF |
| | | | | | Pg | |
| 69 | ACCAGAUGGGCUGCGAGUG | 2305 | CACUCGCAGCCCAUCUGGU | 2422 | Rh, Ck, Rb, Rt | [744-762] ORF |
| 70 | ACAGGUACCAGAUGGGCUG | 2306 | CAGCCCAUCUGGUACCUGU | 2423 | Rh, Rb, Cw, Rt, Ms, Pg | [738-756] ORF |
| 71 | GAAGAGCCUGAACCACAGG | 2307 | CCUGUGGUUCAGGCUCUUC | 2424 | Rh, Rb, Cw, Ms, Pg | [724-742] ORF |
| 72 | GUGCACCCGCAACAGGCGU | 2308 | ACGCCUGUUGCGGGUGCAC | 2425 | Cw, Dg, Rt, Ms | [395-413] ORF |
| 73 | UGGAUGGACUGGGUCACAG | 2309 | CUGUGACCCAGUCCAUCCA | 2426 | Rh, Rt, Ms, Pg | [821-839] ORF |
| 74 | CAAGCAGAUAAAGAUGUUC | 2310 | GAACAUCUUUAUCUGCUUG | 2427 | Cw, Dg, Rt, Ms, Pg | [520-538] ORF |
| 75 | GUGCCUCUGGAUGGACUGG | 2311 | CCAGUCCAUCCAGAGGCAC | 2428 | Rh, Rb, Cw, Dg, Rt, Ms | [814-832] ORF |
| 76 | CAUCCCUUCCUGGAAACAG | 2312 | CUGUUUCCAGGAAGGGAUG | 2429 | Rh, Rb, Rt, Ms | [1035-1053] 3′UTR |
| 77 | CACCAGGCCAAGUUCUUCG | 2313 | CGAAGAACUUGGCCUGGUG | 2430 | Rh, Rb, Cw, Ms | [857-875] ORF |
| 78 | CCUGAACCACAGGUACCAG | 2314 | CUGGUACCUGUGGUUCAGG | 2431 | Rh, Rb, Cw, Ms, Pg | [730-748] ORF |
| 79 | AACCACAGGUACCAGAUGG | 2315 | CCAUCUGGUACCUGUGGUU | 2432 | Rh, Rb, Cw, Rt, Ms, Pg | [734-752] ORF |
| 80 | GUCUCGCUGGACGUUGGAG | 2316 | CUCCAACGUCCAGCGAGAC | 2433 | Rt, Ms | [599-617] ORF |
| 81 | AGAGUUUAUCUACACGGCC | 2317 | GGCCGUGUAGAUAAACUCU | 2434 | Dg, Ms, Pg | [559-577] ORF |
| 82 | UUCCUGGAAACAGCAUGAA | 2318 | UUCAUGCUGUUUCCAGGAA | 2435 | Rh, Rb, Rt, Ms | [1041-1059] 3′UTR |
| 83 | GAUAAAGAUGUUCAAAGGG | 2319 | CCCUUUGAACAUCUUUAUC | 2436 | Dg, Rt, Ms | [526-544] ORF |
| 84 | UCUUCGCCUGCAUCAAGAG | 2320 | CUCUUGAUGCAGGCGAAGA | 2437 | Rh, Rb, Cw, Dg, Ms | [870-888] ORF |
| 85 | UGGCGCUCGGCCUCCUGCU | 2321 | AGCAGGAGGCCGAGCGCCA | 2438 | Dg, Rt, Ms | [330-348] ORF |
| 86 | CCCGGUGCACCCGCAACAG | 2322 | CUGUUGCGGGUGCACCGGG | 2439 | Cw, Dg, Rt, Ms | [391-409] ORF |
| 87 | CCCGCAACAGGCGUUUUGC | 2323 | GCAAAACGCCUGUUGCGGG | 2440 | Cw, Rt, Ms | [400-418] ORF |
| 88 | CAGGGCCAAAGCGGUCAGU | 2324 | ACUGACCGCUUUGGCCCUG | 2441 | Rb, Dg | [436-454] ORF |
| 89 | AAGAGCCUGAACCACAGGU | 2325 | ACCUGUGGUUCAGGCUCUU | 2442 | Rh, Rb, Cw, Ms, Pg | [725-743] ORF |
| 90 | ACCACAGGUACCAGAUGGG | 2326 | CCCAUCUGGUACCUGUGGU | 2443 | Rh, Rb, Cw, Rt, Ms, Pg | [735-753] ORF |
| 91 | CAGGUACCAGAUGGGCUGC | 2327 | GCAGCCCAUCUGGUACCUG | 2444 | Rh, Rb, Cw, Dg, Rt, Ms, | [739-757] ORF |
| | | | | | Pg | |
| 92 | CGGUGCACCCGCAACAGGC | 2328 | GCCUGUUGCGGGUGCACCG | 2445 | Cw, Dg, Rt, Ms | [393-411] ORF |
| 93 | GCUCGGCCUCCUGCUGCUG | 2329 | CAGCAGCAGGAGGCCGAGC | 2446 | Dg, Rt, Ms | [334-352] ORF |
| 94 | GACGCCUGCAGCUGCUCCC | 2330 | GGGAGCAGCUGCAGGCGUC | 2447 | Cw, Dg, Rt, Ms | [374-392] ORF |
| 95 | ACCCGCAACAGGCGUUUUG | 2331 | CAAAACGCCUGUUGCGGGU | 2448 | Cw, Rt, Ms | [399-417] ORF |
| 96 | CCGGUGCACCCGCAACAGG | 2332 | CCUGUUGCGGGUGCACCGG | 2449 | Cw, Dg, Rt, Ms | [392-410] ORF |
| 97 | UCUCGCUGGACGUUGGAGG | 2333 | CCUCCAACGUCCAGCGAGA | 2450 | Rt, Ms | [600-618] ORF |
| 98 | AACAGCAUGAAUAAAACAC | 2334 | GUGUUUUAUUCAUGCUGUU | 2451 | Rh, Rt, Ms | [1049-1067] 3′UTR |
| 99 | CCACAGGUACCAGAUGGGC | 2335 | GCCCAUCUGGUACCUGUGG | 2452 | Rh, Rb, Cw, Rt, Ms, Pg | [736-754] ORF |
| 100 | CCGACGCCUGCAGCUGCUC | 2336 | GAGCAGCUGCAGGCGUCGG | 2453 | Cw, Dg, Rt, Ms | [372-390] ORF |
| 101 | UUCAUCGUGCCCUGGGACA | 2337 | UGUCCCAGGGCACGAUGAA | 2454 | Rh, Rb, Cw, Dg, Pg | [686-704] ORF |
| 102 | CUUCAUCGUGCCCUGGGAC | 2338 | GUCCCAGGGCACGAUGAAG | 2455 | Rh, Rb, Cw, Dg, Pg | [685-703] ORF |
| 103 | CGACGCCUGCAGCUGCUCC | 2339 | GGAGCAGCUGCAGGCGUCG | 2456 | Cw, Dg, Rt, Ms | [373-391] ORF |
| 104 | UGCCUCUGGAUGGACUGGG | 2340 | CCCAGUCCAUCCAGAGGCA | 2457 | Rh, Rb, Cw, Dg, Rt, Ms | [815-833] ORF |
| 105 | ACCAGGCCAAGUUCUUCGC | 2341 | GCGAAGAACUUGGCCUGGU | 2458 | Rh, Rb, Cw, Ms | [858-876] ORF |
| 106 | AGGUACCAGAUGGGCUGCG | 2342 | CGCAGCCCAUCUGGUACCU | 2459 | Rh, Ck, Rb, Rt | [740-758] ORF |
| 107 | CUCGGCCUCCUGCUGCUGG | 2343 | CCAGCAGCAGGAGGCCGAG | 2460 | Dg, Rt | [335-353] ORF |
| 108 | AACAGGCGUUUUGCAAUGC | 2344 | GCAUUGCAAAACGCCUGUU | 2461 | Cw, Rt, Ms | [405-423] ORF |
| 109 | UCGGCCUCCUGCUGCUGGC | 2345 | GCCAGCAGCAGGAGGCCGA | 2462 | Dg, Rt | [336-354] ORF |
| 110 | CCAGGCCAAGUUCUUCGCC | 2346 | GGCGAAGAACUUGGCCUGG | 2463 | Rh, Rb, Cw, Dg, Ms | [859-877] ORF |
| 111 | UGUGACUUCAUCGUGCCCU | 2347 | AGGGCACGAUGAAGUCACA | 2464 | Rh, Rb, Cw, Dg, Pg | [680-698] ORF |
| 112 | GACUUCAUCGUGCCCUGGG | 2348 | CCCAGGGCACGAUGAAGUC | 2465 | Rh, Rb, Cw, Dg, Pg | [683-701] ORF |
| 113 | CUGUGACUUCAUCGUGCCC | 2349 | GGGCACGAUGAAGUCACAG | 2466 | Rh, Rb, Cw, Dg, Pg | [679-697] ORF |
| 114 | UGACUUCAUCGUGCCCUGG | 2350 | CCAGGGCACGAUGAAGUCA | 2467 | Rh, Rb, Cw, Dg, Pg | [682-700] ORF |
| 616 | GCAGGAGUUUCUCGACAUC | 2351 | GAUGUCGAGAAACUCCUGC | 2468 | Dg, Ck | [934-952] ORF |
| 617 | GCACCACCCAGAAGAAGAG | 2352 | CUCUUCUUCUGGGUGGUGC | 2469 | Pg, Rh | [711-729] ORF |
| 618 | AGCUCUGACAUCCCUUCCU | 2353 | AGGAAGGGAUGUCAGAGCU | 2470 | Rh | [1027-1045] 3′UTR |

TABLE B3
Preferred 19-mer siTIMP2

| | | | | | | |
|---|---|---|---|---|---|---|
| | | SEQ | | SEQ | | |
| | | ID | | ID | | |
| siTIMP2_pNo. | Sense (5′ > 3′) | NO: | Antisense (5′ > 3′) | NO: | length | position |

| | | | | | | |
|---|---|---|---|---|---|---|
| siTIMP2_p4 | GGCUGCGAGUGCAAGAUCA | 2471 | UGAUCUUGCACUCGCAGCC | 2524 | 19 | [752-770] ORF |
| siTIMP2_p16 | GUAUGAGAUCAAGCAGAUA | 2472 | UAUCUGCUUGAUCUCAUAC | 2525 | 19 | [511-529] ORF |
| siTIMP2_p17 | GAGGAAAGAAGGAAUAUCU | 2473 | AGAUAUUCCUUCUUUCCUC | 2526 | 19 | [615-633] ORF |
| siTIMP2_p18 | CCUGCAUCAAGAGAAGUGA | 2474 | UCACUUCUCUUGAUGCAGG | 2527 | 19 | [876-894] ORF |
| siTIMP2_p20 | AUGAGAUCAAGCAGAUAAA | 2475 | UUUAUCUGCUUGAUCUCAU | 2528 | 19 | [513-531] ORF |
| siTIMP2_p24 | GUUGGAGGAAAGAAGGAAU | 2476 | AUUCCUUCUUUCCUCCAAC | 2529 | 19 | [611-629] ORF |
| siTIMP2_p25 | ACUGGGUCACAGAGAAGAA | 2477 | UUCUUCUCUGUGACCCAGU | 2530 | 19 | [828-846] ORF |
| siTIMP2_p27 | CUCUGGAUGGACUGGGUCA | 2478 | UGACCCAGUCCAUCCAGAG | 2531 | 19 | [818-836] ORF |
| siTIMP2_p29 | GGCGUUUUGCAAUGCAGAU | 2479 | AUCUGCAUUGCAAAACGCC | 2532 | 19 | [409-427] ORF |
| siTIMP2_p30 | GCCUCUGGAUGGACUGGGU | 2480 | ACCCAGUCCAUCCAGAGGC | 2533 | 19 | [816-834] ORF |
| siTIMP2_p33 | GGAGGAAAGAAGGAAUAUC | 2481 | GAUAUUCCUUCUUUCCUCC | 2534 | 19 | [614-632] ORF |
| siTIMP2_p35 | GGACUGGGUCACAGAGAAG | 2482 | CUUCUCUGUGACCCAGUCC | 2535 | 19 | [826-844] ORF |
| siTIMP2_p37 | GGACGUUGGAGGAAAGAAG | 2483 | CUUCUUUCCUCCAACGUCC | 2536 | 19 | [607-625] ORF |
| siTIMP2_p38 | CGUUGGAGGAAAGAAGGAA | 2484 | UUCCUUCUUUCCUCCAACG | 2537 | 19 | [610-628] ORF |
| siTIMP2_p39 | CUGACAUCCCUUCCUGGAA | 2485 | UUCCAGGAAGGGAUGUCAG | 2538 | 19 | [1031-1049]3′UTR |
| siTIMP2_p40 | UGACAUCCCUUCCUGGAAA | 2486 | UUUCCAGGAAGGGAUGUCA | 2539 | 19 | [1032-1050]3′UTR |
| siTIMP2_p41 | AGAUGGGCUGCGAGUGCAA | 2487 | UUGCACUCGCAGCCCAUCU | 2540 | 19 | [747-765] ORF |
| siTIMP2_p44 | GGGUCUCGCUGGACGUUGG | 2488 | CCAACGUCCAGCGAGACCC | 2541 | 19 | [597-615] ORF |
| siTIMP2_p46 | GAGUGCCUCUGGAUGGACU | 2489 | AGUCCAUCCAGAGGCACUC | 2542 | 19 | [812-830] ORF |
| siTIMP2_p51 | AGCAGAUAAAGAUGUUCAA | 2490 | UUGAACAUCUUUAUCUGCU | 2543 | 19 | [522-540] ORF |
| siTIMP2_p55 | GCAACAGGCGUUUUGCAAU | 2491 | AUUGCAAAACGCCUGUUGC | 2544 | 19 | [403-421] ORF |
| siTIMP2_p61 | GCUGGACGUUGGAGGAAAG | 2492 | CUUUCCUCCAACGUCCAGC | 2545 | 19 | [604-622] ORF |
| siTIMP2_p62 | UGUGCAUUUUGCAGAAACU | 2493 | AGUUUCUGCAAAAUGCACA | 2546 | 19 | [1331-1349]3′UTR |
| siTIMP2_p64 | GGCACCAGGCCAAGUUCUU | 2494 | AAGAACUUGGCCUGGUGCC | 2547 | 19 | [855-873] ORF |
| siTIMP2_p65 | GCCUGCAUCAAGAGAAGUG | 2495 | CACUUCUCUUGAUGCAGGC | 2548 | 19 | [875-893] ORF |
| siTIMP2_p67 | CUGGAUGGACUGGGUCACA | 2496 | UGUGACCCAGUCCAUCCAG | 2549 | 19 | [820-838] ORF |
| siTIMP2_p68 | CUGCAUCAAGAGAAGUGAC | 2497 | GUCACUUCUCUUGAUGCAG | 2550 | 19 | [877-895] ORF |
| siTIMP2_p69 | CUCGCUGGACGUUGGAGGA | 2498 | UCCUCCAACGUCCAGCGAG | 2551 | 19 | [601-619] ORF |
| siTIMP2_p71 | CAGAUGGGCUGCGAGUGCA | 2499 | UGCACUCGCAGCCCAUCUG | 2552 | 19 | [746-764] ORF |
| siTIMP2_p75 | GUGCAUUUUGCAGAAACUU | 2500 | AAGUUUCUGCAAAAUGCAC | 2553 | 19 | [1332-1350]3′UTR |
| siTIMP2_p76 | GGUACCAGAUGGGCUGCGA | 2501 | UCGCAGCCCAUCUGGUACC | 2554 | 19 | [741-759] ORF |
| siTIMP2_p78 | GGUCUCGCUGGACGUUGGA | 2502 | UCCAACGUCCAGCGAGACC | 2555 | 19 | [598-616] ORF |
| siTIMP2_p79 | CUUCCUGGAAACAGCAUGA | 2503 | UCAUGCUGUUUCCAGGAAG | 2556 | 19 | [1040-1058]3′UTR |
| siTIMP2_p82 | GGGCACCAGGCCAAGUUCU | 2504 | AGAACUUGGCCUGGUGCCC | 2557 | 19 | [854-872] ORF |
| siTIMP2_p83 | GUCACAGAGAAGAACAUCA | 2505 | UGAUGUUCUUCUCUGUGAC | 2558 | 19 | [833-851] ORF |
| siTIMP2_p84 | AGGAGUUUCUCGACAUCGA | 2506 | UCGAUGUCGAGAAACUCCU | 2559 | 19 | [936-954] ORF |
| siTIMP2_p85 | CCCAGAAGAAGAGCCUGAA | 2507 | UUCAGGCUCUUCUUCUGGG | 2560 | 19 | [717-735] ORF |
| siTIMP2_p86 | GGGUCACAGAGAAGAACAU | 2508 | AUGUUCUUCUCUGUGACCC | 2561 | 19 | [831-849] ORF |
| siTIMP2_p87 | CCAAGUUCUUCGCCUGCAU | 2509 | AUGCAGGCGAAGAACUUGG | 2562 | 19 | [864-882] ORF |
| siTIMP2_p88 | AGCACCACCCAGAAGAAGA | 2510 | UCUUCUUCUGGGUGGUGCU | 2563 | 19 | [710-728] ORF |
| siTIMP2_p89 | GUUUUGCAAUGCAGAUGUA | 2511 | UACAUCUGCAUUGCAAAAC | 2564 | 19 | [412-430] ORF |
| siTIMP2_p90 | AGCAGGAGUUUCUCGACAU | 2512 | AUGUCGAGAAACUCCUGCU | 2565 | 19 | [933-951] ORF |
| siTIMP2_p91 | GGAGUUUCUCGACAUCGAG | 2513 | CUCGAUGUCGAGAAACUCC | 2566 | 19 | [937-955] ORF |
| siTIMP2_p92 | GCCUGAACCACAGGUACCA | 2514 | UGGUACCUGUGGUUCAGGC | 2567 | 19 | [729-747] ORF |
| siTIMP2_p93 | UUCGCCUGCAUCAAGAGAA | 2515 | UUCUCUUGAUGCAGGCGAA | 2568 | 19 | [872-890] ORF |
| siTIMP2_p94 | AGAGCCUGAACCACAGGUA | 2516 | UACCUGUGGUUCAGGCUCU | 2569 | 19 | [726-744] ORF |
| siTIMP2_p95 | GCAGGAGUUUCUCGACAUC | 2517 | GAUGUCGAGAAACUCCUGC | 2570 | 19 | [934-952] ORF |
| siTIMP2_p96 | GCACCACCCAGAAGAAGAG | 2518 | CUCUUCUUCUGGGUGGUGC | 2571 | 19 | [711-729] ORF |
| siTIMP2_p97 | GGACGAGUGCCUCUGGAUG | 2519 | CAUCCAGAGGCACUCGUCC | 2572 | 19 | [808-826] ORF |
| siTIMP2_p98 | GACUGGUCCAGCUCUGACA | 2520 | UGUCAGAGCUGGACCAGUC | 2573 | 19 | [1018-1036] 3′UTR |
| siTIMP2_p99 | ACAUCACCCUCUGUGACUU | 2521 | AAGUCACAGAGGGUGAUGU | 2574 | 19 | [669-687] ORF |
| siTIMP2_p100 | CCGGACGAGUGCCUCUGGA | 2522 | UCCAGAGGCACUCGUCCGG | 2575 | 19 | [806-824] ORF |
| siTIMP2_p101 | UCGCCUGCAUCAAGAGAAG | 2523 | CUUCUCUUGAUGCAGGCGA | 2576 | 19 | [873-891] ORF |
| SiTIMP2_p102 | GGAAGAACUUUCUCGGUAA | 1007 | UUACCGAGAAAGUUCUUCC | 1622 | 19 | [2332-2350]3′UTR |

TABLE B4
19 mer siTIMP2 with lowest predicted OT effect

| | | | | | | |
|---|---|---|---|---|---|---|
| | | | SEQ | | SEQ | |
| | | | ID | | ID | No. in Table |
| Cross species: | Ranking | Sense (5′ > 3′) | NO: | Antisense (5′ > 3′) | NO: | B3 |

| | | | | | | |
|---|---|---|---|---|---|---|
| H/Rt | 4 | CUCUGGAUGGACUGGGUCA | 2478 | UGACCCAGUCCAUCCAGAG | 2531 | siTIMP2_p27 |
| H/Rt | 3 | GGCGUUUUGCAAUGCAGAU | 2479 | AUCUGCAUUGCAAAACGCC | 2532 | siTIMP2_p29 |
| H/Rt | 4 | GCCUCUGGAUGGACUGGGU | 2480 | ACCCAGUCCAUCCAGAGGC | 2533 | siTIMP2_p30 |
| H/Rt | 4 | CUGACAUCCCUUCCUGGAA | 2485 | UUCCAGGAAGGGAUGUCAG | 2538 | siTIMP2_p39 |
| H/Rt | 3 | UGACAUCCCUUCCUGGAAA | 2486 | UUUCCAGGAAGGGAUGUCA | 2539 | siTIMP2_p40 |
| H/Rt | 4 | AGAUGGGCUGCGAGUGCAA | 2487 | UUGCACUCGCAGCCCAUCU | 2540 | siTIMP2_p41 |
| H/Rt | 4 | GAGUGCCUCUGGAUGGACU | 2489 | AGUCCAUCCAGAGGCACUC | 2542 | siTIMP2_p46 |
| H/Rt | 2 | GCAACAGGCGUUUUGCAAU | 2491 | AUUGCAAAACGCCUGUUGC | 2544 | siTIMP2_p55 |
| H/Rt | 4 | UGUGCAUUUUGCAGAAACU | 2493 | AGUUUCUGCAAAAUGCACA | 2546 | siTIMP2_p62 |
| H/Rt | 3 | CUGCAUCAAGAGAAGUGAC | 2497 | GUCACUUCUCUUGAUGCAG | 2550 | siTIMP2_p68 |
| H/Rt | 1 | CUCGCUGGACGUUGGAGGA | 2498 | UCCUCCAACGUCCAGCGAG | 2551 | siTIMP2_p69 |
| H/Rt | 4 | CAGAUGGGCUGCGAGUGCA | 2499 | UGCACUCGCAGCCCAUCUG | 2552 | siTIMP2_p71 |
| H/Rt | 2 | GGUACCAGAUGGGCUGCGA | 2501 | UCGCAGCCCAUCUGGUACC | 2554 | siTIMP2_p76 |
| H/Rt | 2 | GGUCUCGCUGGACGUUGGA | 2502 | UCCAACGUCCAGCGAGACC | 2555 | siTIMP2_p78 |
| H/Rt (Rt Cross 1MM) | 4 | GUUUUGCAAUGCAGAUGUA | 2511 | UACAUCUGCAUUGCAAAAC | 2564 | siTIMP2_p89 |
| H/Rt (Rt Cross 1MM) | 3 | GGAGUUUCUCGACAUCGAG | 2513 | CUCGAUGUCGAGAAACUCC | 2566 | siTIMP2_p91 |
| H/Rt (Rt Cross 1MM) | 2 | UUCGCCUGCAUCAAGAGAA | 2515 | UUCUCUUGAUGCAGGCGAA | 2568 | siTIMP2_p93 |
| H/Rt (Rt Cross 1MM) | 4 | GCAGGAGUUUCUCGACAUC | 2517 | GAUGUCGAGAAACUCCUGC | 2570 | siTIMP2_p95 |
| H/Rt (Rt Cross 1MM) | 3 | GGACGAGUGCCUCUGGAUG | 2519 | CAUCCAGAGGCACUCGUCC | 2572 | siTIMP2_p97 |
| H/Rt (Rt Cross 1MM) | 4 | GACUGGUCCAGCUCUGACA | 2520 | UGUCAGAGCUGGACCAGUC | 2573 | siTIMP2_p98 |
| H/Rt (Rt Cross 1MM) | 2 | CCGGACGAGUGCCUCUGGA | 2522 | UCCAGAGGCACUCGUCCGG | 2575 | siTIMP2_p100 |

TABLE B5
18-mer siTIMP2

| | | | | | | |
|---|---|---|---|---|---|---|
| | | SEQ | | SEQ | | |
| | | ID | | ID | | human-73858577 |
| No. | Sense (5′ > 3′) | NO: | Antisense (5′ > 3′) | NO: | Other Sp | ORF: 303-965 |

| | | | | | | |
|---|---|---|---|---|---|---|
| 1 | CCAUGUGAUUUCAGUAUA | 2577 | UAUACUGAAAUCACAUGG | 3612 | Rh | [2718-2735] 3′UTR |
| 2 | CUCUGAGCCUUGUAGAAA | 2578 | UUUCUACAAGGCUCAGAG | 3613 | Rh | [1606-1623] 3′UTR |
| 3 | GGUAAUGAUAAGGAGAAU | 2579 | AUUCUCCUUAUCAUUACC | 3614 | | [2346-2363] 3′UTR |
| 4 | GAUGCUUUGUAUCAUUCU | 2580 | AGAAUGAUACAAAGCAUC | 3615 | | [3589-3606] 3′UTR |
| 5 | GGGUCUGGAGGGAGACGU | 2581 | ACGUCUCCCUCCAGACCC | 3616 | | [1130-1147] 3′UTR |
| 6 | GAUCCAGUAUGAGAUCAA | 2582 | UUGAUCUCAUACUGGAUC | 3617 | Rh, Rb | [505-522] ORF |
| 7 | CUGAGAAGGAUAUAGAGU | 2583 | ACUCUAUAUCCUUCUCAG | 3618 | | [546-563] ORF |
| 8 | GUAUCAUUCUUGAGCAAU | 2584 | AUUGCUCAAGAAUGAUAC | 3619 | | [3597-3614] 3′UTR |
| 9 | GCCUGAGAAGGAUAUAGA | 2585 | UCUAUAUCCUUCUCAGGC | 3620 | | [544-561] ORF |
| 10 | GGCUAGUUCUUGAAGGAG | 2586 | CUCCUUCAAGAACUAGCC | 3621 | | [1544-1561] 3′UTR |
| 11 | GGGUCACAGAGAAGAACA | 2587 | UGUUCUUCUCUGUGACCC | 3622 | Rh | [831-848] ORF |
| 12 | GGAUCUUUGAGUAGGUUC | 2588 | GAACCUACUCAAAGAUCC | 3623 | | [3093-3110] 3′UTR |
| 13 | GGAAGUGGACUCUGGAAA | 2589 | UUUCCAGAGUCCACUUCC | 3624 | | [460-477] ORF |
| 14 | GAACCUGAGUUGCAGAUA | 2590 | UAUCUGCAACUCAGGUUC | 3625 | Rh | [2187-2204] 3′UTR |
| 15 | GGUCCAAGGUCCUCAUCC | 2591 | GGAUGAGGACCUUGGACC | 3626 | | [1149-1166] 3′UTR |
| 16 | GUAUUAGACUUGCACUUU | 2592 | AAAGUGCAAGUCUAAUAC | 3627 | | [2914-2931] 3′UTR |
| 17 | CCUGCAAGCAACUCAAAA | 2593 | UUUUGAGUUGCUUGCAGG | 3628 | | [3343-3360] 3′UTR |
| 18 | GGAGGAAAGAAGGAAUAU | 2594 | AUAUUCCUUCUUUCCUCC | 3629 | Rt | [614-631] ORF |
| 19 | GGAAUAUGAAGUCUGAGA | 2595 | UCUCAGACUUCAUAUUCC | 3630 | Ms | [3508-3525] 3′UTR |
| 20 | GGACGUUGGAGGAAAGAA | 2596 | UUCUUUCCUCCAACGUCC | 3631 | Rt, Ms | [607-624] ORF |
| 21 | CAGUGAGAAGGAAGUGGA | 2597 | UCCACUUCCUUCUCACUG | 3632 | | [451-468] ORF |
| 22 | AGAGGAUCCAGUAUGAGA | 2598 | UCUCAUACUGGAUCCUCU | 3633 | Rh, Rb | [501-518] ORF |
| 23 | GGUCAGUGAGAAGGAAGU | 2599 | ACUUCCUUCUCACUGACC | 3634 | | [448-465] ORF |
| 24 | CUUGGUAGGUAUUAGACU | 2600 | AGUCUAAUACCUACCAAG | 3635 | | [2906-2923] 3′UTR |
| 25 | GGGCAGACUGGGAGGGUA | 2601 | UACCCUCCCAGUCUGCCC | 3636 | Rh | [2619-2636] 3′UTR |
| 26 | CCAGUAUGAGAUCAAGCA | 2602 | UGCUUGAUCUCAUACUGG | 3637 | Rh, Rb, Cw, Dg, Ms | [508-525] ORF |
| 27 | GGGUCUCGCUGGACGUUG | 2603 | CAACGUCCAGCGAGACCC | 3638 | Rt, Ms | [597-614] ORF |
| 28 | GCAGAUAUACCAACUUCU | 2604 | AGAAGUUGGUAUAUCUGC | 3639 | Rh | [2198-2215] 3′UTR |
| 29 | AGACGUGGGUCCAAGGUC | 2605 | GACCUUGGACCCACGUCU | 3640 | | [1142-1159] 3′UTR |
| 30 | CACAGAUCUUGAUGACUU | 2606 | AAGUCAUCAAGAUCUGUG | 3641 | Rh | [2592-2609] 3′UTR |
| 31 | GGAUUGAGUUGCACAGCU | 2607 | AGCUGUGCAACUCAAUCC | 3642 | | [1853-1870] 3′UTR |
| 32 | GGGUCCAAAUUAAUAUGA | 2608 | UCAUAUUAAUUUGGACCC | 3643 | | [1077-1094] 3′UTR |
| 33 | GUGAGAAGGAAGUGGACU | 2609 | AGUCCACUUCCUUCUCAC | 3644 | | [453-470] ORF |
| 34 | ACCUUAGCCUGUUCUAUU | 2610 | AAUAGAACAGGCUAAGGU | 3645 | Rh | [2493-2510] 3′UTR |
| 35 | GGUGCUGGGAACACACAA | 2611 | UUGUGUGUUCCCAGCACC | 3646 | | [3532-3549] 3′UTR |
| 36 | GCAUGUCUCUGAUGCUUU | 2612 | AAAGCAUCAGAGACAUGC | 3647 | | [3579-3596] 3′UTR |
| 37 | GCAAGCAACUCAAAAUAU | 2613 | AUAUUUUGAGUUGCUUGC | 3648 | | [3346-3363] 3′UTR |
| 38 | GGCGUGGUCUUGCAAAAU | 2614 | AUUUUGCAAGACCACGCC | 3649 | | [2463-2480] 3′UTR |
| 39 | CGUCUUUGGUUCUCCAGU | 2615 | ACUGGAGAACCAAAGACG | 3650 | | [3055-3072] 3′UTR |
| 40 | GUUCUCCAGUUCAAAUUA | 2616 | UAAUUUGAACUGGAGAAC | 3651 | | [3063-3080] 3′UTR |
| 41 | AGUAUGAGAUCAAGCAGA | 2617 | UCUGCUUGAUCUCAUACU | 3652 | Rh, Rb, Cw, Dg, Ms | [510-527] ORF |
| 42 | GCCUGUUUUAAGAGACAU | 2618 | AUGUCUCUUAAAACAGGC | 3653 | Rh | [2133-2150] 3′UTR |
| 43 | GGGCCUGAGAAGGAUAUA | 2619 | UAUAUCCUUCUCAGGCCC | 3654 | | [542-559] ORF |
| 44 | GCCUGGAACCAGUGGCUA | 2620 | UAGCCACUGGUUCCAGGC | 3655 | | [1531-1548] 3′UTR |
| 45 | CCAACUUCUGCUUGUAUU | 2621 | AAUACAAGCAGAAGUUGG | 3656 | Rh | [2207-2224] 3′UTR |
| 46 | CGAUAUACAGGCACAUUA | 2622 | UAAUGUGCCUGUAUAUCG | 3657 | | [2403-2420] 3′UTR |
| 47 | GGCCUAUGCAGGUGGAUU | 2623 | AAUCCACCUGCAUAGGCC | 3658 | Rh | [2985-3002] 3′UTR |
| 48 | GGGAGGGUAUCCAGGAAU | 2624 | AUUCCUGGAUACCCUCCC | 3659 | Rh | [2628-2645] 3′UTR |
| 49 | CCUAUUAAUCCUCAGAAU | 2625 | AUUCUGAGGAUUAAUAGG | 3660 | Rh | [1572-1589] 3′UTR |
| 50 | GGGAGACGUGGGUCCAAG | 2626 | CUUGGACCCACGUCUCCC | 3661 | | [1139-1156] 3′UTR |
| 51 | GCUCAAAUACCUUCACAA | 2627 | UUGUGAAGGUAUUUGAGC | 3662 | | [3224-3241] 3′UTR |
| 52 | CCAAGGUCCUCAUCCCAU | 2628 | AUGGGAUGAGGACCUUGG | 3663 | | [1152-1169] 3′UTR |
| 53 | AGUAAGAAGUCCAGCCUA | 2629 | UAGGCUGGACUUCUUACU | 3664 | Rh | [2042-2059] 3′UTR |
| 54 | GGACUGGGUCACAGAGAA | 2630 | UUCUCUGUGACCCAGUCC | 3665 | Rh, Rt, Ms, Pg | [826-843] ORF |
| 55 | AGCUAAGCAUAGUAAGAA | 2631 | UUCUUACUAUGCUUAGCU | 3666 | | [2032-2049] 3′UTR |
| 56 | GGUUUGUUUUUGACAUCA | 2632 | UGAUGUCAAAAACAAACC | 3667 | Rh | [2853-2870] 3′UTR |
| 57 | GGAGUUUCUCGACAUCGA | 2633 | UCGAUGUCGAGAAACUCC | 3668 | Ck, Dg | [937-954] ORF |
| 58 | GAGUCUUUUUGGUCUGCA | 2634 | UGCAGACCAAAAAGACUC | 3669 | | [1667-1684] 3′UTR |
| 59 | AGGAGAAUCUCUUGUUUC | 2635 | GAAACAAGAGAUUCUCCU | 3670 | | [2356-2373] 3′UTR |
| 60 | CGGUAAUGAUAAGGAGAA | 2636 | UUCUCCUUAUCAUUACCG | 3671 | | [2345-2362] 3′UTR |
| 61 | GGUAUUAGACUUGCACUU | 2637 | AAGUGCAAGUCUAAUACC | 3672 | | [2913-2930] 3′UTR |
| 62 | CGGUCAGUGAGAAGGAAG | 2638 | CUUCCUUCUCACUGACCG | 3673 | | [447-464] ORF |
| 63 | GCGGUCAGUGAGAAGGAA | 2639 | UUCCUUCUCACUGACCGC | 3674 | | [446-463] ORF |
| 64 | UCCUGAAGCCAGUGAUAU | 2640 | AUAUCACUGGCUUCAGGA | 3675 | | [2301-2318] 3′UTR |
| 65 | AGAAGAGCCUGAACCACA | 2641 | UGUGGUUCAGGCUCUUCU | 3676 | Rh, Rb, Cw, Ms, Pg | [723-740] ORF |
| 66 | GAAAGAAGGAAUAUCUCA | 2642 | UGAGAUAUUCCUUCUUUC | 3677 | | [618-635] ORF |
| 67 | GCCGUAAUUUAAAGCUCU | 2643 | AGAGCUUUAAAUUACGGC | 3678 | | [3436-3453] 3′UTR |
| 68 | CGUGGACAAUAAACAGUA | 2644 | UACUGUUUAUUGUCCACG | 3679 | | [3624-3641] 3′UTR |
| 69 | GUGAAUUCUCAGAUGAUA | 2645 | UAUCAUCUGAGAAUUCAC | 3680 | | [2166-2183] 3′UTR |
| 70 | GGAACACACAAGAGUUGU | 2646 | ACAACUCUUGUGUGUUCC | 3681 | | [3539-3556] 3′UTR |
| 71 | GAGGAUCCAGUAUGAGAU | 2647 | AUCUCAUACUGGAUCCUC | 3682 | Rh, Rb | [502-519] ORF |
| 72 | UGAGAAGGAUAUAGAGUU | 2648 | AACUCUAUAUCCUUCUCA | 3683 | | [547-564] ORF |
| 73 | GCGUGGUCUUGCAAAAUG | 2649 | CAUUUUGCAAGACCACGC | 3684 | | [2464-2481] 3′UTR |
| 74 | CUGUUUUAAGAGACAUCU | 2650 | AGAUGUCUCUUAAAACAG | 3685 | Rh | [2135-2152] 3′UTR |
| 75 | CCCUCAGUGUGGUUUCCU | 2651 | AGGAAACCACACUGAGGG | 3686 | | [2287-2304] 3′UTR |
| 76 | GAGUUGCAGAUAUACCAA | 2652 | UUGGUAUAUCUGCAACUC | 3687 | Rh | [2193-2210] 3′UTR |
| 77 | CUGGGAACACACAAGAGU | 2653 | ACUCUUGUGUGUUCCCAG | 3688 | | [3536-3553] 3′UTR |
| 78 | GCUGAGUCUUUUUGGUCU | 2654 | AGACCAAAAAGACUCAGC | 3689 | Rh | [1664-1681] 3′UTR |
| 79 | CUGCAUCAAGAGAAGUGA | 2655 | UCACUUCUCUUGAUGCAG | 3690 | Rt, Ms | [877-894] ORF |
| 80 | GAGGAAAGAAGGAAUAUC | 2656 | GAUAUUCCUUCUUUCCUC | 3691 | Rt | [615-632] ORF |
| 81 | GCUGGACGUUGGAGGAAA | 2657 | UUUCCUCCAACGUCCAGC | 3692 | Rt, Ms | [604-621] ORF |
| 82 | GGUGUGGCCUUUAUAUUU | 2658 | AAAUAUAAAGGCCACACC | 3693 | | [3120-3137] 3′UTR |
| 83 | GCAUUUUGCAGAAACUUU | 2659 | AAAGUUUCUGCAAAAUGC | 3694 | Rh | [1334-1351] 3′UTR |
| 84 | GGACCAGUCCAUGUGAUU | 2660 | AAUCACAUGGACUGGUCC | 3695 | Rh | [2710-2727] 3′UTR |
| 85 | CACCUUAGCCUGUUCUAU | 2661 | AUAGAACAGGCUAAGGUG | 3696 | Rh | [2492-2509] 3′UTR |
| 86 | UGAUAAGGAGAAUCUCUU | 2662 | AAGAGAUUCUCCUUAUCA | 3697 | Rh | [2351-2368] 3′UTR |
| 87 | CUCAAAGACUGACAGCCA | 2663 | UGGCUGUCAGUCUUUGAG | 3698 | Rh | [1981-1998] 3′UTR |
| 88 | GGGACAUGGCCCUUGUUU | 2664 | AAACAAGGGCCAUGUCCC | 3699 | | [1407-1424] 3′UTR |
| 89 | CCAUCAAUCCUAUUAAUC | 2665 | GAUUAAUAGGAUUGAUGG | 3700 | Rh | [1564-1581] 3′UTR |
| 90 | GAACCAGUGGCUAGUUCU | 2666 | AGAACUAGCCACUGGUUC | 3701 | | [1536-1553] 3′UTR |
| 91 | AGACAUGGUUGUGGGUCU | 2667 | AGACCCACAACCAUGUCU | 3702 | | [1118-1135] 3′UTR |
| 92 | GUUUAAGAAGGCUCUCCA | 2668 | UGGAGAGCCUUCUUAAAC | 3703 | | [3265-3282] 3′UTR |
| 93 | CUUCCUGUAUGGUGAUAU | 2669 | AUAUCACCAUACAGGAAG | 3704 | | [2786-2803] 3′UTR |
| 94 | GGACCUGGUCAGCACAGA | 2670 | UCUGUGCUGACCAGGUCC | 3705 | Rh | [2580-2597] 3′UTR |
| 95 | GAGACGUGGGUCCAAGGU | 2671 | ACCUUGGACCCACGUCUC | 3706 | | [1141-1158] 3′UTR |
| 96 | GGAACCAGUGGCUAGUUC | 2672 | GAACUAGCCACUGGUUCC | 3707 | | [1535-1552] 3′UTR |
| 97 | GGGCAGCCUGGAACCAGU | 2673 | ACUGGUUCCAGGCUGCCC | 3708 | | [1526-1543] 3′UTR |
| 98 | GCAUCAGGCACCUGGAUU | 2674 | AAUCCAGGUGCCUGAUGC | 3709 | | [1840-1857] 3′UTR |
| 99 | GGCGUUUUGCAAUGCAGA | 2675 | UCUGCAUUGCAAAACGCC | 3710 | Cw, Rt, Ms | [409-426] ORF |
| 100 | CCUCCAACCCAUAUAACA | 2676 | UGUUAUAUGGGUUGGAGG | 3711 | | [2753-2770] 3′UTR |
| 101 | GCCUUUAUAUUUGAUCCA | 2677 | UGGAUCAAAUAUAAAGGC | 3712 | | [3126-3143] 3′UTR |
| 102 | GCACAGAUCUUGAUGACU | 2678 | AGUCAUCAAGAUCUGUGC | 3713 | Rh | [2591-2608] 3′UTR |
| 103 | GUCCCUUUCAUCUUGAGA | 2679 | UCUCAAGAUGAAAGGGAC | 3714 | Rh | [1389-1406] 3′UTR |
| 104 | GGAAGCAUUUGACCCAGA | 2680 | UCUGGGUCAAAUGCUUCC | 3715 | | [2956-2973] 3′UTR |
| 105 | GGCAGACUGGGAGGGUAU | 2681 | AUACCCUCCCAGUCUGCC | 3716 | Rh | [2620-2637] 3′UTR |
| 106 | GCUUUGUAUCAUUCUUGA | 2682 | UCAAGAAUGAUACAAAGC | 3717 | | [3592-3609] 3′UTR |
| 107 | GAGGAAGCCGCUCAAAUA | 2683 | UAUUUGAGCGGCUUCCUC | 3718 | | [3215-3232] 3′UTR |
| 108 | CCUUCUCCUUUUAGACAU | 2684 | AUGUCUAAAAGGAGAAGG | 3719 | | [1106-1123] 3′UTR |
| 109 | GGGCGUGGUCUUGCAAAA | 2685 | UUUUGCAAGACCACGCCC | 3720 | | [2462-2479] 3′UTR |
| 110 | GCUGAGCAGAAAACAAAA | 2686 | UUUUGUUUUCUGCUCAGC | 3721 | | [3166-3183] 3′UTR |
| 111 | GACCAGUCCAUGUGAUUU | 2687 | AAAUCACAUGGACUGGUC | 3722 | Rh | [2711-2728] 3′UTR |
| 112 | GAGAAUCUCUUGUUUCCU | 2688 | AGGAAACAAGAGAUUCUC | 3723 | | [2358-2375] 3′UTR |
| 113 | UCCUAUUAAUCCUCAGAA | 2689 | UUCUGAGGAUUAAUAGGA | 3724 | Rh | [1571-1588] 3′UTR |
| 114 | CACAGAGAAGAACAUCAA | 2690 | UUGAUGUUCUUCUCUGUG | 3725 | Rh | [835-852] ORF |
| 115 | GCCUGCAUCAAGAGAAGU | 2691 | ACUUCUCUUGAUGCAGGC | 3726 | Rt, Ms | [875-892] ORF |
| 116 | GGUGACACACUCACUUCU | 2692 | AGAAGUGAGUGUGUCACC | 3727 | | [2092-2109] 3′UTR |
| 117 | AGGAAAGAAGGAAUAUCU | 2693 | AGAUAUUCCUUCUUUCCU | 3728 | Rt | [616-633] ORF |
| 118 | CCACCUGUGUUGUAAAGA | 2694 | UCUUUACAACACAGGUGG | 3729 | Rh | [2377-2394] 3′UTR |
| 119 | GUCCAUGUGAUUUCAGUA | 2695 | UACUGAAAUCACAUGGAC | 3730 | Rh | [2716-2733] 3′UTR |
| 120 | AGUGGACUCUGGAAACGA | 2696 | UCGUUUCCAGAGUCCACU | 3731 | Rh | [463-480] ORF |
| 121 | GACAUCAGCUGUAAUCAU | 2697 | AUGAUUACAGCUGAUGUC | 3732 | | [2864-2881] 3′UTR |
| 122 | GCCUGUUCUAUUCAGCGG | 2698 | CCGCUGAAUAGAACAGGC | 3733 | | [2499-2516] 3′UTR |
| 123 | AGAUCAAGCAGAUAAAGA | 2699 | UCUUUAUCUGCUUGAUCU | 3734 | Cw, Dg, Rt, Ms | [516-533] ORF |
| 124 | GUCUCUGAUGCUUUGUAU | 2700 | AUACAAAGCAUCAGAGAC | 3735 | | [3583-3600] 3′UTR |
| 125 | GUUCAAAGGGCCUGAGAA | 2701 | UUCUCAGGCCCUUUGAAC | 3736 | | [535-552] ORF |
| 126 | GGGAACUAGGGAACCUAU | 2702 | AUAGGUUCCCUAGUUCCC | 3737 | Rh | [2264-2281] 3′UTR |
| 127 | AUGAUAAGGAGAAUCUCU | 2703 | AGAGAUUCUCCUUAUCAU | 3738 | | [2350-2367] 3′UTR |
| 128 | CUUAGCCUGUUCUAUUCA | 2704 | UGAAUAGAACAGGCUAAG | 3739 | Rh | [2495-2512] 3′UTR |
| 129 | GUAAUGAUAAGGAGAAUC | 2705 | GAUUCUCCUUAUCAUUAC | 3740 | | [2347-2364] 3′UTR |
| 130 | GGACGAGUGCCUCUGGAU | 2706 | AUCCAGAGGCACUCGUCC | 3741 | Rh, Rb, Cw | [808-825] ORF |
| 131 | CACAAUAAAUAGUGGCAA | 2707 | UUGCCACUAUUUAUUGUG | 3742 | | [3237-3254] 3′UTR |
| 132 | GUUGGAGGAAAGAAGGAA | 2708 | UUCCUUCUUUCCUCCAAC | 3743 | Rt | [611-628] ORF |
| 133 | AGGUAUUAGACUUGCACU | 2709 | AGUGCAAGUCUAAUACCU | 3744 | | [2912-2929] 3′UTR |
| 134 | ACACAAGAGUUGUUGAAA | 2710 | UUUCAACAACUCUUGUGU | 3745 | | [3544-3561] 3′UTR |
| 135 | CCUAUGUGUUCCCUCAGU | 2711 | ACUGAGGGAACACAUAGG | 3746 | | [2277-2294] 3′UTR |
| 136 | CAAUGCAGAUGUAGUGAU | 2712 | AUCACUACAUCUGCAUUG | 3747 | | [418-435] ORF |
| 137 | GAAUAUGAAGUCUGAGAC | 2713 | GUCUCAGACUUCAUAUUC | 3748 | | [3509-3526] 3′UTR |
| 138 | CCUCCAAGGGUUUCGACU | 2714 | AGUCGAAACCCUUGGAGG | 3749 | Rh | [1004-1021] 3′UTR |
| 139 | GUGGUUUCCUGAAGCCAG | 2715 | CUGGCUUCAGGAAACCAC | 3750 | | [2295-2312] 3′UTR |
| 140 | UGUUCUAUUCAGCGGCAA | 2716 | UUGCCGCUGAAUAGAACA | 3751 | | [2502-2519] 3′UTR |
| 141 | GAUGAUAGGUGAACCUGA | 2717 | UCAGGUUCACCUAUCAUC | 3752 | | [2177-2194] 3′UTR |
| 142 | GCCUCAGCUGAGUCUUUU | 2718 | AAAAGACUCAGCUGAGGC | 3753 | Rh | [1658-1675] 3′UTR |
| 143 | AGGUGAAUUCUCAGAUGA | 2719 | UCAUCUGAGAAUUCACCU | 3754 | | [2164-2181] 3′UTR |
| 144 | AGGGAGACGUGGGUCCAA | 2720 | UUGGACCCACGUCUCCCU | 3755 | | [1138-1155] 3′UTR |
| 145 | AGAAGGAAGUGGACUCUG | 2721 | CAGAGUCCACUUCCUUCU | 3756 | | [456-473] ORF |
| 146 | GGAAGCCGCUCAAAUACC | 2722 | GGUAUUUGAGCGGCUUCC | 3757 | | [3217-3234] 3′UTR |
| 147 | GCUGUACAGUGACCUAAA | 2723 | UUUAGGUCACUGUACAGC | 3758 | | [2673-2690] 3′UTR |
| 148 | GGAUAGGAAGAACUUUCU | 2724 | AGAAAGUUCUUCCUAUCC | 3759 | | [2327-2344] 3′UTR |
| 149 | CCCUUCUCCUUUUAGACA | 2725 | UGUCUAAAAGGAGAAGGG | 3760 | | [1105-1122] 3′UTR |
| 150 | CCACCUUAGCCUGUUCUA | 2726 | UAGAACAGGCUAAGGUGG | 3761 | Rh | [2491-2508] 3′UTR |
| 151 | GGGCUUCGAUCCUUGGGU | 2727 | ACCCAAGGAUCGAAGCCC | 3762 | Rh | [1198-1215] 3′UTR |
| 152 | AGUGUUCCCUCCCUCAAA | 2728 | UUUGAGGGAGGGAACACU | 3763 | | [1969-1986] 3′UTR |
| 153 | GAACUUUCUCGGUAAUGA | 2729 | UCAUUACCGAGAAAGUUC | 3764 | | [2336-2353] 3′UTR |
| 154 | GAGAAGGAAGUGGACUCU | 2730 | AGAGUCCACUUCCUUCUC | 3765 | | [455-472] ORF |
| 155 | CAUCAAUCCUAUUAAUCC | 2731 | GGAUUAAUAGGAUUGAUG | 3766 | Rh | [1565-1582] 3′UTR |
| 156 | GCAGGAGUUUCUCGACAU | 2732 | AUGUCGAGAAACUCCUGC | 3767 | Ck, Dg | [934-951] ORF |
| 157 | GGGUUAGGAUAGGAAGAA | 2733 | UUCUUCCUAUCCUAACCC | 3768 | | [2321-2338] 3′UTR |
| 158 | CUGAUGCUUUGUAUCAUU | 2734 | AAUGAUACAAAGCAUCAG | 3769 | | [3587-3604] 3′UTR |
| 159 | CAGCCUCAGCUGAGUCUU | 2735 | AAGACUCAGCUGAGGCUG | 3770 | Rh | [1656-1673] 3′UTR |
| 160 | GGCCUGUUUUAAGAGACA | 2736 | UGUCUCUUAAAACAGGCC | 3771 | Rh | [2132-2149] 3′UTR |
| 161 | CAUACACACGCAAUGAAA | 2737 | UUUCAUUGCGUGUGUAUG | 3772 | Rh | [2427-2444] 3′UTR |
| 162 | GCACCACCCAGAAGAAGA | 2738 | UCUUCUUCUGGGUGGUGC | 3773 | Rh, Pg | [711-728] ORF |
| 163 | CUCUGAUGCUUUGUAUCA | 2739 | UGAUACAAAGCAUCAGAG | 3774 | | [3585-3602] 3′UTR |
| 164 | GGGCUUUCUGCAUGUGAC | 2740 | GUCACAUGCAGAAAGCCC | 3775 | | [2011-2028] 3′UTR |
| 165 | CUCUGGAAACGACAUUUA | 2741 | UAAAUGUCGUUUCCAGAG | 3776 | Rh | [469-486] ORF |
| 166 | GAUUGAGUUGCACAGCUU | 2742 | AAGCUGUGCAACUCAAUC | 3777 | | [1854-1871] 3′UTR |
| 167 | GUCAGUGAGAAGGAAGUG | 2743 | CACUUCCUUCUCACUGAC | 3778 | | [449-466] ORF |
| 168 | CCAGCUUGCAGGAGGAAU | 2744 | AUUCCUCCUGCAAGCUGG | 3779 | | [1723-1740] 3′UTR |
| 169 | CCCUGUUCGCUUCCUGUA | 2745 | UACAGGAAGCGAACAGGG | 3780 | | [2777-2794] 3′UTR |
| 170 | CAUGGGUCCAAAUUAAUA | 2746 | UAUUAAUUUGGACCCAUG | 3781 | | [1074-1091] 3′UTR |
| 171 | CUCUGUUGAUUUUGUUUC | 2747 | GAAACAAAAUCAACAGAG | 3782 | | [3450-3467] 3′UTR |
| 172 | GGAAGGAUUUUGGAGGUA | 2748 | UACCUCCAAAAUCCUUCC | 3783 | Rh | [2065-2082] 3′UTR |
| 173 | GGAACUAGGGAACCUAUG | 2749 | CAUAGGUUCCCUAGUUCC | 3784 | Rh | [2265-2282] 3′UTR |
| 174 | CCUGGAUUGAGUUGCACA | 2750 | UGUGCAACUCAAUCCAGG | 3785 | | [1850-1867] 3′UTR |
| 175 | GCCUGGAAAUGUGCAUUU | 2751 | AAAUGCACAUUUCCAGGC | 3786 | Rh | [1322-1339] 3′UTR |
| 176 | GGCCAAAGCGGUCAGUGA | 2752 | UCACUGACCGCUUUGGCC | 3787 | | [439-456] ORF |
| 177 | ACUUCUGCUUGUAUUUCU | 2753 | AGAAAUACAAGCAGAAGU | 3788 | Rh | [2210-2227] 3′UTR |
| 178 | CCCUUUCUAGGGCAGACU | 2754 | AGUCUGCCCUAGAAAGGG | 3789 | Rh | [2610-2627] 3′UTR |
| 179 | CCUGGUCAGCACAGAUCU | 2755 | AGAUCUGUGCUGACCAGG | 3790 | Rh | [2583-2600] 3′UTR |
| 180 | GGGUCCAAGGUCCUCAUC | 2756 | GAUGAGGACCUUGGACCC | 3791 | | [1148-1165] 3′UTR |
| 181 | CUGUAUGGUGAUAUCAUA | 2757 | UAUGAUAUCACCAUACAG | 3792 | | [2790-2807] 3′UTR |
| 182 | UGGACUUGCUGCCGUAAU | 2758 | AUUACGGCAGCAAGUCCA | 3793 | | [3426-3443] 3′UTR |
| 183 | CCUGUUCGCUUCCUGUAU | 2759 | AUACAGGAAGCGAACAGG | 3794 | | [2778-2795] 3′UTR |
| 184 | GUGACACACUCACUUCUU | 2760 | AAGAAGUGAGUGUGUCAC | 3795 | | [2093-2110] 3′UTR |
| 185 | GGUGGAUUCCUUCAGGUC | 2761 | GACCUGAAGGAAUCCACC | 3796 | Rh | [2995-3012] 3′UTR |
| 186 | CCCAUCAAUCCUAUUAAU | 2762 | AUUAAUAGGAUUGAUGGG | 3797 | Rh | [1563-1580] 3′UTR |
| 187 | GCUAGUUCUUGAAGGAGC | 2763 | GCUCCUUCAAGAACUAGC | 3798 | | [1545-1562] 3′UTR |
| 188 | GUGGGUCCAAGGUCCUCA | 2764 | UGAGGACCUUGGACCCAC | 3799 | | [1146-1163] 3′UTR |
| 189 | GCUUCCAAAGCCACCUUA | 2765 | UAAGGUGGCUUUGGAAGC | 3800 | Rh | [2481-2498] 3′UTR |
| 190 | GGUCACAGAGAAGAACAU | 2766 | AUGUUCUUCUCUGUGACC | 3801 | Rh | [832-849] ORF |
| 191 | GCAUCAAGAGAAGUGACG | 2767 | CGUCACUUCUCUUGAUGC | 3802 | | [879-896] ORF |
| 192 | UGGUCUUGCAAAAUGCUU | 2768 | AAGCAUUUUGCAAGACCA | 3803 | | [2467-2484] 3′UTR |
| 193 | AGCAGGAGUUUCUCGACA | 2769 | UGUCGAGAAACUCCUGCU | 3804 | Ck, Dg | [933-950] ORF |
| 194 | GUUGAAAGUUGACAAGCA | 2770 | UGCUUGUCAACUUUCAAC | 3805 | | [3555-3572] 3′UTR |
| 195 | GGUCUUGCAAAAUGCUUC | 2771 | GAAGCAUUUUGCAAGACC | 3806 | | [2468-2485] 3′UTR |
| 196 | GUGUUUAUGCUGGAAUAU | 2772 | AUAUUCCAGCAUAAACAC | 3807 | | [3497-3514] 3′UTR |
| 197 | GCAGAAAACAAAACAGGU | 2773 | ACCUGUUUUGUUUUCUGC | 3808 | | [3171-3188] 3′UTR |
| 198 | ACCUAAAGUUGGUAAGAU | 2774 | AUCUUACCAACUUUAGGU | 3809 | | [2684-2701] 3′UTR |
| 199 | AGCAGACUGCGCAUGUCU | 2775 | AGACAUGCGCAGUCUGCU | 3810 | | [3569-3586] 3′UTR |
| 200 | AGCCUGUUCUAUUCAGCG | 2776 | CGCUGAAUAGAACAGGCU | 3811 | | [2498-2515] 3′UTR |
| 201 | GGCAGCACUUAGGGAUCU | 2777 | AGAUCCCUAAGUGCUGCC | 3812 | Rh | [1284-1301] 3′UTR |
| 202 | CAUUUAUGGCAACCCUAU | 2778 | AUAGGGUUGCCAUAAAUG | 3813 | | [481-498] ORF |
| 203 | GGCUCUCCAUUUGGCAUC | 2779 | GAUGCCAAAUGGAGAGCC | 3814 | | [3274-3291] 3′UTR |
| 204 | UGUUUCUGCUGAUUGUUU | 2780 | AAACAAUCAGCAGAAACA | 3815 | | [2823-2840] 3′UTR |
| 205 | GCUGCGAGUGCAAGAUCA | 2781 | UGAUCUUGCACUCGCAGC | 3816 | Rt | [753-770] ORF |
| 206 | CUUUCUCGGUAAUGAUAA | 2782 | UUAUCAUUACCGAGAAAG | 3817 | | [2339-2356] 3′UTR |
| 207 | GUUUCCGUUUGGAUUUUU | 2783 | AAAAAUCCAAACGGAAAC | 3818 | | [3463-3480] 3′UTR |
| 208 | GGGCUGCGAGUGCAAGAU | 2784 | AUCUUGCACUCGCAGCCC | 3819 | Ck, Rb, Rt | [751-768] ORF |
| 209 | CCUGAGUUGCAGAUAUAC | 2785 | GUAUAUCUGCAACUCAGG | 3820 | Rh | [2190-2207] 3′UTR |
| 210 | GUUUCCUGAAGCCAGUGA | 2786 | UCACUGGCUUCAGGAAAC | 3821 | | [2298-2315] 3′UTR |
| 211 | GGAUUUUGGAGGUAGGUG | 2787 | CACCUACCUCCAAAAUCC | 3822 | Rh | [2069-2086] 3′UTR |
| 212 | GCAAAAAAAGCCUCCAAG | 2788 | CUUGGAGGCUUUUUUUGC | 3823 | | [994-1011] 3′UTR |
| 213 | CCAAGUUCUUCGCCUGCA | 2789 | UGCAGGCGAAGAACUUGG | 3824 | Rh, Rb, Cw, Dg, Ms | [864-881] ORF |
| 214 | GUUCCCUCAGUGUGGUUU | 2790 | AAACCACACUGAGGGAAC | 3825 | | [2284-2301] 3′UTR |
| 215 | GGAGCACUGUGUUUAUGC | 2791 | GCAUAAACACAGUGCUCC | 3826 | | [3489-3506] 3′UTR |
| 216 | UAAGAAGGCUCUCCAUUU | 2792 | AAAUGGAGAGCCUUCUUA | 3827 | | [3268-3285] 3′UTR |
| 217 | GCGUUUUCAUGCUGUACA | 2793 | UGUACAGCAUGAAAACGC | 3828 | Rh | [2663-2680] 3′UTR |
| 218 | GGUUCGGUCUGAAAGGUG | 2794 | CACCUUUCAGACCGAACC | 3829 | | [3106-3123] 3′UTR |
| 219 | GAUUACCUAGCUAAGAAA | 2795 | UUUCUUAGCUAGGUAAUC | 3830 | | [2239-2256] 3′UTR |
| 220 | CUUUCAUCUUGAGAGGGA | 2796 | UCCCUCUCAAGAUGAAAG | 3831 | | [1393-1410] 3′UTR |
| 221 | AGGGCAGCCUGGAACCAG | 2797 | CUGGUUCCAGGCUGCCCU | 3832 | | [1525-1542] 3′UTR |
| 222 | GGGACACGCGGCUUCCCU | 2798 | AGGGAAGCCGCGUGUCCC | 3833 | | [1228-1245] 3′UTR |
| 223 | CCUAGGAAGGGAAGGAUU | 2799 | AAUCCUUCCCUUCCUAGG | 3834 | Rh | [2056-2073] 3′UTR |
| 224 | CCAAGGGCAGCCUGGAAC | 2800 | GUUCCAGGCUGCCCUUGG | 3835 | | [1522-1539] 3′UTR |
| 225 | AUAUGAAGUCUGAGACCU | 2801 | AGGUCUCAGACUUCAUAU | 3836 | | [3511-3528] 3′UTR |
| 226 | GGACUCUGGAAACGACAU | 2802 | AUGUCGUUUCCAGAGUCC | 3837 | Rh | [466-483] ORF |
| 227 | CCUGAAGCCAGUGAUAUG | 2803 | CAUAUCACUGGCUUCAGG | 3838 | | [2302-2319] 3′UTR |
| 228 | GGACUUGCUGCCGUAAUU | 2804 | AAUUACGGCAGCAAGUCC | 3839 | | [3427-3444] 3′UTR |
| 229 | ACGGCAAGAUGCACAUCA | 2805 | UGAUGUGCAUCUUGCCGU | 3840 | Dg, Pg | [657-674] ORF |
| 230 | CAAGAGUUGUUGAAAGUU | 2806 | AACUUUCAACAACUCUUG | 3841 | | [3547-3564] 3′UTR |
| 231 | GGUCAGCACAGAUCUUGA | 2807 | UCAAGAUCUGUGCUGACC | 3842 | Rh | [2586-2603] 3′UTR |
| 232 | AGGGAACUAGGGAACCUA | 2808 | UAGGUUCCCUAGUUCCCU | 3843 | Rh | [2263-2280] 3′UTR |
| 233 | CGGACGAGUGCCUCUGGA | 2809 | UCCAGAGGCACUCGUCCG | 3844 | Rh, Rb, Cw | [807-824] ORF |
| 234 | CUCCAGUUCAAAUUAUUG | 2810 | CAAUAAUUUGAACUGGAG | 3845 | | [3066-3083] 3′UTR |
| 235 | CAUGGUUGUGGGUCUGGA | 2811 | UCCAGACCCACAACCAUG | 3846 | | [1121-1138] 3′UTR |
| 236 | AGGUGAACCUGAGUUGCA | 2812 | UGCAACUCAGGUUCACCU | 3847 | Rh | [2183-2200] 3′UTR |
| 237 | GGUGAGGUCCUGUCCUGA | 2813 | UCAGGACAGGACCUCACC | 3848 | Rh | [1742-1759] 3′UTR |
| 238 | CAGUGUGGUUUCCUGAAG | 2814 | CUUCAGGAAACCACACUG | 3849 | | [2291-2308] 3′UTR |
| 239 | GAAGAAGAGCCUGAACCA | 2815 | UGGUUCAGGCUCUUCUUC | 3850 | Rh, Rb, Cw, Ms, Pg | [721-738] ORF |
| 240 | GGUAGGUGGCUUUGGUGA | 2816 | UCACCAAAGCCACCUACC | 3851 | Rh | [2079-2096] 3′UTR |
| 241 | CCCUCAAGGUCCCUUCCC | 2817 | GGGAAGGGACCUUGAGGG | 3852 | | [1785-1802] 3′UTR |
| 242 | UCGCCUGCAUCAAGAGAA | 2818 | UUCUCUUGAUGCAGGCGA | 3853 | Rh, Cw, Dg, Ms | [873-890] ORF |
| 243 | AGCAUUUGACCCAGAGUG | 2819 | CACUCUGGGUCAAAUGCU | 3854 | | [2959-2976] 3′UTR |
| 244 | GGGUCUUGCUGUGCCCUC | 2820 | GAGGGCACAGCAAGACCC | 3855 | Rh | [1943-1960] 3′UTR |
| 245 | GCCUCCUGCUGCUGGCGA | 2821 | UCGCCAGCAGCAGGAGGC | 3856 | Dg | [339-356] ORF |
| 246 | CAGGCUUAGUGUUCCCUC | 2822 | GAGGGAACACUAAGCCUG | 3857 | | [1962-1979] 3′UTR |
| 247 | CCAGAAGAAGAGCCUGAA | 2823 | UUCAGGCUCUUCUUCUGG | 3858 | Rh, Rb, Cw, Ms, Pg | [718-735] ORF |
| 248 | GACCCAGAGUGGAACGCG | 2824 | CGCGUUCCACUCUGGGUC | 3859 | | [2966-2983] 3′UTR |
| 249 | AGGUGUGGCCUUUAUAUU | 2825 | AAUAUAAAGGCCACACCU | 3860 | | [3119-3136] 3′UTR |
| 250 | CAGUGGGAGCCUCCCUCU | 2826 | AGAGGGAGGCUCCCACUG | 3861 | Rh | [1592-1609] 3′UTR |
| 251 | UGGUUCUCCAGUUCAAAU | 2827 | AUUUGAACUGGAGAACCA | 3862 | | [3061-3078] 3′UTR |
| 252 | GGUUAAGAAGAGCCGGGU | 2828 | ACCCGGCUCUUCUUAACC | 3863 | | [3186-3203] 3′UTR |
| 253 | AAGGAGAAUCUCUUGUUU | 2829 | AAACAAGAGAUUCUCCUU | 3864 | | [2355-2372] 3′UTR |
| 254 | UCUGAUGCUUUGUAUCAU | 2830 | AUGAUACAAAGCAUCAGA | 3865 | | [3586-3603] 3′UTR |
| 255 | CCAGGUCCCUUUCAUCUU | 2831 | AAGAUGAAAGGGACCUGG | 3866 | Rh | [1385-1402] 3′UTR |
| 256 | CACCCUCUGUGACUUCAU | 2832 | AUGAAGUCACAGAGGGUG | 3867 | Rh, Cw, Ms | [673-690] ORF |
| 257 | CCCUUGGUAGGUAUUAGA | 2833 | UCUAAUACCUACCAAGGG | 3868 | | [2904-2921] 3′UTR |
| 258 | CUAUGUGUUCCCUCAGUG | 2834 | CACUGAGGGAACACAUAG | 3869 | | [2278-2295] 3′UTR |
| 259 | GAGCCUGAACCACAGGUA | 2835 | UACCUGUGGUUCAGGCUC | 3870 | Rh, Rb, Cw, Ms, Pg | [727-744] ORF |
| 260 | GGCAAGUGCUCCCAUCGC | 2836 | GCGAUGGGAGCACUUGCC | 3871 | | [1460-1477] 3′UTR |
| 261 | GAAUUCCAGUGGGAGCCU | 2837 | AGGCUCCCACUGGAAUUC | 3872 | Rh | [1586-1603] 3′UTR |
| 262 | GCAAUGCAGAUGUAGUGA | 2838 | UCACUACAUCUGCAUUGC | 3873 | | [417-434] ORF |
| 263 | CCUGAGAAGGAUAUAGAG | 2839 | CUCUAUAUCCUUCUCAGG | 3874 | | [545-562] ORF |
| 264 | ACCUGAGUUGCAGAUAUA | 2840 | UAUAUCUGCAACUCAGGU | 3875 | Rh | [2189-2206] 3′UTR |
| 265 | GACCUAAAGUUGGUAAGA | 2841 | UCUUACCAACUUUAGGUC | 3876 | | [2683-2700] 3′UTR |
| 266 | GUCUUUGGUUCUCCAGUU | 2842 | AACUGGAGAACCAAAGAC | 3877 | | [3056-3073] 3′UTR |
| 267 | GCCUAGGAAGGGAAGGAU | 2843 | AUCCUUCCCUUCCUAGGC | 3878 | Rh | [2055-2072] 3′UTR |
| 268 | CGCAUGUCUCUGAUGCUU | 2844 | AAGCAUCAGAGACAUGCG | 3879 | | [3578-3595] 3′UTR |
| 269 | CAAUCCUAUUAAUCCUCA | 2845 | UGAGGAUUAAUAGGAUUG | 3880 | Rh | [1568-1585] 3′UTR |
| 270 | AGCCUCAGCUGAGUCUUU | 2846 | AAAGACUCAGCUGAGGCU | 3881 | Rh | [1657-1674] 3′UTR |
| 271 | GCUCUGUUGAUUUUGUUU | 2847 | AAACAAAAUCAACAGAGC | 3882 | | [3449-3466] 3′UTR |
| 272 | GUGGGAGCCUCCCUCUGA | 2848 | UCAGAGGGAGGCUCCCAC | 3883 | Rh | [1594-1611] 3′UTR |
| 273 | GACUCUGGAAACGACAUU | 2849 | AAUGUCGUUUCCAGAGUC | 3884 | Rh | [467-484] ORF |
| 274 | AGCAUAGUAAGAAGUCCA | 2850 | UGGACUUCUUACUAUGCU | 3885 | | [2037-2054] 3′UTR |
| 275 | CAGAGGAAGCCGCUCAAA | 2851 | UUUGAGCGGCUUCCUCUG | 3886 | | [3213-3230] 3′UTR |
| 276 | GUUGGUAAGAUGUCAUAA | 2852 | UUAUGACAUCUUACCAAC | 3887 | Rh | [2691-2708] 3′UTR |
| 277 | CUAUUUUCAUCCUGCAAG | 2853 | CUUGCAGGAUGAAAAUAG | 3888 | | [3333-3350] 3′UTR |
| 278 | AGUUGCAGAUAUACCAAC | 2854 | GUUGGUAUAUCUGCAACU | 3889 | Rh | [2194-2211] 3′UTR |
| 279 | AAUGAUAAGGAGAAUCUC | 2855 | GAGAUUCUCCUUAUCAUU | 3890 | | [2349-2366] 3′UTR |
| 280 | GGAGAAUCUCUUGUUUCC | 2856 | GGAAACAAGAGAUUCUCC | 3891 | | [2357-2374] 3′UTR |
| 281 | GUAAGAUGUCAUAAUGGA | 2857 | UCCAUUAUGACAUCUUAC | 3892 | Rh | [2695-2712] 3′UTR |
| 282 | CCUUGGUAGGUAUUAGAC | 2858 | GUCUAAUACCUACCAAGG | 3893 | | [2905-2922] 3′UTR |
| 283 | GGCUGGGACACGCGGCUU | 2859 | AAGCCGCGUGUCCCAGCC | 3894 | | [1224-1241] 3′UTR |
| 284 | CACAAGAGUUGUUGAAAG | 2860 | CUUUCAACAACUCUUGUG | 3895 | | [3545-3562] 3′UTR |
| 285 | GAGGGUCGUUGCAAGACU | 2861 | AGUCUUGCAACGACCCUC | 3896 | | [1353-1370] 3′UTR |
| 286 | AAACGACAUUUAUGGCAA | 2862 | UUGCCAUAAAUGUCGUUU | 3897 | | [475-492] ORF |
| 287 | UGAUGACUUCCCUUUCUA | 2863 | UAGAAAGGGAAGUCAUCA | 3898 | Rh | [2601-2618] 3′UTR |
| 288 | GGCUUAGUGUUCCCUCCC | 2864 | GGGAGGGAACACUAAGCC | 3899 | | [1964-1981] 3′UTR |
| 289 | CAGAGAAGAACAUCAACG | 2865 | CGUUGAUGUUCUUCUCUG | 3900 | Rh | [837-854] ORF |
| 290 | CCAGCCUCAGCUGAGUCU | 2866 | AGACUCAGCUGAGGCUGG | 3901 | Rh | [1655-1672] 3′UTR |
| 291 | GGGACACCCUGAGCACCA | 2867 | UGGUGCUCAGGGUGUCCC | 3902 | Rh, Pg | [699-716] ORF |
| 292 | CUCACUUCUUUCUCAGCC | 2868 | GGCUGAGAAAGAAGUGAG | 3903 | | [2101-2118] 3′UTR |
| 293 | GACAUCCCUUCCUGGAAA | 2869 | UUUCCAGGAAGGGAUGUC | 3904 | Rh, Rt, Ms | [1033-1050] 3′UTR |
| 294 | CCUGGCAAGUGCUCCCAU | 2870 | AUGGGAGCACUUGCCAGG | 3905 | | [1457-1474] 3′UTR |
| 295 | AGAAAUGGGAGCGAGAAA | 2871 | UUUCUCGCUCCCAUUUCU | 3906 | | [1619-1636] 3′UTR |
| 296 | GCAAUGAAACCGAAGCUU | 2872 | AAGCUUCGGUUUCAUUGC | 3907 | | [2436-2453] 3′UTR |
| 297 | AUGUCAUAAUGGACCAGU | 2873 | ACUGGUCCAUUAUGACAU | 3908 | Rh | [2700-2717] 3′UTR |
| 298 | UGUGGUUUCCUGAAGCCA | 2874 | UGGCUUCAGGAAACCACA | 3909 | | [2294-2311] 3′UTR |
| 299 | GAUAUACAGGCACAUUAU | 2875 | AUAAUGUGCCUGUAUAUC | 3910 | | [2404-2421] 3′UTR |
| 300 | AAAAAAGCCUCCAAGGGU | 2876 | ACCCUUGGAGGCUUUUUU | 3911 | | [997-1014] 3′UTR |
| 301 | GUAUGGUGAUAUCAUAUG | 2877 | CAUAUGAUAUCACCAUAC | 3912 | | [2792-2809] 3′UTR |
| 302 | CCUGUGCUGUGUUUUUUA | 2878 | UAAAAAACACAGCACAGG | 3913 | Rh | [2883-2900] 3′UTR |
| 303 | AGGCCAAGUUCUUCGCCU | 2879 | AGGCGAAGAACUUGGCCU | 3914 | Rh, Rb, Cw, Dg, Ms | [861-878] ORF |
| 304 | UGCAGAUAUACCAACUUC | 2880 | GAAGUUGGUAUAUCUGCA | 3915 | Rh | [2197-2214] 3′UTR |
| 305 | AGAAGGAAUAUCUCAUUG | 2881 | CAAUGAGAUAUUCCUUCU | 3916 | | [621-638] ORF |
| 306 | GAAGGAUUUUGGAGGUAG | 2882 | CUACCUCCAAAAUCCUUC | 3917 | Rh | [2066-2083] 3′UTR |
| 307 | GCGGCUUCCCUCCCAGUC | 2883 | GACUGGGAGGGAAGCCGC | 3918 | | [1235-1252] 3′UTR |
| 308 | CUCAGAAUUCCAGUGGGA | 2884 | UCCCACUGGAAUUCUGAG | 3919 | Rh | [1582-1599] 3′UTR |
| 309 | GAUGGACUGGGUCACAGA | 2885 | UCUGUGACCCAGUCCAUC | 3920 | Rh, Rt, Ms, Pg | [823-840] ORF |
| 310 | AUAUCUCAUUGCAGGAAA | 2886 | UUUCCUGCAAUGAGAUAU | 3921 | | [628-645] ORF |
| 311 | ACAUUUAUGGCAACCCUA | 2887 | UAGGGUUGCCAUAAAUGU | 3922 | | [480-497] ORF |
| 312 | UUAAGAAGGCUCUCCAUU | 2888 | AAUGGAGAGCCUUCUUAA | 3923 | | [3267-3284] 3′UTR |
| 313 | GCAAAAUGCUUCCAAAGC | 2889 | GCUUUGGAAGCAUUUUGC | 3924 | Rh | [2474-2491] 3′UTR |
| 314 | CUCCCUCAAAGACUGACA | 2890 | UGUCAGUCUUUGAGGGAG | 3925 | Rh | [1977-1994] 3′UTR |
| 315 | GCCUCUGGAUGGACUGGG | 2891 | CCCAGUCCAUCCAGAGGC | 3926 | Rh, Rb, Cw, Dg, Rt, Ms | [816-833] ORF |
| 316 | CGUUGGUCUUUUAACCGU | 2892 | ACGGUUAAAAGACCAACG | 3927 | | [3148-3165] 3′UTR |
| 317 | AGGAAUAUCUCAUUGCAG | 2893 | CUGCAAUGAGAUAUUCCU | 3928 | | [624-641] ORF |
| 318 | GAGUUUAUCUACACGGCC | 2894 | GGCCGUGUAGAUAAACUC | 3929 | Ms, Pg | [560-577] ORF |
| 319 | UUUUCAUCCUGCAAGCAA | 2895 | UUGCUUGCAGGAUGAAAA | 3930 | | [3336-3353] 3′UTR |
| 320 | CAAAGCGGUCAGUGAGAA | 2896 | UUCUCACUGACCGCUUUG | 3931 | | [442-459] ORF |
| 321 | GUUUCUGCUGAUUGUUUU | 2897 | AAAACAAUCAGCAGAAAC | 3932 | | [2824-2841] 3′UTR |
| 322 | AAAGGUGAAUUCUCAGAU | 2898 | AUCUGAGAAUUCACCUUU | 3933 | | [2162-2179] 3′UTR |
| 323 | GAGUGCCUCUGGAUGGAC | 2899 | GUCCAUCCAGAGGCACUC | 3934 | Rh, Rb, Cw, Dg, Rt, Ms | [812-829] ORF |
| 324 | CAAAGAUUACCUAGCUAA | 2900 | UUAGCUAGGUAAUCUUUG | 3935 | | [2235-2252] 3′UTR |
| 325 | CCAGCUCUGACAUCCCUU | 2901 | AAGGGAUGUCAGAGCUGG | 3936 | Rh | [1025-1042] 3′UTR |
| 326 | GUUCUUCGCCUGCAUCAA | 2902 | UUGAUGCAGGCGAAGAAC | 3937 | Rh, Rb, Cw, Dg, Ms | [868-885] ORF |
| 327 | GGCUCCUGUGCGUGGUAC | 2903 | GUACCACGCACAGGAGCC | 3938 | Rh | [896-913] ORF |
| 328 | UAGACAUGGUUGUGGGUC | 2904 | GACCCACAACCAUGUCUA | 3939 | | [1117-1134] 3′UTR |
| 329 | AGAAGUCCAGCCUAGGAA | 2905 | UUCCUAGGCUGGACUUCU | 3940 | Rh | [2046-2063] 3′UTR |
| 330 | GCAAGACUGUGUAGCAGG | 2906 | CCUGCUACACAGUCUUGC | 3941 | Rh | [1363-1380] 3′UTR |
| 331 | GCUCUCUUCUCCUAUUUU | 2907 | AAAAUAGGAGAAGAGAGC | 3942 | | [3322-3339] 3′UTR |
| 332 | GGCAAGAUGCACAUCACC | 2908 | GGUGAUGUGCAUCUUGCC | 3943 | Rh, Dg | [659-676] ORF |
| 333 | GAAGAACUUUCUCGGUAA | 2909 | UUACCGAGAAAGUUCUUC | 3944 | Rh | [2333-2350] 3′UTR |
| 334 | AGAGUUGUUGAAAGUUGA | 2910 | UCAACUUUCAACAACUCU | 3945 | | [3549-3566] 3′UTR |
| 335 | GUAUAUACAACUCCACCA | 2911 | UGGUGGAGUUGUAUAUAC | 3946 | Rh | [2731-2748] 3′UTR |
| 336 | GCUUAGUGUUCCCUCCCU | 2912 | AGGGAGGGAACACUAAGC | 3947 | | [1965-1982] 3′UTR |
| 337 | GCUCUGACAUCCCUUCCU | 2913 | AGGAAGGGAUGUCAGAGC | 3948 | Rh | [1028-1045] 3′UTR |
| 338 | CCCAUGGGUCCAAAUUAA | 2914 | UUAAUUUGGACCCAUGGG | 3949 | | [1072-1089] 3′UTR |
| 339 | UGGCCAACUGCAAAAAAA | 2915 | UUUUUUUGCAGUUGGCCA | 3950 | | [985-1002] 3′UTR |
| 340 | GGACACUAUGGCCUGUUU | 2916 | AAACAGGCCAUAGUGUCC | 3951 | | [2123-2140] 3′UTR |
| 341 | GCCAGCUAAGCAUAGUAA | 2917 | UUACUAUGCUUAGCUGGC | 3952 | | [2029-2046] 3′UTR |
| 342 | CCAAGGGUUUCGACUGGU | 2918 | ACCAGUCGAAACCCUUGG | 3953 | Rh | [1007-1024] 3′UTR |
| 343 | AGAUGCACAUCACCCUCU | 2919 | AGAGGGUGAUGUGCAUCU | 3954 | Rh | [663-680] ORF |
| 344 | GGCAGGGCCUGGAAAUGU | 2920 | ACAUUUCCAGGCCCUGCC | 3955 | | [1316-1333] 3′UTR |
| 345 | GGUCCUCAUCCCAUCCUC | 2921 | GAGGAUGGGAUGAGGACC | 3956 | Rh | [1156-1173] 3′UTR |
| 346 | CGACAUUUAUGGCAACCC | 2922 | GGGUUGCCAUAAAUGUCG | 3957 | | [478-495] ORF |
| 347 | GCCUUGUAGAAAUGGGAG | 2923 | CUCCCAUUUCUACAAGGC | 3958 | Rh | [1612-1629] 3′UTR |
| 348 | CAGUCCAUGUGAUUUCAG | 2924 | CUGAAAUCACAUGGACUG | 3959 | Rh | [2714-2731] 3′UTR |
| 349 | GGAGACGUGGGUCCAAGG | 2925 | CCUUGGACCCACGUCUCC | 3960 | | [1140-1157] 3′UTR |
| 350 | CAGCUUUGCUUUAUCCGG | 2926 | CCGGAUAAAGCAAAGCUG | 3961 | | [1866-1883] 3′UTR |
| 351 | UGCAAGCAACUCAAAAUA | 2927 | UAUUUUGAGUUGCUUGCA | 3962 | | [3345-3362] 3′UTR |
| 352 | CCUUUCUAGGGCAGACUG | 2928 | CAGUCUGCCCUAGAAAGG | 3963 | Rh | [2611-2628] 3′UTR |
| 353 | CCUGGAAAUGUGCAUUUU | 2929 | AAAAUGCACAUUUCCAGG | 3964 | Rh | [1323-1340] 3′UTR |
| 354 | AUGGCAACCCUAUCAAGA | 2930 | UCUUGAUAGGGUUGCCAU | 3965 | | [486-503] ORF |
| 355 | GCCAUUGCUUCUUGCCUG | 2931 | CAGGCAAGAAGCAAUGGC | 3966 | | [1817-1834] 3′UTR |
| 356 | GGAACCUAUGUGUUCCCU | 2932 | AGGGAACACAUAGGUUCC | 3967 | Rh | [2273-2290] 3′UTR |
| 357 | GAUAUACCAACUUCUGCU | 2933 | AGCAGAAGUUGGUAUAUC | 3968 | Rh | [2201-2218] 3′UTR |
| 358 | GUUUGUUUUUGACAUCAG | 2934 | CUGAUGUCAAAAACAAAC | 3969 | | [2854-2871] 3′UTR |
| 359 | UGCACAGCUUUGCUUUAU | 2935 | AUAAAGCAAAGCUGUGCA | 3970 | | [1862-1879] 3′UTR |
| 360 | CCUAUUUUCAUCCUGCAA | 2936 | UUGCAGGAUGAAAAUAGG | 3971 | | [3332-3349] 3′UTR |
| 361 | UGCCAUUGCUUCUUGCCU | 2937 | AGGCAAGAAGCAAUGGCA | 3972 | | [1816-1833] 3′UTR |
| 362 | ACCAACUUCUGCUUGUAU | 2938 | AUACAAGCAGAAGUUGGU | 3973 | Rh | [2206-2223] 3′UTR |
| 363 | GGUCCUGUCCUGAGGCUG | 2939 | CAGCCUCAGGACAGGACC | 3974 | Rh | [1747-1764] 3′UTR |
| 364 | AUGCAGAUGUAGUGAUCA | 2940 | UGAUCACUACAUCUGCAU | 3975 | | [420-437] ORF |
| 365 | GCUAAGCAUAGUAAGAAG | 2941 | CUUCUUACUAUGCUUAGC | 3976 | | [2033-2050] 3′UTR |
| 366 | GUUCCCUCCCUCAAAGAC | 2942 | GUCUUUGAGGGAGGGAAC | 3977 | | [1972-1989] 3′UTR |
| 367 | AGCUGUAAUCAUUCCUGU | 2943 | ACAGGAAUGAUUACAGCU | 3978 | | [2870-2887] 3′UTR |
| 368 | GCAUGUGACGCCAGCUAA | 2944 | UUAGCUGGCGUCACAUGC | 3979 | | [2020-2037] 3′UTR |
| 369 | GCACAGCUUUGCUUUAUC | 2945 | GAUAAAGCAAAGCUGUGC | 3980 | | [1863-1880] 3′UTR |
| 370 | GAGCCUCCCUCUGAGCCU | 2946 | AGGCUCAGAGGGAGGCUC | 3981 | Rh | [1598-1615] 3′UTR |
| 371 | GGCACCAGGCCAAGUUCU | 2947 | AGAACUUGGCCUGGUGCC | 3982 | Rh, Rb, Rt, Ms | [855-872] ORF |
| 372 | CAGCACAGAUCUUGAUGA | 2948 | UCAUCAAGAUCUGUGCUG | 3983 | Rh | [2589-2606] 3′UTR |
| 373 | UGUUCUAAGCACAGCUCU | 2949 | AGAGCUGUGCUUAGAACA | 3984 | | [3309-3326] 3′UTR |
| 374 | UGAGCAGAAAACAAAACA | 2950 | UGUUUUGUUUUCUGCUCA | 3985 | | [3168-3185] 3′UTR |
| 375 | GGGAACACACAAGAGUUG | 2951 | CAACUCUUGUGUGUUCCC | 3986 | | [3538-3555] 3′UTR |
| 376 | CCCUCAAAGACUGACAGC | 2952 | GCUGUCAGUCUUUGAGGG | 3987 | Rh | [1979-1996] 3′UTR |
| 377 | CCUUGUUUUCUGCAGCUU | 2953 | AAGCUGCAGAAAACAAGG | 3988 | Rh | [1417-1434] 3′UTR |
| 378 | CUGGAAACGACAUUUAUG | 2954 | CAUAAAUGUCGUUUCCAG | 3989 | | [471-488] ORF |
| 379 | GCAAGAUGCACAUCACCC | 2955 | GGGUGAUGUGCAUCUUGC | 3990 | Rh, Dg | [660-677] ORF |
| 380 | UCAGAAUUCCAGUGGGAG | 2956 | CUCCCACUGGAAUUCUGA | 3991 | Rh | [1583-1600] 3′UTR |
| 381 | UGUUGAUUUUGUUUCCGU | 2957 | ACGGAAACAAAAUCAACA | 3992 | | [3453-3470] 3′UTR |
| 382 | UGCUGGAAUAUGAAGUCU | 2958 | AGACUUCAUAUUCCAGCA | 3993 | Ms | [3504-3521] 3′UTR |
| 383 | GUUGUUGAAAGUUGACAA | 2959 | UUGUCAACUUUCAACAAC | 3994 | | [3552-3569] 3′UTR |
| 384 | GGUCGUUGCAAGACUGUG | 2960 | CACAGUCUUGCAACGACC | 3995 | | [1356-1373] 3′UTR |
| 385 | GUAGGUAUUAGACUUGCA | 2961 | UGCAAGUCUAAUACCUAC | 3996 | | [2910-2927] 3′UTR |
| 386 | CUUUGUAUCAUUCUUGAG | 2962 | CUCAAGAAUGAUACAAAG | 3997 | | [3593-3610] 3′UTR |
| 387 | GGGAGCACUGUGUUUAUG | 2963 | CAUAAACACAGUGCUCCC | 3998 | | [3488-3505] 3′UTR |
| 388 | UGUCUCUGAUGCUUUGUA | 2964 | UACAAAGCAUCAGAGACA | 3999 | | [3582-3599] 3′UTR |
| 389 | GUUCCAGCCUCAGCUGAG | 2965 | CUCAGCUGAGGCUGGAAC | 4000 | | [1652-1669] 3′UTR |
| 390 | GGUUAGGAUAGGAAGAAC | 2966 | GUUCUUCCUAUCCUAACC | 4001 | | [2322-2339] 3′UTR |
| 391 | AUAUACAACUCCACCAGA | 2967 | UCUGGUGGAGUUGUAUAU | 4002 | Rh | [2733-2750] 3′UTR |
| 392 | UCUGAGCCUUGUAGAAAU | 2968 | AUUUCUACAAGGCUCAGA | 4003 | Rh | [1607-1624] 3′UTR |
| 393 | GUGAGGUCCUGUCCUGAG | 2969 | CUCAGGACAGGACCUCAC | 4004 | Rh | [1743-1760] 3′UTR |
| 394 | GGGUGGCAGCUGACAGAG | 2970 | CUCUGUCAGCUGCCACCC | 4005 | | [3200-3217] 3′UTR |
| 395 | CAAGCAGACUGCGCAUGU | 2971 | ACAUGCGCAGUCUGCUUG | 4006 | | [3567-3584] 3′UTR |
| 396 | CUCCCUCUGAGCCUUGUA | 2972 | UACAAGGCUCAGAGGGAG | 4007 | Rh | [1602-1619] 3′UTR |
| 397 | GGAGGUAGGUGGCUUUGG | 2973 | CCAAAGCCACCUACCUCC | 4008 | Rh | [2076-2093] 3′UTR |
| 398 | GAGCAGAAAACAAAACAG | 2974 | CUGUUUUGUUUUCUGCUC | 4009 | | [3169-3186] 3′UTR |
| 399 | AGAAGGCUCUCCAUUUGG | 2975 | CCAAAUGGAGAGCCUUCU | 4010 | | [3270-3287] 3′UTR |
| 400 | UAUGAAGUCUGAGACCUU | 2976 | AAGGUCUCAGACUUCAUA | 4011 | | [3512-3529] 3′UTR |
| 401 | AACAUUUACUCCUGUUUC | 2977 | GAAACAGGAGUAAAUGUU | 4012 | Rh | [2811-2828] 3′UTR |
| 402 | GACGAGUGCCUCUGGAUG | 2978 | CAUCCAGAGGCACUCGUC | 4013 | Rh, Rb, Cw | [809-826] ORF |
| 403 | CUGGGUCACAGAGAAGAA | 2979 | UUCUUCUCUGUGACCCAG | 4014 | Rh | [829-846] ORF |
| 404 | CUAGGAAGGGAAGGAUUU | 2980 | AAAUCCUUCCCUUCCUAG | 4015 | Rh | [2057-2074] 3′UTR |
| 405 | CGGCUUCCCUCCCAGUCC | 2981 | GGACUGGGAGGGAAGCCG | 4016 | | [1236-1253] 3′UTR |
| 406 | CCAGUGGGAGCCUCCCUC | 2982 | GAGGGAGGCUCCCACUGG | 4017 | Rh | [1591-1608] 3′UTR |
| 407 | GAGUUUCUCGACAUCGAG | 2983 | CUCGAUGUCGAGAAACUC | 4018 | Ck, Dg | [938-955] ORF |
| 408 | AGAAGAGCCGGGUGGCAG | 2984 | CUGCCACCCGGCUCUUCU | 4019 | | [3191-3208] 3′UTR |
| 409 | ACAUCAGCUGUAAUCAUU | 2985 | AAUGAUUACAGCUGAUGU | 4020 | | [2865-2882] 3′UTR |
| 410 | CUCAGAUGAUAGGUGAAC | 2986 | GUUCACCUAUCAUCUGAG | 4021 | | [2173-2190] 3′UTR |
| 411 | GCAGGUGGAUUCCUUCAG | 2987 | CUGAAGGAAUCCACCUGC | 4022 | Rh | [2992-3009] 3′UTR |
| 412 | GCGCAUGUCUCUGAUGCU | 2988 | AGCAUCAGAGACAUGCGC | 4023 | | [3577-3594] 3′UTR |
| 413 | CAGUAUAUACAACUCCAC | 2989 | GUGGAGUUGUAUAUACUG | 4024 | Rh | [2729-2746] 3′UTR |
| 414 | CAUUCUUGAGCAAUCGCU | 2990 | AGCGAUUGCUCAAGAAUG | 4025 | | [3601-3618] 3′UTR |
| 415 | CCUCCUCGGCAGUGUGUG | 2991 | CACACACUGCCGAGGAGG | 4026 | | [579-596] ORF |
| 416 | UCCUGUUUCUGCUGAUUG | 2992 | CAAUCAGCAGAAACAGGA | 4027 | | [2820-2837] 3′UTR |
| 417 | CCCUCCUCGGCAGUGUGU | 2993 | ACACACUGCCGAGGAGGG | 4028 | | [578-595] ORF |
| 418 | UACCCUUGGUAGGUAUUA | 2994 | UAAUACCUACCAAGGGUA | 4029 | | [2902-2919] 3′UTR |
| 419 | GCUUCCUGUAUGGUGAUA | 2995 | UAUCACCAUACAGGAAGC | 4030 | | [2785-2802] 3′UTR |
| 420 | GGACAUGGCCCUUGUUUU | 2996 | AAAACAAGGGCCAUGUCC | 4031 | | [1408-1425] 3′UTR |
| 421 | GCAUGAAUAAAACACUCA | 2997 | UGAGUGUUUUAUUCAUGC | 4032 | Rh | [1053-1070] 3′UTR |
| 422 | CCUGUUUCUGCUGAUUGU | 2998 | ACAAUCAGCAGAAACAGG | 4033 | | [2821-2838] 3′UTR |
| 423 | GCACAUCACCCUCUGUGA | 2999 | UCACAGAGGGUGAUGUGC | 4034 | Rh | [667-684] ORF |
| 424 | GCCGCUCAAAUACCUUCA | 3000 | UGAAGGUAUUUGAGCGGC | 4035 | | [3221-3238] 3′UTR |
| 425 | GCAACAGGCGUUUUGCAA | 3001 | UUGCAAAACGCCUGUUGC | 4036 | Cw, Rt, Ms | [403-420] ORF |
| 426 | GGGACGGCAAGAUGCACA | 3002 | UGUGCAUCUUGCCGUCCC | 4037 | | [654-671] ORF |
| 427 | GCCAGGCACUAUGUGUCU | 3003 | AGACACAUAGUGCCUGGC | 4038 | | [1179-1196] 3′UTR |
| 428 | GACACACUCACUUCUUUC | 3004 | GAAAGAAGUGAGUGUGUC | 4039 | | [2095-2112] 3′UTR |
| 429 | CUGAGACCUUCCGGUGCU | 3005 | AGCACCGGAAGGUCUCAG | 4040 | | [3520-3537] 3′UTR |
| 430 | CAGAAGAAGAGCCUGAAC | 3006 | GUUCAGGCUCUUCUUCUG | 4041 | Rh, Rb, Cw, Ms, Pg | [719-736] ORF |
| 431 | GAUAGGUGAACCUGAGUU | 3007 | AACUCAGGUUCACCUAUC | 4042 | | [2180-2197] 3′UTR |
| 432 | GUUCUAUUCAGCGGCAAC | 3008 | GUUGCCGCUGAAUAGAAC | 4043 | | [2503-2520] 3′UTR |
| 433 | AGACUGGGAGGGUAUCCA | 3009 | UGGAUACCCUCCCAGUCU | 4044 | Rh | [2623-2640] 3′UTR |
| 434 | GGCCAACUGCAAAAAAAG | 3010 | CUUUUUUUGCAGUUGGCC | 4045 | | [986-1003] 3′UTR |
| 435 | UGGCCUUUAUAUUUGAUC | 3011 | GAUCAAAUAUAAAGGCCA | 4046 | | [3124-3141] 3′UTR |
| 436 | GCUGGGAACACACAAGAG | 3012 | CUCUUGUGUGUUCCCAGC | 4047 | | [3535-3552] 3′UTR |
| 437 | GUGGAAGCAUUUGACCCA | 3013 | UGGGUCAAAUGCUUCCAC | 4048 | | [2954-2971] 3′UTR |
| 438 | CCAUGAUCCCGUGCUACA | 3014 | UGUAGCACGGGAUCAUGG | 4049 | Rh, Rb | [780-797] ORF |
| 439 | CAGGCAGCACUUAGGGAU | 3015 | AUCCCUAAGUGCUGCCUG | 4050 | Rh | [1282-1299] 3′UTR |
| 440 | GCAAAGUAAAGGAUCUUU | 3016 | AAAGAUCCUUUACUUUGC | 4051 | | [3083-3100] 3′UTR |
| 441 | GUGGACAAUAAACAGUAU | 3017 | AUACUGUUUAUUGUCCAC | 4052 | | [3625-3642] 3′UTR |
| 442 | UGCAAAAAAAGCCUCCAA | 3018 | UUGGAGGCUUUUUUUGCA | 4053 | | [993-1010] 3′UTR |
| 443 | CAAGCAGGAGUUUCUCGA | 3019 | UCGAGAAACUCCUGCUUG | 4054 | Ck, Dg | [931-948] ORF |
| 444 | CGUUCCAGCCUCAGCUGA | 3020 | UCAGCUGAGGCUGGAACG | 4055 | | [1651-1668] 3′UTR |
| 445 | CGGUCCGUGGACAAUAAA | 3021 | UUUAUUGUCCACGGACCG | 4056 | | [3619-3636] 3′UTR |
| 446 | UAGGAUAGGAAGAACUUU | 3022 | AAAGUUCUUCCUAUCCUA | 4057 | | [2325-2342] 3′UTR |
| 447 | AGCUGAGUCUUUUUGGUC | 3023 | GACCAAAAAGACUCAGCU | 4058 | Rh | [1663-1680] 3′UTR |
| 448 | UGGCUUUGGUGACACACU | 3024 | AGUGUGUCACCAAAGCCA | 4059 | | [2085-2102] 3′UTR |
| 449 | GCCGGUGGCUGCCCUCAA | 3025 | UUGAGGGCAGCCACCGGC | 4060 | Rh | [1774-1791] 3′UTR |
| 450 | CCUGGAACCAGUGGCUAG | 3026 | CUAGCCACUGGUUCCAGG | 4061 | | [1532-1549] 3′UTR |
| 451 | GCCUGUUCUGGCAUCAGG | 3027 | CCUGAUGCCAGAACAGGC | 4062 | | [1830-1847] 3′UTR |
| 452 | GACAGAAAAAGCUGGGUC | 3028 | GACCCAGCUUUUUCUGUC | 4063 | Rh | [1930-1947] 3′UTR |
| 453 | CAGCCUCCAGGACACUAU | 3029 | AUAGUGUCCUGGAGGCUG | 4064 | | [2114-2131] 3′UTR |
| 454 | GAAGCAUUUGACCCAGAG | 3030 | CUCUGGGUCAAAUGCUUC | 4065 | | [2957-2974] 3′UTR |
| 455 | GGCAUCAGGCACCUGGAU | 3031 | AUCCAGGUGCCUGAUGCC | 4066 | | [1839-1856] 3′UTR |
| 456 | AGGAUAGGAAGAACUUUC | 3032 | GAAAGUUCUUCCUAUCCU | 4067 | | [2326-2343] 3′UTR |
| 457 | AGUGCAAGAUCACGCGCU | 3033 | AGCGCGUGAUCUUGCACU | 4068 | Dg, Pg | [759-776] ORF |
| 458 | CUUCGAUCCUUGGGUGCA | 3034 | UGCACCCAAGGAUCGAAG | 4069 | Rh | [1201-1218] 3′UTR |
| 459 | GUAAAGGAUCUUUGAGUA | 3035 | UACUCAAAGAUCCUUUAC | 4070 | | [3088-3105] 3′UTR |
| 460 | CAGGCACCUGGAUUGAGU | 3036 | ACUCAAUCCAGGUGCCUG | 4071 | Rh | [1844-1861] 3′UTR |
| 461 | AUAAGGAGAAUCUCUUGU | 3037 | ACAAGAGAUUCUCCUUAU | 4072 | | [2353-2370] 3′UTR |
| 462 | UUCCCUCCCUCAAAGACU | 3038 | AGUCUUUGAGGGAGGGAA | 4073 | | [1973-1990] 3′UTR |
| 463 | UCUGGAAACGACAUUUAU | 3039 | AUAAAUGUCGUUUCCAGA | 4074 | | [470-487] ORF |
| 464 | GUAAGAAGUCCAGCCUAG | 3040 | CUAGGCUGGACUUCUUAC | 4075 | Rh | [2043-2060] 3′UTR |
| 465 | CAAAACAGGUUAAGAAGA | 3041 | UCUUCUUAACCUGUUUUG | 4076 | | [3179-3196] 3′UTR |
| 466 | GGUUGCCAUUGCUUCUUG | 3042 | CAAGAAGCAAUGGCAACC | 4077 | Rh | [1813-1830] 3′UTR |
| 467 | AGGUCCUCAUCCCAUCCU | 3043 | AGGAUGGGAUGAGGACCU | 4078 | Rh | [1155-1172] 3′UTR |
| 468 | GUCCGUGGACAAUAAACA | 3044 | UGUUUAUUGUCCACGGAC | 4079 | | [3621-3638] 3′UTR |
| 469 | UGUGUUUAUGCUGGAAUA | 3045 | UAUUCCAGCAUAAACACA | 4080 | | [3496-3513] 3′UTR |
| 470 | GGGCGUUUUCAUGCUGUA | 3046 | UACAGCAUGAAAACGCCC | 4081 | Rh | [2661-2678] 3′UTR |
| 471 | GGCACCUGGAUUGAGUUG | 3047 | CAACUCAAUCCAGGUGCC | 4082 | | [1846-1863] 3′UTR |
| 472 | CUGUAAUCAUUCCUGUGC | 3048 | GCACAGGAAUGAUUACAG | 4083 | Rh | [2872-2889] 3′UTR |
| 473 | GGCCUGGAAAUGUGCAUU | 3049 | AAUGCACAUUUCCAGGCC | 4084 | Rh | [1321-1338] 3′UTR |
| 474 | GGGUCGUUGCAAGACUGU | 3050 | ACAGUCUUGCAACGACCC | 4085 | | [1355-1372] 3′UTR |
| 475 | CAGGCGUUUUGCAAUGCA | 3051 | UGCAUUGCAAAACGCCUG | 4086 | Cw, Rt, Ms | [407-424] ORF |
| 476 | AGGGUAUCCAGGAAUCGG | 3052 | CCGAUUCCUGGAUACCCU | 4087 | | [2631-2648] 3′UTR |
| 477 | CUGGAAAUGUGCAUUUUG | 3053 | CAAAAUGCACAUUUCCAG | 4088 | Rh | [1324-1341] 3′UTR |
| 478 | GGUGGCUGCCCUCAAGGU | 3054 | ACCUUGAGGGCAGCCACC | 4089 | Rh | [1777-1794] 3′UTR |
| 479 | GGUCCAGCUCUGACAUCC | 3055 | GGAUGUCAGAGCUGGACC | 4090 | Rh | [1022-1039] 3′UTR |
| 480 | GCGGCCUGGGCGUGGUCU | 3056 | AGACCACGCCCAGGCCGC | 4091 | | [2455-2472] 3′UTR |
| 481 | UGCAUUUUGCAGAAACUU | 3057 | AAGUUUCUGCAAAAUGCA | 4092 | Rh | [1333-1350] 3′UTR |
| 482 | GUCUUUUAACCGUGCUGA | 3058 | UCAGCACGGUUAAAAGAC | 4093 | | [3153-3170] 3′UTR |
| 483 | CUCAAGGUCCCUUCCCUA | 3059 | UAGGGAAGGGACCUUGAG | 4094 | | [1787-1804] 3′UTR |
| 484 | AUCCAGUAUGAGAUCAAG | 3060 | CUUGAUCUCAUACUGGAU | 4095 | Rh, Rb | [506-523] ORF |
| 485 | GUCUGAAAGGUGUGGCCU | 3061 | AGGCCACACCUUUCAGAC | 4096 | | [3112-3129] 3′UTR |
| 486 | GACGUUGGAGGAAAGAAG | 3062 | CUUCUUUCCUCCAACGUC | 4097 | Rt, Ms | [608-625] ORF |
| 487 | CUUGUUUCCUCCCACCUG | 3063 | CAGGUGGGAGGAAACAAG | 4098 | | [2366-2383] 3′UTR |
| 488 | GACCUGGUCAGCACAGAU | 3064 | AUCUGUGCUGACCAGGUC | 4099 | Rh | [2581-2598] 3′UTR |
| 489 | GUUGCAGAUAUACCAACU | 3065 | AGUUGGUAUAUCUGCAAC | 4100 | Rh | [2195-2212] 3′UTR |
| 490 | CCACACACGUUGGUCUUU | 3066 | AAAGACCAACGUGUGUGG | 4101 | | [3141-3158] 3′UTR |
| 491 | CCCUCUGUGACUUCAUCG | 3067 | CGAUGAAGUCACAGAGGG | 4102 | Rh, Cw | [675-692] ORF |
| 492 | GAUCCUUGGGUGCAGGCA | 3068 | UGCCUGCACCCAAGGAUC | 4103 | Rh | [1205-1222] 3′UTR |
| 493 | CAAUGAAACCGAAGCUUG | 3069 | CAAGCUUCGGUUUCAUUG | 4104 | | [2437-2454] 3′UTR |
| 494 | AGCCUUGUAGAAAUGGGA | 3070 | UCCCAUUUCUACAAGGCU | 4105 | Rh | [1611-1628] 3′UTR |
| 495 | CUGUUCGCUUCCUGUAUG | 3071 | CAUACAGGAAGCGAACAG | 4106 | | [2779-2796] 3′UTR |
| 496 | AUGUGUUCCCUCAGUGUG | 3072 | CACACUGAGGGAACACAU | 4107 | | [2280-2297] 3′UTR |
| 497 | CCAAGCAGGCAGCACUUA | 3073 | UAAGUGCUGCCUGCUUGG | 4108 | | [1277-1294] 3′UTR |
| 498 | GCGAGUGCAAGAUCACGC | 3074 | GCGUGAUCUUGCACUCGC | 4109 | | [756-773] ORF |
| 499 | AUAGUUUAAGAAGGCUCU | 3075 | AGAGCCUUCUUAAACUAU | 4110 | | [3262-3279] 3′UTR |
| 500 | CAGACUGCGCAUGUCUCU | 3076 | AGAGACAUGCGCAGUCUG | 4111 | | [3571-3588] 3′UTR |
| 501 | CCUGUUUUAAGAGACAUC | 3077 | GAUGUCUCUUAAAACAGG | 4112 | Rh | [2134-2151] 3′UTR |
| 502 | UCAGUAUAUACAACUCCA | 3078 | UGGAGUUGUAUAUACUGA | 4113 | Rh | [2728-2745] 3′UTR |
| 503 | CGGCAAGAUGCACAUCAC | 3079 | GUGAUGUGCAUCUUGCCG | 4114 | Rh, Ck, Dg, Pg | [658-675] ORF |
| 504 | CAUCAGCUGUAAUCAUUC | 3080 | GAAUGAUUACAGCUGAUG | 4115 | | [2866-2883] 3′UTR |
| 505 | GCACCUGUUAAGACUCCU | 3081 | AGGAGUCUUAACAGGUGC | 4116 | Rh | [2527-2544] 3′UTR |
| 506 | GUCUGAGACCUUCCGGUG | 3082 | CACCGGAAGGUCUCAGAC | 4117 | | [3518-3535] 3′UTR |
| 507 | CUUCUUUCUCAGCCUCCA | 3083 | UGGAGGCUGAGAAAGAAG | 4118 | | [2105-2122] 3′UTR |
| 508 | GAUAAGGAGAAUCUCUUG | 3084 | CAAGAGAUUCUCCUUAUC | 4119 | | [2352-2369] 3′UTR |
| 509 | AGAUAUACCAACUUCUGC | 3085 | GCAGAAGUUGGUAUAUCU | 4120 | Rh | [2200-2217] 3′UTR |
| 510 | CUAUGCAGGUGGAUUCCU | 3086 | AGGAAUCCACCUGCAUAG | 4121 | Rh | [2988-3005] 3′UTR |
| 511 | AGGAAGCCGCUCAAAUAC | 3087 | GUAUUUGAGCGGCUUCCU | 4122 | | [3216-3233] 3′UTR |
| 512 | CGUGCUACAUCUCCUCCC | 3088 | GGGAGGAGAUGUAGCACG | 4123 | Rh | [789-806] ORF |
| 513 | AAAAAAGGUUUCUGCAUC | 3089 | GAUGCAGAAACCUUUUUU | 4124 | | [2936-2953] 3′UTR |
| 514 | GGACACGCGGCUUCCCUC | 3090 | GAGGGAAGCCGCGUGUCC | 4125 | | [1229-1246] 3′UTR |
| 515 | UAGAGUUUAUCUACACGG | 3091 | CCGUGUAGAUAAACUCUA | 4126 | Dg, Pg | [558-575] ORF |
| 516 | UCAAAGACUGACAGCCAU | 3092 | AUGGCUGUCAGUCUUUGA | 4127 | Rh | [1982-1999] 3′UTR |
| 517 | UGACAUCAGCUGUAAUCA | 3093 | UGAUUACAGCUGAUGUCA | 4128 | | [2863-2880] 3′UTR |
| 518 | AGUUGCACAGCUUUGCUU | 3094 | AAGCAAAGCUGUGCAACU | 4129 | | [1859-1876] 3′UTR |
| 519 | AGUGGCUAGUUCUUGAAG | 3095 | CUUCAAGAACUAGCCACU | 4130 | | [1541-1558] 3′UTR |
| 520 | UGGCAACCCUAUCAAGAG | 3096 | CUCUUGAUAGGGUUGCCA | 4131 | | [487-504] ORF |
| 521 | GUGGCUGCCCUCAAGGUC | 3097 | GACCUUGAGGGCAGCCAC | 4132 | Rh | [1778-1795] 3′UTR |
| 522 | GCGUUUUGCAAUGCAGAU | 3098 | AUCUGCAUUGCAAAACGC | 4133 | | [410-427] ORF |
| 523 | GAUCAAGCAGAUAAAGAU | 3099 | AUCUUUAUCUGCUUGAUC | 4134 | Cw, Dg, Rt, Ms, Pg | [517-534] ORF |
| 524 | GCAGGCAGCACUUAGGGA | 3100 | UCCCUAAGUGCUGCCUGC | 4135 | Rh | [1281-1298] 3′UTR |
| 525 | CUCCAACCCAUAUAACAC | 3101 | GUGUUAUAUGGGUUGGAG | 4136 | | [2754-2771] 3′UTR |
| 526 | GAACUAGGGAACCUAUGU | 3102 | ACAUAGGUUCCCUAGUUC | 4137 | Rh | [2266-2283] 3′UTR |
| 527 | GACGAUAUACAGGCACAU | 3103 | AUGUGCCUGUAUAUCGUC | 4138 | | [2401-2418] 3′UTR |
| 528 | GAAAUAUUGGACUUGCUG | 3104 | CAGCAAGUCCAAUAUUUC | 4139 | | [3419-3436] 3′UTR |
| 529 | GAAGCCGCUCAAAUACCU | 3105 | AGGUAUUUGAGCGGCUUC | 4140 | | [3218-3235] 3′UTR |
| 530 | GGCAGCCUGGAACCAGUG | 3106 | CACUGGUUCCAGGCUGCC | 4141 | | [1527-1544] 3′UTR |
| 531 | GUCUGGAGGGAGACGUGG | 3107 | CCACGUCUCCCUCCAGAC | 4142 | | [1132-1149] 3′UTR |
| 532 | CAUAGUAAGAAGUCCAGC | 3108 | GCUGGACUUCUUACUAUG | 4143 | | [2039-2056] 3′UTR |
| 533 | CUCCUGUUUCUGCUGAUU | 3109 | AAUCAGCAGAAACAGGAG | 4144 | | [2819-2836] 3′UTR |
| 534 | CAGAAUUCCAGUGGGAGC | 3110 | GCUCCCACUGGAAUUCUG | 4145 | Rh | [1584-1601] 3′UTR |
| 535 | AGCACUGUGUUUAUGCUG | 3111 | CAGCAUAAACACAGUGCU | 4146 | | [3491-3508] 3′UTR |
| 536 | GUAACAUUUACUCCUGUU | 3112 | AACAGGAGUAAAUGUUAC | 4147 | Rh | [2809-2826] 3′UTR |
| 537 | UGAGCUGCGUUCCAGCCU | 3113 | AGGCUGGAACGCAGCUCA | 4148 | | [1644-1661] 3′UTR |
| 538 | AGGUGGAUUCCUUCAGGU | 3114 | ACCUGAAGGAAUCCACCU | 4149 | Rh | [2994-3011] 3′UTR |
| 539 | UUUGUUUCCGUUUGGAUU | 3115 | AAUCCAAACGGAAACAAA | 4150 | | [3460-3477] 3′UTR |
| 540 | AAAGGAUCUUUGAGUAGG | 3116 | CCUACUCAAAGAUCCUUU | 4151 | | [3090-3107] 3′UTR |
| 541 | GGUCUGGAGGGAGACGUG | 3117 | CACGUCUCCCUCCAGACC | 4152 | | [1131-1148] 3′UTR |
| 542 | UGAGAUCAAGCAGAUAAA | 3118 | UUUAUCUGCUUGAUCUCA | 4153 | Rh, Cw, Dg, Rt, Ms | [514-531] ORF |
| 543 | GGAGGGUAUCCAGGAAUC | 3119 | GAUUCCUGGAUACCCUCC | 4154 | | [2629-2646] 3′UTR |
| 544 | GAGUUGCACAGCUUUGCU | 3120 | AGCAAAGCUGUGCAACUC | 4155 | | [1858-1875] 3′UTR |
| 545 | CCUUCACAAUAAAUAGUG | 3121 | CACUAUUUAUUGUGAAGG | 4156 | | [3233-3250] 3′UTR |
| 546 | CUUGUUUUCUGCAGCUUC | 3122 | GAAGCUGCAGAAAACAAG | 4157 | Rh | [1418-1435] 3′UTR |
| 547 | AUUGAGUUGCACAGCUUU | 3123 | AAAGCUGUGCAACUCAAU | 4158 | | [1855-1872] 3′UTR |
| 548 | UGAUUUUGUUUCCGUUUG | 3124 | CAAACGGAAACAAAAUCA | 4159 | | [3456-3473] 3′UTR |
| 549 | CAGCUCUCUUCUCCUAUU | 3125 | AAUAGGAGAAGAGAGCUG | 4160 | | [3320-3337] 3′UTR |
| 550 | GGCCUACCAGGUCCCUUU | 3126 | AAAGGGACCUGGUAGGCC | 4161 | Rh | [1379-1396] 3′UTR |
| 551 | UGUUAUGUUCUAAGCACA | 3127 | UGUGCUUAGAACAUAACA | 4162 | | [3304-3321] 3′UTR |
| 552 | GAGCCGGGUGGCAGCUGA | 3128 | UCAGCUGCCACCCGGCUC | 4163 | | [3195-3212] 3′UTR |
| 553 | GGAGGAAUCGGUGAGGUC | 3129 | GACCUCACCGAUUCCUCC | 4164 | | [1733-1750] 3′UTR |
| 554 | CCUGGGACACCCUGAGCA | 3130 | UGCUCAGGGUGUCCCAGG | 4165 | Rh, Dg, Pg | [696-713] ORF |
| 555 | UGUGCAUUUUGCAGAAAC | 3131 | GUUUCUGCAAAAUGCACA | 4166 | Rh, Rt, Ms | [1331-1348] 3′UTR |
| 556 | CCCUCUGCCAGGCACUAU | 3132 | AUAGUGCCUGGCAGAGGG | 4167 | | [1173-1190] 3′UTR |
| 557 | AGUCUGAGACCUUCCGGU | 3133 | ACCGGAAGGUCUCAGACU | 4168 | | [3517-3534] 3′UTR |
| 558 | CAACUUCUGCUUGUAUUU | 3134 | AAAUACAAGCAGAAGUUG | 4169 | Rh | [2208-2225] 3′UTR |
| 559 | CAGCCUGGAACCAGUGGC | 3135 | GCCACUGGUUCCAGGCUG | 4170 | | [1529-1546] 3′UTR |
| 560 | GCUUUGGUGACACACUCA | 3136 | UGAGUGUGUCACCAAAGC | 4171 | | [2087-2104] 3′UTR |
| 561 | CGCCUGCAUCAAGAGAAG | 3137 | CUUCUCUUGAUGCAGGCG | 4172 | Rh, Cw, Dg, Rt, Ms | [874-891] ORF |
| 562 | CUCCAAGGGUUUCGACUG | 3138 | CAGUCGAAACCCUUGGAG | 4173 | Rh | [1005-1022] 3′UTR |
| 563 | UGCUGGGAACACACAAGA | 3139 | UCUUGUGUGUUCCCAGCA | 4174 | | [3534-3551] 3′UTR |
| 564 | ACAUUUACUCCUGUUUCU | 3140 | AGAAACAGGAGUAAAUGU | 4175 | Rh | [2812-2829] 3′UTR |
| 565 | GGUUUCGACUGGUCCAGC | 3141 | GCUGGACCAGUCGAAACC | 4176 | Rh | [1012-1029] 3′UTR |
| 566 | CAGCUGUAAUCAUUCCUG | 3142 | CAGGAAUGAUUACAGCUG | 4177 | | [2869-2886] 3′UTR |
| 567 | CCAUCUGCACAUCCUGAG | 3143 | CUCAGGAUGUGCAGAUGG | 4178 | Rh | [1912-1929] 3′UTR |
| 568 | CAUCCCAUGGGUCCAAAU | 3144 | AUUUGGACCCAUGGGAUG | 4179 | | [1069-1086] 3′UTR |
| 569 | CUGUUUCUGCUGAUUGUU | 3145 | AACAAUCAGCAGAAACAG | 4180 | | [2822-2839] 3′UTR |
| 570 | GUGGCUUUGGUGACACAC | 3146 | GUGUGUCACCAAAGCCAC | 4181 | | [2084-2101] 3′UTR |
| 571 | GGCAGCUGACAGAGGAAG | 3147 | CUUCCUCUGUCAGCUGCC | 4182 | | [3204-3221] 3′UTR |
| 572 | AGAUCUUGAUGACUUCCC | 3148 | GGGAAGUCAUCAAGAUCU | 4183 | Rh | [2595-2612] 3′UTR |
| 573 | UGCAAGACUGUGUAGCAG | 3149 | CUGCUACACAGUCUUGCA | 4184 | Rh | [1362-1379] 3′UTR |
| 574 | CAGAUGUAGUGAUCAGGG | 3150 | CCCUGAUCACUACAUCUG | 4185 | | [423-440] ORF |
| 575 | CAUUUGGCAUCGUUUAAU | 3151 | AUUAAACGAUGCCAAAUG | 4186 | | [3281-3298] 3′UTR |
| 576 | CAGAAAAAGCUGGGUCUU | 3152 | AAGACCCAGCUUUUUCUG | 4187 | Rh | [1932-1949] 3′UTR |
| 577 | CCCAGAGUGGAACGCGUG | 3153 | CACGCGUUCCACUCUGGG | 4188 | | [2968-2985] 3′UTR |
| 578 | ACCUGUUAAGACUCCUGA | 3154 | UCAGGAGUCUUAACAGGU | 4189 | Rh | [2529-2546] 3′UTR |
| 579 | CAUCACCCUCUGUGACUU | 3155 | AAGUCACAGAGGGUGAUG | 4190 | Rh, Cw | [670-687] ORF |
| 580 | GGCUUCGAUCCUUGGGUG | 3156 | CACCCAAGGAUCGAAGCC | 4191 | Rh | [1199-1216] 3′UTR |
| 581 | CCUUGGCACCGUCACAGA | 3157 | UCUGUGACGGUGCCAAGG | 4192 | Rh | [1257-1274] 3′UTR |
| 582 | CAAGAGAAGUGACGGCUC | 3158 | GAGCCGUCACUUCUCUUG | 4193 | | [883-900] ORF |
| 583 | AGCUCUCUUCUCCUAUUU | 3159 | AAAUAGGAGAAGAGAGCU | 4194 | | [3321-3338] 3′UTR |
| 584 | GCAGCCUGGAACCAGUGG | 3160 | CCACUGGUUCCAGGCUGC | 4195 | | [1528-1545] 3′UTR |
| 585 | UGGACUCUGGAAACGACA | 3161 | UGUCGUUUCCAGAGUCCA | 4196 | Rh | [465-482] ORF |
| 586 | CACUCACUUCUUUCUCAG | 3162 | CUGAGAAAGAAGUGAGUG | 4197 | | [2099-2116] 3′UTR |
| 587 | ACCGUGCUGAGCAGAAAA | 3163 | UUUUCUGCUCAGCACGGU | 4198 | | [3161-3178] 3′UTR |
| 588 | AGAAUCUCUUGUUUCCUC | 3164 | GAGGAAACAAGAGAUUCU | 4199 | | [2359-2376] 3′UTR |
| 589 | ACAGCUCUCUUCUCCUAU | 3165 | AUAGGAGAAGAGAGCUGU | 4200 | | [3319-3336] 3′UTR |
| 590 | UCCUCAGAAUUCCAGUGG | 3166 | CCACUGGAAUUCUGAGGA | 4201 | Rh | [1580-1597] 3′UTR |
| 591 | GGCCCUUGUUUUCUGCAG | 3167 | CUGCAGAAAACAAGGGCC | 4202 | Rh | [1414-1431] 3′UTR |
| 592 | GUGGGUCUGGAGGGAGAC | 3168 | GUCUCCCUCCAGACCCAC | 4203 | Rh | [1128-1145] 3′UTR |
| 593 | AUCUCUUGUUUCCUCCCA | 3169 | UGGGAGGAAACAAGAGAU | 4204 | | [2362-2379] 3′UTR |
| 594 | GAACCACAGGUACCAGAU | 3170 | AUCUGGUACCUGUGGUUC | 4205 | Rh, Rb, Cw, Rt, Ms, Pg | [733-750] ORF |
| 595 | GCCUCCAAGGGUUUCGAC | 3171 | GUCGAAACCCUUGGAGGC | 4206 | Rh | [1003-1020] 3′UTR |
| 596 | CAGCAUGAAUAAAACACU | 3172 | AGUGUUUUAUUCAUGCUG | 4207 | Rh | [1051-1068] 3′UTR |
| 597 | CACCCAGAAGAAGAGCCU | 3173 | AGGCUCUUCUUCUGGGUG | 4208 | Rh, Ck, Cw, Rt, Ms, Pg | [715-732] ORF |
| 598 | UCAUAAUGGACCAGUCCA | 3174 | UGGACUGGUCCAUUAUGA | 4209 | Rh | [2703-2720] 3′UTR |
| 599 | ACAUCACCCUCUGUGACU | 3175 | AGUCACAGAGGGUGAUGU | 4210 | Rh | [669-686] ORF |
| 600 | CCUUCCUGGAAACAGCAU | 3176 | AUGCUGUUUCCAGGAAGG | 4211 | Rh, Rb, Rt, Ms | [1039-1056] 3′UTR |
| 601 | AGCCUCCAAGGGUUUCGA | 3177 | UCGAAACCCUUGGAGGCU | 4212 | Rh | [1002-1019] 3′UTR |
| 602 | UGACACACUCACUUCUUU | 3178 | AAAGAAGUGAGUGUGUCA | 4213 | | [2094-2111] 3′UTR |
| 603 | UGUGGGUCUGGAGGGAGA | 3179 | UCUCCCUCCAGACCCACA | 4214 | Rh | [1127-1144] 3′UTR |
| 604 | AGGCUCUCCAUUUGGCAU | 3180 | AUGCCAAAUGGAGAGCCU | 4215 | | [3273-3290] 3′UTR |
| 605 | CCAGUCCAUGUGAUUUCA | 3181 | UGAAAUCACAUGGACUGG | 4216 | Rh | [2713-2730] 3′UTR |
| 606 | GACGUGGGUCCAAGGUCC | 3182 | GGACCUUGGACCCACGUC | 4217 | | [1143-1160] 3′UTR |
| 607 | GGACAGAAAAAGCUGGGU | 3183 | ACCCAGCUUUUUCUGUCC | 4218 | Rh | [1929-1946] 3′UTR |
| 608 | CUACCAGGUCCCUUUCAU | 3184 | AUGAAAGGGACCUGGUAG | 4219 | Rh | [1382-1399] 3′UTR |
| 609 | AAUCCUCAGAAUUCCAGU | 3185 | ACUGGAAUUCUGAGGAUU | 4220 | Rh | [1578-1595] 3′UTR |
| 610 | CUUUGAGUAGGUUCGGUC | 3186 | GACCGAACCUACUCAAAG | 4221 | | [3097-3114] 3′UTR |
| 611 | GCCGGGUGGCAGCUGACA | 3187 | UGUCAGCUGCCACCCGGC | 4222 | | [3197-3214] 3′UTR |
| 612 | GCAGAAACUUUUGAGGGU | 3188 | ACCCUCAAAAGUUUCUGC | 4223 | Rh | [1341-1358] 3′UTR |
| 613 | CAGUGGCUAGUUCUUGAA | 3189 | UUCAAGAACUAGCCACUG | 4224 | | [1540-1557] 3′UTR |
| 614 | GAAAUGGGAGCGAGAAAC | 3190 | GUUUCUCGCUCCCAUUUC | 4225 | | [1620-1637] 3′UTR |
| 615 | GCAGACUGCGCAUGUCUC | 3191 | GAGACAUGCGCAGUCUGC | 4226 | | [3570-3587] 3′UTR |
| 616 | UGUAAUCAUUCCUGUGCU | 3192 | AGCACAGGAAUGAUUACA | 4227 | Rh | [2873-2890] 3′UTR |
| 617 | UGGUAGGUAUUAGACUUG | 3193 | CAAGUCUAAUACCUACCA | 4228 | | [2908-2925] 3′UTR |
| 618 | CAUUUACUCCUGUUUCUG | 3194 | CAGAAACAGGAGUAAAUG | 4229 | Rh | [2813-2830] 3′UTR |
| 619 | AUGAAGUCUGAGACCUUC | 3195 | GAAGGUCUCAGACUUCAU | 4230 | | [3513-3530] 3′UTR |
| 620 | CUUGAUGACUUCCCUUUC | 3196 | GAAAGGGAAGUCAUCAAG | 4231 | Rh | [2599-2616] 3′UTR |
| 621 | CAACAGGCGUUUUGCAAU | 3197 | AUUGCAAAACGCCUGUUG | 4232 | Cw, Rt, Ms | [404-421] ORF |
| 622 | CCAUAAGCAGGCCUCCAA | 3198 | UUGGAGGCCUGCUUAUGG | 4233 | | [959-976] |
| | | | | | | ORF + 3′UTR |
| 623 | GUACAGUGACCUAAAGUU | 3199 | AACUUUAGGUCACUGUAC | 4234 | | [2676-2693] 3′UTR |
| 624 | GCAUAGUAAGAAGUCCAG | 3200 | CUGGACUUCUUACUAUGC | 4235 | | [2038-2055] 3′UTR |
| 625 | ACACUCACUUCUUUCUCA | 3201 | UGAGAAAGAAGUGAGUGU | 4236 | | [2098-2115] 3′UTR |
| 626 | AUCAAGAGGAUCCAGUAU | 3202 | AUACUGGAUCCUCUUGAU | 4237 | Rh, Rb | [497-514] ORF |
| 627 | AGUGAGAAGGAAGUGGAC | 3203 | GUCCACUUCCUUCUCACU | 4238 | | [452-469] ORF |
| 628 | GCCUAUGCAGGUGGAUUC | 3204 | GAAUCCACCUGCAUAGGC | 4239 | Rh | [2986-3003] 3′UTR |
| 629 | CACCGUCACAGAUGCCAA | 3205 | UUGGCAUCUGUGACGGUG | 4240 | | [1263-1280] 3′UTR |
| 630 | CUUUCUAGGGCAGACUGG | 3206 | CCAGUCUGCCCUAGAAAG | 4241 | Rh | [2612-2629] 3′UTR |
| 631 | CCCUCCCUCAAAGACUGA | 3207 | UCAGUCUUUGAGGGAGGG | 4242 | | [1975-1992] 3′UTR |
| 632 | CUAAGCAUAGUAAGAAGU | 3208 | ACUUCUUACUAUGCUUAG | 4243 | | [2034-2051] 3′UTR |
| 633 | CAGAUAUACCAACUUCUG | 3209 | CAGAAGUUGGUAUAUCUG | 4244 | Rh | [2199-2216] 3′UTR |
| 634 | UUGCAGAUAUACCAACUU | 3210 | AAGUUGGUAUAUCUGCAA | 4245 | Rh | [2196-2213] 3′UTR |
| 635 | GCCUCCCUCUGAGCCUUG | 3211 | CAAGGCUCAGAGGGAGGC | 4246 | Rh | [1600-1617] 3′UTR |
| 636 | GGAAAUGUGCAUUUUGCA | 3212 | UGCAAAAUGCACAUUUCC | 4247 | Rh | [1326-1343] 3′UTR |
| 637 | CUCCCAGGCUUAGUGUUC | 3213 | GAACACUAAGCCUGGGAG | 4248 | | [1958-1975] 3′UTR |
| 638 | CUUUGGUGACACACUCAC | 3214 | GUGAGUGUGUCACCAAAG | 4249 | | [2088-2105] 3′UTR |
| 639 | CAUCAAGAGAAGUGACGG | 3215 | CCGUCACUUCUCUUGAUG | 4250 | | [880-897] ORF |
| 640 | CAGCCAUCGUUCUGCACG | 3216 | CGUGCAGAACGAUGGCUG | 4251 | Rh | [1993-2010] 3′UTR |
| 641 | CAAAGACUGACAGCCAUC | 3217 | GAUGGCUGUCAGUCUUUG | 4252 | Rh | [1983-2000] 3′UTR |
| 642 | AGCAGAAAACAAAACAGG | 3218 | CCUGUUUUGUUUUCUGCU | 4253 | | [3170-3187] 3′UTR |
| 643 | GCACUUAGGGAUCUCCCA | 3219 | UGGGAGAUCCCUAAGUGC | 4254 | Rh | [1288-1305] 3′UTR |
| 644 | UGGACCAGUCCAUGUGAU | 3220 | AUCACAUGGACUGGUCCA | 4255 | Rh | [2709-2726] 3′UTR |
| 645 | AUGUGCAUUUUGCAGAAA | 3221 | UUUCUGCAAAAUGCACAU | 4256 | Rh, Ms | [1330-1347] 3′UTR |
| 646 | UGUAACAUUUACUCCUGU | 3222 | ACAGGAGUAAAUGUUACA | 4257 | Rh | [2808-2825] 3′UTR |
| 647 | GCUGUAAUCAUUCCUGUG | 3223 | CACAGGAAUGAUUACAGC | 4258 | Rh | [2871-2888] 3′UTR |
| 648 | UAAGGAGAAUCUCUUGUU | 3224 | AACAAGAGAUUCUCCUUA | 4259 | | [2354-2371] 3′UTR |
| 649 | UGACAAGCAGACUGCGCA | 3225 | UGCGCAGUCUGCUUGUCA | 4260 | | [3564-3581] 3′UTR |
| 650 | UCCUGUAUGGUGAUAUCA | 3226 | UGAUAUCACCAUACAGGA | 4261 | | [2788-2805] 3′UTR |
| 651 | GAUAGGAAGAACUUUCUC | 3227 | GAGAAAGUUCUUCCUAUC | 4262 | Rh | [2328-2345] 3′UTR |
| 652 | CAUUCCUGUGCUGUGUUU | 3228 | AAACACAGCACAGGAAUG | 4263 | Rh | [2879-2896] 3′UTR |
| 653 | CAAGGUCCCUUCCCUAGC | 3229 | GCUAGGGAAGGGACCUUG | 4264 | | [1789-1806] 3′UTR |
| 654 | CCGUCUUUGGUUCUCCAG | 3230 | CUGGAGAACCAAAGACGG | 4265 | | [3054-3071] 3′UTR |
| 655 | GCUUCUUGCCUGUUCUGG | 3231 | CCAGAACAGGCAAGAAGC | 4266 | | [1823-1840] 3′UTR |
| 656 | UGGGCUGCGAGUGCAAGA | 3232 | UCUUGCACUCGCAGCCCA | 4267 | Ck, Rb, Rt | [750-767] ORF |
| 657 | GAUGGGCUGCGAGUGCAA | 3233 | UUGCACUCGCAGCCCAUC | 4268 | Ck, Rb, Rt | [748-765] ORF |
| 658 | CUGUUCUGGCAUCAGGCA | 3234 | UGCCUGAUGCCAGAACAG | 4269 | | [1832-1849] 3′UTR |
| 659 | GGGUAUCCAGGAAUCGGC | 3235 | GCCGAUUCCUGGAUACCC | 4270 | | [2632-2649] 3′UTR |
| 660 | GCAACCCUAUCAAGAGGA | 3236 | UCCUCUUGAUAGGGUUGC | 4271 | | [489-506] ORF |
| 661 | CCCAUCUGCACAUCCUGA | 3237 | UCAGGAUGUGCAGAUGGG | 4272 | Rh | [1911-1928] 3′UTR |
| 662 | UAUAGUUUAAGAAGGCUC | 3238 | GAGCCUUCUUAAACUAUA | 4273 | | [3261-3278] 3′UTR |
| 663 | AGCCUGAACCACAGGUAC | 3239 | GUACCUGUGGUUCAGGCU | 4274 | Rh, Rb, Cw, Ms, Pg | [728-745] ORF |
| 664 | CGUUGCAAGACUGUGUAG | 3240 | CUACACAGUCUUGCAACG | 4275 | | [1359-1376] 3′UTR |
| 665 | CGUCACAGAUGCCAAGCA | 3241 | UGCUUGGCAUCUGUGACG | 4276 | | [1266-1283] 3′UTR |
| 666 | UGAGAAGGAAGUGGACUC | 3242 | GAGUCCACUUCCUUCUCA | 4277 | | [454-471] ORF |
| 667 | AUCCCUUCCUGGAAACAG | 3243 | CUGUUUCCAGGAAGGGAU | 4278 | Rh, Rb, Rt, Ms | [1036-1053] 3′UTR |
| 668 | CAAAGCACCUGUUAAGAC | 3244 | GUCUUAACAGGUGCUUUG | 4279 | Rh | [2523-2540] 3′UTR |
| 669 | AGGUCCCUUUCAUCUUGA | 3245 | UCAAGAUGAAAGGGACCU | 4280 | Rh | [1387-1404] 3′UTR |
| 670 | CCUUUUAGACAUGGUUGU | 3246 | ACAACCAUGUCUAAAAGG | 4281 | | [1112-1129] 3′UTR |
| 671 | AGCCUAGGAAGGGAAGGA | 3247 | UCCUUCCCUUCCUAGGCU | 4282 | Rh | [2054-2071] 3′UTR |
| 672 | GCUUUAUCCGGGCUUGUG | 3248 | CACAAGCCCGGAUAAAGC | 4283 | | [1873-1890] 3′UTR |
| 673 | CUCUUCUCCUAUUUUCAU | 3249 | AUGAAAAUAGGAGAAGAG | 4284 | | [3325-3342] 3′UTR |
| 674 | CGUAAUUUAAAGCUCUGU | 3250 | ACAGAGCUUUAAAUUACG | 4285 | | [3438-3455] 3′UTR |
| 675 | CCUAAAGUUGGUAAGAUG | 3251 | CAUCUUACCAACUUUAGG | 4286 | Rh | [2685-2702] 3′UTR |
| 676 | CUGUGCUGUGUUUUUUAU | 3252 | AUAAAAAACACAGCACAG | 4287 | Rh | [2884-2901] 3′UTR |
| 677 | ACCCAGAGUGGAACGCGU | 3253 | ACGCGUUCCACUCUGGGU | 4288 | | [2967-2984] 3′UTR |
| 678 | GUUCUAAGCACAGCUCUC | 3254 | GAGAGCUGUGCUUAGAAC | 4289 | | [3310-3327] 3′UTR |
| 679 | CUGUGUUUUUUAUUACCC | 3255 | GGGUAAUAAAAAACACAG | 4290 | Rh | [2889-2906] 3′UTR |
| 680 | GGACGGCAAGAUGCACAU | 3256 | AUGUGCAUCUUGCCGUCC | 4291 | | [655-672] ORF |
| 681 | GUUGAUUUUGUUUCCGUU | 3257 | AACGGAAACAAAAUCAAC | 4292 | | [3454-3471] 3′UTR |
| 682 | CUCCUUUUAGACAUGGUU | 3258 | AACCAUGUCUAAAAGGAG | 4293 | | [1110-1127] 3′UTR |
| 683 | UGAUGCUUUGUAUCAUUC | 3259 | GAAUGAUACAAAGCAUCA | 4294 | | [3588-3605] 3′UTR |
| 684 | CUUUAUCCGGGCUUGUGU | 3260 | ACACAAGCCCGGAUAAAG | 4295 | | [1874-1891] 3′UTR |
| 685 | AUAGAGUUUAUCUACACG | 3261 | CGUGUAGAUAAACUCUAU | 4296 | Dg, Pg | [557-574] ORF |
| 686 | GGGAUCUCCCAGCUGGGU | 3262 | ACCCAGCUGGGAGAUCCC | 4297 | | [1295-1312] 3′UTR |
| 687 | GUGAACCUGAGUUGCAGA | 3263 | UCUGCAACUCAGGUUCAC | 4298 | Rh | [2185-2202] 3′UTR |
| 688 | AGUGCCUCUGGAUGGACU | 3264 | AGUCCAUCCAGAGGCACU | 4299 | Rh, Rb, Cw, Dg, Rt, Ms | [813-830] ORF |
| 689 | GAAAGUUGACAAGCAGAC | 3265 | GUCUGCUUGUCAACUUUC | 4300 | | [3558-3575] 3′UTR |
| 690 | UUGCAAAAUGCUUCCAAA | 3266 | UUUGGAAGCAUUUUGCAA | 4301 | Rh | [2472-2489] 3′UTR |
| 691 | GUAAAGAUAAACUGACGA | 3267 | UCGUCAGUUUAUCUUUAC | 4302 | Rh | [2388-2405] 3′UTR |
| 692 | CCAAAGCCACCUUAGCCU | 3268 | AGGCUAAGGUGGCUUUGG | 4303 | Rh | [2485-2502] 3′UTR |
| 693 | AGAAAAAGCUGGGUCUUG | 3269 | CAAGACCCAGCUUUUUCU | 4304 | Rh | [1933-1950] 3′UTR |
| 694 | CUGCCGUAAUUUAAAGCU | 3270 | AGCUUUAAAUUACGGCAG | 4305 | | [3434-3451] 3′UTR |
| 695 | UCCCUUUCAUCUUGAGAG | 3271 | CUCUCAAGAUGAAAGGGA | 4306 | Rh | [1390-1407] 3′UTR |
| 696 | GAUCUUGAUGACUUCCCU | 3272 | AGGGAAGUCAUCAAGAUC | 4307 | Rh | [2596-2613] 3′UTR |
| 697 | UCAGUGUGGUUUCCUGAA | 3273 | UUCAGGAAACCACACUGA | 4308 | | [2290-2307] 3′UTR |
| 698 | GGAGCCUCCCUCUGAGCC | 3274 | GGCUCAGAGGGAGGCUCC | 4309 | Rh | [1597-1614] 3′UTR |
| 699 | UGGUAAGAUGUCAUAAUG | 3275 | CAUUAUGACAUCUUACCA | 4310 | Rh | [2693-2710] 3′UTR |
| 700 | AAGCAUUUGACCCAGAGU | 3276 | ACUCUGGGUCAAAUGCUU | 4311 | | [2958-2975] 3′UTR |
| 701 | AGCUAAGAAACUUCCUAG | 3277 | CUAGGAAGUUUCUUAGCU | 4312 | | [2247-2264] 3′UTR |
| 702 | CAGGUUAAGAAGAGCCGG | 3278 | CCGGCUCUUCUUAACCUG | 4313 | | [3184-3201] 3′UTR |
| 703 | GACUGCGCAUGUCUCUGA | 3279 | UCAGAGACAUGCGCAGUC | 4314 | | [3573-3590] 3′UTR |
| 704 | GUAGGUUCGGUCUGAAAG | 3280 | CUUUCAGACCGAACCUAC | 4315 | | [3103-3120] 3′UTR |
| 705 | UGAAAGUUGACAAGCAGA | 3281 | UCUGCUUGUCAACUUUCA | 4316 | | [3557-3574] 3′UTR |
| 706 | AACUUCUGCUUGUAUUUC | 3282 | GAAAUACAAGCAGAAGUU | 4317 | Rh | [2209-2226] 3′UTR |
| 707 | GACAAGCAGACUGCGCAU | 3283 | AUGCGCAGUCUGCUUGUC | 4318 | | [3565-3582] 3′UTR |
| 708 | AAGUCUGAGACCUUCCGG | 3284 | CCGGAAGGUCUCAGACUU | 4319 | | [3516-3533] 3′UTR |
| 709 | CUCUGACAUCCCUUCCUG | 3285 | CAGGAAGGGAUGUCAGAG | 4320 | Rh | [1029-1046] 3′UTR |
| 710 | GUAAACAUACACACGCAA | 3286 | UUGCGUGUGUAUGUUUAC | 4321 | Rh | [2422-2439] 3′UTR |
| 711 | CCUCUGGAUGGACUGGGU | 3287 | ACCCAGUCCAUCCAGAGG | 4322 | Rh, Rb, Cw, Dg, Rt, Ms, | [817-834] ORF |
| | | | | | Pg | |
| 712 | GAUGACUUCCCUUUCUAG | 3288 | CUAGAAAGGGAAGUCAUC | 4323 | Rh | [2602-2619] 3′UTR |
| 713 | CCUACCAGGUCCCUUUCA | 3289 | UGAAAGGGACCUGGUAGG | 4324 | Rh | [1381-1398] 3′UTR |
| 714 | CAAGGUCCUCAUCCCAUC | 3290 | GAUGGGAUGAGGACCUUG | 4325 | | [1153-1170] 3′UTR |
| 715 | GAUCUUUGAGUAGGUUCG | 3291 | CGAACCUACUCAAAGAUC | 4326 | | [3094-3111] 3′UTR |
| 716 | AGAAAUAUUGGACUUGCU | 3292 | AGCAAGUCCAAUAUUUCU | 4327 | | [3418-3435] 3′UTR |
| 717 | CUGCCCUCAAGGUCCCUU | 3293 | AAGGGACCUUGAGGGCAG | 4328 | Rh | [1782-1799] 3′UTR |
| 718 | CUAGGGAACCUAUGUGUU | 3294 | AACACAUAGGUUCCCUAG | 4329 | Rh | [2269-2286] 3′UTR |
| 719 | CAAGCAGGCAGCACUUAG | 3295 | CUAAGUGCUGCCUGCUUG | 4330 | | [1278-1295] 3′UTR |
| 720 | CCCACCGGGACCUGGUCA | 3296 | UGACCAGGUCCCGGUGGG | 4331 | | [2573-2590] 3′UTR |
| 721 | GUUUCUGCAUCGUGGAAG | 3297 | CUUCCACGAUGCAGAAAC | 4332 | Rh | [2943-2960] 3′UTR |
| 722 | GUGACCUAAAGUUGGUAA | 3298 | UUACCAACUUUAGGUCAC | 4333 | | [2681-2698] 3′UTR |
| 723 | UGGGAACACACAAGAGUU | 3299 | AACUCUUGUGUGUUCCCA | 4334 | | [3537-3554] 3′UTR |
| 724 | GAAUCGGUGAGGUCCUGU | 3300 | ACAGGACCUCACCGAUUC | 4335 | | [1737-1754] 3′UTR |
| 725 | CCCUCCCAGGCUUAGUGU | 3301 | ACACUAAGCCUGGGAGGG | 4336 | | [1956-1973] 3′UTR |
| 726 | CCCACAUCCAAGGGCAGC | 3302 | GCUGCCCUUGGAUGUGGG | 4337 | | [1515-1532] 3′UTR |
| 727 | AGCCUCCCUCUGAGCCUU | 3303 | AAGGCUCAGAGGGAGGCU | 4338 | Rh | [1599-1616] 3′UTR |
| 728 | CAUGAUCCCGUGCUACAU | 3304 | AUGUAGCACGGGAUCAUG | 4339 | Rh, Rb | [781-798] ORF |
| 729 | CAUAAUGGACCAGUCCAU | 3305 | AUGGACUGGUCCAUUAUG | 4340 | Rh | [2704-2721] 3′UTR |
| 730 | GUCACAGAUGCCAAGCAG | 3306 | CUGCUUGGCAUCUGUGAC | 4341 | | [1267-1284] 3′UTR |
| 731 | UGCAUCAAGAGAAGUGAC | 3307 | GUCACUUCUCUUGAUGCA | 4342 | | [878-895] ORF |
| 732 | GAGACCUUCCGGUGCUGG | 3308 | CCAGCACCGGAAGGUCUC | 4343 | | [3522-3539] 3′UTR |
| 733 | UACCUAGCUAAGAAACUU | 3309 | AAGUUUCUUAGCUAGGUA | 4344 | Rh | [2242-2259] 3′UTR |
| 734 | GCUGCGUUCCAGCCUCAG | 3310 | CUGAGGCUGGAACGCAGC | 4345 | | [1647-1664] 3′UTR |
| 735 | UGGUUUCCUGAAGCCAGU | 3311 | ACUGGCUUCAGGAAACCA | 4346 | | [2296-2313] 3′UTR |
| 736 | GCAGAUGUAGUGAUCAGG | 3312 | CCUGAUCACUACAUCUGC | 4347 | | [422-439] ORF |
| 737 | CACCUGGAUUGAGUUGCA | 3313 | UGCAACUCAAUCCAGGUG | 4348 | | [1848-1865] 3′UTR |
| 738 | UUGGUUCUCCAGUUCAAA | 3314 | UUUGAACUGGAGAACCAA | 4349 | | [3060-3077] 3′UTR |
| 739 | CUUGGCACCGUCACAGAU | 3315 | AUCUGUGACGGUGCCAAG | 4350 | Rh | [1258-1275] 3′UTR |
| 740 | CUUCCAAAGCCACCUUAG | 3316 | CUAAGGUGGCUUUGGAAG | 4351 | Rh | [2482-2499] 3′UTR |
| 741 | GGGCCUGGAAAUGUGCAU | 3317 | AUGCACAUUUCCAGGCCC | 4352 | Rh | [1320-1337] 3′UTR |
| 742 | CACGCGGCUUCCCUCCCA | 3318 | UGGGAGGGAAGCCGCGUG | 4353 | | [1232-1249] 3′UTR |
| 743 | CAAGUUCUUCGCCUGCAU | 3319 | AUGCAGGCGAAGAACUUG | 4354 | Rh, Rb, Cw, Dg, Ms | [865-882] ORF |
| 744 | CUGAAGCCAGUGAUAUGG | 3320 | CCAUAUCACUGGCUUCAG | 4355 | | [2303-2320] 3′UTR |
| 745 | UUCUCAGCCUCCAGGACA | 3321 | UGUCCUGGAGGCUGAGAA | 4356 | | [2110-2127] 3′UTR |
| 746 | UAUUACCCUUGGUAGGUA | 3322 | UACCUACCAAGGGUAAUA | 4357 | | [2899-2916] 3′UTR |
| 747 | AGGAUCUUUGAGUAGGUU | 3323 | AACCUACUCAAAGAUCCU | 4358 | | [3092-3109] 3′UTR |
| 748 | GGCUUCCCUCCCAGUCCC | 3324 | GGGACUGGGAGGGAAGCC | 4359 | | [1237-1254] 3′UTR |
| 749 | UCUCGGUAAUGAUAAGGA | 3325 | UCCUUAUCAUUACCGAGA | 4360 | | [2342-2359] 3′UTR |
| 750 | GCUUGCAGGAGGAAUCGG | 3326 | CCGAUUCCUCCUGCAAGC | 4361 | | [1726-1743] 3′UTR |
| 751 | GUGGUCUUGCAAAAUGCU | 3327 | AGCAUUUUGCAAGACCAC | 4362 | | [2466-2483] 3′UTR |
| 752 | CCUCAGCUGAGUCUUUUU | 3328 | AAAAAGACUCAGCUGAGG | 4363 | Rh | [1659-1676] 3′UTR |
| 753 | AGAAGUGACGGCUCCUGU | 3329 | ACAGGAGCCGUCACUUCU | 4364 | | [887-904] ORF |
| 754 | CGUGGUCUUGCAAAAUGC | 3330 | GCAUUUUGCAAGACCACG | 4365 | | [2465-2482] 3′UTR |
| 755 | ACCGGGACCUGGUCAGCA | 3331 | UGCUGACCAGGUCCCGGU | 4366 | | [2576-2593] 3′UTR |
| 756 | GAUCCACACACGUUGGUC | 3332 | GACCAACGUGUGUGGAUC | 4367 | | [3138-3155] 3′UTR |
| 757 | UAUAUUUGAUCCACACAC | 3333 | GUGUGUGGAUCAAAUAUA | 4368 | | [3131-3148] 3′UTR |
| 758 | ACCUAUGUGUUCCCUCAG | 3334 | CUGAGGGAACACAUAGGU | 4369 | | [2276-2293] 3′UTR |
| 759 | GUUUUUUAUUACCCUUGG | 3335 | CCAAGGGUAAUAAAAAAC | 4370 | Rh | [2893-2910] 3′UTR |
| 760 | GAAGUGGACUCUGGAAAC | 3336 | GUUUCCAGAGUCCACUUC | 4371 | | [461-478] ORF |
| 761 | CGCGGCUUCCCUCCCAGU | 3337 | ACUGGGAGGGAAGCCGCG | 4372 | | [1234-1251] 3′UTR |
| 762 | CCCUAUCAAGAGGAUCCA | 3338 | UGGAUCCUCUUGAUAGGG | 4373 | | [493-510] ORF |
| 763 | CCCGGACGAGUGCCUCUG | 3339 | CAGAGGCACUCGUCCGGG | 4374 | Rh, Rb, Cw | [805-822] ORF |
| 764 | CGGUUGCCAUUGCUUCUU | 3340 | AAGAAGCAAUGGCAACCG | 4375 | Rh | [1812-1829] 3′UTR |
| 765 | GCUGUGUUUUUUAUUACC | 3341 | GGUAAUAAAAAACACAGC | 4376 | Rh | [2888-2905] 3′UTR |
| 766 | CUUCCACGCCUCUGCACU | 3342 | AGUGCAGAGGCGUGGAAG | 4377 | | [1432-1449] 3′UTR |
| 767 | GGACCCAUAAGCAGGCCU | 3343 | AGGCCUGCUUAUGGGUCC | 4378 | Rh | [955-972] |
| | | | | | | ORF + 3′UTR |
| 768 | CUGGCAAGUGCUCCCAUC | 3344 | GAUGGGAGCACUUGCCAG | 4379 | | [1458-1475] 3′UTR |
| 769 | AAAGGUGUGGCCUUUAUA | 3345 | UAUAAAGGCCACACCUUU | 4380 | | [3117-3134] 3′UTR |
| 770 | ACAGGCACAUUAUGUAAA | 3346 | UUUACAUAAUGUGCCUGU | 4381 | | [2409-2426] 3′UTR |
| 771 | ACUUCCCUUUCUAGGGCA | 3347 | UGCCCUAGAAAGGGAAGU | 4382 | Rh | [2606-2623] 3′UTR |
| 772 | CUGUUGAUUUUGUUUCCG | 3348 | CGGAAACAAAAUCAACAG | 4383 | | [3452-3469] 3′UTR |
| 773 | CAAAGGGCCUGAGAAGGA | 3349 | UCCUUCUCAGGCCCUUUG | 4384 | | [538-555] ORF |
| 774 | CCUGAGCACCACCCAGAA | 3350 | UUCUGGGUGGUGCUCAGG | 4385 | Rh, Pg | [706-723] ORF |
| 775 | UCUUGAUGACUUCCCUUU | 3351 | AAAGGGAAGUCAUCAAGA | 4386 | Rh | [2598-2615] 3′UTR |
| 776 | GAGUGCAAGAUCACGCGC | 3352 | GCGCGUGAUCUUGCACUC | 4387 | Dg, Pg | [758-775] ORF |
| 777 | UUGUUUCCGUUUGGAUUU | 3353 | AAAUCCAAACGGAAACAA | 4388 | | [3461-3478] 3′UTR |
| 778 | CCUCCCAGUCCCUGCCUU | 3354 | AAGGCAGGGACUGGGAGG | 4389 | Rh | [1243-1260] 3′UTR |
| 779 | AGGCCUACCAGGUCCCUU | 3355 | AAGGGACCUGGUAGGCCU | 4390 | Rh | [1378-1395] 3′UTR |
| 780 | CAAGAUGCACAUCACCCU | 3356 | AGGGUGAUGUGCAUCUUG | 4391 | Rh, Dg | [661-678] ORF |
| 781 | UGGAGGAAAGAAGGAAUA | 3357 | UAUUCCUUCUUUCCUCCA | 4392 | Rt | [613-630] ORF |
| 782 | UGACAAAGAUUACCUAGC | 3358 | GCUAGGUAAUCUUUGUCA | 4393 | | [2232-2249] 3′UTR |
| 783 | GGUGGCUUUGGUGACACA | 3359 | UGUGUCACCAAAGCCACC | 4394 | | [2083-2100] 3′UTR |
| 784 | UCACUUCUUUCUCAGCCU | 3360 | AGGCUGAGAAAGAAGUGA | 4395 | | [2102-2119] 3′UTR |
| 785 | UAAUCAUUCCUGUGCUGU | 3361 | ACAGCACAGGAAUGAUUA | 4396 | Rh | [2875-2892] 3′UTR |
| 786 | AGUAGGUUCGGUCUGAAA | 3362 | UUUCAGACCGAACCUACU | 4397 | | [3102-3119] 3′UTR |
| 787 | CGUUUUGCAAUGCAGAUG | 3363 | CAUCUGCAUUGCAAAACG | 4398 | | [411-428] ORF |
| 788 | GGGCACCAGGCCAAGUUC | 3364 | GAACUUGGCCUGGUGCCC | 4399 | Rh, Rb, Rt, Ms | [854-871] ORF |
| 789 | ACAGAGAAGAACAUCAAC | 3365 | GUUGAUGUUCUUCUCUGU | 4400 | Rh | [836-853] ORF |
| 790 | CAAAAAAAGCCUCCAAGG | 3366 | CCUUGGAGGCUUUUUUUG | 4401 | | [995-1012] 3′UTR |
| 791 | UUGGUAGGUAUUAGACUU | 3367 | AAGUCUAAUACCUACCAA | 4402 | | [2907-2924] 3′UTR |
| 792 | GAGCACUGUGUUUAUGCU | 3368 | AGCAUAAACACAGUGCUC | 4403 | | [3490-3507] 3′UTR |
| 793 | UGUACAGUGACCUAAAGU | 3369 | ACUUUAGGUCACUGUACA | 4404 | | [2675-2692] 3′UTR |
| 794 | AAGAAAUAUUGGACUUGC | 3370 | GCAAGUCCAAUAUUUCUU | 4405 | | [3417-3434] 3′UTR |
| 795 | CAGAAACUUUUGAGGGUC | 3371 | GACCCUCAAAAGUUUCUG | 4406 | Rh | [1342-1359] 3′UTR |
| 796 | UUCCCUUUCUAGGGCAGA | 3372 | UCUGCCCUAGAAAGGGAA | 4407 | Rh | [2608-2625] 3′UTR |
| 797 | CACAUCCAAGGGCAGCCU | 3373 | AGGCUGCCCUUGGAUGUG | 4408 | | [1517-1534] 3′UTR |
| 798 | CAUCUUGAGAGGGACAUG | 3374 | CAUGUCCCUCUCAAGAUG | 4409 | | [1397-1414] 3′UTR |
| 799 | GGCUUUCUGCAUGUGACG | 3375 | CGUCACAUGCAGAAAGCC | 4410 | | [2012-2029] 3′UTR |
| 800 | UAGCUAAGAAACUUCCUA | 3376 | UAGGAAGUUUCUUAGCUA | 4411 | | [2246-2263] 3′UTR |
| 801 | ACAUCCAAGGGCAGCCUG | 3377 | CAGGCUGCCCUUGGAUGU | 4412 | | [1518-1535] 3′UTR |
| 802 | UGUUCCCUCCCUCAAAGA | 3378 | UCUUUGAGGGAGGGAACA | 4413 | | [1971-1988] 3′UTR |
| 803 | GUCUUUUUGGUCUGCACC | 3379 | GGUGCAGACCAAAAAGAC | 4414 | | [1669-1686] 3′UTR |
| 804 | CCAUGAGCUCCCAGCACC | 3380 | GGUGCUGGGAGCUCAUGG | 4415 | | [1489-1506] 3′UTR |
| 805 | AGAUGUAGUGAUCAGGGC | 3381 | GCCCUGAUCACUACAUCU | 4416 | | [424-441] ORF |
| 806 | GAGGUAGGUGGCUUUGGU | 3382 | ACCAAAGCCACCUACCUC | 4417 | Rh | [2077-2094] 3′UTR |
| 807 | AAGCCGCUCAAAUACCUU | 3383 | AAGGUAUUUGAGCGGCUU | 4418 | | [3219-3236] 3′UTR |
| 808 | UGAGCCUUGUAGAAAUGG | 3384 | CCAUUUCUACAAGGCUCA | 4419 | Rh | [1609-1626] 3′UTR |
| 809 | GACGCCAGCUAAGCAUAG | 3385 | CUAUGCUUAGCUGGCGUC | 4420 | | [2026-2043] 3′UTR |
| 810 | GCAGCUUCCACGCCUCUG | 3386 | CAGAGGCGUGGAAGCUGC | 4421 | Rh | [1428-1445] 3′UTR |
| 811 | AGAAUUCCAGUGGGAGCC | 3387 | GGCUCCCACUGGAAUUCU | 4422 | Rh | [1585-1602] 3′UTR |
| 812 | GAACGCGUGGCCUAUGCA | 3388 | UGCAUAGGCCACGCGUUC | 4423 | Rh | [2977-2994] 3′UTR |
| 813 | ACAGAAAAAGCUGGGUCU | 3389 | AGACCCAGCUUUUUCUGU | 4424 | Rh | [1931-1948] 3′UTR |
| 814 | AAGGAAUAUCUCAUUGCA | 3390 | UGCAAUGAGAUAUUCCUU | 4425 | | [623-640] ORF |
| 815 | CAAGAGGAUCCAGUAUGA | 3391 | UCAUACUGGAUCCUCUUG | 4426 | Rh, Rb | [499-516] ORF |
| 816 | AGCUGACAGAGGAAGCCG | 3392 | CGGCUUCCUCUGUCAGCU | 4427 | | [3207-3224] 3′UTR |
| 817 | AUAAAACACUCAUCCCAU | 3393 | AUGGGAUGAGUGUUUUAU | 4428 | | [1059-1076] 3′UTR |
| 818 | GAAUCUCUUGUUUCCUCC | 3394 | GGAGGAAACAAGAGAUUC | 4429 | | [2360-2377] 3′UTR |
| 819 | GUUAUGUUCUAAGCACAG | 3395 | CUGUGCUUAGAACAUAAC | 4430 | | [3305-3322] 3′UTR |
| 820 | ACACGUUGGUCUUUUAAC | 3396 | GUUAAAAGACCAACGUGU | 4431 | | [3145-3162] 3′UTR |
| 821 | GGUGCACCCGCAACAGGC | 3397 | GCCUGUUGCGGGUGCACC | 4432 | Cw, Dg, Rt, Ms | [394-411] ORF |
| 822 | UCUGCAUCGUGGAAGCAU | 3398 | AUGCUUCCACGAUGCAGA | 4433 | Rh | [2946-2963] 3′UTR |
| 823 | CUGCCAGGCACUAUGUGU | 3399 | ACACAUAGUGCCUGGCAG | 4434 | | [1177-1194] 3′UTR |
| 824 | GAAGUCUGAGACCUUCCG | 3400 | CGGAAGGUCUCAGACUUC | 4435 | | [3515-3532] 3′UTR |
| 825 | CUGGUCAGCACAGAUCUU | 3401 | AAGAUCUGUGCUGACCAG | 4436 | Rh | [2584-2601] 3′UTR |
| 826 | GUGCAUUUUGCAGAAACU | 3402 | AGUUUCUGCAAAAUGCAC | 4437 | Rh, Rt, Ms | [1332-1349] 3′UTR |
| 827 | UCAUCUUGAGAGGGACAU | 3403 | AUGUCCCUCUCAAGAUGA | 4438 | | [1396-1413] 3′UTR |
| 828 | CACUUAGGGAUCUCCCAG | 3404 | CUGGGAGAUCCCUAAGUG | 4439 | Rh | [1289-1306] 3′UTR |
| 829 | CACGCAAUGAAACCGAAG | 3405 | CUUCGGUUUCAUUGCGUG | 4440 | | [2433-2450] 3′UTR |
| 830 | GACUGACAGCCAUCGUUC | 3406 | GAACGAUGGCUGUCAGUC | 4441 | Rh | [1987-2004] 3′UTR |
| 831 | AUUGCAAAGUAAAGGAUC | 3407 | GAUCCUUUACUUUGCAAU | 4442 | | [3080-3097] 3′UTR |
| 832 | AGUGGAACGCGUGGCCUA | 3408 | UAGGCCACGCGUUCCACU | 4443 | | [2973-2990] 3′UTR |
| 833 | GUUCGGUCUGAAAGGUGU | 3409 | ACACCUUUCAGACCGAAC | 4444 | | [3107-3124] 3′UTR |
| 834 | UGCAGAUGUAGUGAUCAG | 3410 | CUGAUCACUACAUCUGCA | 4445 | | [421-438] ORF |
| 835 | GUGUUCCCUCAGUGUGGU | 3411 | ACCACACUGAGGGAACAC | 4446 | | [2282-2299] 3′UTR |
| 836 | UGCCGUAAUUUAAAGCUC | 3412 | GAGCUUUAAAUUACGGCA | 4447 | | [3435-3452] 3′UTR |
| 837 | CUGCGGUUGCCAUUGCUU | 3413 | AAGCAAUGGCAACCGCAG | 4448 | | [1809-1826] 3′UTR |
| 838 | CGGCUCCUGUGCGUGGUA | 3414 | UACCACGCACAGGAGCCG | 4449 | Rh | [895-912] ORF |
| 839 | GGGUUUCGACUGGUCCAG | 3415 | CUGGACCAGUCGAAACCC | 4450 | Rh | [1011-1028] 3′UTR |
| 840 | AGAGAAGAACAUCAACGG | 3416 | CCGUUGAUGUUCUUCUCU | 4451 | Rh, Rb, Cw | [838-855] ORF |
| 841 | AUGGACCAGUCCAUGUGA | 3417 | UCACAUGGACUGGUCCAU | 4452 | Rh | [2708-2725] 3′UTR |
| 842 | CCGUGCUACAUCUCCUCC | 3418 | GGAGGAGAUGUAGCACGG | 4453 | Rh | [788-805] ORF |
| 843 | CCAAAGCACCUGUUAAGA | 3419 | UCUUAACAGGUGCUUUGG | 4454 | Rh | [2522-2539] 3′UTR |
| 844 | CAACUGCAAAAAAAGCCU | 3420 | AGGCUUUUUUUGCAGUUG | 4455 | | [989-1006] 3′UTR |
| 845 | CUUUCUCAGCCUCCAGGA | 3421 | UCCUGGAGGCUGAGAAAG | 4456 | | [2108-2125] 3′UTR |
| 846 | UCUAAAGGUGAAUUCUCA | 3422 | UGAGAAUUCACCUUUAGA | 4457 | Ms | [2159-2176] 3′UTR |
| 847 | GCAGACUGGGAGGGUAUC | 3423 | GAUACCCUCCCAGUCUGC | 4458 | Rh | [2621-2638] 3′UTR |
| 848 | CUGGAACCAGUGGCUAGU | 3424 | ACUAGCCACUGGUUCCAG | 4459 | | [1533-1550] 3′UTR |
| 849 | GACUGUGUAGCAGGCCUA | 3425 | UAGGCCUGCUACACAGUC | 4460 | Rh | [1367-1384] 3′UTR |
| 850 | GGCCAAGUUCUUCGCCUG | 3426 | CAGGCGAAGAACUUGGCC | 4461 | Rh, Rb, Cw, Dg, Ms | [862-879] ORF |
| 851 | GUUCGCUUCCUGUAUGGU | 3427 | ACCAUACAGGAAGCGAAC | 4462 | | [2781-2798] 3′UTR |
| 852 | AAUUCCAGUGGGAGCCUC | 3428 | GAGGCUCCCACUGGAAUU | 4463 | Rh | [1587-1604] 3′UTR |
| 853 | UGCAAAAUGCUUCCAAAG | 3429 | CUUUGGAAGCAUUUUGCA | 4464 | Rh | [2473-2490] 3′UTR |
| 854 | CUGGAAUAUGAAGUCUGA | 3430 | UCAGACUUCAUAUUCCAG | 4465 | Ms | [3506-3523] 3′UTR |
| 855 | GCUGUGCCCUCCCAGGCU | 3431 | AGCCUGGGAGGGCACAGC | 4466 | | [1950-1967] 3′UTR |
| 856 | CCAGAUGGGCUGCGAGUG | 3432 | CACUCGCAGCCCAUCUGG | 4467 | Rh, Ck, Rb, Rt | [745-762] ORF |
| 857 | GCGGUUGCCAUUGCUUCU | 3433 | AGAAGCAAUGGCAACCGC | 4468 | | [1811-1828] 3′UTR |
| 858 | AGCUCUGUUGAUUUUGUU | 3434 | AACAAAAUCAACAGAGCU | 4469 | | [3448-3465] 3′UTR |
| 859 | UAUCAUUCUUGAGCAAUC | 3435 | GAUUGCUCAAGAAUGAUA | 4470 | | [3598-3615] 3′UTR |
| 860 | UGGAGGGAGACGUGGGUC | 3436 | GACCCACGUCUCCCUCCA | 4471 | | [1135-1152] 3′UTR |
| 861 | AAGAAACUUCCUAGGGAA | 3437 | UUCCCUAGGAAGUUUCUU | 4472 | | [2251-2268] 3′UTR |
| 862 | AAAGCUGGGUCUUGCUGU | 3438 | ACAGCAAGACCCAGCUUU | 4473 | Rh | [1937-1954] 3′UTR |
| 863 | AAUAUGAAGUCUGAGACC | 3439 | GGUCUCAGACUUCAUAUU | 4474 | | [3510-3527] 3′UTR |
| 864 | UUCCUGAAGCCAGUGAUA | 3440 | UAUCACUGGCUUCAGGAA | 4475 | | [2300-2317] 3′UTR |
| 865 | ACUCCUGUUUCUGCUGAU | 3441 | AUCAGCAGAAACAGGAGU | 4476 | | [2818-2835] 3′UTR |
| 866 | CCAGGCUUAGUGUUCCCU | 3442 | AGGGAACACUAAGCCUGG | 4477 | | [1961-1978] 3′UTR |
| 867 | CCAGAGUGGAACGCGUGG | 3443 | CCACGCGUUCCACUCUGG | 4478 | | [2969-2986] 3′UTR |
| 868 | ACCAGGUCCCUUUCAUCU | 3444 | AGAUGAAAGGGACCUGGU | 4479 | Rh | [1384-1401] 3′UTR |
| 869 | CGAGUGCCUCUGGAUGGA | 3445 | UCCAUCCAGAGGCACUCG | 4480 | Rh, Rb, Cw, Dg, Rt, Ms | [811-828] ORF |
| 870 | UAUGUGUUCCCUCAGUGU | 3446 | ACACUGAGGGAACACAUA | 4481 | | [2279-2296] 3′UTR |
| 871 | GUUUUCAUGCUGUACAGU | 3447 | ACUGUACAGCAUGAAAAC | 4482 | Rh | [2665-2682] 3′UTR |
| 872 | GACUGGGAGGGUAUCCAG | 3448 | CUGGAUACCCUCCCAGUC | 4483 | Rh | [2624-2641] 3′UTR |
| 873 | GUCAGCACAGAUCUUGAU | 3449 | AUCAAGAUCUGUGCUGAC | 4484 | Rh | [2587-2604] 3′UTR |
| 874 | CGGCCUGGGCGUGGUCUU | 3450 | AAGACCACGCCCAGGCCG | 4485 | | [2456-2473] 3′UTR |
| 875 | CUGCGAGUGCAAGAUCAC | 3451 | GUGAUCUUGCACUCGCAG | 4486 | Rt | [754-771] ORF |
| 876 | UGAAAGGUGUGGCCUUUA | 3452 | UAAAGGCCACACCUUUCA | 4487 | | [3115-3132] 3′UTR |
| 877 | UGUUCUGGCAUCAGGCAC | 3453 | GUGCCUGAUGCCAGAACA | 4488 | | [1833-1850] 3′UTR |
| 878 | GGGCUUGUGUGCAGGGCC | 3454 | GGCCCUGCACACAAGCCC | 4489 | Rh | [1882-1899] 3′UTR |
| 879 | CCACCCAGAAGAAGAGCC | 3455 | GGCUCUUCUUCUGGGUGG | 4490 | Rh, Ck, Cw, Rt, Ms, Pg | [714-731] ORF |
| 880 | CAGCUGAGCUGCGUUCCA | 3456 | UGGAACGCAGCUCAGCUG | 4491 | | [1640-1657] 3′UTR |
| 881 | UCUGGAUGGACUGGGUCA | 3457 | UGACCCAGUCCAUCCAGA | 4492 | Rh, Rb, Cw, Dg, Rt, Ms, | [819-836] ORF |
| | | | | | Pg | |
| 882 | CCCAAGCAGGAGUUUCUC | 3458 | GAGAAACUCCUGCUUGGG | 4493 | Dg | [929-946] ORF |
| 883 | AAGGUGUGGCCUUUAUAU | 3459 | AUAUAAAGGCCACACCUU | 4494 | | [3118-3135] 3′UTR |
| 884 | CCUCCCAGGCUUAGUGUU | 3460 | AACACUAAGCCUGGGAGG | 4495 | | [1957-1974] 3′UTR |
| 885 | CCAACUGCAAAAAAAGCC | 3461 | GGCUUUUUUUGCAGUUGG | 4496 | | [988-1005] 3′UTR |
| 886 | CUGGAUUGAGUUGCACAG | 3462 | CUGUGCAACUCAAUCCAG | 4497 | | [1851-1868] 3′UTR |
| 887 | AUGGCCUGUUUUAAGAGA | 3463 | UCUCUUAAAACAGGCCAU | 4498 | | [2130-2147] 3′UTR |
| 888 | UGUAUCAUUCUUGAGCAA | 3464 | UUGCUCAAGAAUGAUACA | 4499 | | [3596-3613] 3′UTR |
| 889 | AGAGUUUAUCUACACGGC | 3465 | GCCGUGUAGAUAAACUCU | 4500 | Dg, Ms, Pg | [559-576] ORF |
| 890 | CGGGACCUGGUCAGCACA | 3466 | UGUGCUGACCAGGUCCCG | 4501 | Rh | [2578-2595] 3′UTR |
| 891 | AUGACAAAGAUUACCUAG | 3467 | CUAGGUAAUCUUUGUCAU | 4502 | | [2231-2248] 3′UTR |
| 892 | UGAAGCCAGUGAUAUGGG | 3468 | CCCAUAUCACUGGCUUCA | 4503 | | [2304-2321] 3′UTR |
| 893 | GUGGCCUUUAUAUUUGAU | 3469 | AUCAAAUAUAAAGGCCAC | 4504 | | [3123-3140] 3′UTR |
| 894 | UAAGCAUAGUAAGAAGUC | 3470 | GACUUCUUACUAUGCUUA | 4505 | | [2035-2052] 3′UTR |
| 895 | CUGCACAUCCUGAGGACA | 3471 | UGUCCUCAGGAUGUGCAG | 4506 | Rh | [1916-1933] 3′UTR |
| 896 | AGUUGACAAGCAGACUGC | 3472 | GCAGUCUGCUUGUCAACU | 4507 | | [3561-3578] 3′UTR |
| 897 | UGAGCUCCCAGCACCUGA | 3473 | UCAGGUGCUGGGAGCUCA | 4508 | | [1492-1509] 3′UTR |
| 898 | CUGUUAAGACUCCUGACC | 3474 | GGUCAGGAGUCUUAACAG | 4509 | Rh | [2531-2548] 3′UTR |
| 899 | CACAUCACCCUCUGUGAC | 3475 | GUCACAGAGGGUGAUGUG | 4510 | Rh | [668-685] ORF |
| 900 | AAAGUUGACAAGCAGACU | 3476 | AGUCUGCUUGUCAACUUU | 4511 | | [3559-3576] 3′UTR |
| 901 | CCAAGUGGCAUGCAGCCC | 3477 | GGGCUGCAUGCCACUUGG | 4512 | | [2550-2567] 3′UTR |
| 902 | GUACCAGAUGGGCUGCGA | 3478 | UCGCAGCCCAUCUGGUAC | 4513 | Rh, Ck, Rb, Rt | [742-759] ORF |
| 903 | UGUUUCCGUUUGGAUUUU | 3479 | AAAAUCCAAACGGAAACA | 4514 | | [3462-3479] 3′UTR |
| 904 | AUCUUUGAGUAGGUUCGG | 3480 | CCGAACCUACUCAAAGAU | 4515 | | [3095-3112] 3′UTR |
| 905 | UGAUCCCGUGCUACAUCU | 3481 | AGAUGUAGCACGGGAUCA | 4516 | Rh, Rb | [783-800] ORF |
| 906 | ACCUGGUCAGCACAGAUC | 3482 | GAUCUGUGCUGACCAGGU | 4517 | Rh | [2582-2599] 3′UTR |
| 907 | CACUUCUUUCUCAGCCUC | 3483 | GAGGCUGAGAAAGAAGUG | 4518 | | [2103-2120] 3′UTR |
| 908 | GCUGCUGCGGUUGCCAUU | 3484 | AAUGGCAACCGCAGCAGC | 4519 | | [1805-1822] 3′UTR |
| 909 | CAUUGCUUCUUGCCUGUU | 3485 | AACAGGCAAGAAGCAAUG | 4520 | | [1819-1836] 3′UTR |
| 910 | CUGGAGGGAGACGUGGGU | 3486 | ACCCACGUCUCCCUCCAG | 4521 | | [1134-1151] 3′UTR |
| 911 | UGGUGACACACUCACUUC | 3487 | GAAGUGAGUGUGUCACCA | 4522 | | [2091-2108] 3′UTR |
| 912 | GGCAGGGCUGGGACACGC | 3488 | GCGUGUCCCAGCCCUGCC | 4523 | | [1219-1236] 3′UTR |
| 913 | UGAACCACAGGUACCAGA | 3489 | UCUGGUACCUGUGGUUCA | 4524 | Rh, Rb, Cw, Ms, Pg | [732-749] ORF |
| 914 | GUUAGGAUAGGAAGAACU | 3490 | AGUUCUUCCUAUCCUAAC | 4525 | | [2323-2340] 3′UTR |
| 915 | AGCUUUGCUUUAUCCGGG | 3491 | CCCGGAUAAAGCAAAGCU | 4526 | | [1867-1884] 3′UTR |
| 916 | CUGUCCUGAGGCUGCUGU | 3492 | ACAGCAGCCUCAGGACAG | 4527 | Rh | [1751-1768] 3′UTR |
| 917 | AUUGCUUCUUGCCUGUUC | 3493 | GAACAGGCAAGAAGCAAU | 4528 | | [1820-1837] 3′UTR |
| 918 | GAGCACCACCCAGAAGAA | 3494 | UUCUUCUGGGUGGUGCUC | 4529 | Rh, Pg | [709-726] ORF |
| 919 | GUAGUGAUCAGGGCCAAA | 3495 | UUUGGCCCUGAUCACUAC | 4530 | Cw, Rt, Pg | [428-445] ORF |
| 920 | CCUGUUAAGACUCCUGAC | 3496 | GUCAGGAGUCUUAACAGG | 4531 | Rh | [2530-2547] 3′UTR |
| 921 | AGGGUUUCGACUGGUCCA | 3497 | UGGACCAGUCGAAACCCU | 4532 | Rh | [1010-1027] 3′UTR |
| 922 | UCAUCCCAUGGGUCCAAA | 3498 | UUUGGACCCAUGGGAUGA | 4533 | | [1068-1085] 3′UTR |
| 923 | GCCCUUGUUUUCUGCAGC | 3499 | GCUGCAGAAAACAAGGGC | 4534 | Rh | [1415-1432] 3′UTR |
| 924 | GUUGACAAGCAGACUGCG | 3500 | CGCAGUCUGCUUGUCAAC | 4535 | | [3562-3579] 3′UTR |
| 925 | AGGGAACCUAUGUGUUCC | 3501 | GGAACACAUAGGUUCCCU | 4536 | Rh | [2271-2288] 3′UTR |
| 926 | CCCAGCUGGGUUAGGGCA | 3502 | UGCCCUAACCCAGCUGGG | 4537 | | [1302-1319] 3′UTR |
| 927 | GUUGGUCUUUUAACCGUG | 3503 | CACGGUUAAAAGACCAAC | 4538 | | [3149-3166] 3′UTR |
| 928 | UUAUGGCAACCCUAUCAA | 3504 | UUGAUAGGGUUGCCAUAA | 4539 | | [484-501] ORF |
| 929 | CUAAAGUUGGUAAGAUGU | 3505 | ACAUCUUACCAACUUUAG | 4540 | Rh | [2686-2703] 3′UTR |
| 930 | UGAAUUCUCAGAUGAUAG | 3506 | CUAUCAUCUGAGAAUUCA | 4541 | | [2167-2184] 3′UTR |
| 931 | UGCAGGUGGAUUCCUUCA | 3507 | UGAAGGAAUCCACCUGCA | 4542 | Rh | [2991-3008] 3′UTR |
| 932 | CUGCGCAUGUCUCUGAUG | 3508 | CAUCAGAGACAUGCGCAG | 4543 | | [3575-3592] 3′UTR |
| 933 | GAAGAACAUCAACGGGCA | 3509 | UGCCCGUUGAUGUUCUUC | 4544 | Rh, Rb | [841-858] ORF |
| 934 | GGCGCUCGGCCUCCUGCU | 3510 | AGCAGGAGGCCGAGCGCC | 4545 | Dg, Rt, Ms | [331-348] ORF |
| 935 | CUGAGGACAGAAAAAGCU | 3511 | AGCUUUUUCUGUCCUCAG | 4546 | Rh | [1925-1942] 3′UTR |
| 936 | AUCUUGAUGACUUCCCUU | 3512 | AAGGGAAGUCAUCAAGAU | 4547 | Rh | [2597-2614] 3′UTR |
| 937 | UAGGUAUUAGACUUGCAC | 3513 | GUGCAAGUCUAAUACCUA | 4548 | | [2911-2928] 3′UTR |
| 938 | UGAAGUCUGAGACCUUCC | 3514 | GGAAGGUCUCAGACUUCA | 4549 | | [3514-3531] 3′UTR |
| 939 | CCGUAAUUUAAAGCUCUG | 3515 | CAGAGCUUUAAAUUACGG | 4550 | | [3437-3454] 3′UTR |
| 940 | AAGGCUCUCCAUUUGGCA | 3516 | UGCCAAAUGGAGAGCCUU | 4551 | | [3272-3289] 3′UTR |
| 941 | UAGGGAACCUAUGUGUUC | 3517 | GAACACAUAGGUUCCCUA | 4552 | Rh | [2270-2287] 3′UTR |
| 942 | GCUCCCAGCACCUGACUC | 3518 | GAGUCAGGUGCUGGGAGC | 4553 | | [1495-1512] 3′UTR |
| 943 | UGGAUUGAGUUGCACAGC | 3519 | GCUGUGCAACUCAAUCCA | 4554 | | [1852-1869] 3′UTR |
| 944 | CUGCUGGCGACGCUGCUU | 3520 | AAGCAGCGUCGCCAGCAG | 4555 | | [347-364] ORF |
| 945 | CUGCGUUCCAGCCUCAGC | 3521 | GCUGAGGCUGGAACGCAG | 4556 | | [1648-1665] 3′UTR |
| 946 | GUGACUUCAUCGUGCCCU | 3522 | AGGGCACGAUGAAGUCAC | 4557 | Rh, Rb, Cw, Dg, Pg | [681-698] ORF |
| 947 | GCCCUGGGACACCCUGAG | 3523 | CUCAGGGUGUCCCAGGGC | 4558 | Rh, Cw, Dg, Pg | [694-711] ORF |
| 948 | UAUACAACUCCACCAGAC | 3524 | GUCUGGUGGAGUUGUAUA | 4559 | Rh | [2734-2751] 3′UTR |
| 949 | ACCUUCCGGUGCUGGGAA | 3525 | UUCCCAGCACCGGAAGGU | 4560 | | [3525-3542] 3′UTR |
| 950 | GCUUUCUGCAUGUGACGC | 3526 | GCGUCACAUGCAGAAAGC | 4561 | | [2013-2030] 3′UTR |
| 951 | CCACAUCCAAGGGCAGCC | 3527 | GGCUGCCCUUGGAUGUGG | 4562 | | [1516-1533] 3′UTR |
| 952 | GCAGCACUUAGGGAUCUC | 3528 | GAGAUCCCUAAGUGCUGC | 4563 | Rh | [1285-1302] 3′UTR |
| 953 | UGGGUCCAAGGUCCUCAU | 3529 | AUGAGGACCUUGGACCCA | 4564 | | [1147-1164] 3′UTR |
| 954 | UCUAGGGCAGACUGGGAG | 3530 | CUCCCAGUCUGCCCUAGA | 4565 | Rh | [2615-2632] 3′UTR |
| 955 | GAAGGGAAGGAUUUUGGA | 3531 | UCCAAAAUCCUUCCCUUC | 4566 | Rh | [2061-2078] 3′UTR |
| 956 | GAGGAAUCGGUGAGGUCC | 3532 | GGACCUCACCGAUUCCUC | 4567 | | [1734-1751] 3′UTR |
| 957 | AUUUGACCCAGAGUGGAA | 3533 | UUCCACUCUGGGUCAAAU | 4568 | | [2962-2979] 3′UTR |
| 958 | GGCGACGCUGCUUCGCCC | 3534 | GGGCGAAGCAGCGUCGCC | 4569 | | [352-369] ORF |
| 959 | UUAGCCUGUUCUAUUCAG | 3535 | CUGAAUAGAACAGGCUAA | 4570 | Rh | [2496-2513] 3′UTR |
| 960 | AGCUGAGCUGCGUUCCAG | 3536 | CUGGAACGCAGCUCAGCU | 4571 | | [1641-1658] 3′UTR |
| 961 | AAGUUGACAAGCAGACUG | 3537 | CAGUCUGCUUGUCAACUU | 4572 | | [3560-3577] 3′UTR |
| 962 | AGCACAGAUCUUGAUGAC | 3538 | GUCAUCAAGAUCUGUGCU | 4573 | Rh | [2590-2607] 3′UTR |
| 963 | GAGGGUAUCCAGGAAUCG | 3539 | CGAUUCCUGGAUACCCUC | 4574 | | [2630-2647] 3′UTR |
| 964 | AGUGUGGUUUCCUGAAGC | 3540 | GCUUCAGGAAACCACACU | 4575 | | [2292-2309] 3′UTR |
| 965 | UCCUUUUAGACAUGGUUG | 3541 | CAACCAUGUCUAAAAGGA | 4576 | | [1111-1128] 3′UTR |
| 966 | GUUUCCUCCCACCUGUGU | 3542 | ACACAGGUGGGAGGAAAC | 4577 | | [2369-2386] 3′UTR |
| 967 | CACUAUGGCCUGUUUUAA | 3543 | UUAAAACAGGCCAUAGUG | 4578 | | [2126-2143] 3′UTR |
| 968 | CCAGCUGGGUUAGGGCAG | 3544 | CUGCCCUAACCCAGCUGG | 4579 | | [1303-1320] 3′UTR |
| 969 | UGCGUUCCAGCCUCAGCU | 3545 | AGCUGAGGCUGGAACGCA | 4580 | | [1649-1666] 3′UTR |
| 970 | CCAGCCUAGGAAGGGAAG | 3546 | CUUCCCUUCCUAGGCUGG | 4581 | Rh | [2052-2069] 3′UTR |
| 971 | GCUGGGUCUUGCUGUGCC | 3547 | GGCACAGCAAGACCCAGC | 4582 | Rh | [1940-1957] 3′UTR |
| 972 | GUUUCUCGACAUCGAGGA | 3548 | UCCUCGAUGUCGAGAAAC | 4583 | Ck, Dg | [940-957] ORF |
| 973 | GCUGGGACACGCGGCUUC | 3549 | GAAGCCGCGUGUCCCAGC | 4584 | | [1225-1242] 3′UTR |
| 974 | UACCAACUUCUGCUUGUA | 3550 | UACAAGCAGAAGUUGGUA | 4585 | Rh | [2205-2222] 3′UTR |
| 975 | UCCAAGGGCAGCCUGGAA | 3551 | UUCCAGGCUGCCCUUGGA | 4586 | | [1521-1538] 3′UTR |
| 976 | CAACCCUAUCAAGAGGAU | 3552 | AUCCUCUUGAUAGGGUUG | 4587 | | [490-507] ORF |
| 977 | CAGCUGAGUCUUUUUGGU | 3553 | ACCAAAAAGACUCAGCUG | 4588 | Rh | [1662-1679] 3′UTR |
| 978 | UGUUAAGACUCCUGACCC | 3554 | GGGUCAGGAGUCUUAACA | 4589 | Rh | [2532-2549] 3′UTR |
| 979 | UAUCAAGAGGAUCCAGUA | 3555 | UACUGGAUCCUCUUGAUA | 4590 | Rh, Rb | [496-513] ORF |
| 980 | GCACCAGGCCAAGUUCUU | 3556 | AAGAACUUGGCCUGGUGC | 4591 | Rh, Rb, Cw, Rt, Ms | [856-873] ORF |
| 981 | GGGCUGGGACACGCGGCU | 3557 | AGCCGCGUGUCCCAGCCC | 4592 | | [1223-1240] 3′UTR |
| 982 | GGUCCCUUCCCUAGCUGC | 3558 | GCAGCUAGGGAAGGGACC | 4593 | | [1792-1809] 3′UTR |
| 983 | UGAUAGGUGAACCUGAGU | 3559 | ACUCAGGUUCACCUAUCA | 4594 | | [2179-2196] 3′UTR |
| 984 | AUUCCUGUGCUGUGUUUU | 3560 | AAAACACAGCACAGGAAU | 4595 | Rh | [2880-2897] 3′UTR |
| 985 | GAUGCACAUCACCCUCUG | 3561 | CAGAGGGUGAUGUGCAUC | 4596 | Rh | [664-681] ORF |
| 986 | CCAGGCACUAUGUGUCUG | 3562 | CAGACACAUAGUGCCUGG | 4597 | | [1180-1197] 3′UTR |
| 987 | GCUGCUGGCGACGCUGCU | 3563 | AGCAGCGUCGCCAGCAGC | 4598 | | [346-363] ORF |
| 988 | UCCAGUGGGAGCCUCCCU | 3564 | AGGGAGGCUCCCACUGGA | 4599 | Rh | [1590-1607] 3′UTR |
| 989 | AGCUCUGACAUCCCUUCC | 3565 | GGAAGGGAUGUCAGAGCU | 4600 | Rh | [1027-1044] 3′UTR |
| 990 | ACAGCUUUGCUUUAUCCG | 3566 | CGGAUAAAGCAAAGCUGU | 4601 | | [1865-1882] 3′UTR |
| 991 | GGGAACCUAUGUGUUCCC | 3567 | GGGAACACAUAGGUUCCC | 4602 | Rh | [2272-2289] 3′UTR |
| 992 | CAGGAGUUUCUCGACAUC | 3568 | GAUGUCGAGAAACUCCUG | 4603 | Ck, Dg | [935-952] ORF |
| 993 | GUCCCUUCCCUAGCUGCU | 3569 | AGCAGCUAGGGAAGGGAC | 4604 | | [1793-1810] 3′UTR |
| 994 | AGGUUCGGUCUGAAAGGU | 3570 | ACCUUUCAGACCGAACCU | 4605 | | [3105-3122] 3′UTR |
| 995 | UGACGAUAUACAGGCACA | 3571 | UGUGCCUGUAUAUCGUCA | 4606 | | [2400-2417] 3′UTR |
| 996 | AGUCUUUUUGGUCUGCAC | 3572 | GUGCAGACCAAAAAGACU | 4607 | | [1668-1685] 3′UTR |
| 997 | CACCCUGAGCACCACCCA | 3573 | UGGGUGGUGCUCAGGGUG | 4608 | Rh, Pg | [703-720] ORF |
| 998 | GAGAAGAACAUCAACGGG | 3574 | CCCGUUGAUGUUCUUCUC | 4609 | Rh, Rb | [839-856] ORF |
| 999 | GAGAAGUGACGGCUCCUG | 3575 | CAGGAGCCGUCACUUCUC | 4610 | | [886-903] ORF |
| 1000 | UGCGAGUGCAAGAUCACG | 3576 | CGUGAUCUUGCACUCGCA | 4611 | | [755-772] ORF |
| 1001 | AAGUAAAGGAUCUUUGAG | 3577 | CUCAAAGAUCCUUUACUU | 4612 | | [3086-3103] 3′UTR |
| 1002 | AGGCACAUUAUGUAAACA | 3578 | UGUUUACAUAAUGUGCCU | 4613 | Rh | [2411-2428] 3′UTR |
| 1003 | CGCUCGGUCCGUGGACAA | 3579 | UUGUCCACGGACCGAGCG | 4614 | | [3615-3632] 3′UTR |
| 1004 | GUUAAGAAGAGCCGGGUG | 3580 | CACCCGGCUCUUCUUAAC | 4615 | | [3187-3204] 3′UTR |
| 1005 | GUGGAACGCGUGGCCUAU | 3581 | AUAGGCCACGCGUUCCAC | 4616 | | [2974-2991] 3′UTR |
| 1006 | AAAGUUGGUAAGAUGUCA | 3582 | UGACAUCUUACCAACUUU | 4617 | Rh | [2688-2705] 3′UTR |
| 1007 | AGCUGCUGCGGUUGCCAU | 3583 | AUGGCAACCGCAGCAGCU | 4618 | | [1804-1821] 3′UTR |
| 1008 | CUGCAUCGUGGAAGCAUU | 3584 | AAUGCUUCCACGAUGCAG | 4619 | Rh | [2947-2964] 3′UTR |
| 1009 | UACUCCUGUUUCUGCUGA | 3585 | UCAGCAGAAACAGGAGUA | 4620 | | [2817-2834] 3′UTR |
| 1010 | CCUUGUAGAAAUGGGAGC | 3586 | GCUCCCAUUUCUACAAGG | 4621 | Rh | [1613-1630] 3′UTR |
| 1011 | AACCUAUGUGUUCCCUCA | 3587 | UGAGGGAACACAUAGGUU | 4622 | | [2275-2292] 3′UTR |
| 1012 | UAGUUUAAGAAGGCUCUC | 3588 | GAGAGCCUUCUUAAACUA | 4623 | | [3263-3280] 3′UTR |
| 1013 | UGCGCAUGUCUCUGAUGC | 3589 | GCAUCAGAGACAUGCGCA | 4624 | | [3576-3593] 3′UTR |
| 1014 | AGAGAAGUGACGGCUCCU | 3590 | AGGAGCCGUCACUUCUCU | 4625 | | [885-902] ORF |
| 1015 | CAAGGGUUUCGACUGGUC | 3591 | GACCAGUCGAAACCCUUG | 4626 | Rh | [1008-1025] 3′UTR |
| 1016 | UCUAAGCACAGCUCUCUU | 3592 | AAGAGAGCUGUGCUUAGA | 4627 | | [3312-3329] 3′UTR |
| 1017 | AUUUGAUCCACACACGUU | 3593 | AACGUGUGUGGAUCAAAU | 4628 | | [3134-3151] 3′UTR |
| 1018 | CUUCCCUCCCAGUCCCUG | 3594 | CAGGGACUGGGAGGGAAG | 4629 | | [1239-1256] 3′UTR |
| 1019 | AAGAAGGCUCUCCAUUUG | 3595 | CAAAUGGAGAGCCUUCUU | 4630 | | [3269-3286] 3′UTR |
| 1020 | CGGGUGGCAGCUGACAGA | 3596 | UCUGUCAGCUGCCACCCG | 4631 | | [3199-3216] 3′UTR |
| 1021 | UGGGAGCCUCCCUCUGAG | 3597 | CUCAGAGGGAGGCUCCCA | 4632 | Rh | [1595-1612] 3′UTR |
| 1022 | UAUACCAACUUCUGCUUG | 3598 | CAAGCAGAAGUUGGUAUA | 4633 | Rh | [2203-2220] 3′UTR |
| 1023 | UGGAUGGACUGGGUCACA | 3599 | UGUGACCCAGUCCAUCCA | 4634 | Rh, Rt, Ms, Pg | [821-838] ORF |
| 1024 | CAGAGUGGAACGCGUGGC | 3600 | GCCACGCGUUCCACUCUG | 4635 | | [2970-2987] 3′UTR |
| 1025 | UCUCUUGUUUCCUCCCAC | 3601 | GUGGGAGGAAACAAGAGA | 4636 | | [2363-2380] 3′UTR |
| 1026 | CACCAUGAGCUCCCAGCA | 3602 | UGCUGGGAGCUCAUGGUG | 4637 | | [1487-1504] 3′UTR |
| 1027 | AUCUGCACAUCCUGAGGA | 3603 | UCCUCAGGAUGUGCAGAU | 4638 | Rh | [1914-1931] 3′UTR |
| 1028 | CCCUUGUUUUCUGCAGCU | 3604 | AGCUGCAGAAAACAAGGG | 4639 | Rh | [1416-1433] 3′UTR |
| 1029 | CCCUUCCUGGAAACAGCA | 3605 | UGCUGUUUCCAGGAAGGG | 4640 | Rh, Rb, Rt, Ms | [1038-1055] 3′UTR |
| 1030 | ACCAUGAGCUCCCAGCAC | 3606 | GUGCUGGGAGCUCAUGGU | 4641 | | [1488-1505] 3′UTR |
| 1031 | CACACGUUGGUCUUUUAA | 3607 | UUAAAAGACCAACGUGUG | 4642 | | [3144-3161] 3′UTR |
| 1032 | CUGAGUCUUUUUGGUCUG | 3608 | CAGACCAAAAAGACUCAG | 4643 | | [1665-1682] 3′UTR |
| 1033 | CCCUCCCAGUCCCUGCCU | 3609 | AGGCAGGGACUGGGAGGG | 4644 | Rh | [1242-1259] 3′UTR |
| 1034 | UCAGCCUCCAGGACACUA | 3610 | UAGUGUCCUGGAGGCUGA | 4645 | | [2113-2130] 3′UTR |
| 1035 | UGCUUUAUCCGGGCUUGU | 3611 | ACAAGCCCGGAUAAAGCA | 4646 | | [1872-1889] 3′UTR |

TABLE B6
18 -mer siTIMP2 Cross-Species

| | | SEQ | | SEQ | | |
|---|---|---|---|---|---|---|
| | | ID | | ID | | human-73858577 |
| No. | Sense (5′ > 3′) | NO: | Antisense (5′ > 3′) | NO: | Other Sp | ORF: 303-965 |
| 1 | ACCAGAUGGGCUGCGAGU | 4647 | ACUCGCAGCCCAUCUGGU | 4707 | Rh, Ck, Rb, Rt | [744-761] ORF |
| 2 | ACAGGUACCAGAUGGGCU | 4648 | AGCCCAUCUGGUACCUGU | 4708 | Rh, Rb, Cw, Rt, Ms, Pg | [738-755] ORF |
| 3 | GAAGAGCCUGAACCACAG | 4649 | CUGUGGUUCAGGCUCUUC | 4709 | Rh, Rb, Cw, Ms, Pg | [724-741] ORF |
| 4 | UCUUCGCCUGCAUCAAGA | 4650 | UCUUGAUGCAGGCGAAGA | 4710 | Rh, Rb, Cw, Dg, Ms | [870-887] ORF |
| 5 | CGGGCACCAGGCCAAGUU | 4651 | AACUUGGCCUGGUGCCCG | 4711 | Rh, Rb, Rt, Ms | [853-870] ORF |
| 6 | CGCUCGGCCUCCUGCUGC | 4652 | GCAGCAGGAGGCCGAGCG | 4712 | Dg, Rt, Ms | [333-350] ORF |
| 7 | CAUCCCUUCCUGGAAACA | 4653 | UGUUUCCAGGAAGGGAUG | 4713 | Rh, Rb, Rt, Ms | [1035-1052] 3′UTR |
| 8 | CACCCGCAACAGGCGUUU | 4654 | AAACGCCUGUUGCGGGUG | 4714 | Cw, Rt, Ms | [398-415] ORF |
| 9 | GGCCGACGCCUGCAGCUG | 4655 | CAGCUGCAGGCGUCGGCC | 4715 | Cw, Dg, Rt, Ms | [370-387] ORF |
| 10 | GUGCCUCUGGAUGGACUG | 4656 | CAGUCCAUCCAGAGGCAC | 4716 | Rh, Rb, Cw, Dg, Rt, Ms | [814-831] ORF |
| 11 | CCUGAACCACAGGUACCA | 4657 | UGGUACCUGUGGUUCAGG | 4717 | Rh, Rb, Cw, Ms, Pg | [730-747] ORF |
| 12 | UCCUGGAAACAGCAUGAA | 4658 | UUCAUGCUGUUUCCAGGA | 4718 | Rh, Rb, Rt, Ms | [1042-1059] 3′UTR |
| 13 | GUCUCGCUGGACGUUGGA | 4659 | UCCAACGUCCAGCGAGAC | 4719 | Rt, Ms | [599-616] ORF |
| 14 | GCCGACGCCUGCAGCUGC | 4660 | GCAGCUGCAGGCGUCGGC | 4720 | Cw, Dg, Rt, Ms | [371-388] ORF |
| 15 | AACCACAGGUACCAGAUG | 4661 | CAUCUGGUACCUGUGGUU | 4721 | Rh, Rb, Cw, Rt, Ms, Pg | [734-751] ORF |
| 16 | CCCGGUGCACCCGCAACA | 4662 | UGUUGCGGGUGCACCGGG | 4722 | Cw, Dg, Rt, Ms | [391-408] ORF |
| 17 | CACAGGUACCAGAUGGGC | 4663 | GCCCAUCUGGUACCUGUG | 4723 | Rh, Rb, Cw, Rt, Ms, Pg | [737-754] ORF |
| 18 | CCCGCAACAGGCGUUUUG | 4664 | CAAAACGCCUGUUGCGGG | 4724 | Cw, Rt, Ms | [400-417] ORF |
| 19 | UCAAGCAGAUAAAGAUGU | 4665 | ACAUCUUUAUCUGCUUGA | 4725 | Cw, Dg, Rt, Ms, Pg | [519-536] ORF |
| 20 | CACCAGGCCAAGUUCUUC | 4666 | GAAGAACUUGGCCUGGUG | 4726 | Rh, Rb, Cw, Ms | [857-874] ORF |
| 21 | ACCACAGGUACCAGAUGG | 4667 | CCAUCUGGUACCUGUGGU | 4727 | Rh, Rb, Cw, Rt, Ms, Pg | [735-752] ORF |
| 22 | UCUCGCUGGACGUUGGAG | 4668 | CUCCAACGUCCAGCGAGA | 4728 | Rt, Ms | [600-617] ORF |
| 23 | CAAGCAGAUAAAGAUGUU | 4669 | AACAUCUUUAUCUGCUUG | 4729 | Cw, Dg, Rt, Ms, Pg | [520-537] ORF |
| 24 | GAUAAAGAUGUUCAAAGG | 4670 | CCUUUGAACAUCUUUAUC | 4730 | Dg, Rt, Ms | [526-543] ORF |
| 25 | GCACCCGCAACAGGCGUU | 4671 | AACGCCUGUUGCGGGUGC | 4731 | Cw, Dg, Rt, Ms | [397-414] ORF |
| 26 | CAGGCCAAGUUCUUCGCC | 4672 | GGCGAAGAACUUGGCCUG | 4732 | Rh, Rb, Cw, Dg, Ms | [860-877] ORF |
| 27 | UGCACCCGCAACAGGCGU | 4673 | ACGCCUGUUGCGGGUGCA | 4733 | Cw, Dg, Rt, Ms | [396-413] ORF |
| 28 | CAGGUACCAGAUGGGCUG | 4674 | CAGCCCAUCUGGUACCUG | 4734 | Rh, Rb, Cw, Dg, Rt, Ms, Pg | [739-756] ORF |
| 29 | GCUGGCGCUCGGCCUCCU | 4675 | AGGAGGCCGAGCGCCAGC | 4735 | Dg, Rt, Ms | [328-345] ORF |
| 30 | GCGCUCGGCCUCCUGCUG | 4676 | CAGCAGGAGGCCGAGCGC | 4736 | Dg, Rt, Ms | [332-349] ORF |
| 31 | GACGCCUGCAGCUGCUCC | 4677 | GGAGCAGCUGCAGGCGUC | 4737 | Cw, Dg, Rt, Ms | [374-391] ORF |
| 32 | UACCAGAUGGGCUGCGAG | 4678 | CUCGCAGCCCAUCUGGUA | 4738 | Rh, Ck, Rb, Rt | [743-760] ORF |
| 33 | GCUCGGCCUCCUGCUGCU | 4679 | AGCAGCAGGAGGCCGAGC | 4739 | Dg, Rt, Ms | [334-351] ORF |
| 34 | CGGUGCACCCGCAACAGG | 4680 | CCUGUUGCGGGUGCACCG | 4740 | Cw, Dg, Rt, Ms | [393-410] ORF |
| 35 | CCGGUGCACCCGCAACAG | 4681 | CUGUUGCGGGUGCACCGG | 4741 | Cw, Dg, Rt, Ms | [392-409] ORF |
| 36 | ACCCGCAACAGGCGUUUU | 4682 | AAAACGCCUGUUGCGGGU | 4742 | Cw, Rt, Ms | [399-416] ORF |
| 37 | AUCAAGCAGAUAAAGAUG | 4683 | CAUCUUUAUCUGCUUGAU | 4743 | Cw, Dg, Rt, Ms, Pg | [518-535] ORF |
| 38 | CCACAGGUACCAGAUGGG | 4684 | CCCAUCUGGUACCUGUGG | 4744 | Rh, Rb, Cw, Rt, Ms, Pg | [736-753] ORF |
| 39 | CCGACGCCUGCAGCUGCU | 4685 | AGCAGCUGCAGGCGUCGG | 4745 | Cw, Dg, Rt, Ms | [372-389] ORF |
| 40 | CUUCAUCGUGCCCUGGGA | 4686 | UCCCAGGGCACGAUGAAG | 4746 | Rh, Rb, Cw, Dg, Pg | [685-702] ORF |
| 41 | CGGCCGACGCCUGCAGCU | 4687 | AGCUGCAGGCGUCGGCCG | 4747 | Cw, Dg, Rt, Ms | [369-386] ORF |
| 42 | GUGCACCCGCAACAGGCG | 4688 | CGCCUGUUGCGGGUGCAC | 4748 | Cw, Dg, Rt, Ms | [395-412] ORF |
| 43 | CGACGCCUGCAGCUGCUC | 4689 | GAGCAGCUGCAGGCGUCG | 4749 | Cw, Dg, Rt, Ms | [373-390] ORF |
| 44 | ACCAGGCCAAGUUCUUCG | 4690 | CGAAGAACUUGGCCUGGU | 4750 | Rh, Rb, Cw, Ms | [858-875] ORF |
| 45 | UGCCUCUGGAUGGACUGG | 4691 | CCAGUCCAUCCAGAGGCA | 4751 | Rh, Rb, Cw, Dg, Rt, Ms | [815-832] ORF |
| 46 | AACAGGCGUUUUGCAAUG | 4692 | CAUUGCAAAACGCCUGUU | 4752 | Cw, Rt, Ms | [405-422] ORF |
| 47 | CUCGGCCUCCUGCUGCUG | 4693 | CAGCAGCAGGAGGCCGAG | 4753 | Dg, Rt | [335-352] ORF |
| 48 | CGGCCUCCUGCUGCUGGC | 4694 | GCCAGCAGCAGGAGGCCG | 4754 | Dg, Rt | [337-354] ORF |
| 49 | CCAGGCCAAGUUCUUCGC | 4695 | GCGAAGAACUUGGCCUGG | 4755 | Rh, Rb, Cw, Dg, Ms | [859-876] ORF |
| 50 | CUGGCGCUCGGCCUCCUG | 4696 | CAGGAGGCCGAGCGCCAG | 4756 | Dg, Rt, Ms | [329-346] ORF |
| 51 | UCGGCCUCCUGCUGCUGG | 4697 | CCAGCAGCAGGAGGCCGA | 4757 | Dg, Rt | [336-353] ORF |
| 52 | UGGCGCUCGGCCUCCUGC | 4698 | GCAGGAGGCCGAGCGCCA | 4758 | Dg, Rt, Ms | [330-347] ORF |
| 53 | AGGUACCAGAUGGGCUGC | 4699 | GCAGCCCAUCUGGUACCU | 4759 | Rh, Ck, Rb, Rt | [740-757] ORF |
| 54 | CUGUGACUUCAUCGUGCC | 4700 | GGCACGAUGAAGUCACAG | 4760 | Rh, Rb, Cw, Dg, Pg | [679-696] ORF |
| 55 | GACUUCAUCGUGCCCUGG | 4701 | CCAGGGCACGAUGAAGUC | 4761 | Rh, Rb, Cw, Dg, Pg | [683-700] ORF |
| 56 | ACGCCUGCAGCUGCUCCC | 4702 | GGGAGCAGCUGCAGGCGU | 4762 | Cw, Dg, Rt, Ms | [375-392] ORF |
| 57 | AAGAGCCUGAACCACAGG | 4703 | CCUGUGGUUCAGGCUCUU | 4763 | Rh, Rb, Cw, Ms, Pg | [725-742] ORF |
| 58 | CAGGGCCAAAGCGGUCAG | 4704 | CUGACCGCUUUGGCCCUG | 4764 | Rb, Dg | [436-453] ORF |
| 59 | UGACUUCAUCGUGCCCUG | 4705 | CAGGGCACGAUGAAGUCA | 4765 | Rh, Rb, Cw, Dg, Pg | [682-699] ORF |
| 60 | UGUGACUUCAUCGUGCCC | 4706 | GGGCACGAUGAAGUCACA | 4766 | Rh, Rb, Cw, Dg, Pg | [680-697] ORF |

TABLE B7
Preferred 18 + 1 -mer siTIMP2

| | | SEQ | | SEQ | | |
|---|---|---|---|---|---|---|
| | | ID | | ID | | |
| SiTIMP2_p# | Sense (5′ > 3′) | NO: | Antisense (5′ > 3′) | NO: | Length | Position |
| siTIMP2_p1 | GGAGGAAAGAAGGAAUAUA | 4767 | UAUAUUCCUUCUUUCCUCC | 4815 | 18 + 1 | [614-631] ORF |
| siTIMP2_p2 | GGACGUUGGAGGAAAGAAA | 4768 | UUUCUUUCCUCCAACGUCC | 4816 | 18 + 1 | [607-624] ORF |
| siTIMP2-p3 | GGGUCUCGCUGGACGUUGA | 4769 | UCAACGUCCAGCGAGACCC | 4817 | 18 + 1 | [597-614] ORF |
| siTIMP2_p5 | GGACUGGGUCACAGAGAAA | 4770 | UUUCUCUGUGACCCAGUCC | 4818 | 18 + 1 | [826-843] ORF |
| siTIMP2_p6 | CUGCAUCAAGAGAAGUGAA | 4771 | UUCACUUCUCUUGAUGCAG | 4819 | 18 + 1 | [877-894] ORF |
| siTIMP2_p7 | GAGGAAAGAAGGAAUAUCA | 4772 | UGAUAUUCCUUCUUUCCUC | 4820 | 18 + 1 | [615-632] ORF |
| siTIMP2_p8 | GCUGGACGUUGGAGGAAAA | 4773 | UUUUCCUCCAACGUCCAGC | 4821 | 18 + 1 | [604-621] ORF |
| siTIMP2_p9 | GGCGUUUUGCAAUGCAGAA | 4774 | UUCUGCAUUGCAAAACGCC | 4822 | 18 + 1 | [409-426] ORF |
| siTIMP2_p10 | GCCUGCAUCAAGAGAAGUA | 4775 | UACUUCUCUUGAUGCAGGC | 4823 | 18 + 1 | [875-892] ORF |
| siTIMP2_p11 | AGGAAAGAAGGAAUAUCUA | 4776 | UAGAUAUUCCUUCUUUCCU | 4824 | 18 + 1 | [616-633] ORF |
| siTIMP2_p12 | AGAUCAAGCAGAUAAAGAA | 4777 | UUCUUUAUCUGCUUGAUCU | 4825 | 18 + 1 | [516-533] ORF |
| siTIMP2_p13 | GUUGGAGGAAAGAAGGAAA | 4778 | UUUCCUUCUUUCCUCCAAC | 4826 | 18 + 1 | [611-628] ORF |
| siTIMP2_p14 | GCUGCGAGUGCAAGAUCAA | 4779 | UUGAUCUUGCACUCGCAGC | 4827 | 18 + 1 | [753-770] ORF |
| siTIMP2_p15 | GGGCUGCGAGUGCAAGAUA | 4780 | UAUCUUGCACUCGCAGCCC | 4828 | 18 + 1 | [751-768] ORF |
| siTIMP2_p19 | GACAUCCCUUCCUGGAAAA | 4781 | UUUUCCAGGAAGGGAUGUC | 4829 | 18 + 1 | [1033-1050] 3′UTR |
| siTIMP2-p21 | GAUGGACUGGGUCACAGAA | 4782 | UUCUGUGACCCAGUCCAUC | 4830 | 18 + 1 | [823-840] ORF |
| siTIMP2_p22 | GCCUCUGGAUGGACUGGGA | 4783 | UCCCAGUCCAUCCAGAGGC | 4831 | 18 + 1 | [816-833] ORF |
| siTIMP2_p23 | GAGUGCCUCUGGAUGGACA | 4784 | UGUCCAUCCAGAGGCACUC | 4832 | 18 + 1 | [812-829] ORF |
| siTIMP2_p26 | GGCACCAGGCCAAGUUCUA | 4785 | UAGAACUUGGCCUGGUGCC | 4833 | 18 + 1 | [855-872] ORF |
| siTIMP2_p28 | GCAACAGGCGUUUUGCAAA | 4786 | UUUGCAAAACGCCUGUUGC | 4834 | 18 + 1 | [403-420] ORF |
| siTIMP2_p31 | GACGUUGGAGGAAAGAAGA | 4787 | UCUUCUUUCCUCCAACGUC | 4835 | 18 + 1 | [608-625] ORF |
| siTIMP2_p32 | GAUCAAGCAGAUAAAGAUA | 4788 | UAUCUUUAUCUGCUUGAUC | 4836 | 18 + 1 | [517-534] ORF |
| siTIMP2_p34 | UGAGAUCAAGCAGAUAAAA | 4789 | UUUUAUCUGCUUGAUCUCA | 4837 | 18 + 1 | [514-531] ORF |
| siTIMP2_p36 | UGUGCAUUUUGCAGAAACA | 4790 | UGUUUCUGCAAAAUGCACA | 4838 | 18 + 1 | [1331-1348] 3′UTR |
| siTIMP2_p42 | GAACCACAGGUACCAGAUA | 4791 | UAUCUGGUACCUGUGGUUC | 4839 | 18 + 1 | [733-750] ORF |
| siTIMP2_p43 | CACCCAGAAGAAGAGCCUA | 4792 | UAGGCUCUUCUUCUGGGUG | 4840 | 18 + 1 | [715-732] ORF |
| siTIMP2_p45 | CCUUCCUGGAAACAGCAUA | 4793 | UAUGCUGUUUCCAGGAAGG | 4841 | 18 + 1 | [1039-1056] 3′UTR |
| siTIMP2_p47 | UGGGCUGCGAGUGCAAGAA | 4794 | UUCUUGCACUCGCAGCCCA | 4842 | 18 + 1 | [750-767] ORF |
| siTIMP2_p48 | GAUGGGCUGCGAGUGCAAA | 4795 | UUUGCACUCGCAGCCCAUC | 4843 | 18 + 1 | [748-765] ORF |
| siTIMP2_p49 | AUCCCUUCCUGGAAACAGA | 4796 | UCUGUUUCCAGGAAGGGAU | 4844 | 18 + 1 | [1036-1053] 3′UTR |
| siTIMP2_p50 | AGUGCCUCUGGAUGGACUA | 4797 | UAGUCCAUCCAGAGGCACU | 4845 | 18 + 1 | [813-830] ORF |
| siTIMP2_p52 | UGGAGGAAAGAAGGAAUAA | 4798 | UUAUUCCUUCUUUCCUCCA | 4846 | 18 + 1 | [613-630] ORF |
| siTIMP2_p53 | GGGCACCAGGCCAAGUUCA | 4799 | UGAACUUGGCCUGGUGCCC | 4847 | 18 + 1 | [854-871] ORF |
| siTIMP2_p54 | GUGCAUUUUGCAGAAACUA | 4800 | UAGUUUCUGCAAAAUGCAC | 4848 | 18 + 1 | [1332-1349] 3′UTR |
| siTIMP2_p56 | CCAGAUGGGCUGCGAGUGA | 4801 | UCACUCGCAGCCCAUCUGG | 4849 | 18 + 1 | [745-762] ORF |
| siTIMP2_p57 | CGAGUGCCUCUGGAUGGAA | 4802 | UUCCAUCCAGAGGCACUCG | 4850 | 18 + 1 | [811-828] ORF |
| siTIMP2_p58 | CUGCGAGUGCAAGAUCACA | 4803 | UGUGAUCUUGCACUCGCAG | 4851 | 18 + 1 | [754-771] ORF |
| siTIMP2_p59 | UCUGGAUGGACUGGGUCAA | 4804 | UUGACCCAGUCCAUCCAGA | 4852 | 18 + 1 | [819-836] ORF |
| siTIMP2_p60 | GUACCAGAUGGGCUGCGAA | 4805 | UUCGCAGCCCAUCUGGUAC | 4853 | 18 + 1 | [742-759] ORF |
| siTIMP2_p63 | GUAGUGAUCAGGGCCAAAA | 4806 | UUUUGGCCCUGAUCACUAC | 4854 | 18 + 1 | [428-445] ORF |
| siTIMP2_p66 | GGCGCUCGGCCUCCUGCUA | 4807 | UAGCAGGAGGCCGAGCGCC | 4855 | 18 + 1 | [331-348] ORF |
| siTIMP2_p70 | UGGAUGGACUGGGUCACAA | 4808 | UUGUGACCCAGUCCAUCCA | 4856 | 18 + 1 | [821-838] ORF |
| siTIMP2_p72 | CCCUUCCUGGAAACAGCAA | 4809 | UUGCUGUUUCCAGGAAGGG | 4857 | 18 + 1 | [1038-1055] 3′UTR |
| siTIMP2_p73 | ACCAGAUGGGCUGCGAGUA | 4810 | UACUCGCAGCCCAUCUGGU | 4858 | 18 + 1 | [744-761] ORF |
| siTIMP2_p74 | ACAGGUACCAGAUGGGCUA | 4811 | UAGCCCAUCUGGUACCUGU | 4859 | 18 + 1 | [738-755] ORF |
| siTIMP2_p77 | CGGGCACCAGGCCAAGUUA | 4812 | UAACUUGGCCUGGUGCCCG | 4860 | 18 + 1 | [853-870] ORF |
| siTIMP2_p80 | CAUCCCUUCCUGGAAACAA | 4813 | UUGUUUCCAGGAAGGGAUG | 4861 | 18 + 1 | [1035-1052] 3′UTR |
| siTIMP2_p81 | CACCCGCAACAGGCGUUUA | 4814 | UAAACGCCUGUUGCGGGUG | 4862 | 18 + 1 | [398-415] ORF |

TABLE B8
18 + 1 -mer siTIMP2 with lowest predicted OT effect

| | | | SEQ | | SEQ | |
|---|---|---|---|---|---|---|
| Cross | | | ID | | ID | No. in |
| species: | Ranking | Sense (5′ > 3′) | NO: | Antisense (5′ > 3′) | NO: | Table B7 |
| H/Rt | 3 | CUGCAUCAAGAGAAGUGAA | 4771 | UUCACUUCUCUUGAUGCAG | 4819 | siTIMP2_p6 |
| H/Rt | 3 | GGCGUUUUGCAAUGCAGAA | 4774 | UUCUGCAUUGCAAAACGCC | 4822 | siTIMP2_p9 |
| H/Rt | 4 | GGGCUGCGAGUGCAAGAUA | 4780 | UUCUGCAUUGCAAAACGCC | 4828 | siTIMP2_p15 |
| H/Rt | 4 | GACAUCCCUUCCUGGAAAA | 4781 | UUCUGCAUUGCAAAACGCC | 4829 | siTIMP2_p19 |
| H/Rt | 4 | GAUGGACUGGGUCACAGAA | 4782 | UUCUGCAUUGCAAAACGCC | 4830 | siTIMP2_p21 |
| H/Rt | 4 | GCCUCUGGAUGGACUGGGA | 4783 | UUCUGCAUUGCAAAACGCC | 4831 | siTIMP2_p22 |
| H/Rt | 4 | GAGUGCCUCUGGAUGGACA | 4784 | UUCUGCAUUGCAAAACGCC | 4832 | siTIMP2_p23 |
| H/Rt | 2 | GCAACAGGCGUUUUGCAAA | 4786 | UUUGCAAAACGCCUGUUGC | 4834 | siTIMP2_p28 |
| H/Rt | 3 | GACGUUGGAGGAAAGAAGA | 4787 | UCUUCUUUCCUCCAACGUC | 4835 | siTIMP2_p31 |
| H/Rt | 4 | UGUGCAUUUUGCAGAAACA | 4790 | UGUUUCUGCAAAAUGCACA | 4838 | siTIMP2_p36 |
| H/Rt | 4 | GAACCACAGGUACCAGAUA | 4791 | UAUCUGGUACCUGUGGUUC | 4839 | siTIMP2_p42 |
| H/Rt | 4 | UGGGCUGCGAGUGCAAGAA | 4794 | UUCUUGCACUCGCAGCCCA | 4842 | siTIMP2_p47 |
| H/Rt | 4 | AGUGCCUCUGGAUGGACUA | 4797 | UAGUCCAUCCAGAGGCACU | 4845 | siTIMP2_p50 |
| H/Rt | 4 | CCAGAUGGGCUGCGAGUGA | 4801 | UCACUCGCAGCCCAUCUGG | 4849 | siTIMP2_p56 |
| H/Rt | 4 | CGAGUGCCUCUGGAUGGAA | 4802 | UUCCAUCCAGAGGCACUCG | 4850 | siTIMP2_p57 |
| H/Rt | 2 | CUGCGAGUGCAAGAUCACA | 4803 | UGUGAUCUUGCACUCGCAG | 4851 | siTIMP2_p58 |
| H/Rt | 3 | GUACCAGAUGGGCUGCGAA | 4805 | UUCGCAGCCCAUCUGGUAC | 4853 | siTIMP2_p60 |
| H/Rt | 2 | GUAGUGAUCAGGGCCAAAA | 4806 | UUUUGGCCCUGAUCACUAC | 4854 | siTIMP2_p63 |
| H/Rt | 3 | UGGAUGGACUGGGUCACAA | 4808 | UUGUGACCCAGUCCAUCCA | 4856 | siTIMP2_p70 |
| H/Rt | 4 | ACCAGAUGGGCUGCGAGUA | 4810 | UACUCGCAGCCCAUCUGGU | 4858 | siTIMP2_p73 |
| H/Rt | 4 | ACAGGUACCAGAUGGGCUA | 4811 | UAGCCCAUCUGGUACCUGU | 4859 | siTIMP2_p74 |
| H/Rt | 2 | CACCCGCAACAGGCGUUUA | 4814 | UAAACGCCUGUUGCGGGUG | 4862 | siTIMP2_p81 |

### Example 6

### In Vitro Testing of the siRNA Compounds for the Target Genes

Low-Throughput-Screen (LTS) for siRNA oligos directed to human and rat TIMP1 and TIMP2 gene.

About 2×10⁵ human cell lines (HeLa, LX2, hHSC or PC3) endogenously expressing TIMP1 or TIMP2 gene, are inoculated in 1.5 mL growth medium in order to reach 30-50% confluence after 24 hours. Cells are transfected with Lipofectamine-2000® reagent to a final concentration of 0.01-5 nM per transfected cells. Cells aere incubated at 37±1° C., 5% CO₂ for 48 hours. siRNA transfected cells are harvested and RNA is isolated using EZ-RNA® kit [Biological Industries (#20-410-100)].

Reverse transcription is performed as follows: Synthesis of cDNA is performed and human TIMP1 and TIMP2 mRNA levels are determined by Real Time qPCR and normalized to those of the Cyclophilin A (CYNA, PPIA) mRNA for each sample. siRNA activity is determined based on the ratio of the TIMP1 or TIMP2 mRNA quantity in siRNA-treated samples versus non-transfected control samples.

The most active sequences are selected from additional, assays.

IC50 Values for the LTS Selected TIMP1 or TIMP2 siRNA Oligos

Cells are grown as described above. The IC50 value of the tested RNAi activity is determined by constructing a dose-response curve using the activity results obtained with the various final siRNA concentrations. The dose response curve is constructed by plotting the relative amount of residual TIMP1 or TIMP2 mRNA versus the logarithm of transfected siRNA concentration. The curve is calculated by fitting the best sigmoid curve to the measured data. The method for the sigmoid fit is also known as a 3-point curve fit.

$$Y + Bot + \frac{100 - Bot}{1 + {10}^{( LogIC 50 - X ) \times HillSlope}}$$

where Y is the residual TIMP1 or TIMP2 mRNA response, X is the logarithm of transfected siRNA concentration, Bot is the Y value at the bottom plateau, LogIC50 is the X value when Y is halfway between bottom and top plateaus and HillSlope is the steepness of the curve.

The percent of inhibition of gene expression using specific siRNAs was determined using qPCR analysis of target gene in cells expressing the endogenous gene. Other siRNA compounds according to Tables A1, A2, A3, A4, A5, A6, A7, A8, B1, B2, B3, B4, B5, B6, B7, B8 (Tables A1-B8) are tested in vitro where it is shown that these siRNA compounds inhibit gene expression. Activity is shown as percent residual mRNA; accordingly, a lower value reflects better activity.

In order to test the stability of the siRNA compounds in serum, specific siRNA molecules are incubated in four different batches of human serum (100% concentration) at 37° C. for up to 24 hours. Samples are collected at 0.5, 1, 3, 6, 8, 10, 16 and 24 hours. The migration patterns as an indication of are determined at each collection time by polyacrylamide gel electrophoresis (PAGE).

### Example 7

### Validation of siTIMP1 and siTIMP2 Knock Down Effect at the Protein Level

The inhibitory effect of different siTIMP1 and siTIMP2 siNA molecules on TIMP1 and TIMP2 mRNA expression are validated at the protein level by measuring TIMP1 and TIMP2 in hTERT cells transfected with different siTIMP1 and siTIMP2. Transfection of hTERT cells with different siTIMP1 and siTIMP2 are performed as described above. Transfected hTERT cells are lysed and the cell lysate are clarified by centrifugation. Proteins in the clarified cell lysate are resolved by SDS polyacrylamide gel electrophoresis. The level of TIMP1 and TIMP2 protein in the cell lysate are determined using anti-TIMP or anti-TIMP2 antibodies as the primary antibody HRP conjugated antibodies (Millipore) as the secondary antibody, and subsequently detection by Supersignal West Pico Chemiluminescence kit (Pierce). Anti-actin antibody (Abcam) is used as a protein loading control.

### Example 8

### Downregulation of Collagen I Expression by siTIMP1 and siTIMP2 siRNA Duplexes

To determine the effect of siTIMP1 and siTIMP2, alone or in combination on collagen I expression level, collagen I mRNA level in hTERT cells treated with different siTIMP1 and or siTIMP2. Briefly, hTERT cells are transfected with different siTIMP1, and or siTIMP2 as described in Example 2. The cells are lysed after 72 hours and mRNA were isolated using RNeasy mini kit according to the manual (Qiagen). The level of collagen 1 mRNA is determined by reverse transcription coupled with quantitative PCR using TaqMan® probes. Briefly, cDNA synthesis is carried out using High-Capacity cDNA Reverse Transcription Kit (ABI) according to the manual, and subjected to TaqMan Gene Expression Assay (ABI, COL1A1 assay ID Hs01076780_g1). The level of collagen I mRNA is normalized to the level of GAPDH mRNA according to the manufacturer's instruction (ABI). The signals are normalized to the signal obtained from cells transfected with scrambled siNA.

### Example 9

### Immunofluorescence Staining of siTIMP1 and or siTIMP2 treated hTERT Cells

To visualize the expression of two fibrosis markers, collagen I and alpha-smooth muscle actin (SMA), in hTERT cells transfected, the cells are stained with rabbit anti-collagen I antibody (Abcam) and mouse anti-alpha-SMA antibody (Sigma). Alexa Fluor 594 goat anti-mouse IgG and Alexa Fluor 488 goat anti-rabbit IgG (Invitrogen (Molecular Probes)) are used as secondary antibodies to visualize collagen I (green) and alpha-SMA (red). Hoescht is used to visualize nuleus (blue).

### Example 10

### Animal Models: Model Systems of Fibrotic Conditions

siRNAs provided herein may be tested in predictive animal models. Rat diabetic and aging models of kidney fibrosis include Zucker diabetic fatty (ZDF) rats, aged fa/fa (obese Zucker) rats, aged Sprague-Dawley (SD) rats, and Goto Kakizaki (GK) rats; GK rats are an inbred strain derived from Wistar rats, selected for spontaneous development of NIDDM (diabetes type II). Induced models of kidney fibrosis include the permanent unilateral ureteral obstruction (UUO) model which is a model of acute interstitial fibrosis occurring in healthy non-diabetic animals; renal fibrosis develops within days following the obstruction. Another induced model of kidney fibrosis is 5/6 nephrectomy model.

Two models of liver fibrosis in rats are the Bile Duct Ligation (BDL) with sham operation as controls, and CCl4 poisoning, with olive oil fed animals as controls, as described in the following references: Lotersztajn S, et al Hepatic Fibrosis: Molecular Mechanisms and Drug Targets. Annu Rev Pharmacol Toxicol. 2004 Oct. 7; Uchio K, et al., Down-regulation of connective tissue growth factor and type I collagen mRNA expression by connective tissue growth factor antisense oligonucleotide during experimental liver fibrosis. Wound Repair Regen. 2004 January-February; 12(1):60-6; Xu X Q, et al., Molecular classification of liver cirrhosis in a rat model by proteomics and bioinformatics Proteomics. 2004 October; 4(10):3235-45.

Models for ocular scarring are well known in the art e.g. Sherwood M B et al., J Glaucoma. 2004 October; 13(5):407-12. A new model of glaucoma filtering surgery in the rat; Miller M H et al., Ophthalmic Surg. 1989 May; 20(5):350-7. Wound healing in an animal model of glaucoma fistulizing surgery in the Rb; vanBockxmeer F M et al., Retina. 1985 Fall-Winter; 5(4): 239-52. Models for assessing scar tissue inhibitors; Wiedemann P et al., J Pharmacol Methods. 1984 August; 12(1): 69-78. Proliferative vitreoretinopathy: the Rb cell injection model for screening of antiproliferative drugs.

Models of cataract are described in the following publications: The role of Src family kinases in cortical cataract formation. Zhou J, Menko A S. Invest Ophthalmol V is Sci. 2002 July; 43(7):2293-300; Bioavailability and anticataract effects of a topical ocular drug delivery system containing disulfuram and hydroxypropyl-beta-cyclodextrin on selenite-treated rats. Wang S, et al. Curr Eye Res. 2004 July; 29(1):51-8; and Long-term organ culture system to study the effects of UV-A irradiation on lens transglutaminase. Weinreb O, Dovrat A.; Curr Eye Res. 2004 July; 29(1):51-8.

The compounds disclosed herein are tested in these models of fibrotic conditions, in which it is found that they are effective in treating liver fibrosis and other fibrotic conditions. The compounds as described herein are tested in this animal model and the results show that these siRNA compounds are useful in treating and/or preventing ischemia reperfusion injury following lung transplantation.

The contents of the articles, patents, and patent applications, and all other documents and electronically available information mentioned or cited herein, are hereby incorporated by reference in their entirety to the same extent as if each individual publication was specifically and individually indicated to be incorporated by reference.

Applicants reserve the right to physically incorporate into this application any and all materials and information from any such articles, patents, patent applications, or other physical and electronic documents.

It will be readily apparent to one skilled in the art that varying substitutions and modifications can be made to the invention disclosed herein without departing from the scope and spirit of the invention. Thus, such additional embodiments are within the scope of the present invention and the following claims. The present disclosure teaches one skilled in the art to test various combinations and/or substitutions of chemical modifications described herein toward generating nucleic acid constructs with improved activity for mediating RNAi activity. Such improved activity can include improved stability, improved bioavailability, and/or improved activation of cellular responses mediating RNAi. Therefore, the specific embodiments described herein are not limiting and one skilled in the art can readily appreciate that specific combinations of the modifications described herein can be tested without undue experimentation toward identifying nucleic acid molecules with improved RNAi activity.

The inventions illustratively described herein may suitably be practiced in the absence of any element or elements, limitation or limitations, not specifically disclosed herein. Thus, for example, the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having,” “including,” containing”, etc. shall be read expansively and without limitation (e.g., meaning “including, but not limited to,”). Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

1. A nucleic acid molecule, wherein:
(a) the nucleic acid molecule comprises a sense strand and an antisense strand;
(b) each strand of the nucleic acid molecule is independently 15 to 49 nucleotides in length;
(c) a 15 to 49 nucleotide sequence of the antisense strand is complementary to a sequence of an mRNA encoding TIMP1 (SEQ ID NO:1) or TIMP2 (SEQ ID NO:2); and
(d) a 15 to 49 nucleotide sequence of the sense strand is complementary to the antisense strand thereby generating a duplex region and includes a 15 to 49 nucleotide sequence of an mRNA encoding TIMP1 (SEQ ID NO:1) or TIMP2 (SEQ ID NO:2).

2. The nucleic acid molecule of claim 1 wherein the sequence of the antisense strand is complementary to a sequence of an mRNA encoding human TIMP1 (SEQ ID NO:1), and wherein the antisense strand and the sense strand comprise a sequence pair selected from the group consisting of siTIMP1_p2 (SEQ ID NOS:267 and 299); siTIMP1_p6 (SEQ ID NOS:268 and 300); siTIMP1_p14 (SEQ ID NOS:269 and 301); siTIMP1_p16 (SEQ ID NOS:270 and 302); siTIMP1_p17 (SEQ ID NOS:271 and 303); siTIMP1_p19 (SEQ ID NOS:272 and 304); siTIMP1_p20 (SEQ ID NOS:273 and 305); siTIMP1_p21 (SEQ ID NOS:274 and 306); siTIMP1_p23 (SEQ ID NOS:275 and 307; siTIMP1_p29 (278 and 310); siTIMP1_p33 (280 and 312); siTIMP1_p38 (SEQ ID NOS:281 and 313); siTIMP1_p42 (282 and 314); siTIMP1_p43 (SEQ ID NOS:283 and 315); siTIMP1_p45 (284 and 316); siTIMP1_p60 (SEQ ID NOS:286 and 318); siTIMP1_p71 (SEQ ID NOS:287 and 319); siTIMP1_p73 (SEQ ID NOS:288 and 320); siTIMP1_p78 (290 and 322); siTIMP1_p79 (SEQ ID NOS:291 and 323); siTIMP1_p85 (SEQ ID NOS:292 and 324); siTIMP1_p89 (SEQ ID NOS:293 and 325); siTIMP1_p91 (SEQ ID NOS:294 and 326); siTIMP1_p96 (SEQ ID NOS:295 and 327); siTIMP1_p98 (SEQ ID NOS:296 and 328); siTIMP1_p99 (SEQ ID NOS:297 and 329) and siTIMP1_p108 (SEQ ID NOS:298 and 330).

3. The nucleic acid molecule of claim 1, wherein the sequence of the antisense strand is complementary to a sequence of an mRNA encoding human TIMP1, and wherein the sense strand and the antisense strand are selected from the sequence pairs shown in Table C set forth as TIMP1-A (SEQ ID NOS:5 and 6); TIMP1-B (SEQ ID NOS:7 and 8) and TIMP1-C (SEQ ID NO:9 and 10).

4. The nucleic acid molecule of claim 1, wherein the sequence of the antisense strand that is complementary to a sequence of an mRNA encoding human TIMP1 comprises a sequence complimentary to a sequence between nucleotides 300-400 of SEQ ID NO: 1, 355-373 of SEQ ID NO: 1, 600-750 of SEQ ID NO: 1, 620-638 of SEQ ID NO: 1 or 640-658 of SEQ ID NO: 1.

5. (canceled)

6. (canceled)

7. (canceled)

8. (canceled)

9. The nucleic acid molecule of claim 1, wherein the sequence of the antisense strand is complementary to a sequence of an mRNA encoding human TIMP2, and wherein the sense strand and the antisense strand are selected from the sequence pairs shown in Table D.

10. The nucleic acid molecule of claim 1, wherein the sequence of the antisense strand is complementary to a sequence of an mRNA encoding human TIMP2 and comprises a sequence complimentary to a sequence between nucleotides 400-500 of SEQ ID NO: 2, 500-600 of SEQ ID NO: 2, 600-700 of SEQ ID NO: 2, and 698-716 of SEQ ID NO: 2.

11. (canceled)

12. (canceled)

13. (canceled)

14. (canceled)

15. (canceled)

16. (canceled)

17. (canceled)

18. The nucleic acid molecule of claim 1, wherein the antisense strand and the sense strand are independently 17 to 49 nucleotides in length.

19. (canceled)

20. (canceled)

21. (canceled)

22. (canceled)

23. (canceled)

24. The nucleic acid molecule of claim 1, wherein the antisense strand and the sense strand are each 19 nucleotides in length.

25. (canceled)

26. (canceled)

27. (canceled)

28. (canceled)

29. (canceled)

30. (canceled)

31. (canceled)

32. The nucleic acid molecule of claim 1, wherein the duplex region is 19 nucleotides in length.

33. The nucleic acid molecule of claim 1, wherein the antisense strand and the sense strand are separate polynucleotide strands.

34. (canceled)

35. (canceled)

36. The nucleic acid molecule of claim 1, wherein the sense strand and the antisense strands are part of a single polynucleotide strand having both a sense region and an antisense region.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The nucleic acid molecule of claim 1, wherein the nucleic acid molecule comprises one or more modifications or modified nucleotides.

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. A method for treating an individual suffering from a disease associated with TIMP1 or TIMP2, wherein
(a) when the disease is associated with TIMP1, the method comprises administering to said individual a nucleic acid molecule of claim 1 comprising a sequence of the antisense strand complementary to a sequence of a mRNA encoding human TIMP1, in an amount sufficient to reduce expression of TIMP1; and
(b) when the disease is associated with TIMP2, the method comprises administering to said individual a nucleic acid molecule of claim 1—comprising a sequence of the antisense strand complementary to a sequence of a mRNA encoding human TIMP2, in an amount sufficient to reduce expression of TIMP2.

87. (canceled)

88. The method of claim 86, wherein said disease associated with TIMP1 or TIMP2 is fibrosis.

89. A composition comprising a nucleic acid molecule of any of claim 1-4, 9, 10, 24, 32, 33, 36, 57, 98, 99, 105, 125-128 or 133 and a pharmaceutically acceptable carrier.

90. (canceled)

91. (canceled)

92. (canceled)

93. (canceled)

94. (canceled)

95. (canceled)

96. (canceled)

97. (canceled)

98. A double stranded nucleic acid molecule having the structure (A 1):

| | | | |
|---|---|---|---|
| (A1) | 5′ | (N)x-Z 3′ | (antisense strand) |
| | 3′ | Z′-(N′)y -z″ 5′ | (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein each of Z and Z′ is independently present or absent, but if present is independently 1-5 consecutive nucleotides or unconventional moieties or a combination thereof covalently attached at the 3′ terminus of the strand in which it is present.
wherein z″ may be present or absent, but if present is a capping moiety covalently attached at the 5′ terminus of (N′)y;
wherein each of x and y is independently an integer from 18 to 40;
wherein the sequence of (N′)y has complementarity to the sequence of (N)x; and wherein (N)x comprises an antisense sequence to an mRNA set forth in SEQ ID NO:1 or SEQ ID NO:2.

99. The nucleic acid molecule of claim 98 having the structure (A1), wherein (N)x comprises an antisense sequence to an mRNA set forth in SEQ ID NO:1 and wherein (N)x comprises an antisense oligonucleotide present in any one of Tables A 1, A2, A3 or A4; or wherein (N)x comprises an antisense sequence to an mRNA set forth in SEQ ID NO:2 and wherein (N)x comprises an antisense oligonucleotide present in any one of Tables B1, B2, B3 or B4.

100. (canceled)

101. (canceled)

102. (canceled)

103. (canceled)

104. (canceled)

105. The nucleic acid molecule of claim 98, wherein x=y=19.

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. A double stranded nucleic acid molecule having a structure (A2) set forth below:

| | | | |
|---|---|---|---|
| (A2) | 5′ | N1-(N)x - Z 3′ | (antisense strand) |
| | 3′ | Z′-N2-(N′)y -z″ 5′ | (sense strand) |

wherein each of N2, N and N′ is independently an unmodified or modified ribonucleotide, or an unconventional moiety;
wherein each of (N)x and (N′)y is an oligonucleotide in which each consecutive N or N′ is joined to the adjacent N or N′ by a covalent bond;
wherein each of x and y is independently an integer from 17 to 39;
wherein the sequence of (N′)y has complementarity to the sequence of (N)x and (N)x has complementarity to a consecutive sequence in the mRNA set forth in SEQ ID NO:1 or SEQ ID NO:2;
wherein N1 is covalently bound to (N)x and is mismatched to the mRNA set forth in SEQ ID NO:1 or SEQ ID NO:2;
wherein N1 is a moiety selected from the group consisting of ribouridine, modified ribouridine deoxyribouridine, modified deoxyribouridine, riboadenine, modified riboadenine deoxyriboadenine, and modified deoxyriboadenine;
wherein z″ may be present or absent, but if present is a capping moiety covalently attached at the 5′ terminus of (N′)y; and
wherein each of Z and Z′ is independently present or absent, but if present is independently 1-5 consecutive nucleotides or unconventional moieties or a combination thereof covalently attached at the 3′ terminus of the strand in which it is present.

126. The nucleic acid molecule of claim 125 wherein x=y=18.

127. The nucleic acid molecule of claim 125, wherein (N)x comprises an antisense sequence to an mRNA set forth in SEQ ID NO:1 or SEQ ID NO:2.

128. The nucleic acid molecule of claim 127, having the structure (A2),
wherein (N)x comprises an antisense sequence to an mRNA set forth in SEQ ID NO:1, comprising an antisense oligonucleotide present in any one of Tables A5, A6, A7, A8; or
wherein (N)x comprises an antisense sequence to an mRNA set forth in SEQ ID NO:2, comprising an antisense oligonucleotide present in any one of Tables B5, B6, B7, or B8.

129. (canceled)

130. (canceled)

131. (canceled)

132. (canceled)

133. A nucleic acid molecule consisting of four ribonucleotide strands forming three siRNA duplexes having the general structure: 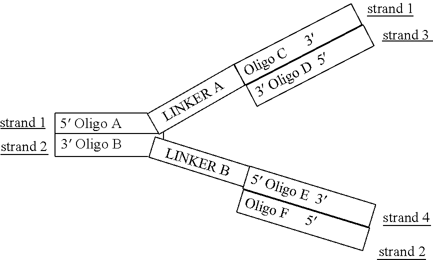
wherein each of oligo A, oligo B, oligo C, oligo D, oligo E and oligo F represents at least 19 consecutive ribonucleotides, wherein from 19 to 40 of such consecutive ribonucleotides, in each of oligo A, B, C, D, E and F comprise a strand of a siRNA duplex, wherein each ribonucleotide may be modified or unmodified;
wherein strand 1 comprises oligo A which is either a sense portion or an antisense portion of a first siRNA duplex of the nucleic acid molecule, strand 2 comprises oligo B which is complementary to at least 19 nucleotides in oligo A, and oligo A and oligo B together form a first siRNA duplex that targets a first target mRNA;
wherein strand 1 further comprises oligo C which is either a sense portion or an antisense strand portion of a second siRNA duplex of the nucleic acid molecule, strand 3 comprises oligo D which is complementary to at least 19 nucleotides in oligo C and oligo C and oligo D together form a second siRNA duplex that targets a second target mRNA;
wherein strand 4 comprises oligo E which is either a sense portion or an antisense strand portion of a third siRNA duplex of the nucleic acid molecule, strand 2 further comprises oligo F which is complementary to at least 19 nucleotides in oligo E and oligo E and oligo F together form a third siRNA duplex that targets a third target mRNA;
wherein linker A is a moiety that covalently links oligo A and oligo C; linker B is a moiety that covalently links oligo B and oligo F, and linker A and linker B can be the same or different; and
wherein the nucleic acid molecule includes at least one antisense strand and sense strand pair set forth in any one of Tables A1-A8 and Tables B1-B8.

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)
